(19) United States
(12) Patent Application Publication
(10) Pub. No.: US 2026/0191989 A1
(43) Pub. Date: Jul. 9, 2026

(54) GENE THERAPY COMPOSITIONS AND METHODS OF USE THEREOF

(71) Applicants: LogicBio Therapeutics, Inc., Lexington, MA (US); AstraZeneca AB, Södertälje (SE)

(72) Inventors: Chih-Chieh WANG, Natick, MA (US); Shaobin WANG, Lexington, MA (US); Donald James FOSTER, Lexington, MA (US); Annette BAK, Södertälje (SE); Liping ZHOU, Södertälje (SE); Bei CHENG, Södertälje (SE)

(21) Appl. No.: 18/868,278

(22) PCT Filed: May 23, 2023
(86) PCT No.: PCT/US2023/023285

§ 371 Date: Nov. 22, 2024

Related U.S. Application Data
(60) Provisional application No. 63/345,003, filed on May 23, 2022.

(51) Int. Cl. A61K 48/00; A61K 9/1271; A61K 9/51; A61K 38/38; C12N 9/22; C12N 15/11; C12N 15/86; C12N 15/88; C12N 15/90

(52) U.S. Cl. CPC A61K 48/005; A61K 9/1271; A61K 9/5123; A61K 38/385; C12N 9/226; C12N 15/111; C12N 15/86; C12N 15/88; C12N 15/907; C12N 2310/20; C12N 2750/14143; C12N 2840/203

(57) ABSTRACT

Presented herein are compositions and methods for gene therapy.

### BACKGROUND

There is a subset of human diseases that can be traced to changes in the DNA that are either inherited or acquired early in embryonic development. Of particular interest for developers of genetic therapies are diseases caused by a mutation in a single gene, known as monogenic diseases. There are believed to be over 6,000 monogenic diseases. Typically, any particular genetic disease caused by inherited mutations is relatively rare, but taken together, the toll of genetic-related disease is high. Well-known genetic diseases include cystic fibrosis, Duchenne muscular dystrophy, Huntington's disease, and sickle cell disease. Other classes of genetic diseases include metabolic disorders, such as organic acidemias, and lysosomal storage diseases where dysfunctional genes result in defects in metabolic processes and the accumulation of toxic byproducts that can lead to serious morbidity and mortality both in the short-term and long-term.

### SUMMARY

Genetic diseases caused by dysfunctional genes account for a large fraction of diseases worldwide. Gene therapy is emerging as a promising form of treatment aiming to mitigate the effects of genetic diseases.

Prior to the present disclosure, certain AAV gene therapies that made use of homologous recombination employed viral vector compositions in the absence of a nuclease to introduce a transgene of interest at a particular site. Among other things, the present disclosure recognizes that combination of such a vector with one or more nucleases (e.g., provided in a single composition or as separate compositions) may improve transgene integration rates and/or efficiency in humans.

Alternatively, or additionally, the present disclosure further encompasses the recognition that a combination of a vector as described herein with one or more nucleases, may provide surprising and unexpected improvements in transgene integration rates and/or efficiency in humans, for example, wherein a target cut site is distal from a site of transgene integration. In part, the present disclosure encompasses the recognition and observation that optimized vector designs and preferred cut sites may be different between species. As demonstrated herein, optimal designs in a human or humanized system may differ significantly from those in another species or model system (e.g., wild-type mouse). In some embodiments, a human or humanized system may allow increased flexibility in selection of a cut site for enhancing homologous recombination and thus the effectiveness of certain gene therapies.

In some embodiments, the present disclosure provides compositions including (i) a nuclease or polynucleotide sequence encoding a nuclease, and (ii) a polynucleotide cassette comprising an expression cassette comprising a first nucleic acid sequence and a second nucleic acid sequence, wherein the first nucleic acid sequence encodes a transgene, and the second nucleic acid sequence is positioned 5′ or 3′ to the first nucleic acid sequence and promotes the production of two independent gene products upon integration into a target integration site in the genome of a cell, a third nucleic acid sequence positioned 5′ to the expression cassette and comprising a sequence that is substantially homologous to a human genomic sequence 5′ of a target integration site in a genome of a cell, and a fourth nucleic acid sequence positioned 3′ to expression cassette and comprising a sequence that is substantially homologous to a human genomic sequence 3′ of a target integration site in the genome of the cell, wherein: the polynucleotide cassette does not include a promoter sequence, the nuclease is capable of inducing a double-stranded break and/or a single-stranded break at a cut site in the genome of the cell, and the cut site is distal from the target integration site.

In some embodiments, the present disclosure provides methods of integrating a transgene into the genome of a cell, the methods including administering to a subject a composition comprising: (i) a nuclease or a polynucleotide sequence encoding a nuclease, and (ii) a polynucleotide cassette comprising: an expression cassette comprising a first nucleic acid sequence and a second nucleic acid sequence, wherein the first nucleic acid sequence encodes a transgene; and the second nucleic acid sequence is positioned 5′ or 3′ to the first nucleic acid sequence and promotes the production of two independent gene products upon integration into a target integration site in the genome of a cell, a third nucleic acid sequence positioned 5′ to the expression cassette and comprising a sequence that is substantially homologous to a human genomic sequence 5′ of a target integration site in a genome of a cell, and a fourth nucleic acid sequence positioned 3′ to expression cassette and comprising a sequence that is substantially homologous to a human genomic sequence 3′ of a target integration site in the genome of the cell, wherein: the polynucleotide cassette does not include a promoter sequence, the nuclease is capable of inducing a double-stranded break and/or a single-stranded break at a cut site in the genome of the cell, and the cut site is distal from the target integration site, wherein, after administering the composition, the transgene is integrated into the genome of the population of cells. In some embodiments, a cell is edited in vivo. In some embodiments, integrating a transgene is conducted ex vivo.

While any of a variety of delivery systems are contemplated, in some embodiments, a composition further comprises a recombinant viral vector. While any of a variety of viral vectors are contemplated, in some embodiments, a recombinant viral vector is a recombinant AAV vector. In some embodiments, a recombinant viral vector is or comprises a capsid polypeptide comprising an amino acid sequence having at least 95% sequence identity with the amino acid sequence of sL65, LK03, AAV8, AAV-DJ; AAV-LK03; or AAVNP59. In some embodiments, a composition further comprises AAV2 ITR sequences.

In accordance with various embodiments, any of a variety of homology arm lengths and/or ratios are contemplated. In some embodiments, a third and fourth nucleic acid sequence are each between 50 nt and 1600 nt in length. In some embodiments, a third and fourth nucleic acid sequence are the same length. In some embodiments, a third and fourth nucleic acid sequence are different lengths.

In some embodiments, a polynucleotide cassette does not comprise a promoter sequence.

In accordance with various embodiments, provided compositions may include integration at a target integration site. In some embodiments, upon integration of a polynucleotide cassette into a target integration site in the genome of the cell, the transgene is expressed under control of an endogenous promoter at the target integration site. In some embodiments, a target integration site is an albumin locus comprising an endogenous albumin promoter and an endogenous albumin gene. In some embodiments, a target integration site is a collagen locus comprising an endogenous collagen promoter and an endogenous collagen gene. In some embodiments, a target integration site is an actin locus comprising an endogenous actin promoter and an endogenous actin gene. In some embodiments, a target integration site is within a coding sequence of the albumin locus and 5′-adjacent to a stop codon. In some embodiments, a target integration site is 5′-adjacent to a stop codon in exon 14 of the albumin locus.

In accordance with various embodiments, any application-appropriate cut site may be used. By way of non-limiting example, in some embodiments a cut site is within a non-coding sequence of the albumin locus. In some embodiments, a cut site is within an intron, untranslated region, enhancer, promoter, silencer, or insulator of the albumin locus. In some embodiments, a cut site is within intron 13 or 14 of the albumin locus. In some embodiments, a cut site is between 1 and 2000 bp from the target integration site. In some embodiments, a cut site is up to 100 bp from the target integration site.

As is described herein, the present disclosure encompasses the recognition that any of a variety of nucleases may be useful in provided methods and in combination with provided compositions. In some embodiments a nuclease is selected from a meganuclease, TALEN, TALE Nickaase, ZFN, ZF Nickase, Cas enzyme, or variant thereof.

In accordance with various embodiments, a variety of second nucleic acids may be used to promote the production of two independent gene products upon integration into a target integration site in the genome of a cell. For example, in some embodiments a second nucleic acid sequence may be or comprise a) a nucleic acid sequence encoding a 2A peptide, b) a nucleic acid sequence encoding an internal ribosome entry site (IRES), c) a nucleic acid sequence encoding an N-terminal intein splicing region and C-terminal intein splicing region, or d) a nucleic acid sequence encoding a splice donor and a splice acceptor. In some embodiments, a second nucleic acid is or comprises a nucleic acid sequence encoding a 2A peptide selected from the group consisting of P2A, T2A, E2A, and F2A.

The present disclosure encompasses the recognition that any of a variety of transgenes may be used with provided compositions and/or in provided methods. For example, in some embodiments, a transgene may be selected from CBS, UGT1A1, MUT, FAH, ATP7B, A1AT, ASL, LIPA, Factor IX, or a variant thereof.

Any of a variety of cell types may be modified through application of one or more provided compositions and/or methods. For example, in some embodiments, a cell is a blood, liver, muscle, or CNS cell.

In accordance with various embodiments, provided methods and compositions may be useful in the context of organ and/or tissue transplant. In some embodiments, a cell is administered in an autologous transplant after transgene integration. In some embodiments, a cell is administered in an allogeneic transplant after transgene integration.

In one aspect, the invention features a composition or set of compositions including: a nuclease or polynucleotide sequence encoding a nuclease, wherein the nuclease is selected from a clustered regularly interspaced short palindromic repeats (CRISPR)-associated (Cas) enzyme, a transcription activator-like effector (TALE) nuclease (TALEN), a TALE nickase, a zinc finger (ZF) nuclease (ZFN), a ZF nickase, or a meganuclease; and a polynucleotide cassette including: an expression cassette including a first nucleic acid sequence and a second nucleic acid sequence, wherein the first nucleic acid sequence encodes a transgene; and the second nucleic acid sequence is positioned 5′ or 3′ to the first nucleic acid sequence and promotes the production of two independent gene products upon integration into a target integration site in the genome of a human cell; a third nucleic acid sequence positioned 5′ to the expression cassette and including a sequence that is substantially homologous to a human genomic sequence 5′ of the target integration site in a genome of a human cell; and a fourth nucleic acid sequence positioned 3′ to expression cassette and including a sequence that is substantially homologous to a human genomic sequence 3′ of the target integration site in the genome of the human cell; wherein the polynucleotide cassette does not include a promoter sequence; and the nuclease is capable of inducing a double-stranded break and/or a single-stranded break at a cut site in the genome of the human cell.

In some embodiments of the foregoing aspect, the target integration site is an albumin locus including an endogenous albumin promoter and an endogenous albumin gene.

In another aspect, the invention features a composition or set of compositions including: a nuclease or polynucleotide sequence encoding a nuclease; and a polynucleotide cassette including: an expression cassette including a first nucleic acid sequence and a second nucleic acid sequence, wherein the first nucleic acid sequence encodes a transgene; and the second nucleic acid sequence is positioned 5′ or 3′ to the first nucleic acid sequence and promotes the production of two independent gene products upon integration into a target integration site in the genome of a human cell, wherein the target integration site is an albumin locus including an endogenous albumin promoter and an endogenous albumin gene; a third nucleic acid sequence positioned 5′ to the expression cassette and including a sequence that is substantially homologous to a human genomic sequence 5′ of the target integration site in a genome of a human cell; and a fourth nucleic acid sequence positioned 3′ to expression cassette and including a sequence that is substantially homologous to a human genomic sequence 3′ of the target integration site in the genome of the human cell; wherein the polynucleotide cassette does not include a promoter sequence; and the nuclease is capable of inducing a double-stranded break and/or a single-stranded break at a cut site in the genome of the human cell.

In some embodiments of any one of the foregoing aspects, the composition or the set of compositions further includes a recombinant viral vector. In some embodiments, the recombinant viral vector is a recombinant AAV vector. In some embodiments, the recombinant viral vector is or includes a capsid polypeptide including an amino acid sequence having at least 95% sequence identity with the amino acid sequence of sL65, LK03, AAV8, AAV-DJ; AAV-LK03; or AAVNP59. In some embodiments, the recombinant viral vector includes the capsid polypeptide and the polynucleotide sequence encoding a nuclease and/or the polynucleotide cassette is encapsidated in the recombinant viral vector. In some embodiments, the polynucleotide cassette is encapsidated in the recombinant viral vector.

In some embodiments of any one of the foregoing aspects, the composition or the set of compositions further includes AAV2 inverted terminal repeat (ITR) sequences. In some embodiments, the AAV2 ITR sequences flank the 5′ and 3′ ends of the polynucleotide sequence encoding the nuclease and/or the polynucleotide cassette.

In some embodiments of any one of the foregoing aspects, the third and fourth nucleic acid sequence are each between 50 nt and 1600 nt in length.

In some embodiments of any one of the foregoing aspects, the third and fourth nucleic acid sequence are the same length.

In some embodiments of any one of the foregoing aspects, the third and fourth nucleic acid sequence are different lengths.

In some embodiments of any one of the foregoing aspects, upon integration of the polynucleotide cassette into the target integration site in the genome of the cell, the transgene is expressed under control of an endogenous promoter at the target integration site.

In some embodiments of any one of the foregoing aspects, the target integration site is within a coding sequence of the albumin locus and 5′-adjacent to a stop codon. In some embodiments, the target integration site is 5′-adjacent to a stop codon in exon 14 of the albumin locus.

In some embodiments of any one of the foregoing aspects, the cut site is within a non-coding sequence of the albumin locus. In some embodiments, the cut site is within an intron, untranslated region, enhancer, promoter, silencer, or insulator of the albumin locus. In some embodiments, the cut site is within intron 12, 13, or 14 of the albumin locus.

In some embodiments of any one of the foregoing aspects, the nuclease is selected from a Cas enzyme, a TALEN, a TALE nickase, a ZFN, a ZF nickase, or a meganuclease.

In some embodiments of any one of the foregoing aspects, the nuclease is a Cas enzyme or a TALEN.

In some embodiments of any one of the foregoing aspects, the nuclease is a Cas enzyme.

In some embodiments of any one of the foregoing aspects, the Cas enzyme is selected from Staphylococcus aureus Cas9 (saCas9), Streptococcus pyogenes (spCas9), AZ Nuclease, HF1-Cas9, HF2-Cas9, or HiFi-Cas9. In some embodiments, the composition or the set of compositions further includes a guide RNA (gRNA). In some embodiments, the gRNA includes a nucleic acid sequence of any one of SEQ ID NOs: 27-45, 71-86, or 93-98, or a nucleic acid sequence having at least 80%, 85%, 90%, 95%, 96%, 97%, 98%, or 99% identity to any one of SEQ ID NOs: 27-45, 71-86, or 93-98.

In some embodiments of any one of the foregoing aspects, the nuclease or polynucleotide sequence encoding the nuclease and the gRNA are co-formulated.

In some embodiments of any one of the foregoing aspects, the nuclease or polynucleotide sequence encoding the nuclease and the gRNA are formulated separately.

In some embodiments of any one of the foregoing aspects, the second nucleic acid sequence is or includes: a nucleic acid sequence encoding a 2A peptide; a nucleic acid sequence encoding an internal ribosome entry site (IRES); a nucleic acid sequence encoding an N-terminal intein splicing region and C-terminal intein splicing region; or a nucleic acid sequence encoding a splice donor and a splice acceptor.

In some embodiments of any one of the foregoing aspects, the second nucleic acid sequence is or includes a nucleic acid sequence encoding a 2A peptide.

In some embodiments of any one of the foregoing aspects, the second nucleic acid is or includes a nucleic acid sequence encoding a 2A peptide selected from the group consisting of P2A, T2A, E2A, and F2A.

In some embodiments of any one of the foregoing aspects, the cut site is between 1 and 2000 bp from the target integration site. In some embodiments, the cut site is up to 100 bp from the target integration site.

In some embodiments of any one of the foregoing aspects, the transgene is selected from CBS, UGT1A1, MUT, FAH, ATP7B, A1AT, ASL, LIPA, PAH, G6PC, Factor IX, or a variant thereof.

In some embodiments of any one of the foregoing aspects, the composition or the set of compositions is a set of compositions, wherein the nuclease or polynucleotide sequence encoding the nuclease is formulated in a lipid nanoparticle (LNP) and the polynucleotide cassette is encapsidated in a recombinant AAV vector.

In some embodiments of any of the foregoing aspects, the cut site is distal from the target integration site.

In other embodiments of any of the foregoing aspects, the cut site is overlapping with the integration site.

In another aspect the invention features a method of integrating a transgene into the genome of a human cell, said method including administering to a subject a composition or set of compositions including: a nuclease or a polynucleotide sequence encoding a nuclease, wherein the nuclease is selected from a Cas enzyme, a TALEN, a TALE nickase, a ZFN, a ZF nickase, or a meganuclease; and a polynucleotide cassette including: an expression cassette including a first nucleic acid sequence and a second nucleic acid sequence, wherein the first nucleic acid sequence encodes a transgene; and the second nucleic acid sequence is positioned 5′ or 3′ to the first nucleic acid sequence and promotes the production of two independent gene products upon integration into a target integration site in the genome of a human cell; a third nucleic acid sequence positioned 5′ to the expression cassette and including a sequence that is substantially homologous to a human genomic sequence 5′ of the target integration site in a genome of a human cell; and a fourth nucleic acid sequence positioned 3′ to expression cassette and including a sequence that is substantially homologous to a human genomic sequence 3′ of the target integration site in the genome of the human cell; wherein: the polynucleotide cassette does not include a promoter sequence; and the nuclease is capable of inducing a double-stranded break and/or a single-stranded break at a cut site in the genome of the cell; wherein, after administering the composition or the set of compositions, the transgene is integrated into the genome of the human cell. In some embodiments, the target integration site is an albumin locus including an endogenous albumin promoter and an endogenous albumin gene.

In another aspect, the invention features a method of integrating a transgene into the genome of a human cell, said method including contacting the human cell with a composition or set of compositions including: a nuclease or a polynucleotide sequence encoding a nuclease, wherein the nuclease is selected from a Cas enzyme, a TALEN, a TALE nickase, a ZFN, a ZF nickase, or a meganuclease; and a polynucleotide cassette including: an expression cassette including a first nucleic acid sequence and a second nucleic acid sequence, wherein the first nucleic acid sequence encodes a transgene; and the second nucleic acid sequence is positioned 5′ or 3′ to the first nucleic acid sequence and promotes the production of two independent gene products upon integration into a target integration site in the genome of a human cell; a third nucleic acid sequence positioned 5′ to the expression cassette and including a sequence that is substantially homologous to a human genomic sequence 5′ of the target integration site in a genome of a human cell; and a fourth nucleic acid sequence positioned 3′ to expression cassette and including a sequence that is substantially homologous to a human genomic sequence 3′ of the target integration site in the genome of the human cell; wherein: the polynucleotide cassette does not include a promoter sequence; and the nuclease is capable of inducing a double-stranded break and/or a single-stranded break at a cut site in the genome of the cell; wherein, after contacting the human cell with the composition or the set of compositions, the transgene is integrated into the genome of the human cell. In some embodiments, the target integration site is an albumin locus including an endogenous albumin promoter and an endogenous albumin gene.

In another aspect, the invention features a method of integrating a transgene into the genome of a human cell, said method including administering to a subject a composition or set of compositions including: a nuclease or a polynucleotide sequence encoding a nuclease; and a polynucleotide cassette including: an expression cassette including a first nucleic acid sequence and a second nucleic acid sequence, wherein the first nucleic acid sequence encodes a transgene; and the second nucleic acid sequence is positioned 5′ or 3′ to the first nucleic acid sequence and promotes the production of two independent gene products upon integration into a target integration site in the genome of a human cell, wherein the target integration site is an albumin locus including an endogenous albumin promoter and an endogenous albumin gene; a third nucleic acid sequence positioned 5′ to the expression cassette and including a sequence that is substantially homologous to a human genomic sequence 5′ of a target integration site in a genome of a human cell; and a fourth nucleic acid sequence positioned 3′ to expression cassette and including a sequence that is substantially homologous to a human genomic sequence 3′ of a target integration site in the genome of the human cell; wherein: the polynucleotide cassette does not include a promoter sequence; the nuclease is capable of inducing a double-stranded break and/or a single-stranded break at a cut site in the genome of the cell; and the cut site is distal from the target integration site; wherein, after administering the composition or the set of compositions, the transgene is integrated into the genome of the human cell.

In another aspect, the invention features a method of integrating a transgene into the genome of a human cell, said method including contacting the human cell with a composition or set of compositions including: a nuclease or a polynucleotide sequence encoding a nuclease; and a polynucleotide cassette including: an expression cassette including a first nucleic acid sequence and a second nucleic acid sequence, wherein the first nucleic acid sequence encodes a transgene; and the second nucleic acid sequence is positioned 5′ or 3′ to the first nucleic acid sequence and promotes the production of two independent gene products upon integration into a target integration site in the genome of a human cell, wherein the target integration site is an albumin locus including an endogenous albumin promoter and an endogenous albumin gene; a third nucleic acid sequence positioned 5′ to the expression cassette and including a sequence that is substantially homologous to a human genomic sequence 5′ of a target integration site in a genome of a human cell; and a fourth nucleic acid sequence positioned 3′ to expression cassette and including a sequence that is substantially homologous to a human genomic sequence 3′ of a target integration site in the genome of the human cell; wherein: the polynucleotide cassette does not include a promoter sequence; the nuclease is capable of inducing a double-stranded break and/or a single-stranded break at a cut site in the genome of the cell; and the cut site is distal from the target integration site; wherein, after contacting the human cell with the composition or the set of compositions, the transgene is integrated into the genome of the human cell.

In some embodiments of any one of the foregoing aspects, the composition or the set of compositions further includes a recombinant viral vector. In some embodiments, the recombinant viral vector is a recombinant AAV vector. In some embodiments, the recombinant viral vector is or includes a capsid polypeptide including an amino acid sequence having at least 95% sequence identity with the amino acid sequence of LK03, AAV8, AAV-DJ; AAV-LK03; or AAVNP59. In some embodiments, the recombinant viral vector includes the capsid polypeptide and the polynucleotide sequence encoding a nuclease and/or the polynucleotide cassette is encapsidated in the recombinant viral vector. In some embodiments, the polynucleotide cassette is encapsidated in the recombinant viral vector.

In some embodiments of any one of the foregoing aspects, the composition or the set of compositions further includes AAV2 ITR sequences. In some embodiments, the AAV2 ITR sequences flank the 5′ and 3′ ends of the polynucleotide sequence encoding the nuclease and/or the polynucleotide cassette.

In some embodiments of any one of the foregoing aspects, the third and fourth nucleic acid sequence are between 50 nt and 1600 nt in length.

In some embodiments of any one of the foregoing aspects, the third and fourth nucleic acid sequence are the same length.

In some embodiments of any one of the foregoing aspects, the third and fourth nucleic acid sequence are different lengths.

In some embodiments of any one of the foregoing aspects, upon integration of the polynucleotide cassette into the target integration site in the genome of the cell, the transgene is expressed under control of an endogenous promoter at the target integration site.

In some embodiments of any one of the foregoing aspects, the target integration site is within a coding sequence of the albumin locus and 5′-adjacent to a stop codon. In some embodiments, the target integration site is 5′-adjacent to a stop codon in exon 14 of the albumin locus.

In some embodiments of any one of the foregoing aspects, the cut site is within a non-coding sequence of the albumin locus. In some embodiments, the cut site is within an intron, untranslated region, enhancer, promoter, silencer, or insulator of the albumin locus. In some embodiments, the cut site is within intron 12, 13, or 14 of the albumin locus.

In some embodiments of any one of the foregoing aspects, the nuclease is selected from a Cas enzyme, a TALEN, a TALE nickase, a ZFN, a ZF nickase, or a meganuclease.

In some embodiments of any one of the foregoing aspects, the nuclease is a Cas enzyme or a TALEN.

In some embodiments of any one of the foregoing aspects, the nuclease is a Cas enzyme.

In some embodiments of any one of the foregoing aspects, the Cas enzyme is selected from Staphylococcus aureus Cas9 (saCas9), Streptococcus pyogenes (spCas9), AZ Nuclease, HF1-Cas9, HF2-Cas9, or HiFi-Cas9.

In some embodiments of any one of the foregoing aspects, the method further includes a guide RNA (gRNA). In some embodiments, the gRNA includes a nucleic acid sequence of any one of SEQ ID NOs: 27-45, 71-86, or 93-98, or a nucleic acid sequence having at least 80%, 85%, 90%, 95%, 96%, 97%, 98%, or 99% identity to any one of SEQ ID NOs: 27-45, 71-86, or 93-98.

In some embodiments of any one of the foregoing aspects, the nuclease or polynucleotide sequence encoding the nuclease and the gRNA are co-formulated.

In some embodiments of any one of the foregoing aspects, the nuclease or polynucleotide sequence encoding the nuclease and the gRNA are formulated separately.

In some embodiments of any one of the foregoing aspects, the second nucleic acid sequence is or includes: a nucleic acid sequence encoding a 2A peptide; a nucleic acid sequence encoding an internal ribosome entry site (IRES); a nucleic acid sequence encoding an N-terminal intein splicing region and C-terminal intein splicing region; or a nucleic acid sequence encoding a splice donor and a splice acceptor.

In some embodiments of any one of the foregoing aspects, the second nucleic acid sequence is or includes a nucleic acid sequence encoding a 2A peptide. In some embodiments, the second nucleic acid sequence is or includes a nucleic acid sequence encoding a 2A peptide selected from the group consisting of P2A, T2A, E2A, and F2A.

In some embodiments of any one of the foregoing aspects, the cut site is between 1 and 1000 bp from the target integration site. In some embodiments, the cut site is up to 100 bp from the target integration site.

In some embodiments of any one of the foregoing aspects, the transgene is selected from CBS, UGT1A1, MUT, FAH, ATP7B, A1AT, ASL, LIPA, PAH, G6PC, Factor IX, or a variant thereof.

In some embodiments of any one of the foregoing aspects, the method, which is a set of compositions, wherein the nuclease or polynucleotide sequence encoding the nuclease is formulated in a lipid nanoparticle and the polynucleotide cassette is encapisdated in a recombinant AAV vector.

In some embodiments of any one of the foregoing aspects, the cell is edited in vivo.

In some embodiments of any one of the foregoing aspects, the method of integrating a transgene is conducted ex vivo.

In some embodiments of any one of the foregoing aspects, the cell is a blood, liver, muscle, or CNS cell. In some embodiments, the cell is administered in an autologous transplant after transgene integration. In some embodiments, the cell is administered in an allogeneic transplant after transgene integration.

In some embodiments of any one of the foregoing aspects, the nuclease or the polynucleotide sequence encoding the nuclease and the polynucleotide cassette are administered to the subject on the same day. In some embodiments of any one of the foregoing aspects, the nuclease or the polynucleotide sequence encoding the nuclease and the polynucleotide cassette are administered to the subject on different days.

In some embodiments, the nuclease or the polynucleotide sequence encoding the nuclease is administered to the subject 1 hour to 3 days after the polynucleotide cassette. In some embodiments, the nuclease or the polynucleotide sequence encoding the nuclease is administered to the subject 1 hour to 24 hours after the polynucleotide cassette. In some embodiments, the nuclease or the polynucleotide sequence encoding the nuclease is administered to the subject 1 hour to 3 days before the polynucleotide cassette. In some embodiments, the nuclease or the polynucleotide sequence encoding the nuclease is administered to the subject 1 hour to 4 hours before the polynucleotide cassette. In some embodiments, the nuclease or the polynucleotide sequence encoding the nuclease is administered to the subject 4 hours before the polynucleotide cassette.

In some embodiments of any one of the foregoing aspects, the nuclease or the polynucleotide sequence encoding the nuclease and the polynucleotide cassette are contacted with the human cell on the same day.

In some embodiments of any one of the foregoing aspects, the nuclease or the polynucleotide sequence encoding the nuclease and the polynucleotide cassette are contacted with the human cell on different days.

In some embodiments, the nuclease or the polynucleotide sequence encoding the nuclease are contacted with the human cell 1 hour to 3 days after the polynucleotide cassette. In some embodiments, the nuclease or the polynucleotide sequence encoding the nuclease are contacted with the human cell 1 hour to 24 hours after the polynucleotide cassette. In some embodiments, the nuclease or the polynucleotide sequence encoding the nuclease are contacted with the human cell 1 hour to 3 days before the polynucleotide cassette. In some embodiments, the nuclease or the polynucleotide sequence encoding the nuclease are contacted with the human cell 1 hour to 4 hours before the polynucleotide cassette. In some embodiments, the nuclease or the polynucleotide sequence encoding the nuclease are contacted with the human cell 4 hours before the polynucleotide cassette.

In some embodiments of any of the foregoing aspects, the cut site is distal from the target integration site.

In other embodiments of any of the foregoing aspects, the cut site is overlapping with the integration site.

As used in this application, the terms “about” and “approximately” are used as equivalents. Any citations to publications, patents, or patent applications herein are incorporated by reference in their entirety. Any numerals used in this application with or without about/approximately are meant to cover any normal fluctuations appreciated by one of ordinary skill in the relevant art.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### Definitions

About: The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Adult: As used herein, the term “adult” refers to a human eighteen years of age or older. In some embodiments, a human adult has a weight within the range of about 90 pounds to about 250 pounds.

Associated: Two events or entities are “associated” with one another, as that term is used herein, if the presence, level and/or form of one is correlated with that of the other. For example, a particular entity (e.g., polypeptide, genetic signature, metabolite, microbe, and the like) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Biological Sample: As used herein, the term “biological sample” typically refers to a sample obtained or derived from a biological source (e.g., a tissue or organism or cell culture) of interest, as described herein. In some embodiments, a source of interest comprises an organism, such as an animal or human. In some embodiments, a biological sample is or comprises biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, and the like. In some embodiments, a biological sample is or comprises cells obtained from an individual. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces, and the like.), and the like. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, and the like.

Biomarker: The term “biomarker” is used herein, consistent with its use in the art, to refer to an entity whose presence, level, or form correlates with a particular biological event or state of interest, so that it is considered to be a “marker” of that event or state. Among other things, the present disclosure encompasses biomarkers for gene therapy (e.g., that are useful to assess one or more features or characteristics of a gene therapy treatment, such as, for instance, extent, level, and/or persistence of payload expression). In some embodiments, a biomarker is a cell surface marker. In some embodiments, a biomarker is intracellular. In some embodiments, a biomarker is found outside of cells (e.g., is secreted or is otherwise generated or present outside of cells, e.g., in a body fluid such as blood, urine, tears, saliva, cerebrospinal fluid, and the like). In certain embodiments, the present disclosure demonstrates effectiveness of biomarkers that can be detected in a sample obtained from a subject who has received gene therapy for use in assessing one or more features or characteristics of that gene therapy; in some such embodiments, the sample is of cells, tissue, and/or fluid other than that to which the gene therapy was delivered and/or other than that where the payload is active.

Codon optimization: As used herein, the term “codon optimization” refers to a process of changing codons of a given gene in such a manner that the polypeptide sequence encoded by the gene remains the same while the changed codons improve the process of expression of the polypeptide sequence. For example, if the polypeptide is of a human protein sequence and expressed in E. coli, expression will often be improved if codon optimization is performed on the DNA sequence to change the human codons to codons that are more effective for expression in E. coli.

Detectable Moiety: The term “detectable moiety” as used herein refers to any entity (e.g., molecule, complex, or portion or component thereof). In some embodiments, a detectable moiety is provided and/or utilizes as a discrete molecular entity; in some embodiments, it is part of and/or associated with another molecular entity. Examples of detectable moieties include, but are not limited to: various ligands, radionuclides (e.g., ³H, ¹⁴C, ¹⁸F, ¹⁹F, ³²P, ³⁵S, ¹³⁵I, ¹²⁵I, ¹²³I, ⁶⁴Cu, ¹⁸⁷Re, ¹¹¹In, ⁹⁰Y, ^{99m}Tc, ¹⁷⁷Lu, ⁸⁹Zr and the like), fluorescent dyes (for specific exemplary fluorescent dyes, see below), chemiluminescent agents (such as, for example, acridinum esters, stabilized dioxetanes, and the like), bioluminescent agents, spectrally resolvable inorganic fluorescent semiconductors nanocrystals (i.e., quantum dots), metal nanoparticles (e.g., gold, silver, copper, platinum, and the like) nanoclusters, paramagnetic metal ions, enzymes (for specific examples of enzymes, see below), colorimetric labels (such as, for example, dyes, colloidal gold, and the like), biotin, dioxigenin, haptens, antibodies, and/or proteins for which antisera or monoclonal antibodies are available.

Child: As used herein, the term “child” refers to a human between two and 18 years of age. Body weight can vary widely across ages and specific children, with a typical range being 30 pounds to 150 pounds.

Combination therapy: As used herein, the term “combination therapy” refers to those situations in which a subject is simultaneously exposed to two or more therapeutic regimens (e.g., two or more therapeutic agents, for example a gene therapy and a non-gene therapy therapeutic modality). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time).

Composition: Those skilled in the art will appreciate that the term “composition”, as used herein, may be used to refer to a discrete physical entity that comprises one or more specified components. In general, unless otherwise specified, a composition may be of any form—e.g., gas, gel, liquid, or solid.

Determine: Many methodologies described herein include a step of “determining”. Those of ordinary skill in the art, reading the present specification, will appreciate that such “determining” can utilize or be accomplished through use of any of a variety of techniques available to those skilled in the art, including for example specific techniques explicitly referred to herein. In some embodiments, determining involves manipulation of a physical sample. In some embodiments, determining involves consideration and/or manipulation of data or information, for example utilizing a computer or other processing unit adapted to perform a relevant analysis. In some embodiments, determining involves receiving relevant information and/or materials from a source. In some embodiments, determining involves comparing one or more features of a sample or entity to a comparable reference.

Distal: As used herein, the term “distal” with respect to the position of a cut site relative to a target integration site means that the cut site and target integration site are not identical and/or do not overlap. In some embodiments, the cut site is within about 2 KB (e.g., 100 bp, 200 bp, 300 bp, 400 bp, 500 bp, 600 bp, 700 bp, 800 bp, 900 bp, 1 kB, 1.1 kB, 1.2 kB, 1.3 KB, 1.4 KB, 1.5 kB, 1.6 kB, 1.7 kB, 1.8 KB, 1.9 kB, or 2.0 kB) of the integration site. In some embodiments, the cut site is within the bounds of the homology arms. It is to be understood that in other embodiments, the cut site may be identical to or overlapping with the target integration site.

Gene: As used herein, the term “gene” refers to a DNA sequence that encodes a gene product (e.g., an RNA product and/or a polypeptide product). In some embodiments, a gene includes a coding sequence (e.g., a sequence that encodes a particular gene product); in some embodiments, a gene includes a non-coding sequence. In some particular embodiments, a gene may include both coding (e.g., exonic) and non-coding (e.g., intronic) sequences. In some embodiments, a gene may include one or more regulatory elements (e.g., promoters, enhancers, silencers, termination signals) that, for example, may control or impact one or more aspects of gene expression (e.g., cell-type-specific expression, inducible expression). In some embodiments, a gene is located or found (or has a nucleotide sequence identical to that located or found) in a genome (e.g., in or on a chromosome or other replicable nucleic acid).

Gene product or expression product: As used herein, the term “gene product” or “expression product” generally refers to an RNA transcribed from the gene (pre- and/or post-processing) or a polypeptide (pre- and/or post-modification) encoded by an RNA transcribed from the gene.

Identity: As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4:11-17), which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

“Improve,” “increase”, “inhibit” or “reduce”: As used herein, the terms “improve”, “increase”, “inhibit”, “reduce”, or grammatical equivalents thereof, indicate values that are relative to a baseline or other reference measurement. In some embodiments, an appropriate reference measurement may be or comprise a measurement in a particular system (e.g., in a single individual) under otherwise comparable conditions absent presence of (e.g., prior to and/or after) a particular agent or treatment, or in presence of an appropriate comparable reference agent. In some embodiments, an appropriate reference measurement may be or comprise a measurement in comparable system known or expected to respond in a particular way, in presence of the relevant agent or treatment.

Infant: As used herein, the term “infant” refers to a human under two years of age. Typical body weights for an infant range from 3 pounds up to 20 pounds.

Neonate: As used herein, the term “neonate” refers to a newborn human.

Nucleic acid: As used herein, in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid residue (e.g., a nucleotide and/or nucleoside); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is partly or wholly single stranded; in some embodiments, a nucleic acid is partly or wholly double stranded. In some embodiments a nucleic acid has a nucleotide sequence comprising at least one element that encodes, or is the complement of a sequence that encodes, a polypeptide. In some embodiments, a nucleic acid has enzymatic activity.

Peptide: As used herein, the term “peptide” or “polypeptide” refers to any polymeric chain of amino acids. In some embodiments, a peptide has an amino acid sequence that occurs in nature. In some embodiments, a peptide has an amino acid sequence that does not occur in nature. In some embodiments, a peptide has an amino acid sequence that is engineered in that it is designed and/or produced through action of the hand of man. In some embodiments, a peptide may comprise or consist of natural amino acids, non-natural amino acids, or both. In some embodiments, a peptide may comprise or consist of only natural amino acids or only non-natural amino acids. In some embodiments, a peptide may comprise D-amino acids, L-amino acids, or both. In some embodiments, a peptide may comprise only D-amino acids. In some embodiments, a peptide may comprise only L-amino acids. In some embodiments, a peptide is linear. In some embodiments, the term “peptide” may be appended to a name of a reference peptide, activity, or structure; in such instances it is used herein to refer to peptides that share the relevant activity or structure and thus can be considered to be members of the same class or family of peptides. For each such class, the present specification provides and/or those skilled in the art will be aware of exemplary peptides within the class whose amino acid sequences and/or functions are known; in some embodiments, such exemplary peptides are reference peptides for the peptide class or family. In some embodiments, a member of a peptide class or family shows significant sequence homology or identity with, shares a common sequence motif (e.g., a characteristic sequence element) with, and/or shares a common activity (in some embodiments at a comparable level or within a designated range) with a reference peptide of the class; in some embodiments with all peptides within the class). For example, in some embodiments, a member peptide shows an overall degree of sequence homology or identity with a reference peptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids.

Safe Harbor: As used herein, the term “safe harbor,” “safe harbor locus,” “safe harbor gene,” or “safe harbor site” refers to one or more regions of a subject's or cellular genome (e.g., mouse genome, human genome, humanized animal genome, chimeric animal genome) that enable stable expression of integrated transgenes without negatively affecting a subject and/or cell. In some embodiments, integration at a safe harbor allows predictable function of integrated transgenes. In some embodiments, integration at a safe harbor does not cause an unintended alteration of a subject or cell genome posing a risk to the subject or cell. In some embodiments, safe harbor genes may be disrupted without integration of transgenes (e.g., through NHEJ) without negatively affecting a subject and/or cell (e.g., without causing oncogenesis).

Subject: As used herein, the term “subject” refers to an organism, typically a mammal (e.g., a human, in some embodiments including prenatal human forms). In some embodiments, a subject is suffering from a relevant disease, disorder, or condition. In some embodiments, a subject is susceptible to a disease, disorder, or condition. In some embodiments, a subject displays one or more symptoms or characteristics of a disease, disorder, or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered. In preferred embodiments, the subject is a human.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Variant: As used herein in the context of molecules, e.g., nucleic acids, proteins, or small molecules, the term “variant” refers to a molecule that shows significant structural identity with a reference molecule but differs structurally from the reference molecule, e.g., in the presence or absence or in the level of one or more chemical moieties as compared to the reference entity. In some embodiments, a variant also differs functionally from its reference molecule. In general, whether a particular molecule is properly considered to be a “variant” of a reference molecule is based on its degree of structural identity with the reference molecule. As will be appreciated by those skilled in the art, any biological or chemical reference molecule has certain characteristic structural elements. A variant, by definition, is a distinct molecule that shares one or more such characteristic structural elements but differs in at least one aspect from the reference molecule. To give but a few examples, a polypeptide may have a characteristic sequence element comprised of a plurality of amino acids having designated positions relative to one another in linear or three-dimensional space and/or contributing to a particular structural motif and/or biological function; a nucleic acid may have a characteristic sequence element comprised of a plurality of nucleotide residues having designated positions relative to another in linear or three-dimensional space. In some embodiments, a variant polypeptide or nucleic acid may differ from a reference polypeptide or nucleic acid as a result of one or more differences in amino acid or nucleotide sequence and/or one or more differences in chemical moieties (e.g., carbohydrates, lipids, phosphate groups) that are covalently components of the polypeptide or nucleic acid (e.g., that are attached to the polypeptide or nucleic acid backbone). In some embodiments, a variant polypeptide or nucleic acid shows an overall sequence identity with a reference polypeptide or nucleic acid that is at least 85%, 86%, 87%, 88%, 89%, 90%, 91%, 92%, 93%, 94%, 95%, 96%, 97%, or 99%. In some embodiments, a variant polypeptide or nucleic acid does not share at least one characteristic sequence element with a reference polypeptide or nucleic acid. In some embodiments, a reference polypeptide or nucleic acid has one or more biological activities. In some embodiments, a variant polypeptide or nucleic acid shares one or more of the biological activities of the reference polypeptide or nucleic acid. In some embodiments, a variant polypeptide or nucleic acid lacks one or more of the biological activities of the reference polypeptide or nucleic acid. In some embodiments, a variant polypeptide or nucleic acid shows a reduced level of one or more biological activities as compared to the reference polypeptide or nucleic acid. In some embodiments, a polypeptide or nucleic acid of interest is considered to be a “variant” of a reference polypeptide or nucleic acid if it has an amino acid or nucleotide sequence that is identical to that of the reference but for a small number of sequence alterations at particular positions. Typically, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. In some embodiments, a variant polypeptide or nucleic acid comprises about 10, about 9, about 8, about 7, about 6, about 5, about 4, about 3, about 2, or about 1 substituted residue as compared to an appropriate reference. Often, a variant polypeptide or nucleic acid comprises a very small number (e.g., fewer than about 5, about 4, about 3, about 2, or about 1) number of substituted, inserted, or deleted, functional residues (i.e., residues that participate in a particular biological activity) relative to the reference. In some embodiments, a variant polypeptide or nucleic acid comprises not more than about 5, about 4, about 3, about 2, or about 1 addition or deletion, and, in some embodiments, comprises no additions or deletions, as compared to the reference. In some embodiments, a variant polypeptide or nucleic acid comprises fewer than about 25, about 20, about 19, about 18, about 17, about 16, about 15, about 14, about 13, about 10, about 9, about 8, about 7, about 6, and commonly fewer than about 5, about 4, about 3, or about 2 additions or deletions as compared to the reference. In some embodiments, a reference polypeptide or nucleic acid is one found in nature. In some embodiments, a reference polypeptide or nucleic acid is a human polypeptide or nucleic acid.

### BRIEF DESCRIPTION OF THE DRAWINGS

FIG. 1A shows an exemplary diagram of potential cut sites, for example, via one or more nucleases, in the human albumin gene (ALB). FIG. 1B shows the sequence of human ALB around STOP codon in exon 14, with the underlined sequence indicating an exemplary region to target for cutting.

FIG. 2A shows that an induced double-strand break (DSB) in intron 14 of human ALB gene enhanced integration efficiency. FIG. 2B shows that an induced DSB in intron 13 of human ALB gene also enhanced integration efficiency. FIG. 2C shows the degree of enhancement of integration efficiency relative to the location of DSB introduced in various places in intron 13 or intron 14 of the ALB gene. FIG. 2D shows a representative fluorescence image depicting that Cas9 protein together with guide RNA may increase GFP positive cells. FIG. 2E shows that an induced DSB in intron 13 and/or 14 of human ALB enhanced the integration efficiency (measured as a percentage of GFP positive cells). FIG. 2F shows that an induced DSB in intron 13 and/or 14 of human ALB enhanced the integration efficiency (measured as levels of fused mRNA).

FIG. 3A shows representative fluorescence images depicting that use of Cas9 and appropriate guide RNAs may increase integration efficiency. FIG. 3B shows different ratios of Cas9 mRNA to gRNA and the integration efficiency (measured as a percentage of GFP positive cells).

FIG. 4A shows a schematic of a GENERIDE™ construct before integration (AAV) and following HR-mediated integration into the genome at a targeted locus (e.g., Albumin). FIG. 4B shows that longer homology alignments may predict higher integration efficiency (measured as a level of fused mRNA). FIG. 4C shows that longer homology alignments may predict higher integration efficiency (measured as a percentage of GFP positive cells).

FIG. 5A shows that a variety of Cas9 (e.g., HF1-Cas9, HF2-Cas9) can enhance on-target integration. FIG. 5B shows that a high-fidelity version of Cas9 (e.g., HiFi-Cas9) can enhance on-target integration.

FIG. 6 shows that Staphylococcus aureus Cas9 (saCas9) can improve integration of human GENERIDE™ vector.

FIG. 7 shows an exemplary plasmid designed to express saCas9 and a gRNA (top panel). The plasmid when packed into an AAV can enhance integration after transfection (bottom panel).

FIG. 8 shows representative fluorescence images depicting that use of Cas9 and appropriate guide RNAs may increase integration efficiency.

FIG. 9A shows that an induced DSB in intron 13 and/or 14 of human ALB enhanced integration efficiency (measured as a percentage of GFP positive cells). FIG. 9B shows that an induced DSB in intron 13 and/or 14 of human ALB enhanced integration efficiency (measured as levels of fused mRNA).

FIG. 10 shows assessment of GENERIDE™ biomarkers 3 weeks after in-vivo administration of GENERIDE™, Cas9, and appropriate guide RNAs. D, day of LNP administration relative to AAV administration.

FIG. 11 shows images depicting that in vivo administration of Cas9 and appropriate guide RNAs may increase integration efficiency.

FIG. 12 shows results of an experiment in which GENERIDE™ constructs (GR-hATP7B or GR-GFP) were administered alone or in combination with Cas9, with a naïve control. The GR-hATP7b construct included varying sizes of homology arms (0.4/0.8 kB; 0.8/0.4 kB, or 0.6/0.6 kB), while the GR-GFP had 1.0/1.0 kB homology arms. GOI, gene of interest. FIG. 12 shows a series of graphs measuring levels of fused mRNA (copies/20 mg) (left panel) and normalized fused mRNA (norm. to 0.6/0.6 GR-hATP7B) (right panel). GR-GFP had a higher baseline, which, without wishing to be bound by any particular theory, may be due to longer homology arms or a smaller GOI.

FIG. 13 shows results of an experiment in which the fused mRNA fold increase between GOIs was measured as normalized fused mRNA (norm. to GR only) for the indicated constructs. The level of fused mRNA is a measure of integration efficiency.

FIG. 14 shows results of an experiment in which a GENERIDE™ construct (GR-hAAT) was administered to human HepG2 cells alone or in combination with spCas9 mRNA, with a naïve and H20 control. FIG. 14 shows a graph measuring levels of fused mRNA (copies/100 ng). spCas9 mRNA increased integration efficiency of GR-hAAT.

FIGS. 15A and 15B show results of an experiment in which integration efficiency of wild type (WT) versus mutant plasmids in circular or linear format with or without Cas9 was measured. The plasmids had restriction enzyme cutting sites to linearize the plasmid, and PAM mutations may further enhance the integration. FIG. 15A shows a graph measuring the levels of fused mRNA (copies/100 ng) in the presence or absence of Cas9 for WT and mutant plasmids in both circular and linear formats, with a naïve control (left panel). FIG. 15B shows a graph measuring the levels of fused mRNA in the presence of Cas9 (norm. to C-WT+Cas9) for WT and mutant plasmids in both circular and linear formats (right panel).

FIGS. 16A and 16B show results of an experiment in which overall editing efficiency (Editing_efficiency), percent reads with HDR donor integration (KI_HDR), and percent reads with NHEJ donor integration in either forward or reverse orientation (NHEJ_Fw+Rv) were measured after transfection with AZ Nuclease (FIG. 16A) or SpCas9 (FIG. 16B) mRNA for different ratios of donor to gRNA respectively. Arrows indicate overlapping gRNAs for SpCas9 and AZ Nuclease; asterisks indicate gRNAs with the highest KI-HDR for AZ Nuclease.

FIGS. 17A and 17B show results of an experiment in which transgene integration efficiency was measured for different human gRNAs in HEPG2 cells. FIG. 17A shows a graph measuring the levels of fused mRNA (copies/2.5 ng) in the presence of spCas9 in HEPG2 cells, with a GR alone (PD128) control. FIG. 17B shows a graph measuring the levels of normalized fused mRNA (norm. to vt only) in the presence of spCas9 in HEPG2 cells, with a GR alone (PD128) control.

FIGS. 18A and 18B show results of an experiment evaluating the effect of the cutting location (i.e., cutting within the GR homology arm (HA), in which donor HA segments 3′ and 5′ of the cut site participate in HDR; in other words, the DSB site is within the bounds of the homology arm, versus cutting outside of the GR homology arm, in which only one end can undergo HDR) for spCas9 in human cells. FIG. 18A shows a graph comparing the levels of fused mRNA (norm.) when the cutting is performed outside the HA (1 arm) versus when the cutting is performed within the HA (2 arms); cutting within the HA resulted in significantly higher integration efficiency for spCas9. FIG. 18B shows a graph measuring the levels of fused mRNA (norm.) as a function of aligned length (intron 12 and 13) when the cutting is performed outside the HA and within the HA.

FIGS. 19A-19C show results of an experiment in which transgene integration efficiency of GENERIDE™ with AZ NucleaseCas9 was measured for different human gRNAs in HEPG2 cells. FIG. 19A shows a graph measuring the levels of fused mRNA (copies/2.5 ng) in the presence of GR-mut and AZ Nuclease Cas9 in HEPG2 cells, with a PD128 control. FIG. 19B shows a graph measuring the levels of fused mRNA (copies/2.5 ng) in the presence of GR-GFP and AZ Nuclease Cas9 in HEPG2 cells, with a naive control and GR alone (vt-0290) control. FIG. 19C shows a graph measuring the levels of fused mRNA (copies/2.5 ng) in the presence of GR-ATP7B and AZ Nuclease Cas9 in HEPG2 cells, with a naive control and GR alone (vt-235) control.

FIGS. 20A and 20B show results of an experiment measuring the efficiency of GENERIDE™ integration with AZ Nuclease Cas9 when cutting within the HA versus outside the HA in HEPG2 cells. FIG. 20A shows a graph comparing the levels of fused mRNA (norm. to vt only) when the cutting is performed outside the HA (1 arm) versus when the cutting is performed within the HA (2 arms) (left panel). FIG. 20B shows a graph measuring the levels of fused mRNA (norm.) as a function of aligned length (intron 12 and 13) when the cutting is performed outside the HA and within the HA (right panel).

FIG. 21 shows results of an experiment in which overall editing efficiency (Editing_efficiency), percent reads with HDR donor integration (KI_HDR), and percent reads with NHEJ donor integration in either forward or reverse orientation (NHEJ_Fw+Rv) were measured after transfection with AZ Nuclease mRNA for different ratios of donor to gRNA respectively. FIG. 21 shows a series of graphs in which Editing_efficiency (%) (top panel), KI_HDR (%) (middle panel), and NHEJ_Fw+Rv (%) (bottom panel) were measured after transfection with AZ Nuclease mRNA for the indicated gRNAs and donors (NoDonor, ssDNA, or dsDNA) in HEPG2 cells. Mean KI_HDR (%) was higher for ssDNA compared to dsDNA and vice-versa for NHEJ_Fw+Rv (%). Arrows indicate overlapping gRNAs for SpCas9 and AZ Nuclease; asterisks indicate gRNAs with the highest KI-HDR for AZ Nuclease.

FIGS. 22A-22E show results of experiments performed to optimize a dosing strategy for GENERIDE™ and GenVoy-ILM lipid nanoparticle (LNP) in liver cells, confirm delivery of Cas9 by GenVoy-ILM LNP, and determine the timing of components for peak integration. Different groups of mice were administered different combinations of either GR (3e13 vg/kg) alone, GR (3e13 vg/kg) in combination with Cas9 (1 mg/kg) after 1, 3, or 7 days, or only LNP containing Cas9+gRNA (1 mg/kg). The GENERIDE™ tool vector used was DJ-GFP (LB-Vt-0298-001). FIG. 22A shows immunohistochemistry (IHC) images of liver cells detecting Cas9-Flag, confirming Cas9 expression after delivery of LNP containing Cas9, with a naïve WT control. FIG. 22B shows a series of graphs measuring the levels of ALB-2A (μg/ml) for up to 6 weeks post LNP dosing (left panel) and measuring the levels of ALB-2A (μg/ml) at 3 weeks post LNP dosing (right panel) for GR dosed with LNP at different time points (D1, D3, and D7), with a GR only control. FIG. 22C shows an IHC image of hepatocytes confirming improved integration in GR2.0 (Cas9) after delivery of GR (3e13)+LNP (1 mg/kg) containing Cas9. For this experiment, FvB/NJ mice were dosed with 3e13 vk of GR-GFP (1.0/1.0) followed by Day 1 Gen Voy-ILM LNP-Cas9 at 1 mg/kg. FIG. 22D shows a bar graph measuring the levels of ALB-2A (μg/ml) 1 week after administration to confirm potency of stored LNP when LNP has been stored for 1 month or 2 months. FIG. 22E shows a series of graphs quantifying the indel frequency via TIDE of D4-1 (top panel) and D4-2 (bottom panel).

FIGS. 23A-23F show results of experiments performed to assess RNA from Integrated DNA Technologies (IDT) to ensure feasibility with AstraZeneca (AZ) LNP. The GR-HDR donor used was DJ-mHA (1.0/1.0) GFP-LB-Vt-0298-001 and the LNP used was AZ LNP. 5 different combinations were administered to different groups of WT mice: WT GR only, WT GR+LNP N:P=3 (Co-formulation) D1 Stagger, WT GR+LNP N:P=6 (Co-formulation) D1 Stagger, WT GR+LNP N:P=6 (Separate (Sep) formulation) D1 Stagger, and WT GR+LNP N:P=6 (Co-formulation) H4 Stagger. FIG. 23A shows results of an experiment performed to measure the levels of ALB-2A (μg/ml) for up to 3 weeks post LNP dosing for the 5 different combinations listed above, with the GR only as control. FIG. 23B is a bar graph measuring the copies of fused mRNA/100 ng of RNA for the 5 different combinations administered, with the GR only as control. FIG. 23C is a set of IHC images with the IHC being performed to label and measure GFP+ cells after administration of the 5 combinations listed above. FIG. 23D is a bar graph measuring the % of GFP positive cells after administration of the 5 combinations listed above. FIG. 23E is a bar graph measuring the levels of ALB-2A (μg/ml) after administration of the 5 combinations listed above. FIG. 23F shows the levels of ALB-2A (μg/ml) as a function of the % of GFP positive cells after administration of the 5 combinations listed above.

FIGS. 24A and 24B show results of experiments performed to optimize a treatment timeline, in which cells were treated according to 2 different protocols to determine which was better at enhancing integration. FIG. 24A shows results of experiments in which cells were treated with GR on the same day as the mRNA transfection (original protocol). Seeding was performed on D0 followed by nuclease mRNA transfection along with GR-GFP treatment on D1 and harvesting was performed on D4 (top panel). FIG. 24A shows a bar graph measuring the % of hemagglutinin (HA)+ cells for the 4 different combinations administered: naïve (control), GR-GFP, GR-GFP+Cas9, and mRNA only (bottom left panel) and another bar graph measuring the % of HA+ cells (norm.) for the same 4 combinations administered (bottom right panel). FIG. 24B shows results of experiments in which cells were treated with GR on the day after the mRNA transfection. Seeding was performed on D0 followed by nuclease mRNA transfection on D1, followed by GR-GFP treatment on D2 and finally, harvesting was performed on D5 (top panel). FIG. 24B shows a bar graph measuring the % of HA+ cells for the 4 different combinations administered: naïve (control), GR-GFP, GR-GFP+Cas9, and mRNA only (bottom left panel) and another bar graph measuring the % of HA+ cells (norm.) for the same 4 combinations administered (bottom right panel).

FIG. 25 shows results of experiments performed to compare the transfection efficiency of different reagents via immunofluorescence. FIG. 25 shows 4 immunofluorescence images (top left panel) testing the transfection efficiency of Lipofectamine™ 3000 for transfection of mCherry and eGFP, with a DAPI channel and an eGFP-mCherry merged image and 4 immunofluorescence images (top right panel) testing the transfection efficiency of MessengerMax™ for transfection of mCherry and eGFP, with a DAPI channel and an eGFP-mCherry merged image. FIG. 25 also shows bar graphs measuring the % Transfection efficiency for eGFP and mCherry for both Lipofectamine™ 3000 and MessengerMax™, bottom left and bottom right panels, respectively.

FIG. 26 shows results of experiments performed to measure the amount of protein expression (% of HA+ cells) when using Cas9 mRNA along with MessengerMax™ as a transfection reagent. Seeding was performed on D0 followed by nuclease mRNA transfection on D1, followed by GR-GFP treatment on D2 and finally, harvesting was performed on D5 (top panel). FIG. 26 shows a bar graph measuring the % of HA+ cells for the 8 different combinations administered: naïve (control), mRNA only, Vehicle+GR-GFP+Cas9 mRNA, Vehicle+dsODN+GR-GFP+Cas9 mRNA, #2 gRNA+GR-GFP+Cas9 mRNA, #2 gRNA+dsODN+GR-GFP+Cas9 mRNA, #10 gRNA+GR-GFP+Cas9 mRNA, and #10 gRNA+dsODN+GR-GFP+Cas9 mRNA (bottom left panel), a bar graph measuring the % of HA+ cells (norm.) for the same 8 combinations administered (bottom middle panel), and another bar graph measuring the cell count/image for the same 8 combinations administered (bottom right panel).

### DETAILED DESCRIPTION OF CERTAIN EMBODIMENTS

### Gene Therapy

Gene therapies alter the gene expression profile of a patient's cells by gene transfer, a process of delivering an exogenous therapeutic gene, called a transgene. Various delivery vehicles are known to be used as vectors to transport transgenes into the nucleus of a cell to alter or augment a cell's capabilities (e.g., proteome, functionality, and the like). Developers have made great strides in introducing genes into cells in tissues such as liver, retina of the eye and blood-forming cells of bone marrow using a variety of vectors. These approaches have in some cases led to approved therapies and, in other cases, have shown very promising results in clinical trials.

There are multiple gene therapy approaches. In conventional AAV gene therapy (also described as traditional or conventional gene therapy herein), a transgene is introduced into a nucleus of a host cell but is not intended to integrate in chromosomal DNA. The transgene is expressed from a non-integrated genetic element called an episome that exists inside the nucleus. A second type of gene therapy employs the use of a different type of virus, such as lentivirus, that inserts itself, along with the transgene, into the chromosomal DNA but at arbitrary sites.

Episomal expression of a gene must be driven by an exogenous promoter, leading to production of a protein that corrects or ameliorates the disease condition.

### Limitations of Previously Known Gene Therapies

In the case of gene therapy based on episomal expression, when cells divide during the process of growth or tissue regeneration, benefits of the therapy typically decline because transgenes were not intended to integrate into a host chromosome and are not replicated during cell division. Each new generation of cells thus further reduces the proportion of cells expressing the transgene in the target tissue, leading to reduction or elimination of therapeutic benefit over time.

While the use of some gene therapy using viral mediated insertion has potential to provide long-term benefit because the gene is inserted into the host chromosome, there is no ability to control where the gene is inserted, which presents a risk of disrupting an essential gene or inserting into a location that can promote undesired effects such as tumor formation. For this reason, these integrating gene therapy approaches are primarily limited to ex vivo approaches, where the cells are treated outside the body, screened for successful integration, and then transplanted into a subject.

A common feature of many previously used gene therapy approaches is that a transgene is introduced into cells together with an exogenous promoter. Promoters are required to initiate transcription and amplification of DNA to messenger RNA, or mRNA, which will ultimately be translated into protein. Expression of high levels of therapeutic proteins from a transgene using previous gene therapy methods required strong, engineered promoters. While these promoters were a necessary component of previous gene therapy systems in order to produce protein expression, studies conducted in animal models have shown that non-specific integration of gene therapy vectors can result in significant increases in development of tumors. Promoter strength may play a crucial role in the increase of development of these tumors using previous gene therapy methods. Thus, attempts to drive high levels of expression with strong, exogenous promoters may have long-term deleterious consequences.

### A. Gene Editing

Gene editing is typically used to refer to the deletion, alteration, or augmentation of genes by introducing breaks in the DNA of cells using exogenously delivered gene editing mechanisms. Many current gene editing approaches have been limited in their efficacy due to high rates of unwanted off-target modifications and low efficiency of gene correction, resulting in part from a cell trying to rapidly repair introduced DNA breaks. Many gene editing techniques focus on disabling a dysfunctional gene or correcting or skipping individual deleterious mutations within a gene. These previous approaches often do not sufficiently treat or prevent genetic diseases due to the number of possible mutations in a gene, as compared to a method comprising insertion of a full corrective gene.

Unlike gene therapy approaches that make use of episomal expression, gene editing allows for a repaired genetic region to propagate to new generations of cells through normal cell division. Furthermore, a desired protein can be expressed using a cell's own regulatory machinery. Traditional approaches to gene editing are nuclease-based and employ nuclease enzymes to introduce a single- or double-stranded break in DNA at a specific cut site in order to cause a deletion, make an alteration, or apply a corrective sequence. These methods are designed to provide gene editing (e.g., deletion, alteration, insertion of corrective sequence, and the like) at the location of a cut site.

Once nucleases have cut the DNA, traditional gene editing techniques modify DNA using two routes: homology-directed repair (HDR) and non-homologous end joining (NHEJ). HDR in these previous methods involves highly precise incorporation of DNA sequences complementary to an integration site, which is at the same location as the cut site. HDR has key advantages in that it can repair DNA with high fidelity and avoids introduction of unwanted mutations at an integration site. NHEJ is a less selective, more error-prone process that rapidly joins ends of broken DNA, resulting in a high frequency of insertions or deletions at a cut site.

### 1. Traditional Nuclease-Based Gene Editing

Nuclease-based gene editing uses nucleases, which are endogenous or engineered enzymes that produce single- or double-stranded breaks in DNA at a cut site. Nuclease-based gene editing is a two-step process. First, an exogenous nuclease, which is capable of cutting one or both strands in double-stranded DNA, is directed to a desired site (e.g., through use of a guide RNA, site-specific residues within the nuclease, and the like) and makes a specific cut at a cut site. Next, endogenous cellular DNA repair machinery is activated and completes the editing process through either NHEJ or, less commonly, HDR.

NHEJ can occur in absence of a DNA template for a cell to copy as it repairs a DNA cut. Although the rates of NHEJ compared to other repair mechanisms (e.g., HDR) are species-dependent, in the absence of exogenous compounds (e.g., repair template sequence, enzymes, and the like) NHEJ is often preferentially employed by cells to repair double-stranded breaks. Gene editing through NHEJ may be used to introduce small insertions or deletions, known as indels, which may result in a knockout or reduction of gene function. NHEJ creates insertions and deletions in DNA due to its mode of repair and can also result in introduction of off-target, unwanted mutations, including chromosomal aberrations.

Nuclease-mediated HDR occurs with delivery of a nuclease and a DNA template with partial or full complementarity to a target integration site. In previous methods, a target integration site and cut site would coincide or overlap. Cells employ the DNA template to construct reparative DNA, resulting in insertion of a corrected and/or alternative genetic sequence.

Traditional gene editing (also described herein as conventional gene editing) has frequently employed three different classes of nucleases for nuclease-based approaches: Transcription activator-like effector nucleases (TALENs), Clustered, Regularly Interspaced Short Palindromic Repeats Associated (CRISPR/Cas) nucleases; and Zinc Finger Nucleases (ZFNs).

### 2. Limitations of Traditional Nuclease-Based Gene Editing

Previous nuclease-based gene editing approaches were limited by use of nuclease enzymes to produce overlapping cut sites and integration sites. Additionally, these traditional methods relied on exogenous promoters for transgene expression.

Nucleases may cause on- and off-target mutations. A major concern for implementing nuclease-based gene editing approaches is the relatively high frequency of off-target effects. Previous gene editing technologies could result in genotoxicity, including chromosomal alterations, based on the error prone NHEJ process, or interrupt native gene expression by targeting exons.

### GENERIDE™ Technology Platform

### 3. GENERIDE™ Features

GENERIDE™ is a novel AAV-based genome editing technology that precisely inserts a therapeutic transgene into a cellular genome via homologous recombination. GENERIDE™ provides durable transgene expression regardless of cell proliferation and tissue growth, and GENERIDE™-corrected cells show selective expansion within tissues affected by a disease (e.g., selective expansion of hepatocytes in a liver of a diseased subject). Without wishing to be bound by any particular theory, it is contemplated that GENERIDE™ is a genome editing technology that harnesses homologous recombination (HR), which is a naturally occurring HDR process that maintains fidelity of the genome. In some embodiments, GENERIDE™ allows insertion of transgenes into specific targeted genomic locations (e.g., through HR) without exogenous nucleases. In some embodiments, GENERIDE™ allows insertion of transgenes into specific targeted genomic locations (e.g., through HR) in combination with one or more exogenous nucleases. GENERIDE™-directed transgene integration is designed to leverage endogenous promoters at a target integration site to drive high levels of tissue-specific gene expression, without producing detrimental issues associated with use of exogenous promoters in traditional gene therapy.

### 4. Advantages of GENERIDE™ Technology

GENERIDE™ technology is designed to precisely integrate corrective genes into a patient's genome to provide a stable, durable therapeutic effect. Among other things, GENERIDE™ can be applied to targeting rare disorders (e.g., disorders of liver, CNS, muscle, blood, and the like) in pediatric patients where it is critical to provide treatment early in a patient's life before irreversible disease pathology can occur.

In some embodiments, GENERIDE™ technology provides improvements over certain key limitations of both traditional gene therapy and conventional gene editing approaches in a way that is well-positioned to treat genetic diseases (e.g., genetic diseases in pediatric patients). In some embodiments, GENERIDE™ uses an AAV vector to deliver a transgene into the nucleus of a cell, followed by insertion of the transgene into a target integration site within the cell genome, where transgene expression is regulated by one or more endogenous promoters. In some embodiments, GENERIDE™ may allow lifelong protein production, even as the body grows and changes over time, which is not feasible with conventional AAV gene therapy.

Among other things, GENERIDE™ technology may also provide surprising and unexpected improvements when combined with targeted nucleases. In some embodiments GENERIDE™ is combined with one or more nucleases targeting a particular cut site in the cell genome, wherein the cut site is distal from the target integration site (e.g., cut site and target integration site are not identical and/or do not overlap). As demonstrated herein, use of such targeted nucleases in combination with additional GENERIDE™ components may produce improved transgene gene integration rates and/or efficiency (e.g., improved levels of modified DNA, modified mRNA, and/or protein expression). In particular, targeted nucleases may be designed (e.g., alone or in combination with a targeting molecule, including but not limited to a guide RNA) to introduce a single- or double-stranded break at a cut site in a DNA sequence, wherein the cut site is distal from the target integration site. Although it has been demonstrated that introduction of a cut site distal from a target site for transgene integration may provide improvements in mice (See, Caneva et al., JCI Insight, 2019), these improvements were shown to be highly dependent on the precise location of a cut site. For example, Caneva showed that cutting within flanking intron 14 of mouse albumin produced marked improvements in cutting and HDR efficiency as compared to limited levels of integration when the cut site was introduced into intron 13 of mouse albumin (Figure S1 of Caneva). Although Caneva demonstrated that certain “hot spot” cut sites distal from an integration site could potentially increase HDR efficiency and transgene integration, there was no demonstration that these improvements led to site-specific integration and expression of a transgene of interest. Furthermore, Caneva limited all experiments to mouse cells comprising wild-type mouse albumin. As one of skill in the art would understand, considerable testing and research would be required to determine whether such a system could be altered for functionality in a different species. For example, it is known that the high degree of sequence variability in particular genes (e.g., albumin) across species such as mouse and human, do not allow one of skill to use the results from one species to determine if and where appropriate cut or integration sites may be found in the other species. Further, it is known in the art that mouse and human genes (e.g., albumin) may comprise significant sequence, epigenetic, and structural differences which can alter efficiency of nuclease cutting and/or homologous recombination at particular sites.

The present disclosure provides the surprising and unexpected insight that combining targeted nucleases with GENERIDE™ components, wherein a nuclease cut site is distal from a target integration site (e.g., cut site and integration site are not identical and/or do not overlap), may provide unexpected improvements in transgene integration rate and/or expression efficiency in human cells and tissues. In some embodiments, such improvements may be observed for multiple cut sites, e.g., within different non-coding sequences in a human DNA sequence of interest (e.g., both intron 13 and 14 of human albumin).

Modular approaches disclosed herein can be applied to allow GENERIDE™ to deliver robust, tissue-specific gene expression that is reproducible across different therapeutics delivered to a tissue (e.g., liver, muscle, CNS, blood).

Previous work on non-disruptive gene targeting is described in WO 2013/158309, incorporated herein by reference. Previous work on genome editing without nucleases is described in WO 2015/143177, incorporated herein by reference. Previous work on non-disruptive gene therapy for the treatment of MMA is described in WO 2020/032986, incorporated herein by reference. Previous work on monitoring of gene therapy is described in WO/2020/214582, incorporated herein by reference.

### B. GENERIDE™ Components

### Delivery Vehicles

There are multiple gene therapy approaches understood in the art. As such there are multiple mechanisms of delivery understood in the art. In some embodiments, a transgene is provided using a delivery vehicle. In some embodiments, compositions of the present disclosure comprise a delivery vehicle. In some embodiments, a delivery vehicle is or comprises a viral particle (e.g., a viral vector). In some embodiments, a delivery vehicle is or comprises a non-viral particle and/or non-particle payload (e.g., a nuclease). In some embodiments, a delivery vehicle is a lipid particle (e.g., a lipid nanoparticle). Various lipid nanoparticles for delivery of nucleic acids are known in the art, for example, those described in WO2015184256; WO2013149140; WO2014089486A1; WO2009127060; WO2011071860; WO2020219941 the contents of each of which is incorporated herein by reference.

In some embodiments, a delivery vehicle is or comprises an exosome. One of skill in the art will recognize various methods of exosome production and use. Examples of such methods and uses are described in Luan et al., Acta Pharmacologica Sinica volume 38, pages 754-763 (2017).

In some embodiments, a combination of one or more different payloads may be delivered with one or more delivery systems described herein (e.g., viral vector, lipid nanoparticle, and the like). In some embodiments, a delivery system may deliver one payload (e.g., one or more nucleases (e.g., Cas proteins, endonucleases, TALENs, ZFNs)) in combination with a second, distinct payload (e.g., a polynucleotide sequence comprising a transgene). In some embodiments, a first delivery system (e.g., a lipid nanoparticle (LNP_may deliver one payload (e.g., one or more nucleases (e.g., Cas proteins, endonucleases, TALENS, ZFNs)) in combination with a second, distinct delivery system (e.g., a viral vector such as a recombinant AAV vector), which delivers a second, distinct payload (e.g., a polynucleotide sequence comprising a transgene).

### Viral Vectors

In some embodiments, a delivery vehicle is or comprises a viral vector. Viral vectors comprise virus or viral chromosomal material, within which a heterologous nucleic acid sequence can be inserted for transfer into a target sequence of interest (e.g., for transfer into genomic DNA within a cell). Various viruses can be used as viral vectors, including, e.g., single-stranded DNA (ssDNA), double-stranded DNA (dsDNA) viruses, and/or RNA viruses with a DNA stage in their lifecycle. In some embodiments, a viral vector is or comprises an adeno-associated virus (AAV) or AAV variant.

In some embodiments, a vector particle is a single unit of virus comprising a capsid encapsidating a virus-based polynucleotide (e.g., a wild-type viral genome or a recombinant viral vector). In some embodiments, a vector particle is or comprises an AAV vector particle. In some embodiments, an AAV vector particle refers to a vector particle comprised of at least one AAV capsid protein and an encapsidated AAV vector. In some embodiments, a vector particle (also referred to as a viral vector) comprises at least one AAV capsid protein and an encapsidated AAV vector, wherein the vector further comprises one or more heterologous polynucleotide sequences.

### Capsid Proteins

In some embodiments, an expression construct comprises polynucleotide sequences encoding capsid proteins from one or more AAV subtypes, including naturally occurring and recombinant AAVs. In some embodiments, an expression construct comprises polynucleotide sequences encoding capsid proteins from AAV1, AAV2, AAV3, AAV4, AAV5, AAV6, AAV7, AAV8, AAV9, AAV10, AAV11, AAVC11.01, AAVC11.02, AAVC11.03, AAVC11.04, AAVC11.05, AAVC11.06, AAVC11.07, AAVC11.08, AAVC11.09, AAVC11.10, AAVC11.11 (referred to interchangeably herein as sL65), AAVC11.12, AAVC11.13, AAVC11.14, AAVC11.15, AAVC11.16, AAVC11.17, AAVC11.18, AAVC11.19, AAV-DJ, AAV-LK03, AAV-LK19, AAVrh.74, AAVrh.10, AAVhu.37, AAVrh.K, AAVrh.39, AAV12, AAV13, AAVrh.8, avian AAV, bovine AAV, canine AAV, equine AAV, primate AAV, non-primate AAV, ovine AAV, a hybrid AAV (e.g., an AAV comprising one more sequences of one AAV subtype and one or more sequences of a second subtype), and/or an AAV comprising a mutant AAV capsid protein or a chimeric AAV capsid (e.g., a capsid with polynucleotide sequences derived from two or more different serotypes of AAV), or variants thereof.

In some embodiments, viral vectors are packaged within capsid proteins (e.g., capsid proteins from one or more AAV subtypes). In some embodiments, capsid proteins provide increased or enhanced transduction of cells (e.g., human or murine cells) relative to a reference capsid protein. In some embodiments, capsid proteins provide increased or enhanced transduction of certain cells or tissue types (e.g., liver tropism, muscle tropism, CNS tropism, lung tropism) relative to a reference capsid protein. In some embodiments, capsid proteins increase or enhance transduction of cells or tissues (e.g., liver, muscle, and/or CNS) by at least about 10%, 20%, 30%, 40%, 50%, 60%, 70%, 80%, 90%, 100%, 200%, 300%, 400%, 500%, 600%, 700%, 800%, 900%, 1000%, or more relative to a reference capsid protein. In some embodiments, capsid proteins increase or enhance transduction of cells or tissues (e.g., liver, muscle, lung, and/or CNS) by at least about 1.2×, 1.5×, 2×, 3×, 4×, 5×, 6×, 7×, 8×, 9×, 10×, 11×, 12×, 13×, 14×, 15×, 16×, 17×, 18×, 19×, 20×, 30×, 40×, 50×, 60×, 70×, 80×, 90×, 100×, or more relative to a reference capsid protein.

In some embodiments, a sequence encoding a capsid protein may be or comprise a sequence having at least 80%, 85%, 90%, 95%, 99%, or 100% identity to a corresponding wild-type capsid protein. In some embodiments, a sequence encoding a capsid protein may be or comprise a sequence having at least 80%, 85%, 90%, 95%, 99%, or 100% identity to a corresponding sequence encoding an engineered capsid protein (e.g., chimeric capsid protein, codon-optimized capsid protein, and the like). In some embodiments, a sequence encoding a capsid protein may be or comprise a sequence having at least 80%, 85%, 90%, 95%, 99%, or 100% identity to an exemplary sequence in Table 1 below.

TABLE 1
Exemplary sequences encoding capsid protein(s)

| | | SEQ |
|---|---|---|
| Description | Sequence | ID NO |
| AAV LK03 | atggctgctgacggttatcttccagattggctcgaggacaacctttctgaaggcattcgagagt | 1 |
| (nucleotide) | ggtgggcgctgcaacctggagcccctaaacccaaggcaaatcaacaacatcaggacaacg | |
| | ctcggggtcttgtgcttccgggttacaaatacctcggacccggcaacggactcgacaagggg | |
| | gaacccgtcaacgcagcggacgcggcagccctcgagcacgacaaggcctacgaccagca | |
| | gctcaaggccggtgacaacccctacctcaagtacaaccacgccgacgccgagttccaggag | |
| | cggctcaaagaagatacgtcttttgggggcaacctcgggcgagcagtcttccaggccaaaaa | |
| | gaggcttcttgaacctcttggtctggttgaggaagcggctaagacggctcctggaaagaaga | |
| | ggcctgtagatcagtctcctcaggaaccggactcatcatctggtgttggcaaatcgggcaaac | |
| | agcctgccagaaaaagactaaatttcggtcagactggcgactcagagtcagtcccagaccct | |
| | caacctctcggagaaccaccagcagcccccacaagtttgggatctaatacaatggcttcagg | |
| | cggtggcgcaccaatggcagacaataacgagggtgccgatggagtgggtaattcctcagga | |
| | aattggcattgcgattcccaatggctgggcgacagagtcatcaccaccagcaccagaacctg | |
| | ggccctgcccacttacaacaaccatctctacaagcaaatctccagccaatcaggagcttcaaa | |
| | cgacaaccactactttggctacagcaccccttgggggtattttgactttaacagattccactgcc | |
| | acttctcaccacgtgactggcagcgactcattaacaacaactggggattccggcccaagaaa | |
| | ctcagcttcaagctcttcaacatccaagttaaagaggtcacgcagaacgatggcacgacgact | |
| | attgccaataaccttaccagcacggttcaagtgtttacggactcggagtatcagctcccgtacgt | |
| | gctcgggtcggcgcaccaaggctgtctcccgccgtttccagcggacgtcttcatggtccctca | |
| | gtatggatacctcaccctgaacaacggaagtcaagcggtgggacgctcatccttttactgcctg | |
| | gagtacttcccttcgcagatgctaaggactggaaataacttccaattcagctataccttcgagga | |
| | tgtaccttttcacagcagctacgctcacagccagagtttggatcgcttgatgaatcctcttattgat | |
| | cagtatctgtactacctgaacagaacgcaaggaacaacctctggaacaaccaaccaatcacg | |
| | gctgctttttagccaggctgggcctcagtctatgtctttgcaggccagaaattggctacctgggc | |
| | cctgctaccggcaacagagactttcaaagactgctaacgacaacaacaacagtaactttccttg | |
| | gacagcggccagcaaatatcatctcaatggccgcgactcgctggtgaatccaggaccagcta | |
| | tggccagtcacaaggacgatgaagaaaaatttttccctatgcacggcaatctaatatttggcaa | |
| | agaagggacaacggcaagtaacgcagaattagataatgtaatgattacggatgaagaagag | |
| | attcgtaccaccaatcctgtggcaacagagcagtatggaactgtggcaaataacttgcagagc | |
| | tcaaatacagctcccacgactagaactgtcaatgatcagggggccttacctggcatggtgtgg | |
| | caagatcgtgacgtgtaccttcaaggacctatctgggcaaagattcctcacacggatggacac | |
| | tttcatccttctcctctgatgggaggctttggactgaaacatccgcctcctcaaatcatgatcaaa | |
| | aatactccggtaccggcaaatcctccgacgactttcagcccggccaagtttgcttcatttatcac | |
| | tcagtactccactggacaggtcagcgtggaaattgagtgggagctacagaaagaaaacagc | |
| | aaacgttggaatccagagattcagtacacttccaactacaacaagtctgttaatgtggactttact | |
| | gtagacactaatggtgtttatagtgaacctcgccccattggcacccgttaccttacccgtcccct | |
| | gtaa | |
| AAV-LK03 | MAADGYLPDWLEDNLSEGIREWWALQPGAPKPKANQQH | 2 |
| (protein) | QDNARGLVLPGYKYLGPGNGLDKGEPVNAADAAALEHDK | |
| | AYDQQLKAGDNPYLKYNHADAEFQERLKEDTSFGGNLGR | |
| | AVFQAKKRLLEPLGLVEEAAKTAPGKKRPVDQSPQEPDSS | |
| | SGVGKSGKQPARKRLNFGQTGDSESVPDPQPLGEPPAAPTS | |
| | LGSNTMASGGGAPMADNNEGADGVGNSSGNWHCDSQWL | |
| | GDRVITTSTRTWALPTYNNHLYKQISSQSGASNDNHYFGYS | |
| | TPWGYFDFNRFHCHFSPRDWQRLINNNWGFRPKKLSFKLF | |
| | NIQVKEVTQNDGTTTIANNLTSTVQVFTDSEYQLPYVLGSA | |
| | HQGCLPPFPADVFMVPQYGYLTLNNGSQAVGRSSFYCLEY | |
| | FPSQMLRTGNNFQFSYTFEDVPFHSSYAHSQSLDRLMNPLI | |
| | DQYLYYLNRTQGTTSGTTNQSRLLFSQAGPQSMSLQARNW | |
| | LPGPCYRQQRLSKTANDNNNSNFPWTAASKYHLNGRDSL | |
| | VNPGPAMASHKDDEEKFFPMHGNLIFGKEGTTASNAELDN | |
| | VMITDEEEIRTTNPVATEQYGTVANNLQSSNTAPTTRTVND | |
| | QGALPGMVWQDRDVYLQGPIWAKIPHTDGHFHPSPLMGG | |
| | FGLKHPPPQIMIKNTPVPANPPTTFSPAKFASFITQYSTGQVS | |
| | VEIEWELQKENSKRWNPEIQYTSNYNKSVNVDFTVDTNGV | |
| | YSEPRPIGTRYLTRPL | |
| AAV8 | atggctgccgatggttatcttccagattggctcgaggacaacctctctgagggcattcgcgagt | 3 |
| (nucleotide) | ggtgggcgctgaaacctggagccccgaagcccaaagccaaccagcaaaagcaggacgac | |
| | ggccggggtctagtgcttcctggctacaagtacctcggacccttcaacggactcgacaaggg | |
| | ggagcccgtcaacgcggcggacgcagcggccctcgagcacgacaaggcctacgaccagc | |
| | agctgcaggcgggtgacaatccgtacctgcggtataaccacgccgacgccgagtttcagga | |
| | gcgtctgcaagaagatacgtcttttgggggcaacctcgggcgagcagtcttccaggccaaga | |
| | agcgggttctcgaacctctcggtctggttgaggaaggcgctaagacggctcctggaaagaag | |
| | agaccggtagagccatcaccccagcgttctccagactcctctacgggcatcggcaagaaag | |
| | gccaacagcccgccagaaaaagactcaattttggtcagactggcgactcagagtcagttcca | |
| | gaccctcaacctctcggagaacctccagcagcgccctctggtgtgggacctaatacaatggct | |
| | gcaggcggtggcgcaccaatggcagacaataacgaaggcgccgacggagtgggtagttcc | |
| | tcgggaaattggcattgcgattccacatggctgggcgacagagtcatcaccaccagcacccg | |
| | aacctgggccctgcccacctacaacaaccacctctacaagcaaatctccaacgggacatcgg | |
| | gaggagccaccaacgacaacacctacttcggctacagcaccccctgggggtattttgacttta | |
| | acagattccactgccacttttcaccacgtgactggcagcgactcatcaacaacaactggggatt | |
| | ccggcccaagagactcagcttcaagctcttcaacatccaggtcaaggaggtcacgcagaatg | |
| | aaggcaccaagaccatcgccaataacctcaccagcaccatccaggtgtttacggactcggag | |
| | taccagctgccgtacgttctcggctctgcccaccagggctgcctgcctccgttcccggcggac | |
| | gtgttcatgattccccagtacggctacctaacactcaacaacggtagtcaggccgtgggacgc | |
| | tcctccttctactgcctggaatactttccttcgcagatgctgagaaccggcaacaacttccagttt | |
| | acttacaccttcgaggacgtgcctttccacagcagctacgcccacagccagagcttggaccg | |
| | gctgatgaatcctctgattgaccagtacctgtactacttgtctcggactcaaacaacaggaggc | |
| | acggcaaatacgcagactctgggcttcagccaaggtgggcctaatacaatggccaatcaggc | |
| | aaagaactggctgccaggaccctgttaccgccaacaacgcgtctcaacgacaaccgggcaa | |
| | aacaacaatagcaactttgcctggactgctgggaccaaataccatctgaatggaagaaattcat | |
| | tggctaatcctggcatcgctatggcaacacacaaagacgacgaggagcgtttttttcccagtaa | |
| | cgggatcctgatttttggcaaacaaaatgctgccagagacaatgcggattacagcgatgtcat | |
| | gctcaccagcgaggaagaaatcaaaaccactaaccctgtggctacagaggaatacggtatc | |
| | gtggcagataacttgcagcagcaaaacacggctcctcaaattggaactgtcaacagccaggg | |
| | ggccttacccggtatggtctggcagaaccgggacgtgtacctgcagggtcccatctgggcca | |
| | agattcctcacacggacggcaacttccacccgtctccgctgatgggcggctttggcctgaaac | |
| | atcctccgcctcagatcctgatcaagaacacgcctgtacctgcggatcctccgaccaccttcaa | |
| | ccagtcaaagctgaactctttcatcacgcaatacagcaccggacaggtcagcgtggaaattga | |
| | atgggagctgcagaaggaaaacagcaagcgctggaaccccgagatccagtacacctccaa | |
| | ctactacaaatctacaagtgtggactttgctgttaatacagaaggcgtgtactctgaaccccgcc | |
| | ccattggcacccgttacctcacccgtaatctgtaa | |
| AAV8 (protein) | MAADGYLPDWLEDNLSEGIREWWALKPGAPKPKANQQK | 4 |
| | QDDGRGLVLPGYKYLGPFNGLDKGEPVNAADAAALEHDK | |
| | AYDQQLQAGDNPYLRYNHADAEFQERLQEDTSFGGNLGR | |
| | AVFQAKKRVLEPLGLVEEGAKTAPGKKRPVEPSPQRSPDSS | |
| | TGIGKKGQQPARKRLNFGQTGDSESVPDPQPLGEPPAAPSG | |
| | VGPNTMAAGGGAPMADNNEGADGVGSSSGNWHCDSTWL | |
| | GDRVITTSTRTWALPTYNNHLYKQISNGTSGGATNDNTYF | |
| | GYSTPWGYFDFNRFHCHFSPRDWQRLINNNWGFRPKRLSF | |
| | KLFNIQVKEVTQNEGTKTIANNLTSTIQVFTDSEYQLPYVL | |
| | GSAHQGCLPPFPADVFMIPQYGYLTLNNGSQAVGRSSFYC | |
| | LEYFPSQMLRTGNNFQFTYTFEDVPFHSSYAHSQSLDRLM | |
| | NPLIDQYLYYLSRTQTTGGTANTQTLGFSQGGPNTMANQA | |
| | KNWLPGPCYRQQRVSTTTGQNNNSNFAWTAGTKYHLNGR | |
| | NSLANPGIAMATHKDDEERFFPSNGILIFGKQNAARDNADY | |
| | SDVMLTSEEEIKTTNPVATEEYGIVADNLQQQNTAPQIGTV | |
| | NSQGALPGMVWQNRDVYLQGPIWAKIPHTDGNFHPSPLM | |
| | GGFGLKHPPPQILIKNTPVPADPPTTFNQSKLNSFITQYSTG | |
| | QVSVEIEWELQKENSKRWNPEIQYTSNYYKSTSVDFAVNT | |
| | EGVYSEPRPIGTRYLTRNL | |
| AAV-DJ | atggctgccgatggttatcttccagattggctcgaggacactctctctgaaggaataagacagt | 5 |
| (nucleotide) | ggtggaagctcaaacctggcccaccaccaccaaagcccgcagagcggcataaggacgac | |
| | agcaggggtcttgtgcttcctgggtacaagtacctcggacccttcaacggactcgacaaggg | |
| | agagccggtcaacgaggcagacgccgcggccctcgagcacgacaaagcctacgaccggc | |
| | agctcgacagcggagacaacccgtacctcaagtacaaccacgccgacgccgagttccagg | |
| | agcggctcaaagaagatacgtcttttgggggcaacctcgggcgagcagtcttccaggccaaa | |
| | aagaggcttcttgaacctcttggtctggttgaggaagcggctaagacggctcctggaaagaag | |
| | aggcctgtagagcactctcctgtggagccagactcctcctcgggaaccggaaaggcgggcc | |
| | agcagcctgcaagaaaaagattgaattttggtcagactggagacgcagactcagtcccagac | |
| | cctcaaccaatcggagaacctcccgcagccccctcaggtgtgggatctcttacaatggctgca | |
| | ggcggtggcgcaccaatggcagacaataacgagggcgccgacggagtgggtaattcctcg | |
| | ggaaattggcattgcgattccacatggatgggcgacagagtcatcaccaccagcacccgaac | |
| | ctgggccctgcccacctacaacaaccacctctacaagcaaatctccaacagcacatctggag | |
| | gatcttcaaatgacaacgcctacttcggctacagcaccccctgggggtattttgactttaacaga | |
| | ttccactgccacttttcaccacgtgactggcagcgactcatcaacaacaactggggattccggc | |
| | ccaagagactcagcttcaagctcttcaacatccaggtcaaggaggtcacgcagaatgaaggc | |
| | accaagaccatcgccaataacctcaccagcaccatccaggtgtttacggactcggagtacca | |
| | gctgccgtacgttctcggctctgcccaccagggctgcctgcctccgttcccggcggacgtgtt | |
| | catgattccccagtacggctacctaacactcaacaacggtagtcaggccgtgggacgctcctc | |
| | cttctactgcctggaatactttccttcgcagatgctgagaaccggcaacaacttccagtttactta | |
| | caccttcgaggacgtgcctttccacagcagctacgcccacagccagagcttggaccggctga | |
| | tgaatcctctgattgaccagtacctgtactacttgtctcggactcaaacaacaggaggcacgac | |
| | aaatacgcagactctgggcttcagccaaggtgggcctaatacaatggccaatcaggcaaaga | |
| | actggctgccaggaccctgttaccgccagcagcgagtatcaaagacatctgcggataacaac | |
| | aacagtgaatactcgtggactggagctaccaagtaccacctcaatggcagagactctctggtg | |
| | aatccgggcccggccatggcaagccacaaggacgatgaagaaaagttttttcctcagagcg | |
| | gggttctcatctttgggaagcaaggctcagagaaaacaaatgtggacattgaaaaggtcatga | |
| | ttacagacgaagaggaaatcaggacaaccaatcccgtggctacggagcagtatggttctgtat | |
| | ctaccaacctccagagaggcaacagacaagcagctaccgcagatgtcaacacacaaggcgt | |
| | tcttccaggcatggtctggcaggacagagatgtgtaccttcaggggcccatctgggcaaagat | |
| | tccacacacggacggacattttcacccctctcccctcatgggtggattcggacttaaacaccct | |
| | ccgcctcagatcctgatcaagaacacgcctgtacctgcggatcctccgaccaccttcaaccag | |
| | tcaaagctgaactctttcatcacccagtattctactggccaagtcagcgtggagatcgagtggg | |
| | agctgcagaaggaaaacagcaagcgctggaaccccgagatccagtacacctccaactacta | |
| | caaatctacaagtgtggactttgctgttaatacagaaggcgtgtactctgaaccccgccccattg | |
| | gcacccgttacctcacccgtaatctgtaa | |
| AAV-DJ | MAADGYLPDWLEDTLSEGIRQWWKLKPGPPPPKPAERHK | 6 |
| (protein) | DDSRGLVLPGYKYLGPFNGLDKGEPVNEADAAALEHDKA | |
| | YDRQLDSGDNPYLKYNHADAEFQERLKEDTSFGGNLGRA | |
| | VFQAKKRLLEPLGLVEEAAKTAPGKKRPVEHSPVEPDSSSG | |
| | TGKAGQQPARKRLNFGQTGDADSVPDPQPIGEPPAAPSGV | |
| | GSLTMAAGGGAPMADNNEGADGVGNSSGNWHCDSTWM | |
| | GDRVITTSTRTWALPTYNNHLYKQISNSTSGGSSNDNAYFG | |
| | YSTPWGYFDFNRFHCHFSPRDWQRLINNNWGFRPKRLSFK | |
| | LFNIQVKEVTQNEGTKTIANNLTSTIQVFTDSEYQLPYVLGS | |
| | AHQGCLPPFPADVFMIPQYGYLTLNNGSQAVGRSSFYCLE | |
| | YFPSQMLRTGNNFQFTYTFEDVPFHSSYAHSQSLDRLMNPL | |
| | IDQYLYYLSRTQTTGGTTNTQTLGFSQGGPNTMANQAKN | |
| | WLPGPCYRQQRVSKTSADNNNSEYSWTGATKYHLNGRDS | |
| | LVNPGPAMASHKDDEEKFFPQSGVLIFGKQGSEKTNVDIEK | |
| | VMITDEEEIRTTNPVATEQYGSVSTNLQRGNRQAATADVN | |
| | TQGVLPGMVWQDRDVYLQGPIWAKIPHTDGHFHPSPLMG | |
| | GFGLKHPPPQILIKNTPVPADPPTTFNQSKLNSFITQYSTGQ | |
| | VSVEIEWELQKENSKRWNPEIQYTSNYYKSTSVDFAVNTE | |
| | GVYSEPRPIGTRYLTRNL | |
| SL65 | atggctgccgatggttatcttccagattggctcgaggacactctctctgaaggcattcgcgagt | 7 |
| (nucleotide) | ggtgggcgctgaaacctggagctccacaacccaaggccaaccaacagcatcaggacaacg | |
| | gcaggggtcttgtgcttcctgggtacaagtacctcggacccttcaacggactcgacaaggga | |
| | gagccggtcaacgaggcagacgccgcggccctcgagcacgacaaggcctacgacaagca | |
| | gctcgagcagggggacaacccgtacctcaagtacaaccacgccgacgccgagtttcagga | |
| | gcgtctgcaagaagatacgtcttttgggggcaacctcgggcgagcagtcttccaggccaaga | |
| | agcgggttctcgaacctctcggtctggttgaggaaggcgctaagacggctcctggaaagaag | |
| | agaccggtagagccgtcacctcagcgttcccccgactcctccacgggcatcggcaagaaag | |
| | gccagcagcccgccagaaagagactcaatttcggtcagactggcgactcagagtcagtccc | |
| | cgaccctcaacctctcggagaacctccagcagcgccctctagtgtgggatctggtacagtgg | |
| | ctgcaggcggtggcgcaccaatggcagacaataacgaaggtgccgacggagtgggtaatg | |
| | cctcaggaaattggcattgcgattccacatggctgggcgacagagtcattaccaccagcaccc | |
| | gaacctgggccctgcccacctacaacaaccacctctacaagcaaatctccagccaatcagga | |
| | gcttcaaacgacaaccactactttggctacagcaccccttgggggtattttgactttaacagattc | |
| | cactgccacttctcaccacgtgactggcagcgactcattaacaacaactggggattccggccc | |
| | aagagactcaacttcaagctcttcaacatccaagtcaaggaggtcacgacgaatgatggcgtc | |
| | acgaccatcgctaataaccttaccagcacggttcaagtcttctcggactcggagtaccagttgc | |
| | cgtacgtcctcggctctgcgcaccagggctgcctccctccgttcccggcggacgtgttcatga | |
| | ttccccagtacggctacctaacactcaacaacggtagtcaggccgtgggacgctcctcctttta | |
| | ctgcctggaatatttcccatcgcagatgctgagaacgggcaataactttgagttcagctacagc | |
| | ttcgaggacgtgcctttccacagcagctacgcacacagccagagcttggaccgactgatgaa | |
| | tcctctcattgaccagtacctgtactacttatccagaactcagtccacaggaggaactcaaggta | |
| | cccagcaattgttattttctcaagctgggcctgcaaacatgtcggctcaggccaagaactggct | |
| | gcctggaccttgctaccggcagcagcgagtctccacgacactgtcgcaaaacaacaacagc | |
| | aactttgcttggactggtgccaccaaatatcacctgaacggcagaaactcgttggttaatcccg | |
| | gcgtcgccatggcaactcacaaggacgacgaggaccgctttttcccatccagcggagtcctg | |
| | atttttggaaaaactggagcaactaacaaaactacattggaaaatgtgttaatgacaaatgaag | |
| | aagaaattcgtcctactaatcctgtagccacggaagaatacgggatagtcagcagcaacttac | |
| | aagcggctaatactgcagcccagacacaagttgtcaacaaccagggagccttacctggcatg | |
| | gtctggcagaaccgggacgtgtacctgcagggtcccatttgggccaaaattcctcacacagat | |
| | ggacactttcacccgtctcctcttatgggcggctttggactcaagaacccgcctcctcagatcct | |
| | catcaaaaacacgcctgttcctgcgaatcctccggcggagttttcagctacaaagtttgcttcatt | |
| | catcacccagtattccacaggacaagtgagcgtggagattgaatgggagctgcagaaagaa | |
| | aacagcaaacgctggaatcccgaagtgcagtatacatctaactatgcaaaatctgccaacgtt | |
| | gatttcactgtggacaacaatggactttatactgagcctcgccccattggcacccgttaccttac | |
| | ccgtcccctgtaa | |
| SL65 | MAADGYLPDWLEDTLSEGIREWWALKPGAPQPKANQQHQ | 8 |
| (protein) | DNGRGLVLPGYKYLGPFNGLDKGEPVNEADAAALEHDKA | |
| | YDKQLEQGDNPYLKYNHADAEFQERLQEDTSFGGNLGRA | |
| | VFQAKKRVLEPLGLVEEGAKTAPGKKRPVEPSPQRSPDSST | |
| | GIGKKGQQPARKRLNFGQTGDSESVPDPQPLGEPPAAPSSV | |
| | GSGTVAAGGGAPMADNNEGADGVGNASGNWHCDSTWL | |
| | GDRVITTSTRTWALPTYNNHLYKQISSQSGASNDNHYFGYS | |
| | TPWGYFDFNRFHCHFSPRDWQRLINNNWGFRPKRLNFKLF | |
| | NIQVKEVTTNDGVTTIANNLTSTVQVFSDSEYQLPYVLGSA | |
| | HQGCLPPFPADVFMIPQYGYLTLNNGSQAVGRSSFYCLEYF | |
| | PSQMLRTGNNFEFSYSFEDVPFHSSYAHSQSLDRLMNPLID | |
| | QYLYYLSRTQSTGGTQGTQQLLFSQAGPANMSAQAKNWL | |
| | PGPCYRQQRVSTTLSQNNNSNFAWTGATKYHLNGRNSLV | |
| | NPGVAMATHKDDEDRFFPSSGVLIFGKTGATNKTTLENVL | |
| | MTNEEEIRPTNPVATEEYGIVSSNLQAANTAAQTQVVNNQ | |
| | GALPGMVWQNRDVYLQGPIWAKIPHTDGHFHPSPLMGGF | |
| | GLKNPPPQILIKNTPVPANPPAEFSATKFASFITQYSTGQVSV | |
| | EIEWELQKENSKRWNPEVQYTSNYAKSANVDFTVDNNGL | |
| | YTEPRPIGTRYLTRPL | |
| AAV-NP59 | atggctgccgatggttatcttccagattggctcgaggacactctctctgaaggaataagacagt | 9 |
| (nucleotide) | ggtggaagctcaaacctggcccaccaccaccaaagcccgcagagcggcataaggacgac | |
| | agcaggggtcttgtgcttcctgggtacaagtacctcggacccttcaacggactcgacaaggg | |
| | agagccggtcaacgaggcagacgccgcggccctcgagcacgacaaagcctacgaccggc | |
| | agctcgacagcggagacaacccgtacctcaagtacaaccacgccgacgcggagtttcagga | |
| | gcgccttaaagaagatacgtcttttgggggcaacctcggacgagcagtcttccaggcgaaaa | |
| | agagggttcttgaacctctgggcctggttgaggaacctgttaagacggctccgggaaaaaag | |
| | aggccggtagagcactctcctgtggagccagactcctcctcgggaaccggcaagacaggcc | |
| | agcagcccgctaaaaagagactcaattttggtcagactggcgactcagagtcagtcccagac | |
| | cctcaacctctcggagaaccaccagcagccccctctggtctgggaactaatacgatggctac | |
| | aggcagtggcgcaccaatggcagacaataacgagggtgccgatggagtgggtaattcctca | |
| | ggaaattggcattgcgattcccaatggctgggcgacagagtcatcaccaccagcacccgaac | |
| | ctgggccctgcccacctacaacaaccatctctacaagcaaatctccagccaatcaggagcttc | |
| | aaacgacaaccactactttggctacagcaccccttgggggtattttgactttaacagattccact | |
| | gccacttctcaccacgtgactggcagcgactcattaacaacaactggggattccggcccaag | |
| | aaactcagcttcaagctcttcaacatccaagttaaagaggtcacgcagaacgatggcacgac | |
| | gactattgccaataaccttaccagcacggttcaagtgtttactgactcggagtaccagctcccgt | |
| | acgtcctcggctcggcgcatcaaggatgcctcccgccgttcccagcagacgtcttcatggtgc | |
| | cacagtatggatacctcaccctgaacaacgggagtcaggcagtaggacgctcttcattttactg | |
| | cctggagtactttccttctcagatgctgcgtaccggaaacaactttaccttcagctacacttttga | |
| | ggacgttcctttccacagcagctacgctcacagccagagtctggaccgtctcatgaatcctctc | |
| | atcgaccagtacctgtattacttgagcagaacaaacactccaagtggaaccaccacgcagtca | |
| | aggcttcagttttctcaggccggagcgagtgacattcgggaccagtctaggaactggcttcct | |
| | ggaccctgttaccgccagcagcgagtatcaaagacatctgcggataacaacaacagtgaata | |
| | ctcgtgggctggagctaccaagtaccacctcaatggcagagactctctggtgaatccgggcc | |
| | cggccatggcaagccacaaggacgatgaagaaaagttttttcctcagagcggggttctcatct | |
| | ttgggaagcaaggctcagagaaaacaaatgtggacattgaaaaggtcatgattacagacgaa | |
| | gaggaaatcaggacaaccaatcccgtggctacggagcagtatggttctgtatctaccaacctc | |
| | cagagaggcaacagacaagcagctaccgcagatgtcgacacacaaggcgttcttccaggca | |
| | tggtatggcaggacagagatgtgtaccttcagggacccatctgggcaaagattccacacacg | |
| | gacggacattttcacccctctcccctcatgggtggattcggacttaaacaccctcctccacagat | |
| | tctcatcaagaacaccccggtacctgcgaatccttcgaccaccttcagtgcggcaaagtttgct | |
| | tccttcatcacacagtactcaacgggacaggtcagcgtggagatcgagtgggagctgcagaa | |
| | ggaaaacagcaaacgctggaatcccgaaattcagtacacttccaactacaacaagtctgttaat | |
| | gtggactttactgtggacactaatggcgtgtattcagagcctcgccccattggcaccagatacc | |
| | tgactcgtaatctgtaa | |
| AAV-NP59 | MAADGYLPDWLEDTLSEGIRQWWKLKPGPPPPKPAERHK | 10 |
| (protein) | DDSRGLVLPGYKYLGPFNGLDKGEPVNEADAAALEHDKA | |
| | YDRQLDSGDNPYLKYNHADAEFQERLKEDTSFGGNLGRA | |
| | VFQAKKRVLEPLGLVEEPVKTAPGKKRPVEHSPVEPDSSSG | |
| | TGKTGQQPAKKRLNFGQTGDSESVPDPQPLGEPPAAPSGL | |
| | GTNTMATGSGAPMADNNEGADGVGNSSGNWHCDSQWLG | |
| | DRVITTSTRTWALPTYNNHLYKQISSQSGASNDNHYFGYST | |
| | PWGYFDFNRFHCHFSPRDWQRLINNNWGFRPKKLSFKLFN | |
| | IQVKEVTQNDGTTTIANNLTSTVQVFTDSEYQLPYVLGSAH | |
| | QGCLPPFPADVFMVPQYGYLTLNNGSQAVGRSSFYCLEYF | |
| | PSQMLRTGNNFTFSYTFEDVPFHSSYAHSQSLDRLMNPLID | |
| | QYLYYLSRTNTPSGTTTQSRLQFSQAGASDIRDQSRNWLPG | |
| | PCYRQQRVSKTSADNNNSEYSWAGATKYHLNGRDSLVNP | |
| | GPAMASHKDDEEKFFPQSGVLIFGKQGSEKTNVDIEKVMIT | |
| | DEEEIRTTNPVATEQYGSVSTNLQRGNRQAATADVDTQGV | |
| | LPGMVWQDRDVYLQGPIWAKIPHTDGHFHPSPLMGGFGL | |
| | KHPPPQILIKNTPVPANPSTTFSAAKFASFITQYSTGQVSVEI | |
| | EWELQKENSKRWNPEIQYTSNYNKSVNVDFTVDINGVYS | |
| | EPRPIGTRYLTRNL | |
| AAV2 | atggctgccgatggttatcttccagattggctcgaggacactctctctgaaggaataagacagt | 11 |
| (nucleotide) | ggtggaagctcaaacctggcccaccaccaccaaagcccgcagagcggcataaggacgac | |
| | agcaggggtcttgtgcttcctgggtacaagtacctcggacccttcaacggactcgacaaggg | |
| | agagccggtcaacgaggcagacgccgcggccctcgagcacgacaaagcctacgaccggc | |
| | agctcgacagcggagacaacccgtacctcaagtacaaccacgccgacgcggagtttcagga | |
| | gcgccttaaagaagatacgtcttttgggggcaacctcggacgagcagtcttccaggcgaaaa | |
| | agagggttcttgaacctctgggcctggttgaggaacctgttaagacggctccgggaaaaaag | |
| | aggccggtagagcactctcctgtggagccagactcctcctcgggaaccggaaaggcgggc | |
| | cagcagcctgcaagaaaaagattgaattttggtcagactggagacgcagactcagtacctga | |
| | cccccagcctctcggacagccaccagcagccccctctggtctgggaactaatacgatggcta | |
| | caggcagtggcgcaccaatggcagacaataacgagggcgccgacggagtgggtaattcct | |
| | cgggaaattggcattgcgattccacatggatgggcgacagagtcatcaccaccagcacccga | |
| | acctgggccctgcccacctacaacaaccacctctacaaacaaatttccagccaatcaggagc | |
| | ctcgaacgacaatcactactttggctacagcaccccttgggggtattttgacttcaacagattcc | |
| | actgccacttttcaccacgtgactggcaaagactcatcaacaacaactggggattccgaccca | |
| | agagactcaacttcaagctctttaacattcaagtcaaagaggtcacgcagaatgacggtacga | |
| | cgacgattgccaataaccttaccagcacggttcaggtgtttactgactcggagtaccagctccc | |
| | gtacgtcctcggctcggcgcatcaaggatgcctcccgccgttcccagcagacgtcttcatggt | |
| | gccacagtatggatacctcaccctgaacaacgggagtcaggcagtaggacgctcttcatttta | |
| | ctgcctggagtactttccttctcagatgctgcgtaccggaaacaactttaccttcagctacactttt | |
| | gaggacgttcctttccacagcagctacgctcacagccagagtctggaccgtctcatgaatcct | |
| | ctcatcgaccagtacctgtattacttgagcagaacaaacactccaagtggaaccaccacgcag | |
| | tcaaggcttcagttttctcaggccggagcgagtgacattcgggaccagtctaggaactggcttc | |
| | ctggaccctgttaccgccagcagcgagtatcaaagacatctgcggataacaacaacagtgaa | |
| | tactcgtggactggagctaccaagtaccacctcaatggcagagactctctggtgaatccgggc | |
| | ccggccatggcaagccacaaggacgatgaagaaaagttttttcctcagagcggggttctcatc | |
| | tttgggaagcaaggctcagagaaaacaaatgtggacattgaaaaggtcatgattacagacga | |
| | agaggaaatcaggacaaccaatcccgtggctacggagcagtatggttctgtatctaccaacct | |
| | ccagagaggcaacagacaagcagctaccgcagatgtcaacacacaaggcgttcttccaggc | |
| | atggtctggcaggacagagatgtgtaccttcaggggcccatctgggcaaagattccacacac | |
| | ggacggacattttcacccctctcccctcatgggtggattcggacttaaacaccctcctccacag | |
| | attctcatcaagaacaccccggtacctgcgaatccttcgaccaccttcagtgcggcaaagtttg | |
| | cttccttcatcacacagtactccacgggacaggtcagcgtggagatcgagtgggagctgcag | |
| | aaggaaaacagcaaacgctggaatcccgaaattcagtacacttccaactacaacaagtctgtt | |
| | aatgtggactttactgtggacactaatggcgtgtattcagagcctcgccccattggcaccagat | |
| | acctgactcgtaatctgtaa | |
| AAV2 | MAADGYLPDWLEDTLSEGIRQWWKLKPGPPPPKPAERHK | 12 |
| (protein) | DDSRGLVLPGYKYLGPFNGLDKGEPVNEADAAALEHDKA | |
| | YDRQLDSGDNPYLKYNHADAEFQERLKEDTSFGGNLGRA | |
| | VFQAKKRVLEPLGLVEEPVKTAPGKKRPVEHSPVEPDSSSG | |
| | TGKAGQQPARKRLNFGQTGDADSVPDPQPLGQPPAAPSGL | |
| | GTNTMATGSGAPMADNNEGADGVGNSSGNWHCDSTWM | |
| | GDRVITTSTRTWALPTYNNHLYKQISSQSGASNDNHYFGYS | |
| | TPWGYFDFNRFHCHFSPRDWQRLINNNWGFRPKRLNFKLF | |
| | NIQVKEVTQNDGTTTIANNLTSTVQVFTDSEYQLPYVLGSA | |
| | HQGCLPPFPADVFMVPQYGYLTLNNGSQAVGRSSFYCLEY | |
| | FPSQMLRTGNNFTFSYTFEDVPFHSSYAHSQSLDRLMNPLI | |
| | DQYLYYLSRTNTPSGTTTQSRLQFSQAGASDIRDQSRNWLP | |
| | GPCYRQQRVSKTSADNNNSEYSWTGATKYHLNGRDSLVN | |
| | PGPAMASHKDDEEKFFPQSGVLIFGKQGSEKTNVDIEKVMI | |
| | TDEEEIRTTNPVATEQYGSVSTNLQRGNRQAATADVNTQG | |
| | VLPGMVWQDRDVYLQGPIWAKIPHTDGHFHPSPLMGGFG | |
| | LKHPPPQILIKNTPVPANPSTTFSAAKFASFITQYSTGQVSVE | |
| | IEWELQKENSKRWNPEIQYTSNYNKSVNVDFTVDTNGVYS | |
| | EPRPIGTRYLTRNL | |
| AAV5 | ATGTCTTTTGTTGATCACCCTCCAGATTGGTTGGAAGAA | 13 |
| (nucleotide) | GTTGGTGAAGGTCTTCGCGAGTTTTTGGGCCTTGAAGCG | |
| | GGCCCACCGAAACCAAAACCCAATCAGCAGCATCAAGA | |
| | TCAAGCCCGTGGTCTTGTGCTGCCTGGTTATAACTATCTC | |
| | GGACCCGGAAACGGTCTCGATCGAGGAGAGCCTGTCAA | |
| | CAGGGCAGACGAGGTCGCGCGAGAGCACGACATCTCGT | |
| | ACAACGAGCAGCTTGAGGCGGGAGACAACCCCTACCTC | |
| | AAGTACAACCACGCGGACGCCGAGTTTCAGGAGAAGCT | |
| | CGCCGACGACACATCCTTCGGGGGAAACCTCGGAAAGG | |
| | CAGTCTTTCAGGCCAAGAAAAGGGTTCTCGAACCTTTTG | |
| | GCCTGGTTGAAGAGGGTGCTAAGACGGCCCCTACCGGA | |
| | AAGCGGATAGACGACCACTTTCCAAAAAGAAAGAAGGC | |
| | TCGGACCGAAGAGGACTCCAAGCCTTCCACCTCGTCAGA | |
| | CGCCGAAGCTGGACCCAGCGGATCCCAGCAGCTGCAAA | |
| | TCCCAGCCCAACCAGCCTCAAGTTTGGGAGCTGATACAA | |
| | TGTCTGCGGGAGGTGGCGGCCCATTGGGCGACAATAACC | |
| | AAGGTGCCGATGGAGTGGGCAATGCCTCGGGAGATTGG | |
| | CATTGCGATTCCACGTGGATGGGGGACAGAGTCGTCACC | |
| | AAGTCCACCCGAACCTGGGTGCTGCCCAGCTACAACAAC | |
| | CACCAGTACCGAGAGATCAAAAGCGGCTCCGTCGACGG | |
| | AAGCAACGCCAACGCCTACTTTGGATACAGCACCCCCTG | |
| | GGGGTACTTTGACTTTAACCGCTTCCACAGCCACTGGAG | |
| | CCCCCGAGACTGGCAAAGACTCATCAACAACTACTGGG | |
| | GCTTCAGACCCCGGTCCCTCAGAGTCAAAATCTTCAACA | |
| | TTCAAGTCAAAGAGGTCACGGTGCAGGACTCCACCACCA | |
| | CCATCGCCAACAACCTCACCTCCACCGTCCAAGTGTTTA | |
| | CGGACGACGACTACCAGCTGCCCTACGTCGTCGGCAACG | |
| | GGACCGAGGGATGCCTGCCGGCCTTCCCTCCGCAGGTCT | |
| | TTACGCTGCCGCAGTACGGTTACGCGACGCTGAACCGCG | |
| | ACAACACAGAAAATCCCACCGAGAGGAGCAGCTTCTTCT | |
| | GCCTAGAGTACTTTCCCAGCAAGATGCTGAGAACGGGCA | |
| | ACAACTTTGAGTTTACCTACAACTTTGAGGAGGTGCCCT | |
| | TCCACTCCAGCTTCGCTCCCAGTCAGAACCTGTTCAAGC | |
| | TGGCCAACCCGCTGGTGGACCAGTACTTGTACCGCTTCG | |
| | TGAGCACAAATAACACTGGCGGAGTCCAGTTCAACAAG | |
| | AACCTGGCCGGGAGATACGCCAACACCTACAAAAACTG | |
| | GTTCCCGGGGCCCATGGGCCGAACCCAGGGCTGGAACCT | |
| | GGGCTCCGGGGTCAACCGCGCCAGTGTCAGCGCCTTCGC | |
| | CACGACCAATAGGATGGAGCTCGAGGGCGCGAGTTACC | |
| | AGGTGCCCCCGCAGCCGAACGGCATGACCAACAACCTC | |
| | CAGGGCAGCAACACCTATGCCCTGGAGAACACTATGATC | |
| | TTCAACAGCCAGCCGGCGAACCCGGGCACCACCGCCAC | |
| | GTACCTCGAGGGCAACATGCTCATCACCAGCGAGAGCG | |
| | AGACGCAGCCGGTGAACCGCGTGGCGTACAACGTCGGC | |
| | GGGCAGATGGCCACCAACAACCAGAGCTCCACCACTGC | |
| | CCCCGCGACCGGCACGTACAACCTCCAGGAAATCGTGCC | |
| | CGGCAGCGTGTGGATGGAGAGGGACGTGTACCTCCAAG | |
| | GACCCATCTGGGCCAAGATCCCAGAGACGGGGGCGCAC | |
| | TTTCACCCCTCTCCGGCCATGGGCGGATTCGGACTCAAA | |
| | CACCCACCGCCCATGATGCTCATCAAGAACACGCCTGTG | |
| | CCCGGAAATATCACCAGCTTCTCGGACGTGCCCGTCAGC | |
| | AGCTTCATCACCCAGTACAGCACCGGGCAGGTCACCGTG | |
| | GAGATGGAGTGGGAGCTCAAGAAGGAAAACTCCAAGAG | |
| | GTGGAACCCAGAGATCCAGTACACAAACAACTACAACG | |
| | ACCCCCAGTTTGTGGACTTTGCCCCGGACAGCACCGGGG | |
| | AATACAGAACCACCAGACCTATCGGAACCCGATACCTTA | |
| | CCCGACCCCTTTAA | |
| AAV5 | MSFVDHPPDWLEEVGEGLREFLGLEAGPPKPKPNQQHQDQ | 14 |
| (protein) | ARGLVLPGYNYLGPGNGLDRGEPVNRADEVAREHDISYNE | |
| | QLEAGDNPYLKYNHADAEFQEKLADDTSFGGNLGKAVFQ | |
| | AKKRVLEPFGLVEEGAKTAPTGKRIDDHFPKRKKARTEED | |
| | SKPSTSSDAEAGPSGSQQLQIPAQPASSLGADTMSAGGGGP | |
| | LGDNNQGADGVGNASGDWHCDSTWMGDRVVTKSTRTW | |
| | VLPSYNNHQYREIKSGSVDGSNANAYFGYSTPWGYFDFNR | |
| | FHSHWSPRDWQRLINNYWGFRPRSLRVKIFNIQVKEVTVQ | |
| | DSTTTIANNLTSTVQVFTDDDYQLPYVVGNGTEGCLPAFPP | |
| | QVFTLPQYGYATLNRDNTENPTERSSFFCLEYFPSKMLRTG | |
| | NNFEFTYNFEEVPFHSSFAPSQNLFKLANPLVDQYLYRFVS | |
| | TNNTGGVQFNKNLAGRYANTYKNWFPGPMGRTQGWNLG | |
| | SGVNRASVSAFATTNRMELEGASYQVPPQPNGMTNNLQG | |
| | SNTYALENTMIFNSQPANPGTTATYLEGNMLITSESETQPV | |
| | NRVAYNVGGQMATNNQSSTTAPATGTYNLQEIVPGSVWM | |
| | ERDVYLQGPIWAKIPETGAHFHPSPAMGGFGLKHPPPMMLI | |
| | KNTPVPGNITSFSDVPVSSFITQYSTGQVTVEMEWELKKEN | |
| | SKRWNPEIQYTNNYNDPQFVDFAPDSTGEYRTTRPIGTRYL | |
| | TRPL | |
| AAV6 | atggctgccgatggttatcttccagattggctcgaggacaacctctctgagggcattcgcgagt | 15 |
| (nucleotide) | ggtgggacttgaaacctggagccccgaaacccaaagccaaccagcaaaagcaggacgac | |
| | ggccggggtctggtgcttcctggctacaagtacctcggacccttcaacggactcgacaaggg | |
| | ggagcccgtcaacgcggcggatgcagcggccctcgagcacgacaaggcctacgaccagc | |
| | agctcaaagcgggtgacaatccgtacctgcggtataaccacgccgacgccgagtttcagga | |
| | gcgtctgcaagaagatacgtcttttgggggcaacctcgggcgagcagtcttccaggccaaga | |
| | agagggttctcgaaccttttggtctggttgaggaaggtgctaagacggctcctggaaagaaac | |
| | gtccggtagagcagtcgccacaagagccagactcctcctcgggcattggcaagacaggcca | |
| | gcagcccgctaaaaagagactcaattttggtcagactggcgactcagagtcagtccccgacc | |
| | cacaacctctcggagaacctccagcaacccccgctgctgtgggacctactacaatggcttca | |
| | ggcggtggcgcaccaatggcagacaataacgaaggcgccgacggagtgggtaatgcctca | |
| | ggaaattggcattgcgattccacatggctgggcgacagagtcatcaccaccagcacccgaac | |
| | atgggccttgcccacctataacaaccacctctacaagcaaatctccagtgcttcaacgggggc | |
| | cagcaacgacaaccactacttcggctacagcaccccctgggggtattttgatttcaacagattc | |
| | cactgccatttctcaccacgtgactggcagcgactcatcaacaacaattggggattccggccc | |
| | aagagactcaacttcaagctcttcaacatccaagtcaaggaggtcacgacgaatgatggcgtc | |
| | acgaccatcgctaataaccttaccagcacggttcaagtcttctcggactcggagtaccagttgc | |
| | cgtacgtcctcggctctgcgcaccagggctgcctccctccgttcccggcggacgtgttcatga | |
| | ttccgcagtacggctacctaacgctcaacaatggcagccaggcagtgggacggtcatcctttt | |
| | actgcctggaatatttcccatcgcagatgctgagaacgggcaataactttaccttcagctacac | |
| | cttcgaggacgtgcctttccacagcagctacgcgcacagccagagcctggaccggctgatg | |
| | aatcctctcatcgaccagtacctgtattacctgaacagaactcagaatcagtccggaagtgccc | |
| | aaaacaaggacttgctgtttagccgggggtctccagctggcatgtctgttcagcccaaaaact | |
| | ggctacctggaccctgttaccggcagcagcgcgtttctaaaacaaaaacagacaacaacaac | |
| | agcaactttacctggactggtgcttcaaaatataaccttaatgggcgtgaatctataatcaaccct | |
| | ggcactgctatggcctcacacaaagacgacgaagacaagttctttcccatgagcggtgtcatg | |
| | atttttggaaaggagagcgccggagcttcaaacactgcattggacaatgtcatgatcacagac | |
| | gaagaggaaatcaaagccactaaccccgtggccaccgaaagatttgggactgtggcagtca | |
| | atctccagagcagcagcacagaccctgcgaccggagatgtgcatgttatgggagccttacct | |
| | ggaatggtgtggcaagacagagacgtatacctgcagggtcctatttgggccaaaattcctcac | |
| | acggatggacactttcacccgtctcctctcatgggcggctttggacttaagcacccgcctcctc | |
| | agatcctcatcaaaaacacgcctgttcctgcgaatcctccggcagagttttcggctacaaagttt | |
| | gcttcattcatcacccagtattccacaggacaagtgagcgtggagattgaatgggagctgcag | |
| | aaagaaaacagcaaacgctggaatcccgaagtgcagtatacatctaactatgcaaaatctgcc | |
| | aacgttgatttcactgtggacaacaatggactttatactgagcctcgccccattggcacccgtta | |
| | cctcacccgtcccctgtaa | |
| AAV6 | MAADGYLPDWLEDNLSEGIREWWDLKPGAPKPKANQQK | 16 |
| (protein) | QDDGRGLVLPGYKYLGPFNGLDKGEPVNAADAAALEHDK | |
| | AYDQQLKAGDNPYLRYNHADAEFQERLQEDTSFGGNLGR | |
| | AVFQAKKRVLEPFGLVEEGAKTAPGKKRPVEQSPQEPDSSS | |
| | GIGKTGQQPAKKRLNFGQTGDSESVPDPQPLGEPPATPAAV | |
| | GPTTMASGGGAPMADNNEGADGVGNASGNWHCDSTWLG | |
| | DRVITTSTRTWALPTYNNHLYKQISSASTGASNDNHYFGYS | |
| | TPWGYFDFNRFHCHFSPRDWQRLINNNWGFRPKRLNFKLF | |
| | NIQVKEVTTNDGVTTIANNLTSTVQVFSDSEYQLPYVLGSA | |
| | HQGCLPPFPADVFMIPQYGYLTLNNGSQAVGRSSFYCLEYF | |
| | PSQMLRTGNNFTFSYTFEDVPFHSSYAHSQSLDRLMNPLID | |
| | QYLYYLNRTQNQSGSAQNKDLLFSRGSPAGMSVQPKNWL | |
| | PGPCYRQQRVSKTKTDNNNSNFTWTGASKYNLNGRESIINP | |
| | GTAMASHKDDEDKFFPMSGVMIFGKESAGASNTALDNVM | |
| | ITDEEEIKATNPVATERFGTVAVNLQSSSTDPATGDVHVMG | |
| | ALPGMVWQDRDVYLQGPIWAKIPHTDGHFHPSPLMGGFG | |
| | LKHPPPQILIKNTPVPANPPAEFSATKFASFITQYSTGQVSVE | |
| | IEWELQKENSKRWNPEVQYTSNYAKSANVDFTVDNNGLY | |
| | TEPRPIGTRYLTRPL | |
| AAV9 | atggctgccgatggttatcttccagattggctcgaggacaaccttagtgaaggaattcgcgagt | 17 |
| (nucleotide) | ggtgggctttgaaacctggagcccctcaacccaaggcaaatcaacaacatcaagacaacgct | |
| | cgaggtcttgtgcttccgggttacaaataccttggacccggcaacggactcgacaaggggga | |
| | gccggtcaacgcagcagacgcggcggccctcgagcacgacaaggcctacgaccagcagc | |
| | tcaaggccggagacaacccgtacctcaagtacaaccacgccgacgccgagttccaggagc | |
| | ggctcaaagaagatacgtcttttgggggcaacctcgggcgagcagtcttccaggccaaaaag | |
| | aggcttcttgaacctcttggtctggttgaggaagcggctaagacggctcctggaaagaagag | |
| | gcctgtagagcagtctcctcaggaaccggactcctccgcgggtattggcaaatcgggtgcac | |
| | agcccgctaaaaagagactcaatttcggtcagactggcgacacagagtcagtcccagaccct | |
| | caaccaatcggagaacctcccgcagccccctcaggtgtgggatctcttacaatggcttcaggt | |
| | ggtggcgcaccagtggcagacaataacgaaggtgccgatggagtgggtagttcctcgggaa | |
| | attggcattgcgattcccaatggctgggggacagagtcatcaccaccagcacccgaacctgg | |
| | gccctgcccacctacaacaatcacctctacaagcaaatctccaacagcacatctggaggatct | |
| | tcaaatgacaacgcctacttcggctacagcaccccctgggggtattttgacttcaacagattcc | |
| | actgccacttctcaccacgtgactggcagcgactcatcaacaacaactggggattccggccta | |
| | agcgactcaacttcaagctcttcaacattcaggtcaaagaggttacggacaacaatggagtca | |
| | agaccatcgccaataaccttaccagcacggtccaggtcttcacggactcagactatcagctcc | |
| | cgtacgtgctcgggtcggctcacgagggctgcctcccgccgttcccagcggacgttttcatga | |
| | ttcctcagtacgggtatctgacgcttaatgatggaagccaggccgtgggtcgttcgtccttttact | |
| | gcctggaatatttcccgtcgcaaatgctaagaacgggtaacaacttccagttcagctacgagttt | |
| | gagaacgtacctttccatagcagctacgctcacagccaaagcctggaccgactaatgaatcca | |
| | ctcatcgaccaatacttgtactatctctcaaagactattaacggttctggacagaatcaacaaac | |
| | gctaaaattcagtgtggccggacccagcaacatggctgtccagggaagaaactacatacctg | |
| | gacccagctaccgacaacaacgtgtctcaaccactgtgactcaaaacaacaacagcgaattt | |
| | gcttggcctggagcttcttcttgggctctcaatggacgtaatagcttgatgaatcctggacctgct | |
| | atggccagccacaaagaaggagaggaccgtttctttcctttgtctggatctttaatttttggcaaa | |
| | caaggaactggaagagacaacgtggatgcggacaaagtcatgataaccaacgaagaagaa | |
| | attaaaactactaacccggtagcaacggagtcctatggacaagtggccacaaaccaccagag | |
| | tgcccaagcacaggcgcagaccggctgggttcaaaaccaaggaatacttccgggtatggttt | |
| | ggcaggacagagatgtgtacctgcaaggacccatttgggccaaaattcctcacacggacgg | |
| | caactttcacccttctccgctgatgggagggtttggaatgaagcacccgcctcctcagatcctc | |
| | atcaaaaacacacctgtacctgcggatcctccaacggccttcaacaaggacaagctgaactct | |
| | ttcatcacccagtattctactggccaagtcagcgtggagatcgagtgggagctgcagaagga | |
| | aaacagcaagcgctggaacccggagatccagtacacttccaactattacaagtctaataatgtt | |
| | gaatttgctgttaatactgaaggtgtatatagtgaaccccgccccattggcaccagatacctgac | |
| | tcgtaatctgtaa | |
| AAV9 | MAADGYLPDWLEDNLSEGIREWWALKPGAPQPKANQQH | 18 |
| (protein) | QDNARGLVLPGYKYLGPGNGLDKGEPVNAADAAALEHDK | |
| | AYDQQLKAGDNPYLKYNHADAEFQERLKEDTSFGGNLGR | |
| | AVFQAKKRLLEPLGLVEEAAKTAPGKKRPVEQSPQEPDSS | |
| | AGIGKSGAQPAKKRLNFGQTGDTESVPDPQPIGEPPAAPSG | |
| | VGSLTMASGGGAPVADNNEGADGVGSSSGNWHCDSQWL | |
| | GDRVITTSTRTWALPTYNNHLYKQISNSTSGGSSNDNAYFG | |
| | YSTPWGYFDFNRFHCHFSPRDWQRLINNNWGFRPKRLNFK | |
| | LFNIQVKEVTDNNGVKTIANNLTSTVQVFTDSDYQLPYVL | |
| | GSAHEGCLPPFPADVFMIPQYGYLTLNDGSQAVGRSSFYCL | |
| | EYFPSQMLRTGNNFQFSYEFENVPFHSSYAHSQSLDRLMNP | |
| | LIDQYLYYLSKTINGSGQNQQTLKFSVAGPSNMAVQGRNY | |
| | IPGPSYRQQRVSTTVTQNNNSEFAWPGASSWALNGRNSLM | |
| | NPGPAMASHKEGEDRFFPLSGSLIFGKQGTGRDNVDADKV | |
| | MITNEEEIKTTNPVATESYGQVATNHQSAQAQAQTGWVQ | |
| | NQGILPGMVWQDRDVYLQGPIWAKIPHTDGNFHPSPLMGG | |
| | FGMKHPPPQILIKNTPVPADPPTAFNKDKLNSFITQYSTGQV | |
| | SVEIEWELQKENSKRWNPEIQYTSNYYKSNNVEFAVNTEG | |
| | VYSEPRPIGTRYLTRNL | |
| AAV-rh74 | atggctgccgatggttatcttccagattggctcgaggacaacctctctgagggcattcgcgagt | 19 |
| (nucleotide) | ggtgggacctgaaacctggagccccgaaacccaaagccaaccagcaaaagcaggacaac | |
| | ggccggggtctggtgcttcctggctacaagtacctcggacccttcaacggactcgacaaggg | |
| | ggagcccgtcaacgcggcggacgcagcggccctcgagcacgacaaggcctacgaccagc | |
| | agctccaagcgggtgacaatccgtacctgcggtataatcacgccgacgccgagtttcaggag | |
| | cgtctgcaagaagatacgtcttttgggggcaacctcgggcgcgcagtcttccaggccaaaaa | |
| | gcgggttctcgaacctctgggcctggttgaatcgccggttaagacggctcctggaaagaaga | |
| | gaccggtagagccatcaccccagcgctctccagactcctctacgggcatcggcaagaaagg | |
| | ccagcagcccgcaaaaaagagactcaattttgggcagactggcgactcagagtcagtcccc | |
| | gaccctcaaccaatcggagaaccaccagcaggcccctctggtctgggatctggtacaatggc | |
| | tgcaggcggtggcgctccaatggcagacaataacgaaggcgccgacggagtgggtagttc | |
| | ctcaggaaattggcattgcgattccacatggctgggcgacagagtcatcaccaccagcaccc | |
| | gcacctgggccctgcccacctacaacaaccacctctacaagcaaatctccaacgggacctcg | |
| | ggaggaagcaccaacgacaacacctacttcggctacagcaccccctgggggtattttgacttc | |
| | aacagattccactgccacttttcaccacgtgactggcagcgactcatcaacaacaactgggga | |
| | ttccggcccaagaggctcaacttcaagctcttcaacatccaagtcaaggaggtcacgcagaat | |
| | gaaggcaccaagaccatcgccaataaccttaccagcacgattcaggtctttacggactcgga | |
| | ataccagctcccgtacgtgctcggctcggcgcaccagggctgcctgcctccgttcccggcgg | |
| | acgtcttcatgattcctcagtacgggtacctgactctgaacaatggcagtcaggctgtgggccg | |
| | gtcgtccttctactgcctggagtactttccttctcaaatgctgagaacgggcaacaactttgaatt | |
| | cagctacaacttcgaggacgtgcccttccacagcagctacgcgcacagccagagcctggac | |
| | cggctgatgaaccctctcatcgaccagtacttgtactacctgtcccggactcaaagcacgggc | |
| | ggtactgcaggaactcagcagttgctattttctcaggccgggcctaacaacatgtcggctcag | |
| | gccaagaactggctacccggtccctgctaccggcagcaacgcgtctccacgacactgtcgc | |
| | agaacaacaacagcaactttgcctggacgggtgccaccaagtatcatctgaatggcagagac | |
| | tctctggtgaatcctggcgttgccatggctacccacaaggacgacgaagagcgattttttccat | |
| | ccagcggagtcttaatgtttgggaaacagggagctggaaaagacaacgtggactatagcagc | |
| | gtgatgctaaccagcgaggaagaaataaagaccaccaacccagtggccacagaacagtac | |
| | ggcgtggtggccgataacctgcaacagcaaaacgccgctcctattgtaggggccgtcaatag | |
| | tcaaggagccttacctggcatggtgtggcagaaccgggacgtgtacctgcagggtcccatct | |
| | gggccaagattcctcatacggacggcaactttcatccctcgccgctgatgggaggctttggac | |
| | tgaagcatccgcctcctcagatcctgattaaaaacacacctgttcccgcggatcctccgaccac | |
| | cttcaatcaggccaagctggcttctttcatcacgcagtacagtaccggccaggtcagcgtgga | |
| | gatcgagtgggagctgcagaaggagaacagcaaacgctggaacccagagattcagtacact | |
| | tccaactactacaaatctacaaatgtggactttgctgtcaatactgagggtacttattccgagcct | |
| | cgccccattggcacccgttacctcacccgtaatctgtaa | |
| AAV-rh74 | MAADGYLPDWLEDNLSEGIREWWDLKPGAPKPKANQQK | 20 |
| (protein) | QDNGRGLVLPGYKYLGPFNGLDKGEPVNAADAAALEHDK | |
| | AYDQQLQAGDNPYLRYNHADAEFQERLQEDTSFGGNLGR | |
| | AVFQAKKRVLEPLGLVESPVKTAPGKKRPVEPSPQRSPDSS | |
| | TGIGKKGQQPAKKRLNFGQTGDSESVPDPQPIGEPPAGPSG | |
| | LGSGTMAAGGGAPMADNNEGADGVGSSSGNWHCDSTWL | |
| | GDRVITTSTRTWALPTYNNHLYKQISNGTSGGSTNDNTYFG | |
| | YSTPWGYFDFNRFHCHFSPRDWQRLINNNWGFRPKRLNFK | |
| | LFNIQVKEVTQNEGTKTIANNLTSTIQVFTDSEYQLPYVLGS | |
| | AHQGCLPPFPADVFMIPQYGYLTLNNGSQAVGRSSFYCLE | |
| | YFPSQMLRTGNNFEFSYNFEDVPFHSSYAHSQSLDRLMNPL | |
| | IDQYLYYLSRTQSTGGTAGTQQLLFSQAGPNNMSAQAKN | |
| | WLPGPCYRQQRVSTTLSQNNNSNFAWTGATKYHLNGRDS | |
| | LVNPGVAMATHKDDEERFFPSSGVLMFGKQGAGKDNVDY | |
| | SSVMLTSEEEIKTTNPVATEQYGVVADNLQQQNAAPIVGA | |
| | VNSQGALPGMVWQNRDVYLQGPIWAKIPHTDGNFHPSPL | |
| | MGGFGLKHPPPQILIKNTPVPADPPTTFNQAKLASFITQYST | |
| | GQVSVEIEWELQKENSKRWNPEIQYTSNYYKSTNVDFAVN | |
| | TEGTYSEPRPIGTRYLTRNL | |

### AAV Structure and Function

Adeno-associated virus (AAV) is a parvovirus composed of an icosahedral protein capsid and a single-stranded DNA genome. The AAV viral capsid comprises three subunits, VP1, VP2, and VP3 and two inverted terminal repeat (ITR) regions, which are at the ends of the genomic sequence. The ITRs serve as origins of replication and play a role in viral packaging. The viral genome also comprises rep and cap genes, which are associated with replication and capsid packaging, respectively. In most wild-type AAV, the rep gene encodes four proteins required for viral replication, Rep78, Rep68, Rep52, and Rep40. The cap gene encodes the capsid subunits as well as the assembly activating protein (AAP), which promotes assembly of viral particles. AAVs are generally replication-deficient, requiring the presence of a helper virus or helper virus functions (e.g., herpes simplex virus (HSV) and/or adenovirus (AdV)) in order to replicate within an infected cell. For example, in some embodiments AAVs require adenoviral E1A, E2A, E4, and VA RNA genes in order to replicate within a host cell.

### Recombinant AAV

In general, recombinant AAV (rAAV) vectors can comprise many of the same elements found in wild-type AAVs, including similar capsid sequences and structures, as well as polynucleotide sequences that are not of AAV origin (e.g., a polynucleotide heterologous to AAV). In some embodiments, rAAVs will replace native, wild-type AAV sequences with polynucleotide sequences encoding a payload. For example, in some embodiments an rAAV will comprise polynucleotide sequences encoding one or more genes intended for therapeutic purposes (e.g., for gene therapy). rAAVs may be modified to remove one or more wild-type viral coding sequences. For example, rAAVs may be engineered to comprise only one ITR, and/or one or more genes necessary for packaging (e.g., rep and cap genes) than would be found in a wild type AAV. Gene expression with rAAVs is generally limited to one or more genes that total 5 kb or less, as larger sequences are not efficiently packaged within the viral capsid. In some embodiments, two or more rAAVs can be used to provide portions of a larger payload, for example, in order to provide an entire coding sequence for a gene that would normally be too large to fit in a single AAV.

Among other things, the present disclosure provides viral vectors comprising one or more polypeptides described herein. In some embodiments, rAAVs may comprise one or more capsid proteins (e.g., one or more capsid proteins from AAV1, AAV2, AAV3, AAV4, AAV5, AAV6, AAV7, AAV8, AAV9, AAV10, AAV11, AAVC11.01, AAVC11.02, AAVC11.03, AAVC11.04, AAVC11.05, AAVC11.06, AAVC11.07, AAVC11.08, AAVC11.09, AAVC11.10, AAVC11.11 (referred to interchangeably herein as sL65), AAVC11.12, AAVC11.13, AAVC11.14, AAVC11.15, AAVC11.16, AAVC11.17, AAVC11.18, AAVC11.19, AAV-DJ, AAV-LK03, AAV-LK19, AAVrh.74, AAVrh.10, AAVhu.37, AAVrh.K, AAVrh.39, AAV12, AAV13, AAVrh.8,), avian AAV, bovine AAV, canine AAV, equine AAV, primate AAV, non-primate AAV, ovine AAV, a hybrid AAV (e.g., an AAV comprising one more sequences of one AAV subtype and one or more sequences of a second subtype), and/or an AAV comprising a mutant AAV capsid protein or a chimeric AAV capsid (e.g., a capsid with polynucleotide sequences derived from two or more different serotypes of AAV). In some embodiments, rAAVs may comprise one or more polynucleotide sequences encoding a gene or nucleic acid of interest (e.g., a gene for treatment of a genetic disease/disorder and/or an inhibitory nucleic acid sequence).

In some embodiments, a recombinant AAV vector may comprise at least one ITR. In some embodiments, a recombinant AAV vector comprises two ITRs. In some embodiments, a recombinant AAV vector comprises a 5′ ITR. In some embodiments, a recombinant AAV vector comprises a 3′ ITR. In some embodiments, a recombinant AAV vector comprises an AAV2 ITR. In some embodiments, a recombinant AAV vector comprises a portion of an AAV2 ITR. In some embodiments, a recombinant AAV vector comprises an ITR having at least 80%, 85%, 90%, 95%, 99%, or 100% sequence identity to an AAV2 ITR. In some embodiments, a recombinant AAV vector comprises an ITR having 90%, 95%, 99%, or 100% sequence identity to one of the exemplary sequences in Table 2 below.

TABLE 2
Exemplary ITR sequences

| ITR Description | Sequence (5′ to 3′) | Size (bp) | SEQ ID NO |
|---|---|---|---|
| Wild type ITR, flip | AGGAACCCCTAGTGATGGAGTTGGCCA | 145 | 21 |
| orientation | CTCCCTCTCTGCGCGCTCGCTCGCTCAC | | |
| | TGAGGCCGCCCGGGCAAAGCCCGGGC | | |
| | GTCGGGCGACCTTTGGTCGCCCGGCCT | | |
| | CAGTGAGCGAGCGAGCGCGCAGAGAG | | |
| | GGAGTGGCCAA | | |
| Wild type ITR, flop | AGGAACCCCTAGTGATGGAGTTGGCCA | 145 | 22 |
| orientation | CTCCCTCTCTGCGCGCTCGCTCGCTCAC | | |
| | TGAGGCCGGGCGACCAAAGGTCGCCC | | |
| | GACGCCCGGGCTTTGCCCGGGCGGCCT | | |
| | CAGTGAGCGAGCGAGCGCGCAGAGAG | | |
| | GGAGTGGCCAA | | |
| B loop deleted ITR | AGGAACCCCTAGTGATGGAGTTGGCCA | 123 | 23 |
| | CTCCCTCTCTGCGCGCTCGCTCGCTCAC | | |
| | TGAGGCCGCCCGGGCTTTGCCCGGGCG | | |
| | GCCTCAGTGAGCGAGCGAGCGCGCAG | | |
| | AGAGGGAGTGGCCAA | | |
| C loop deleted ITR | AGGAACCCCTAGTGATGGAGTTGGCCA | 123 | 24 |
| | CTCCCTCTCTGCGCGCTCGCTCGCTCAC | | |
| | TGAGGCCGGGCGACCAAAGGTCGCCC | | |
| | GGCCTCAGTGAGCGAGCGAGCGCGCA | | |
| | GAGAGGGAGTGGCCAA | | |
| A region deleted | AGGAACCCCTAGTGATGGAGTTGGCCA | 130 | 25 |
| ITR | CTCCCTCTCTGCGCGCTCGCTCGCTCAC | | |
| | TGAGGCCGGGCGACCAAAGGTCGCCC | | |
| | GACGCCCGGGCTTTGCCCGGGCGGCCT | | |
| | CAGTGAGCGAGCGAGCGCGCAG | | |
| D region deleted | CTGCGCGCTCGCTCGCTCACTGAGGCC | 105 | 26 |
| ITR | GCCCGGGCAAAGCCCGGGCGTCGGGC | | |
| | GACCTTTGGTCGCCCGGCCTCAGTGAG | | |
| | CGAGCGAGCGCGCAGAGAGGGAGTG | | |

AAV vectors may be capable of being replicated in an infected host cell (replication competent) or incapable of being replicated in an infected host cell (replication incompetent). A replication competent AAV (rcAAV) requires the presence of one or more functional AAV packaging genes. Recombinant AAV vectors are generally designed to be replication-incompetent in mammalian cells, in order to reduce the possibility that rcAAV are generated through recombination with sequences encoding AAV packaging genes. In some embodiments, rAAV vector preparations as described herein are designed to comprise few, if any, rcAAV vectors. In some embodiments, rAAV vector preparations comprise less than about 1 rcAAV per 10² rAAV vectors. In some embodiments, rAAV vector preparations comprise less than about 1 rcAAV per 10⁴ rAAV vectors. In some embodiments, rAAV vector preparations comprise less than about 1 rcAAV per 10⁸ rAAV vectors. In some embodiments, rAAV vector preparations comprise less than about 1 rcAAV per 10¹² rAAV vectors. In some embodiments, rAAV vector preparations comprise no rcAAV vectors.

### Lipid Nanoparticles (LNPs)

Lipid nanoparticles (LNPs) are delivery systems that, among other things, can achieve intra-cellular delivery of nucleic acids in intact form, allowing for biological changes including therapeutic effects. In one aspect, lipid nanoparticles are lipid compositions that comprise at least one lipid. In some embodiments, lipid nanoparticles may further comprise at least one nucleic acid (e.g., DNA, RNA, and the like). In some embodiments, lipid nanoparticles may comprise a therapeutic nucleic acid (e.g., DNA, RNA, and the like) encapsulated in a lipid portion of the nanoparticle.

The present disclosure provides for compositions that comprise lipid nanoparticles. In some embodiments, lipid nanoparticles comprise one or more components. In some embodiments, lipid nanoparticles comprise one or more components such as ionizable lipids, sterols, conjugate-linker lipids, and phospholipids. In some embodiments, lipid nanoparticles may comprise one or more compounds described herein. Among other things, the present disclosure describes that selection and combination of one or more of the components as described herein may impact characteristics of lipid nanoparticles such as diameter, pKa, stabilization, and ionizability.

Among other things, the present disclosure describes that selection and combination of one or more of the components as described herein impacts functional activity of lipid nanoparticles such as tropism, stabilization, and delivery efficacy. For example, the present disclosure describes that a combination of components may better suit delivery of a particular payload (e.g., DNA (e.g., plasmid DNA, linearized DNA (e.g., linearized, and dimerized DNA (CELID))) as compared to an appropriate reference (e.g., alternative nucleic acid sequences, proteins, and the like).

In some embodiments, lipid nanoparticles may be or comprise a structure produced by an available method (See, Jayaraman et al., Angewandte Chemie (International Ed. In English), 2012; Li et al. PLOS One, 2013; Kulkarni et al. Nanomedicine: Nanotechnology, Biology, and Medicine, 2017; US 2019/0240345; WO 2019/089828; WO/2021/102411; WO/2019/046809; WO/2018089540, each of which is incorporated herein by reference in its entirety). In light of the teachings provided herein, those skilled in the art would be aware of the possibility that alternative, available lipid nanoparticles could be substituted for those described herein.

### Ionizable Lipids

In some embodiments, lipid nanoparticles comprise one or more ionizable lipids as described herein. In some embodiments, an ionizable lipid may include an amine-containing group on the head group. In some embodiments, an ionizable lipid is or comprises a compound described herein (e.g., a compound of Formula II, Formula III, or Formula IV). In some embodiments, an ionizable lipid is present in a lipid nanoparticle (LNP) preparation from about 30 mole percent to about 70 mole percent, based on total moles of components of the lipid nanoparticle. In some embodiments, the ionizable lipid is present from about 33 mol percent to about 60 mole percent, based on total moles of components of the lipid nanoparticle. In some embodiments, the ionizable lipid is present from about 34 mol percent to about 55 mole percent, based on total moles of components of the lipid nanoparticle. In some embodiments, the ionizable lipid is present from about 33 mol percent to about 51 mole percent, based on total moles of components of the lipid nanoparticle. In some embodiments, the ionizable lipid is present at about 34.7 mole percent, based on total moles of components of the lipid nanoparticle. In some embodiments, the ionizable lipid is present at about 50 mole percent, based on total moles of components of the lipid nanoparticle.

### Sterols

In some embodiments, lipid nanoparticles comprise one or more sterols as described herein. In some embodiments, a sterol is a cholesterol, or a variant or derivative thereof. In some embodiments, a cholesterol is modified. In some embodiments, a cholesterol is an oxidized cholesterol. In some embodiments, a cholesterol is esterified cholesterol. Unmodified cholesterol can be acted upon by enzymes to form variants that are side-chain or ring oxidized. In some embodiments, a cholesterol can be oxidized on the beta-ring structure or on the hydrocarbon tail structure. In some embodiments, a sterol is a phytosterol. Exemplary sterols that are considered for use in the disclosed lipid nanoparticles include but are not limited to 25-hydroxycholesterol (25-OH), 20α-hydroxycholesterol (20α-OH), 27-hydroxycholesterol, 6-keto-5α-hydroxycholesterol, 7-ketocholesterol, 7β-hydroxycholesterol, 7α-hydroxycholesterol, 7β-25-dihydroxycholesterol, beta-sitosterol, stigmasterol, brassicasterol, campesterol, or combinations thereof. In some embodiments, a side-chain oxidized cholesterol can enhance cargo delivery relative to other cholesterol variants. In some embodiments, a cholesterol is an unmodified cholesterol.

In some embodiments, the LNP composition comprises from about 20 mol percent to about 50 mol percent sterol. In some embodiments, the LNP composition comprises about 38 mol percent sterol. In some embodiments, the LNP composition comprises about 38.5 mol percent sterol. In some embodiments, LNP composition comprises about 33.8 mol percent cholesterol.

### Conjugate-Linker Lipids

In some embodiments, lipid nanoparticles comprise one or more conjugate-linker lipids as described herein. In some embodiments, a conjugate-linker lipid is or comprises a polyethylene glycol (PEG)-lipid or PEG-modified lipid. In some embodiments, PEG or PEG-modified lipids may be alternately referred to as PEGylated lipids or PEG-lipids. Inclusion of a PEGylating lipid can be used to enhance lipid nanoparticle colloidal stability in vitro and circulation time in vivo. In some embodiments, the PEGylation is reversible in that the PEG moiety is gradually released in blood circulation. Exemplary PEG-lipids include but are not limited to PEG conjugated to saturated or unsaturated alkyl chains having a length of C6-C20. PEG-modified phosphatidylethanolamines, PEG-modified phosphatidic acids, PEG-modified ceramides (PEG-CER), PEG-modified dialkylamines, PEG-modified diacylglycerols (PEG-DAG), PEG-modified dialkylglycerols, and mixtures thereof. For example, in some embodiments, a PEG lipid may be PEG-c-DOMG, PEG-DMG, PEG-DLPE, PEG-DMPE, PEG-DPPE, PEG-DSG or a PEG-DSPE lipid.

In some embodiments, the conjugate-linker lipid comprises a polyethylene glycol lipid. In some embodiments, the conjugate-linker lipid comprises DiMystyrlGlycerol (DMG), 1,2-Dipalmitoyl-rac-glycerol, methoxypolyethylene Glycol (DPG-PEG), or 1,2-Distearoyl-rac-glycero-3-methylpolyoxyethylene (DSG-PEG). In some embodiments, the conjugate-linker lipid has an average molecular mass from about 500 Da to about 5000 Da. In some embodiments, the conjugate-linker lipid has an average molecular mass of about 2000 Da. In some embodiments, the LNP composition comprises from about 0 mol percent to about 5 mol percent conjugate-linker lipid. In some embodiments, the LNP composition comprises about 1.5 mol percent conjugate-linker lipid. In some embodiments, the LNP composition comprises about 3 mol percent conjugate-linker lipid.

### Phospholipids

In some embodiments, lipid nanoparticles comprise one or more phospholipids as described herein. In some embodiments, one or more phospholipids may assemble into one or more lipid bilayers. In some embodiments, one or more phospholipids may include a phospholipid moiety. In some embodiments, one or more phospholipids may include one or more fatty acid moieties. In some embodiments, one or more phospholipids may include a phospholipid moiety and one or more fatty acid moieties. In some embodiments, a phospholipid is or comprises a compound described herein (e.g., a compound of Formula I). In some embodiments, a phospholipid moiety includes but is not limited to phosphatidyl choline, phosphatidyl ethanolamine, phosphatidyl glycerol, phosphatidyl serine, phosphatidic acid, 2-lysophosphatidyl choline, and sphingomyelin. In some embodiments, a fatty acid moiety includes but is not limited to lauric acid, myristic acid, myristoleic acid, palmitic acid, palmitoleic acid, stearic acid, oleic acid, linoleic acid, alphalinolenic acid, erucic acid, phytanic acid, arachidic acid, arachidonic acid, eicosapentaenoic acid, behenic acid, docosapentaenoic acid, and docosahexaenoic acid. Non-natural species including natural species with modifications and substitutions including branching, oxidation, cyclization, and alkynes are also contemplated. For example, a phospholipid may be functionalized with or cross-linked to one or more alkynes (e.g., an alkenyl group in which one or more double bonds is replaced with a triple bond). Under appropriate reaction conditions, an alkyne group may undergo a copper-catalyzed cycloaddition upon exposure to an azide. Such reactions may be useful in functionalizing a lipid bilayer of a nanoparticle composition to facilitate membrane permeation or cellular recognition or in conjugating a nanoparticle composition to a useful component such as a targeting or imaging moiety (e.g., a dye).

Exemplary phospholipids include but are not limited to 1,2-distearoyl-snglycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycerophosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycerophosphocholine (DUPC), 1-palmitoyl-2-oleoyl-sn-glycero-3-phosphocholine (POPC), 1,2-di-0-octadecenyl-sn-glycero-3-phosphocholine (18:0 Diether PC), 1-oleoyl-2-cholesterylhemisuccinoy 1-sn-glycero-3-phosphocholine (OChemsPC), 1-hexadecyl snglycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), dipalmitoylphosphatidylglycerol (DPPG), palmitoyloleoylphosphatidylethanolamine (POPE), distearoyl-phosphatidyl-ethanolamine (DSPE), dipalmitoyl phosphatidyl ethanolamine (DPPE), dimyristoylphosphoethanolamine (DMPE), 1-stearoyl-2-oleoyl-phosphatidy ethanolamine (SOPE), 1-stearoyl-2 oleoylphosphatidylcholine (SOPC), sphingomyelin, phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine (LPE), or combinations thereof. In some embodiments, a phospholipid is DSPC. In some embodiments, a phospholipid is DMPC.

In some embodiments, the phospholipid comprises 1,2-dioleoyl-sn-glycero-3-phosphoethanolamine-N-(succinyl) (succinyl PE), 1,2-distearoyl-sn-glycero-3-phosphocholine (DSPC), cholesterol, 1,2-distearoyl-sn-glycero-3-phosphoethanolamine (DSPE), 1,2-dipalmitoyl-sn-glycero-3-phosphoethanolamine-N-(succinyl) (succinyl-DPPE), 1,2-dioleoyl-sn-glycero-3-phosphoethanolamine (DOPE), 1,2-dimyristoyl-sn-glycero-3-phosphocholine (DMPC), 1,2-dipalmitoyl-sn-glycero-3-phosphocholine (DPPC), or a combination thereof.

### Diameter

Among other things, the present disclosure describes compositions having an average hydrodynamic diameter from about 30 to about 220 nm. In some embodiments, lipid nanoparticles described herein can have an average hydrodynamic diameter from about 30 to about 220 nm. In some embodiments, lipid nanoparticles described herein can have an average hydrodynamic diameter that is about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 155 nm, 160 nm, 165 nm, 170 nm, 175 nm, 180 nm, 185 nm, 190 nm, 195 nm, 200 nm, 205 nm, 210 nm, 215 nm, 220 nm, or any range having endpoints defined by any two of the aforementioned values. For example, in some embodiments, lipid nanoparticles described herein have an average hydrodynamic diameter from between 50 nm to 200 nm.

pKa

Among other things, the present disclosure describes compositions, preparations, nanoparticles, and/or nanomaterials that have a pKa from about 5 to about 9.

In some embodiments, lipid nanoparticles described herein have a pKa from about 5 to about 9. In some embodiments, lipid nanoparticles described herein have a pKa that is about 5.0, 5.5, 6.0, 6.5, 7.0, 7.5, 8.0, 8.5, or any range having endpoints defined by any two of the aforementioned values. In some embodiments, lipid nanoparticles described herein have a pKa that is about 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, or any range having endpoints defined by any two of the aforementioned values.

### Exemplary LNP Compositions

The present invention provides for compositions that comprise lipid nanoparticles. In some embodiments, a lipid nanoparticle composition comprises about 30 mole percent to about 70 mole percent ionizable lipid, about 5 mole percent to about 25 mole percent phospholipid, about 25 mole percent to about 45 mole percent cholesterol, and about 0 mole percent to about 5 mole percent conjugate-linker lipid. In some embodiments, a lipid nanoparticle composition comprises about 50 mole percent ionizable lipid, about 20 mole percent phospholipid, about 39 mole percent cholesterol, and about 1 mole percent conjugate-linker lipid.

In some embodiments, a lipid nanoparticle composition comprises about 30 mole percent to about 70 mole percent ionizable lipid of Formula II, Formula III, or Formula IV, about 5 mole percent to about 25 mole percent DSPC, about 25 mole percent to about 45 mole percent cholesterol, and about 0 mole percent to about 5 mole percent lipid PEG, based on the total moles of these ingredients.

In some embodiments, a lipid nanoparticle (LNP) preparation comprises a mass ratio of (the total of one or more ionizable lipids, sterols, conjugate-linker lipids, and phospholipids):DNA from about 2:1 and 50:1. In some embodiments, a LNP preparation comprises a mass ratio of (the total of one or more ionizable lipids, sterols, conjugate-linker lipids, and phospholipids):DNA of about 2:1, about 3:1, about 4:1, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, about 20:1, about 21:1, about 22:1, about 23:1, about 24:1, about 25:1, about 26:1, about 27:1, about 28:1, about 29:1, about 30:1, about 31:1, about 32:1, about 33:1, about 34:1, about 35:1, about 36:1, about 37:1, about 38:1, about 39:1, about 40:1, about 41:1, about 42:1, about 43:1, about 44:1, about 45:1, about 46:1, about 47:1, about 48:1, about 49:1, about 50:1.

### Exemplary Compounds

Among other things, the present disclosure describes compositions, and/or nanoparticles that comprise one or more compounds as described herein.

In some embodiments, the present disclosure provides lipid nanoparticles comprising compounds comprising one or more partially unsaturated lipid groups. In some embodiments, provided lipid nanoparticles comprise compounds comprising one or more trimethylated amine groups. In some embodiments, provided lipid nanoparticles comprise compounds comprising one or more dimethylated amine groups.

In some embodiments, the present disclosure provides a compound of Formula I:

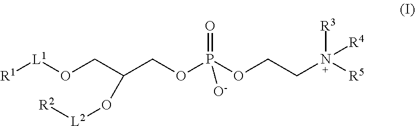

or a pharmaceutically acceptable salt thereof, wherein:
each of L¹ and L² is independently a covalent bond, —C(O)—, —OC—(O)—, or an optionally substituted bivalent saturated or unsaturated, straight or branched C₁-C₁₂ hydrocarbon chain;

each of R¹ and R² is independently an optionally substituted group selected from saturated or unsaturated, straight, or branched C₁-C₂₀ hydrocarbon chain wherein 1-3 methylene units are optionally replaced with —O—; and
each of R³, R⁴, and R⁵ is independently a hydrogen or an optionally substituted bivalent saturated or unsaturated, straight, or branched C₁-C₁₀ hydrocarbon chain.

In some embodiments, the present disclosure provides a compound of Formula II:

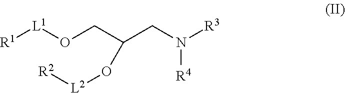

or a pharmaceutically acceptable salt thereof, wherein:
each of L¹ and L² is independently a covalent bond, —C(O)—, —OC—(O)—, or an optionally substituted bivalent saturated or unsaturated, straight or branched C₁-C₁₂ hydrocarbon chain;
each of R¹ and R² is independently an optionally substituted group selected from saturated or unsaturated, straight, or branched C₁-C₂₀ hydrocarbon chain wherein 1-3 methylene units are optionally replaced with —O—; and
each of R³ and R⁴ is independently a hydrogen or an optionally substituted bivalent saturated or unsaturated, straight, or branched C₁-C₁₀ hydrocarbon chain.

In some embodiments, the present disclosure provides a compound of Formula III:

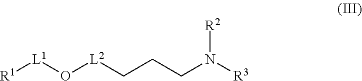

or a pharmaceutically acceptable salt thereof, wherein:
each of L¹ and L² is independently a covalent bond, —C(O)—, —OC—(O)—, or an optionally substituted bivalent saturated or unsaturated, straight or branched C₁-C₁₂ hydrocarbon chain;
R¹ is independently a hydrogen, or an optionally substituted group selected from saturated or unsaturated, straight, or branched C₁-C₂₀ hydrocarbon chain wherein 1-3 methylene units are optionally replaced with —O—; and
each of R² and R³ is independently a hydrogen or an optionally substituted bivalent saturated or unsaturated, straight, or branched C₁-C₁₀ hydrocarbon chain.

In some embodiments, the present disclosure provides a compound of Formula IV:

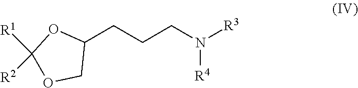

or a pharmaceutically acceptable salt thereof, wherein:
each of R¹ and R² is independently an optionally substituted group selected from saturated or unsaturated, straight, or branched C₁-C₂₀ hydrocarbon chain wherein 1-3 methylene units are optionally replaced with —O—; and
each of R³ and R⁴ is independently a hydrogen or an optionally substituted bivalent saturated or unsaturated, straight, or branched C₁-C₁₀ hydrocarbon chain.

### Targeted Nucleases

There are multiple DNA-targeted nucleases understood in the art. In some embodiments, nucleases described herein may comprise any polypeptide or protein sequence capable of inducing a single- or double-stranded DNA break. In some embodiments, nucleases described herein may be wild-type proteins, fusion proteins, engineered proteins, or variants thereof. In some embodiments, nucleases may be selected from a class of enzymes comprising: TALENs, ZFNs, Meganucleases, TALE Nickases, Zinc Finger Nickases, and Cas nucleases (e.g., Staphylococcus aureus Cas9 (saCas9), Streptococcus pyogenes (spCas9), AZ Nuclease, HF1-Cas9, HF2-Cas9, or HiFi-Cas9). In some embodiments, nucleases are combined with other GENERIDE™ components in order to produce targeted integration of a transgene at a target integration site. In some embodiments, nucleases are combined with other GENERIDE™ components in order to produce targeted integration of a transgene at a target integration site that is distal from a nuclease cut site.

### TALENs (Transcription Activator-Like Effector Nucleases)

TALENs are restriction enzymes that comprise a DNA binding domain (transcription activator-like (TAL) effector domain) directly or indirectly fused to a nuclease. TAL effectors (TALEs) comprise a highly conserved set of 33-34 amino acid sequence repeats, which may be engineered to bind to specific DNA sequences within a cellular genome. Fusion of engineered TALEs to a nuclease cleavage domain (e.g., FokI nuclease) can produce TALE Nucleases (TALENs) capable of targeting specific DNA sequence to produce DNA breaks. Combinations of TALENs (e.g., TALEN pairs) can be used to produce targeted, double-stranded breaks (DSBs) in a DNA sequence.

In some embodiments, TALENs may be engineered to target specific DNA sequences and induce DSBs. In some embodiments, one or more TALENs (e.g., pairs of TALENs) may be combined to induce DSBs through binding to target DNA sequences on strands in genomic DNA. In some embodiments, certain features of TALENs (e.g., DNA-binding regions, linker regions, nuclease fusion region) may be designed or optimized to produce improved DNA binding and/or cutting efficiency. In some embodiments, certain features of TALENs (e.g., DNA-binding regions, linker regions, nuclease fusion region) may be designed or optimized to reduce off-target binding.

### TALE Nickase

TALEs comprise a highly conserved set of 33-34 amino acid sequence repeats, which may be engineered to bind to specific DNA sequences within a cellular genome. Combinations of TALENs (e.g., TALEN pairs) can be used to produce targeted DNA breaks in a DNA sequence. A TALE Nickase may be designed by introducing mutations (e.g., D450A) into a cleavage domain (e.g., FokI nuclease) of one TALEN monomer in engineered TALENs (e.g., TALEN pairs). TALE Nickases are capable of recognizing specific regions of a target DNA sequence and generate targeted single strand breaks (SSB) in a DNA sequence.

In some embodiments, TALE Nickases may be engineered to target specific DNA sequences and induce SSBs. In some embodiments, certain features of TALE Nickases (e.g., DNA-binding regions, linker regions, nuclease fusion region) may be designed or optimized to produce improved DNA binding and/or cutting efficiency. In some embodiments, certain features of TALE Nickases (e.g., DNA-binding regions, linker regions, nuclease fusion region) may be designed or optimized to reduce off-target binding.

### ZFNs (Zinc Finger Nucleases)

ZFNs are restriction enzymes that comprise a DNA binding domain (Zinc Finger Protein (ZFP)) directly or indirectly fused to a nuclease. ZFPs comprise 3-6 individual zinc finger repeats, which are capable of recognizing between 9 bp and 18 bp of a target DNA sequence. Fusion of engineered ZFPs to a nuclease cleavage domain (e.g., FokI nuclease) can produce ZFNs capable of targeting specific DNA sequence to produce DNA breaks. Combinations of ZFNs (e.g., ZFN pairs) can be used to produce targeted, double-stranded breaks (DSBs) in a DNA sequence.

In some embodiments, ZFNs may be engineered to target specific DNA sequences and induce DSBs. In some embodiments, one or more ZFNs (e.g., pairs of ZFNs) may be combined to induce DSBs through binding to target DNA sequences on strands in genomic DNA. In some embodiments, certain features of ZFNs (e.g., DNA-binding regions, linker regions, nuclease fusion region) may be designed or optimized to produce improved DNA binding and/or cutting efficiency. In some embodiments, certain features of ZFNs (e.g., DNA-binding regions, linker regions, nuclease fusion region) may be designed or optimized to reduce off-target binding.

### Zinc Finger Nickase

ZFPs comprise 3-6 individual zinc finger repeats, which are capable of recognizing between 9 bp and 18 bp of a target DNA sequence. Combinations of ZFNs (e.g., ZFN pairs) can be used to produce targeted DNA break in a DNA sequence. A ZF Nickase may be designed by introducing mutations (e.g., D450A) into a cleavage domain (e.g., FokI nuclease) of one ZFN monomer in an engineered ZFN. ZFNickases are capable of recognizing specific regions of a target DNA sequence and generate targeted SSB in a DNA sequence.

In some embodiments, ZFNickase may be engineered to target specific DNA sequences and induce SSBs. In some embodiments, certain features of ZFNickase (e.g., DNA-binding regions, linker regions, nuclease fusion region) may be designed or optimized to produce improved DNA binding and/or cutting efficiency. In some embodiments, certain features of ZFNickase (e.g., DNA-binding regions, linker regions, nuclease fusion region) may be designed or optimized to reduce off-target binding.

### Meganucleases

Meganucleases (also referred to as homing endonuclease) are sequence-specific endonucleases that are capable of recognizing between 12 bp and 40 bp of a target DNA sequence. The largest class of homing endonucleases is the LAGLIDADG family, which includes, but not limited to, the well-characterized and commonly used I-CreI and I-SceI enzymes. Re-engineering of these homing endonucleases can produce homing endonucleases capable of targeting specific DNA sequence to produce DNA break. Chimeric proteins comprising fusions of meganucleases, ZFPs, and TALs have been engineered to generate novel enzymes that take advantage of the binding affinity of ZFs and TALEs and the cleavage specificity of meganucleases.

In some embodiments, meganucleases may be engineered to target specific DNA sequences and induce DSBs. In some embodiments, one or more meganuclease may be combined to induce DSBs through binding to target DNA sequences on strands in genomic DNA. In some embodiments, one or more meganuclease may be combined with ZFPs and/or TALs to induce DSBs through binding to target DNA sequences on strands in genomic DNA. In some embodiments, certain features of meganucleases (e.g., DNA-binding regions, linker regions, nuclease fusion region) may be designed or optimized to produce improved DNA binding and/or cutting efficiency. In some embodiments, certain features of meganucleases e.g., DNA-binding regions, linker regions, nuclease fusion region) may be designed or optimized to reduce off-target binding.

### CRISPR-Associated Systems

CRISPR-associated systems typically comprise a Cas nuclease (e.g., Cas9, Cas13, Cas12a, Cas9 nickase, and the like) or variant thereof and an engineered guide RNA (gRNA) sequence. gRNA sequences are designed to have partial complementarity to a target genomic DNA sequence of interest within a certain distance from a protospacer adjacent motif (PAM). PAM sequences and distance from Cas nuclease cut sites are often specific to particular enzyme types (e.g., Cas9, Cas13, Cas12a, Cas9 nickase). Certain Cas nucleases, or variants thereof, are capable of producing DSBs in a target DNA sequence.

### Cas9

Cas9 is an RNA-guided DNA endonuclease enzyme that acts through base pair complementarity between the first 17-20 nucleotides of an engineered gRNA and a complementary strand of target genomic DNA. Target genomic DNA must present an appropriate PAM sequence (e.g., NGG or NAG) adjacent to the region of gRNA complementarity. Once bound to an appropriate target sequence, Cas9 induces a DSB in a target DNA sequence. Cas9 nucleases may be found in a number of different species, including, e.g., S. pyogenes (SpCas9), S. aureus, and N. meningitidis, among others. Engineered Cas9 nucleases may produce enhanced cutting activity or reduced off-target cutting effects.

### Cas12a/Cpf1

Cas12a (Cpf1) is an RNA-guided DNA endonuclease enzyme that acts through base pair complementarity between the first 20 nucleotides of an engineered gRNA and a complementary strand of target genomic DNA. Target genomic DNA must present an appropriate PAM sequence (e.g., TTN/TTTN/TTTV) adjacent to the region of gRNA complementarity. Once bound to an appropriate target sequence, Cas12a induces a staggered, double-stranded cut in a target DNA sequence.

### Cas9 Nickases

Mutations (e.g., D10A and/or H840A) in one of two wild-type Cas9 nuclease domains results in a Cas9 variant (Cas9 nickase). Similar to wild-type Cas9, nickases act through base pair complementarity between nucleotides of an engineered gRNA and a complementary strand of target genomic DNA. Unlike wild-type Cas9, once bound to an appropriate target sequence, Cas9 nickase cuts only one strand of the DNA generating SSB that can be repaired, without inducing indels.

gRNAs (Guide RNAs)

A guide RNA molecule may be or comprise a nucleic acid that promotes specific targeting or homing of a gRNA/Cas complex to a target. In some embodiments, a gRNA will incorporate functions and structures of a crispr RNA (crRNA) and/or trans-activating crispr RNA (tracrRNA). In some embodiments, a gRNA may be chimeric and comprise both crRNA and tracrRNA features in a single nucleic acid sequence (e.g., single guide RNA, or sgRNA). In some embodiments, a gRNA molecule comprises multiple domains. In some embodiments, a gRNA molecule comprises a targeting domain (complementary to a target nucleic acid), a first complementarity domain, a linking domain, a second complementarity domain (complementary to the first complementarity domain), a proximal domain, and/or a tail domain.

In some embodiments, a gRNA molecule may comprise a targeting domain comprising a nucleic acid sequence that is complementary (e.g., at least 80, 81, 82, 83, 84, 85, 86, 87, 88, 89, 90, 91, 92, 93, 94, 95, 96, 97, 98, 99%, or 100% complementary) to a target DNA sequence. In some embodiments, a gRNA molecule may comprise a targeting domain comprising a nucleic acid sequence that is complementary (e.g., at least 80, 81, 82, 83, 84, 85, 86, 87, 88, 89, 90, 91, 92, 93, 94, 95, 96, 97, 98, 99%, or 100% complementary) to a single strand of a target DNA sequence. In some embodiments, the targeting domain is 5-50 nucleotides in length (e.g., 5-10, 10-15, 15-20, 20-25, 25-30, 30-35, 35-40, 40-45, 45-50, and the like). In some embodiments, the targeting domain is 16 nucleotides in length. In some embodiments, the targeting domain is 16 nucleotides in length. In some embodiments, the targeting domain is 17 nucleotides in length. In some embodiments, the targeting domain is 18 nucleotides in length. In some embodiments, the targeting domain is 19 nucleotides in length. In some embodiments, the targeting domain is 20 nucleotides in length. In some embodiments, the targeting domain is 21 nucleotides in length. In some embodiments, the targeting domain is 22 nucleotides in length. In some embodiments, the targeting domain is 23 nucleotides in length. In some embodiments, the targeting domain is 24 nucleotides in length. In some embodiments, the targeting domain is 25 nucleotides in length. In some embodiments, the targeting domain is 26 nucleotides in length.

In some embodiments, a gRNA molecule may be designed to reduce off-targeting within a cellular genome. In some embodiments, a gRNA molecule may be designed through use of software for a specific target DNA sequence with limited sequence similarity (e.g., homology, identity, and the like) to another region of the genome.

In some embodiments, a gRNA molecule is designed to target a non-coding DNA sequence (e.g., intron, untranslated region, enhancer, promoter, silencer, or insulator sequence). In some embodiments, a gRNA molecule is designed to target a non-coding sequence in a human gene. In some embodiments, a gRNA molecule is designed to target a non-coding sequence in a human safe harbor gene (e.g., albumin, collagen, actin, CCR5, and the like). In some embodiments, a gRNA molecule is designed to target a non-coding sequence in a human albumin gene. In some embodiments, a gRNA molecule is designed to target intron 13 or intron 14 of a human albumin gene.

In some embodiments, a gRNA molecule may be designed to target a sequence and/or include a sequence selected from a sequence in Table 3 and 4 below. In some examples, the sequence in Table 3 or Table 4 may be a spacer sequence for a gRNA.

TABLE 3
Exemplary gRNA sequence for spCas9.

| | | | SEQ ID |
|---|---|---|---|
| Target locus | Strand | Sequence | NO: |
| Human albumin | − | GTGGTCGGTGCTGGTCTATA | 27 |
| Intron 14_1 | | | |
| Human albumin | − | TTTAGAATAGTGGTCGGTGC | 28 |
| Intron 14_2 | | | |
| Human albumin | − | CGCATCATATAAGTCAGAAC | 29 |
| Intron 14_3 | | | |
| Human albumin | − | TGAGCCATCACTTACTTGGA | 30 |
| Intron 14_4 | | | |
| Human albumin Intron 14_5 | − | CTGCTGAGCCATCACTTACT | 31 |
| Human albumin Intron 14_6 | − | TTAAGCCTGTCCTAGACTTA | 32 |
| Human albumin Intron 13_1 | + | TTGCATGTTTGGTTAGGCTA | 33 |
| Human albumin Intron 13_2 | − | ACAGTATGGCACAATAGAGC | 34 |
| Human albumin Intron 13_3 | − | AAGTTACTGTCAGGCCATAA | 35 |
| Human albumin Intron 13_4 | − | AATCTCTGGACGGAAGCTCA | 36 |

TABLE 4
Exemplary gRNA sequence for saCas9.

| | | | SEQ ID |
|---|---|---|---|
| Target locus | Strand | Sequence | NO: |
| Human albumin | + | ATGTTTGGTTAGGCTAGGGCTT | 37 |
| Intron 13_1 | | | |
| Human albumin | + | GGAGGCTTTGTACATGTGGGAC | 38 |
| Intron 13_2 | | | |
| Human albumin | + | TTACAAACAATTGTCTTACAAA | 39 |
| Intron 13_3 | | | |
| Human albumin | − | CACTGAAACTTTGGTTTCAATT | 40 |
| Intron 14_1 | | | |
| Human albumin | − | CCACTGCTGAGCCATCACTTAC | 41 |
| Intron 14_2 | | | |
| Human albumin | + | CAAGTAAGTGATGGCTCAGCAG | 42 |
| Intron 14_3 | | | |
| Human albumin | + | GGCTCAGCAGTGGAATACTCT | 43 |
| Intron 14_4 | | | |
| Human albumin | + | TTCAACTCATCCTTTCCATTG | 44 |
| Intron 14_5 | | | |
| Human albumin | + | TGACTTATATGATGCGGTACA | 45 |
| Intron 14_6 | | | |

### Heterologous Nucleic Acids

### Payloads

In some embodiments, one or more vectors or constructs described herein may comprise a polynucleotide sequence encoding one or more payloads. In accordance with various aspects, any of a variety of payloads may be used (e.g., those with a diagnostic and/or therapeutic purpose), alone or in combination. In some embodiments, a payload may be or comprise a polynucleotide sequence encoding a peptide or polypeptide. In some embodiments, a payload is a peptide that has cell-intrinsic or cell-extrinsic activity that promotes a biological process to treat a medical condition. In some embodiments, a payload may be or comprise a transgene (also referred to herein as a gene of interest (GOI)). In some embodiments, a payload may be or comprise one or more inverted terminal repeat (ITR) sequences (e.g., one or more AAV ITRs). In some embodiments, a payload may be or comprise one or more transgenes with flanking ITR sequences. In some embodiments, a payload may be or comprise one or more heterologous nucleic acid sequences encoding a reporter gene (e.g., a fluorescent or luminescent reporter). In some embodiments, a payload may be or comprise one or more biomarkers (e.g., proxy for payload expression). In some embodiments, a payload may comprise a sequence for polycistronic expression (including, e.g., a 2A peptide, or intronic sequence, internal ribosomal entry site). In some embodiments, 2A peptides are small (e.g., approximately 18-22 amino acids) peptide sequences enabling co-expression of two or more discrete protein products within a single coding sequence. In some embodiments, 2A peptides allow co-expression of two or more discrete protein products regardless of arrangement of protein coding sequences. In some embodiments, 2A peptides are or comprise a consensus motif (e.g., DVEXNPGP). In some embodiments, 2A peptides promote protein cleavage. In some embodiments, 2A peptides are or comprise viral sequences (e.g., foot-and-mouth diseases virus (F2A), equine Rhinitis A virus, porcine teschovirus-1 (P2A), or Thosea asigna virus (T2A)).

In some embodiments, a payload may be or comprise a polynucleotide sequence, which comprises an expression cassette. In some embodiments, an expression cassette comprises a first nucleic acid sequence and a second nucleic acid sequence, wherein the first nucleic acid sequence encodes a transgene, and the second nucleic acid sequence is positioned 5′ or 3′ to the first nucleic acid sequence and promotes the production of two independent gene products (e.g., a sequence encoding a 2A peptide).

In some embodiments, biomarkers are or comprise a 2A peptide (e.g., P2A, T2A, E2A, and/or F2A). In some embodiments, biomarkers are or comprise a Furin cleavage motif (See, Tian et al., FurinDB: A Database of 20-Residue Furin Cleavage Site Motifs, Substrates and Their Associated Drugs, (2011), Int. J. Mol. Sci., vol. 12:1060-1065). In some embodiments, biomarkers are or comprise a tag (e.g., an immunological tag). In some embodiments, a payload may comprise one or more functional nucleic acids (e.g., one or more siRNA or miRNA). In some embodiments, a payload may comprise one or more inhibitory nucleic acids (including, e.g., ribozyme, miRNA, siRNA, or shRNA, among other things). In some embodiments, a payload may comprise one or more nucleases (e.g., Cas proteins, endonucleases, TALENs, ZFNs).

In some embodiments, a sequence encoding a 2A peptide may have at least 80%, 85%, 90%, 95%, 99%, or 100% identity to a corresponding wild-type reference nucleotide sequence (e.g., a wild-type P2A sequence). In some embodiments, a sequence encoding a P2A peptide may be or comprise a sequence having a least 80%, 85%, 90%, 95%, 99%, or 100% identity to a portion of a corresponding wild-type reference nucleotide sequence (e.g., a wild-type gene sequence). In some embodiments, a sequence encoding a P2A peptide may be or comprise a sequence having at least 80%, 85%, 90%, 95%, 99%, or 100% identity to an exemplary sequence in Table 5 below.

TABLE 5
Exemplary 2A sequences

| | | SEQ |
|---|---|---|
| Description | Sequence | ID NO |
| P2A nucleotide | GGAAGCGGCGCCACCAATTTCAGCCTGCTGAAACAGGC | 46 |
| sequence | CGGCGACGTGGAAGAGAACCCTGGCCCT | |
| P2A amino acid | GSGATNFSLLKQAGDVEENPGP | 47 |
| sequence | | |
| P2A nucleotide | GGCAGCGGCGCCACCAACTTCAGCCTGCTGAAACAGGC | 48 |
| sequence | CGGCGACGTGGAAGAGAACCCTGGCCCT | |
| version 2 | | |

### Transgenes

In some embodiments, a transgene is a corrective gene chosen to improve one or more signs and/or symptoms of a disease, disorder, or condition. In some embodiments, a transgene may integrate into a host cell genome through use of vector(s) encompassed by the present disclosure. In some embodiments, transgenes are functional versions of disease associated genes (i.e., gene isoform(s) which are associated with the manifestation or worsening of a disease, disorder, or condition) found in a host cell. In some embodiments, transgenes are an optimized version of disease-associated genes found in a host cell (e.g., codon optimized or expression-optimized variants). In some embodiments, transgenes are variants of disease-associated genes found in a host cell (e.g., functional gene fragment or variant thereof). In some embodiments, a transgene is a gene that causes expression of a peptide that is normally expressed in one or more healthy tissues. In some embodiments, a transgene is a gene that causes expression of a peptide that is normally expressed in liver cells. In some embodiments, a transgene is a gene that causes expression of a peptide that is normally expressed in muscle cells. In some embodiments, a transgene is a gene that causes expression of a peptide that is normally expressed in central nervous system cells.

In some embodiments, a transgene may be or comprise a gene that causes expression of a peptide that is not normally expressed in one or more healthy tissues (e.g., peptide expressed ectopically). In some embodiments, a transgene is a gene that causes expression of a peptide that is ectopically expressed in one or more healthy tissues (e.g., liver, muscle, central nervous system (CNS), lung). In some embodiments, a transgene is a gene that causes expression of a peptide that is ectopically expressed in one or more healthy tissues and normally expressed in one or more healthy tissues (e.g., liver, muscle, central nervous system (CNS), lung).

In some embodiments, a transgene may be or comprise a gene encoding a functional nucleic acid. In some embodiments, a therapeutic agent is or comprises an agent that has a therapeutic effect upon a host cell or subject (including, e.g., a ribozyme, guide RNA (gRNA), antisense oligonucleotide (ASO), miRNA, siRNA, and/or shRNA). For example, in some embodiments, a therapeutic agent promotes a biological process to treat a medical condition, e.g., at least one symptom of a disease, disorder, or condition.

In some embodiments, transgene expression in a subject results substantially from integration at a target integration site. In some embodiments, 75% or more (e.g., 80% or more, 85% or more, 90% or more, 95% or more, 99% or more, 99.5% or more) of total transgene expression in a subject is from transgene integration at a target integration site. In some embodiments, 25% or less (e.g., 20% or less, 15% or less, 10% or less, 5% or less, 1% or less, 0.5% or less, 0.1% or less) of total transgene expression in a subject is from a source other than transgene integration at a target integration site (e.g., episomal expression, integration at a non-target integration site).

In some embodiments, transgenes are transiently expressed in a subject (e.g., episomal expression from plasmids, minicircle DNAs, viruses, and the like). In some embodiments, 75% or more (e.g., 80% or more, 85% or more, 90% or more, 95% or more, 99% or more, 99.5% or more) of total transgene expression in a subject is from transient expression. In some embodiments, 25% or less (e.g., 20% or less, 15% or less, 10% or less, 5% or less, 1% or less, 0.5% or less, 0.1% or less) of total transgene expression in a subject is from a source other than transient expression (e.g., integration at a non-target integration site). In some embodiments, transgenes are transiently expressed in a subject (e.g., episomal expression from plasmids, minicircle DNAs, viruses, and the like) for one or more weeks after treatment. In some embodiments, transgenes are transiently expressed in a subject (e.g., episomal expression from plasmids, minicircle DNAs, viruses, and the like) for one or more months after treatment.

In some embodiments, transgenes are transiently expressed in a subject (e.g., episomal expression from plasmids, minicircle DNAs, viruses, etc.) one or more weeks after treatment at a level comparable to that observed within one or more days after treatment. In some embodiments, transgenes are transiently expressed in a subject (e.g., episomal expression from plasmids, minicircle DNAs, viruses, and the like) one or more months after treatment at a level comparable to that observed within one or more days after treatment.

In some embodiments, transgenes are transiently expressed in a subject (e.g., episomal expression from plasmids, minicircle DNAs, viruses, and the like) one or more weeks after treatment at a level that is reduced relative to that observed within one or more days after treatment. In some embodiments, transgenes are transiently expressed in a subject (e.g., episomal expression from plasmids, minicircle DNAs, viruses, and the like) one or more months after treatment at a level that is reduced relative to that observed within one or more days after treatment.

In some embodiments, transgenes are transiently expressed in a subject (e.g., episomal expression from plasmids, minicircle DNAs, viruses, and the like) for no more than one month after treatment. In some embodiments, transgenes are transiently expressed in a subject (e.g., episomal expression from plasmids, minicircle DNAs, viruses, and the like) for no more than two months after treatment. In some embodiments, transgenes are transiently expressed in a subject (e.g., episomal expression from plasmids, minicircle DNAs, viruses, and the like) for no more than three months after treatment. In some embodiments, transgenes are transiently expressed in a subject (e.g., episomal expression from plasmids, minicircle DNAs, viruses, and the like) for no more than four months after treatment. In some embodiments, transgenes are transiently expressed in a subject (e.g., episomal expression from plasmids, minicircle DNAs, viruses, and the like) for no more than five months after treatment. In some embodiments, transgenes are transiently expressed in a subject (e.g., episomal expression from plasmids, minicircle DNAs, viruses, and the like) for no more than six months after treatment.

In some embodiments, combined size of transgenes and homology arms can be optimized to increase the likelihood that these transgenes are of a suitable sequence length to be efficiently packaged in a delivery vehicle, which can increase the likelihood that the transgenes will ultimately be delivered appropriately in the patient.

In some embodiments, a nucleotide sequence encoding a transgene is codon-optimized. In some embodiments, a nucleotide sequence encoding a transgene is codon-optimized for a certain cell type (e.g., mammalian, insect, bacterial, fungal, and the like). In some embodiments, a nucleotide sequence encoding a transgene is codon-optimized for a human cell. In some embodiments, a nucleotide sequence encoding a transgene is codon-optimized for a human cell of a particular tissue type (e.g., liver, muscle, CNS, lung).

In certain embodiments, a nucleotide sequence encoding a transgene may be codon optimized to have a nucleotide homology with a reference nucleotide sequence (e.g., a wild-type gene sequence) of less than 100%. In certain embodiments, nucleotide homology between a codon-optimized nucleotide sequence encoding a transgene and a reference nucleotide sequence is less than 100%, less than 99%, less than 98%, less than 97%, less than 96%, less than 95%, less than 94%, less than 93%, less than 92%, less than 91%, less than 90%, less than 89%, less than 88%, less than 87%, less than 86%, less than 85%, less than 84%, less than 83%, less than 82%, less than 81%, less than 80%, less than 78%, less than 76%, less than 74%, less than 72%, less than 70%, less than 68%, less than 66%, less than 64%, less than 62%, less than 60%, less than 55%, less than 50%, and less than 40%.

In some embodiments, a transgene may be or comprise a sequence having at least 80%, 85%, 90%, 95%, 99%, or 100% identity to a corresponding wild-type reference nucleotide sequence (e.g., a wild-type gene sequence). In some embodiments, a transgene may be or comprise a sequence having a least 80%, 85%, 90%, 95%, 99%, or 100% identity to a portion of a corresponding wild-type reference nucleotide sequence (e.g., a wild-type gene sequence). In some embodiments, a transgene may be or comprise a sequence having at least 80%, 85%, 90%, 95%, 99%, or 100% identity to an exemplary sequence in Table 6 below.

TABLE 6
Exemplary transgene sequences

| Transgene | | SEQ |
|---|---|---|
| Description | Sequence | ID NO |
| Human FAH | tccttcatcccggtggccgaggattccgacttccccatccacaacctgccctacggcgtcttct | 49 |
| | cgaccagaggcgacccaagaccgaggataggtgtggccattggcgaccagatcctggacc | |
| | tcagcatcatcaagcacctctttactggtcctgtcctctccaaacaccaggatgtcttcaatcagc | |
| | ctacactcaacagcttcatgggcctgggtcaggctgcctggaaggaggcgagagtgttcttgc | |
| | agaacttgctgtctgtgagccaagccaggctcagagatgacaccgaacttcggaagtgtgcat | |
| | tcatctcccaggcttctgccacgatgcaccttccagccaccataggagactacacagacttcta | |
| | ttcctctcggcagcatgctaccaacgtcggaatcatgttcagggacaaggagaatgcgttgat | |
| | gccaaattggctgcacttaccagtgggctaccatggccgtgcctcctctgtcgtggtgtctggc | |
| | accccaatccgaaggcccatgggacagatgaaacctgatgactctaagcctcccgtatatggt | |
| | gcctgcaagctcttggacatggagctggaaatggctttttttgtaggccctggaaacagattgg | |
| | gagagccgatccccatttccaaggcccatgagcacatttttggaatggtccttatgaacgactg | |
| | gagtgcacgagacattcagaagtgggagtatgtccctctcgggccattccttgggaagagtttt | |
| | gggaccactgtctctccgtgggtggtgcccatggatgctctcatgccctttgctgtgcccaacc | |
| | cgaagcaggaccccaggcccctgccgtatctgtgccatgacgagccctacacatttgacatc | |
| | aacctctctgttaacctgaaaggagaaggaatgagccaggcggctaccatatgcaagtccaat | |
| | tttaagtacatgtactggacgatgctgcagcagctcactcaccactctgtcaacggctgcaacc | |
| | tgcggccgggggacctcctggcttctgggaccatcagcgggccggagccagaaaacttcg | |
| | gctccatgttggaactgtcgtggaagggaacgaagcccatagacctggggaatggtcagac | |
| | caggaagtttctgctggacggggatgaagtcatcataacagggtactgccagggggatggtt | |
| | accgcatcggctttggccagtgtgctggaaaagtgctgcctgctctcctgccatca | |
| Human FAH | tccttcattcctgtggccgaggattctgactttcccattcacaacctgccctatggcgtcttttcaa | 50 |
| Codon | ccaggggagatcccaggcccagaatcggggtggccattggagatcagatcctggacctgtc | |
| optimized | aatcatcaagcacctgtttacaggccctgtgctgagcaagcaccaggatgtgttcaaccagcc | |
| variant 3 | aactctgaacagcttcatggggctggggcaggctgcctggaaggaggcaagggtgtttctgc | |
| | agaatctgctgagcgtgtcacaggctaggctgagggatgataccgagctgaggaagtgtgca | |
| | tttatctcacaggcatcagccacaatgcatctgccagctacaatcggcgactataccgactttta | |
| | ctcaagcaggcagcatgccaccaatgtgggcatcatgttcagggacaaagagaatgccctga | |
| | tgccaaattggctgcacctgcctgtgggctatcatggcagagccagctcagtggtggtgtcag | |
| | ggacaccaattaggaggcctatgggccagatgaagcctgatgacagcaaaccacctgtctac | |
| | ggcgcctgcaagctcctggatatggaactggagatggctttttttgtgggcccaggcaataga | |
| | ctgggagagcccattccaatctcaaaggcccatgaacatatctttggcatggtcctgatgaatg | |
| | actggtcagccagagacattcagaagtgggagtacgtgccactgggaccatttctgggaaag | |
| | agctttggcaccacagtgtcaccatgggtggtgcccatggatgccctgatgccctttgctgtgc | |
| | caaatccaaaacaagaccccaggcccctcccctatctctgtcatgatgaaccatatacttttgac | |
| | attaacctgagcgtgaacctgaaaggagaaggcatgagccaagccgccactatctgtaagag | |
| | caacttcaaatatatgtactggacaatgctgcagcagctcacccaccatagcgtgaatgggtgt | |
| | aacctgaggccaggcgacctgctggcatcaggcactatttcagggcctgagcctgaaaatttt | |
| | ggatcaatgctggagctgtcatggaagggaactaaacccatcgacctggggaatggccaga | |
| | ccagaaagtttctcctggatggcgatgaggtgatcattacaggctactgtcagggggatggct | |
| | atagaattggatttggccaatgcgccggcaaagtcctccctgccctgctgcccagc | |
| Human FAH | tccttcatcccagtggctgaagattctgacttccccattcacaacctcccttatggagtctttagca | 51 |
| Codon | caagaggagacccaagacctagaattggcgtggctattggggatcagatcctggacctgagc | |
| optimized | attatcaaacatctctttacaggcccagtcctcagcaagcaccaagatgtgttcaatcaacccac | |
| variant 2 | actgaactcatttatggggctgggccaagctgcctggaaggaggcaagagtgtttctccagaa | |
| | cctcctgtcagtgagccaggccaggctgagagatgacacagagctgaggaagtgtgccttta | |
| | tctcacaagccagcgccacaatgcacctgccagcaaccatcggggactacacagacttttac | |
| | agcagcaggcagcacgccactaatgtgggcatcatgtttagagacaaagagaatgccctcat | |
| | gcctaactggctgcatctgccagtgggctaccatggcagagccagcagcgtggtggtgtctg | |
| | gcacccctatcaggaggcccatgggccagatgaagcctgatgacagcaagccacctgtgtat | |
| | ggggcatgtaagctgctggacatggagctggaaatggctttctttgtggggcctgggaacag | |
| | gctgggcgaaccaattcccatcagcaaagctcatgaacacatttttgggatggtgctcatgaat | |
| | gactggtctgccagagacatccagaagtgggagtatgtccccctgggaccattcctgggcaa | |
| | gagctttgggaccactgtgtcaccatgggtggtgcccatggatgccctgatgccatttgctgtg | |
| | cctaatcccaaacaagatccaaggccactgccctacctctgccacgatgagccctacacattt | |
| | gatatcaacctctctgtgaatctgaagggagaaggaatgagccaagctgctaccatttgcaaa | |
| | agcaactttaagtatatgtattggaccatgctgcagcaactcacccaccacagcgtgaatggct | |
| | gtaacctgaggcctggcgacctgctggcctctggcaccatcagcggccctgaacctgagaa | |
| | ctttggcagcatgctggagctgagctggaagggaaccaagcccattgacctgggcaatgga | |
| | cagaccaggaaattcctcctggacggggacgaggtgatcatcacaggctattgccaggggg | |
| | atgggtataggattggctttggccaatgtgccggcaaagtgctgcctgccctgctccctagc | |
| Human FAH | tcctttattcctgtggccgaagatagcgacttccctatccataacctcccatatggcgtcttctcaa | 52 |
| Codon | ccaggggcgaccccaggcccaggattggggggcaattggagaccagatcctggacctca | |
| optimized | gcatcattaagcacctctttacaggacctgtgctgagcaagcaccaggatgtgttcaaccagc | |
| variant 1 | ctaccctgaatagctttatgggactgggccaggcagcatggaaagaagccagagtgttcctcc | |
| | agaatctgctgagcgtgagccaggccaggctcagggatgacacagaactgaggaaatgtgc | |
| | cttcatctcacaggcatcagccacaatgcacctccctgccacaattggggactacactgacttc | |
| | tacagcagcagacagcatgccactaatgtggggattatgtttagagacaaggaaaatgccctc | |
| | atgccaaattggctgcacctgcctgtgggctaccacggcagagcctcatcagtggtggtgtca | |
| | ggcacaccaattaggaggccaatgggacagatgaagcctgacgactcaaagccaccagtct | |
| | atggcgcctgcaaactgctggacatggagctggagatggctttctttgtggggcctggcaaca | |
| | ggctgggagaaccaatccctatttcaaaggcccacgagcacatttttggcatggtgctcatgaa | |
| | tgactggtctgccagagatatccagaagtgggaatacgtgcccctgggcccttttctgggcaa | |
| | gagctttggcaccacagtgtcaccttgggtggtcccaatggatgccctgatgccctttgccgtg | |
| | cccaaccccaagcaggatcccagaccactgccctacctgtgccacgatgagccctataccttt | |
| | gacatcaacctgtcagtcaacctgaaggggggggcatgagccaggccgccactatttgca | |
| | agagcaactttaaatatatgtactggactatgctccaacagctcactcaccactcagtgaatggc | |
| | tgcaatctgaggcctggcgacctcctggctagcggcactatctctggccctgaacctgagaac | |
| | tttgggagcatgctggagctgtcatggaaagggacaaagcctattgacctggggaatggcca | |
| | gacaaggaagtttctcctggatggcgatgaggtcatcatcactgggtactgccagggcgatg | |
| | gctacaggattggatttggacagtgtgctggcaaagtgctcccagctctgctgccctca | |
| Human | CCTGAGCAGGAGAGACAGATCACAGCCAGAGAAGGGGC | 53 |
| truncated | CAGTCGGAAAATCTTATCTAAGCTTTCTTTGCCTACCCGT | |
| ATP7B | GCCTGGGAACCAGCAATGAAGAAGAGTTTTGCTTTTGAC | |
| | AATGTTGGCTATGAAGGTGGTCTGGATGGCCTGGGCCCT | |
| | TCTTCTCAGCCGCAGAAGTGCTTCTTACAGATCAAAGGC | |
| | ATGACCTGTGCATCCTGTGTGTCTAACATAGAAAGGAAT | |
| | CTGCAGAAAGAAGCTGGTGTTCTCTCCGTGTTGGTTGCC | |
| | TTGATGGCAGGAAAGGCAGAGATCAAGTATGACCCAGA | |
| | GGTCATCCAGCCCCTCGAGATAGCTCAGTTCATCCAGGA | |
| | CCTGGGTTTTGAGGCAGCAGTCATGGAGGACTACGCAGG | |
| | CTCCGATGGCAACATTGAGCTGACAATCACAGGGATGAC | |
| | CTGCGCGTCCTGTGTCCACAACATAGAGTCCAAACTCAC | |
| | GAGGACAAATGGCATCACTTATGCCTCCGTTGCCCTTGC | |
| | CACCAGCAAAGCCCTTGTTAAGTTTGACCCGGAAATTAT | |
| | CGGTCCACGGGATATTATCAAAATTATTGAGGAAATTGG | |
| | CTTTCATGCTTCCCTGGCCCAGAGAAACCCCAACGCTCA | |
| | TCACTTGGACCACAAGATGGAAATAAAGCAGTGGAAGA | |
| | AGTCTTTCCTGTGCAGCCTGGTGTTTGGCATCCCTGTCAT | |
| | GGCCTTAATGATCTATATGCTGATACCCAGCAACGAGCC | |
| | CCACCAGTCCATGGTCCTGGACCACAACATCATTCCAGG | |
| | ACTGTCCATTCTAAATCTCATCTTCTTTATCTTGTGTACC | |
| | TTTGTCCAGCTCCTCGGTGGGTGGTACTTCTACGTTCAGG | |
| | CCTACAAATCTCTGAGACACAGGTCAGCCAACATGGACG | |
| | TGCTCATCGTCCTGGCCACAAGCATTGCTTATGTTTATTC | |
| | TCTGGTCATCCTGGTGGTTGCTGTGGCTGAGAAGGCGGA | |
| | GAGGAGCCCTGTGACATTCTTCGACACGCCCCCCATGCT | |
| | CTTTGTGTTCATTGCCCTGGGCCGGTGGCTGGAACACTT | |
| | GGCAAAGAGCAAAACCTCAGAAGCCCTGGCTAAACTCA | |
| | TGTCTCTCCAAGCCACAGAAGCCACCGTTGTGACCCTTG | |
| | GTGAGGACAATTTAATCATCAGGGAGGAGCAAGTCCCC | |
| | ATGGAGCTGGTGCAGCGGGGCGATATCGTCAAGGTGGT | |
| | CCCTGGGGGAAAGTTTCCAGTGGATGGGAAAGTCCTGG | |
| | AAGGCAATACCATGGCTGATGAGTCCCTCATCACAGGAG | |
| | AAGCCATGCCAGTCACTAAGAAACCCGGAAGCACTGTA | |
| | ATTGCGGGGTCTATAAATGCACATGGCTCTGTGCTCATT | |
| | AAAGCTACCCACGTGGGCAATGACACCACTTTGGCTCAG | |
| | ATTGTGAAACTGGTGGAAGAGGCTCAGATGTCAAAGGC | |
| | ACCCATTCAGCAGCTGGCTGACCGGTTTAGTGGATATTT | |
| | TGTCCCATTTATCATCATCATGTCAACTTTGACGTTGGTG | |
| | GTATGGATTGTAATCGGTTTTATCGATTTTGGTGTTGTTC | |
| | AGAGATACTTTCCTAACCCCAACAAGCACATCTCCCAGA | |
| | CAGAGGTGATCATCCGGTTTGCTTTCCAGACGTCCATCA | |
| | CGGTGCTGTGCATTGCCTGCCCCTGCTCCCTGGGGCTGG | |
| | CCACGCCCACGGCTGTCATGGTGGGCACCGGGGTGGCCG | |
| | CGCAGAACGGCATCCTCATCAAGGGAGGCAAGCCCCTG | |
| | GAGATGGCGCACAAGATAAAGACTGTGATGTTTGACAA | |
| | GACTGGCACCATTACCCATGGCGTCCCCAGGGTCATGCG | |
| | GGTGCTCCTGCTGGGGGATGTGGCCACACTGCCCCTCAG | |
| | GAAGGTTCTGGCTGTGGTGGGGACTGCGGAGGCCAGCA | |
| | GTGAACACCCCTTGGGCGTGGCAGTCACCAAATACTGTA | |
| | AAGAGGAACTTGGAACAGAGACCTTGGGATACTGCACG | |
| | GACTTCCAGGCAGTGCCAGGCTGTGGAATTGGGTGCAAA | |
| | GTCAGCAACGTGGAAGGCATCCTGGCCCACAGTGAGCG | |
| | CCCTTTGAGTGCACCGGCCAGTCACCTGAATGAGGCTGG | |
| | CAGCCTTCCCGCAGAAAAAGATGCAGTCCCCCAGACCTT | |
| | CTCTGTGCTGATTGGAAACCGTGAGTGGCTGAGGCGCAA | |
| | CGGTTTAACCATTTCTAGCGATGTCAGTGACGCTATGAC | |
| | AGACCACGAGATGAAAGGACAGACAGCCATCCTGGTGG | |
| | CTATTGACGGTGTGCTCTGTGGGATGATCGCAATCGCAG | |
| | ACGCTGTCAAGCAGGAGGCTGCCCTGGCTGTGCACACGC | |
| | TGCAGAGCATGGGTGTGGACGTGGTTCTGATCACGGGGG | |
| | ACAACCGGAAGACAGCCAGAGCTATTGCCACCCAGGTT | |
| | GGCATCAACAAAGTCTTTGCAGAGGTGCTGCCTTCGCAC | |
| | AAGGTGGCCAAGGTCCAGGAGCTCCAGAATAAAGGGAA | |
| | GAAAGTCGCCATGGTGGGGGATGGGGTCAATGACTCCC | |
| | CGGCCTTGGCCCAGGCAGACATGGGTGTGGCCATTGGCA | |
| | CCGGCACGGATGTGGCCATCGAGGCAGCCGACGTCGTCC | |
| | TTATCAGAAATGATTTGCTGGATGTGGTGGCTAGCATTC | |
| | ACCTTTCCAAGAGGACTGTCCGAAGGATACGCATCAACC | |
| | TGGTCCTGGCACTGATTTATAACCTGGTTGGGATACCCA | |
| | TTGCAGCAGGTGTCTTCATGCCCATCGGCATTGTGCTGC | |
| | AGCCCTGGATGGGCTCAGCGGCCATGGCAGCCTCCTCTG | |
| | TGTCTGTGGTGCTCTCATCCCTGCAGCTCAAGTGCTATAA | |
| | GAAGCCTGACCTGGAGAGGTATGAGGCACAGGCGCATG | |
| | GCCACATGAAGCCCCTGACGGCATCCCAGGTCAGTGTGC | |
| | ACATAGGCATGGATGACAGGTGGCGGGACTCCCCCAGG | |
| | GCCACACCATGGGACCAGGTCAGCTATGTCAGCCAGGTG | |
| | TCGCTGTCCTCCCTGACGTCCGACAAGCCATCTCGGCAC | |
| | AGCGCTGCAGCAGACGATGATGGGGACAAGTGGTCTCT | |
| | GCTCCTGAATGGCAGGGATGAGGAGCAGTACATC | |
| Human LDLR | gggccctggggctggaaattgcgctggaccgtcgccttgctcctcgccgcggcggggactg | 54 |
| | cagtgggcgacagatgcgaaagaaacgagttccagtgccaagacgggaaatgcatctccta | |
| | caagtgggtctgcgatggcagcgctgagtgccaggatggctctgatgagtcccaggagacg | |
| | tgcttgtctgtcacctgcaaatccggggacttcagctgtgggggccgtgtcaaccgctgcattc | |
| | ctcagttctggaggtgcgatggccaagtggactgcgacaacggctcagacgagcaaggctg | |
| | tccccccaagacgtgctcccaggacgagtttcgctgccacgatgggaagtgcatctctcggc | |
| | agttcgtctgtgactcagaccgggactgcttggacggctcagacgaggcctcctgcccggtg | |
| | ctcacctgtggtcccgccagcttccagtgcaacagctccacctgcatcccccagctgtgggcc | |
| | tgcgacaacgaccccgactgcgaagatggctcggatgagtggccgcagcgctgtaggggt | |
| | ctttacgtgttccaaggggacagtagcccctgctcggccttcgagttccactgcctaagtggcg | |
| | agtgcatccactccagctggcgctgtgatggtggccccgactgcaaggacaaatctgacgag | |
| | gaaaactgcgctgtggccacctgtcgccctgacgaattccagtgctctgatggaaactgcatc | |
| | catggcagccggcagtgtgaccgggaatatgactgcaaggacatgagcgatgaagttggct | |
| | gcgttaatgtgacactctgcgagggacccaacaagttcaagtgtcacagcggcgaatgcatc | |
| | accctggacaaagtctgcaacatggctagagactgccgggactggtcagatgaacccatcaa | |
| | agagtgcgggaccaacgaatgcttggacaacaacggcggctgttcccacgtctgcaatgac | |
| | cttaagatcggctacgagtgcctgtgccccgacggcttccagctggtggcccagcgaagatg | |
| | cgaagatatcgatgagtgtcaggatcccgacacctgcagccagctctgcgtgaacctggagg | |
| | gtggctacaagtgccagtgtgaggaaggcttccagctggacccccacacgaaggcctgcaa | |
| | ggctgtgggctccatcgcctacctcttcttcaccaaccggcacgaggtcaggaagatgacgct | |
| | ggaccggagcgagtacaccagcctcatccccaacctgaggaacgtggtcgctctggacacg | |
| | gaggtggccagcaatagaatctactggtctgacctgtcccagagaatgatctgcagcaccca | |
| | gcttgacagagcccacggcgtctcttcctatgacaccgtcatcagcagagacatccaggccc | |
| | ccgacgggctggctgtggactggatccacagcaacatctactggaccgactctgtcctgggc | |
| | actgtctctgttgcggataccaagggcgtgaagaggaaaacgttattcagggagaacggctc | |
| | caagccaagggccatcgtggtggatcctgttcatggcttcatgtactggactgactggggaac | |
| | tcccgccaagatcaagaaagggggcctgaatggtgtggacatctactcgctggtgactgaaa | |
| | acattcagtggcccaatggcatcaccctagatctcctcagtggccgcctctactgggttgactc | |
| | caaacttcactccatctcaagcatcgatgtcaacgggggcaaccggaagaccatcttggagg | |
| | atgaaaagaggctggcccaccccttctccttggccgtctttgaggacaaagtattttggacaga | |
| | tatcatcaacgaagccattttcagtgccaaccgcctcacaggttccgatgtcaacttgttggctg | |
| | aaaacctactgtccccagaggatatggttctcttccacaacctcacccagccaagaggagtga | |
| | actggtgtgagaggaccaccctgagcaatggcggctgccagtatctgtgcctccctgccccg | |
| | cagatcaacccccactcgcccaagtttacctgcgcctgcccggacggcatgctgctggccag | |
| | ggacatgaggagctgcctcacagaggctgaggctgcagtggccacccaggagacatccac | |
| | cgtcaggctaaaggtcagctccacagccgtaaggacacagcacacaaccacccgacctgtt | |
| | cccgacacctcccggctgcctggggccacccctgggctcaccacggtggagatagtgacaa | |
| | tgtctcaccaagctctgggcgacgttgctggcagaggaaatgagaagaagcccagtagcgt | |
| | gagggctctgtccattgtcctccccatgtgctcctcgtcttcctttgcctgggggtcttccttcta | |
| | tggaagaactggcggcttaagaacatcaacagcatcaactttgacaaccccgtctatcagaag | |
| | accacagaggatgaggtccacatttgccacaaccaggacggctacagctacccctcgagac | |
| | agatggtcagtctggaggatgacgtggcg | |
| Human PAH | tccactgcggtcctggaaaacccaggcttgggcaggaaactctctgactttggacaggaaac | 55 |
| | aagctatattgaagacaactgcaatcaaaatggtgccatatcactgatcttctcactcaaagaag | |
| | aagttggtgcattggccaaagtattgcgcttatttgaggagaatgatgtaaacctgacccacatt | |
| | gaatctagaccttctcgtttaaagaaagatgagtatgaatttttcacccatttggataaacgtagc | |
| | ctgcctgctctgacaaacatcatcaagatcttgaggcatgacattggtgccactgtccatgagct | |
| | ttcacgagataagaagaaagacacagtgccctggttcccaagaaccattcaagagctggaca | |
| | gatttgccaatcagattctcagctatggagcggaactggatgctgaccaccctggttttaaagat | |
| | cctgtgtaccgtgcaagacggaagcagtttgctgacattgcctacaactaccgccatgggcag | |
| | cccatccctcgagtggaatacatggaggaagaaaagaaaacatggggcacagtgttcaaga | |
| | ctctgaagtccttgtataaaacccatgcttgctatgagtacaatcacatttttccacttcttgaaaa | |
| | gtactgtggcttccatgaagataacattccccagctggaagacgtttctcagttcctgcagactt | |
| | gcactggtttccgcctccgacctgtggctggcctgctttcctctcgggatttcttgggtggcctg | |
| | gccttccgagtcttccactgcacacagtacatcagacatggatccaagcccatgtataccccc | |
| | gaacctgacatctgccatgagctgttgggacatgtgcccttgttttcagatcgcagctttgccca | |
| | gttttcccaggaaattggccttgcctctctgggtgcacctgatgaatacattgaaaagctcgcca | |
| | caatttactggtttactgtggagtttgggctctgcaaacaaggagactccataaaggcatatggt | |
| | gctgggctcctgtcatcctttggtgaattacagtactgcttatcagagaagccaaagcttctccc | |
| | cctggagctggagaagacagccatccaaaattacactgtcacggagttccagcccctctatta | |
| | cgtggcagagagttttaatgatgccaaggagaaagtaaggaactttgctgccacaatacctcg | |
| | gcccttctcagttcgctacgacccatacacccaaaggattgaggtcttggacaatacccagca | |
| | gcttaagattttggctgattccattaacagtgaaattggaatcctttgcagtgccctccagaaaat | |
| | aaag | |
| Human ASL | gcctcggagagtgggaagctttggggtggccggtttgtgggtgcagtggaccccatcatgga | 56 |
| | gaagttcaacgcgtccattgcctacgaccggcacctttgggaggtggatgttcaaggcagca | |
| | aagcctacagcaggggcctggagaaggcagggctcctcaccaaggccgagatggaccag | |
| | atactccatggcctagacaaggtggctgaggagtgggcccagggcaccttcaaactgaactc | |
| | caatgatgaggacatccacacagccaatgagcgccgcctgaaggagctcattggtgcaacg | |
| | gcagggaagctgcacacgggacggagccggaatgaccaggtggtcacagacctcaggct | |
| | gtggatgcggcagacctgctccacgctctcgggcctcctctgggagctcattaggaccatggt | |
| | ggatcgggcagaggcggaacgtgatgttctcttcccggggtacacccatttgcagagggccc | |
| | agcccatccgctggagccactggattctgagccacgccgtggcactgacccgagactctga | |
| | gcggctgctggaggtgcggaagcggatcaatgtcctgcccctggggagtggggccattgca | |
| | ggcaatcccctgggtgtggaccgagagctgctccgagcagaactcaactttggggccatcac | |
| | tctcaacagcatggatgccactagtgagcgggactttgtggccgagttcctgttctgggcttcg | |
| | ctgtgcatgacccatctcagcaggatggccgaggacctcatcctctactgcaccaaggaattc | |
| | agcttcgtgcagctctcagatgcctacagcacgggaagcagcctgatgccccagaagaaaa | |
| | accccgacagtttggagctgatccggagcaaggctgggcgtgtgtttgggcggtgtgccgg | |
| | gctcctgatgaccctcaagggacttcccagcacctacaacaaagacttacaggaggacaagg | |
| | aagctgtgtttgaagtgtcagacactatgagtgccgtgctccaggtggccactggcgtcatctc | |
| | tacgctgcagattcaccaagagaacatgggacaggctctcagccccgacatgctggccactg | |
| | accttgcctattacctggtccgcaaagggatgccattccgccaggcccacgaggcctccggg | |
| | aaagctgtgttcatggccgagaccaagggggtcgccctcaaccagctgtcactgcaggagct | |
| | gcagaccatcagccccctgttctcgggcgacgtgatctgcgtgtgggactacgggcacagtg | |
| | tggagcagtatggtgccctgggcggcactgcgcgctccagcgtcgactggcagatccgcca | |
| | ggtgcgggcgctactgcaggcacagcaggcc | |
| Human G6PC | gaggaaggaatgaatgttctccatgactttgggatccagtcaacacattacctccaggtgaatt | 57 |
| | accaagactcccaggactggttcatcttggtgtccgtgatcgcagacctcaggaatgccttcta | |
| | cgtcctcttccccatctggttccatcttcaggaagctgtgggcattaaactcctttgggtagctgt | |
| | gattggagactggctcaacctcgtctttaagtggattctctttggacagcgtccatactggtggg | |
| | ttttggatactgactactacagcaacacttccgtgcccctgataaagcagttccctgtaacctgtg | |
| | agactggaccagggagcccctctggccatgccatgggcacagcaggtgtatactacgtgatg | |
| | gtcacatctactctttccatctttcagggaaagataaagccgacctacagatttcggtgcttgaat | |
| | gtcattttgtggttgggattctgggctgtgcagctgaatgtctgtctgtcacgaatctaccttgctg | |
| | ctcattttcctcatcaagttgttgctggagtcctgtcaggcattgctgttgcagaaactttcagcca | |
| | catccacagcatctataatgccagcctcaagaaatattttctcattaccttcttcctgttcagcttcg | |
| | ccatcggattttatctgctgctcaagggactgggtgtagacctcctgtggactctggagaaagc | |
| | ccagaggtggtgcgagcagccagaatgggtccacattgacaccacaccctttgccagcctcc | |
| | tcaagaacctgggcacgctctttggcctggggctggctctcaactccagcatgtacagggag | |
| | agctgcaaggggaaactcagcaagtggctcccattccgcctcagctctattgtagcctccctc | |
| | gtcctcctgcacgtctttgactccttgaaacccccatcccaagtcgagctggtcttctacgtcttg | |
| | tccttctgcaagagtgcggtagtgcccctggcatccgtcagtgtcatcccctactgcctcgccc | |
| | aggtcctgggccagccgcacaagaagtcgttg | |
| Human | tctgactcagtaattcttcgaagtataaagaaatttggagaggagaatgatggttttgagtcagat | 58 |
| ABCB11 | aaatcatataataatgataagaaatcaaggttacaagatgagaagaaaggtgatggcgttaga | |
| | gttggcttctttcaattgtttcggttttcttcatcaactgacatttggctgatgtttgtgggaagttt | |
| | gtgtgcatttctccatggaatagcccagccaggcgtgctactcatttttggcacaatgacagatgttt | |
| | ttattgactacgacgttgagttacaagaactccagattccaggaaaagcatgtgtgaataacac | |
| | cattgtatggactaacagttccctcaaccagaacatgacaaatggaacacgttgtgggttgctg | |
| | aacatcgagagcgaaatgatcaaatttgccagttactatgctggaattgctgtcgcagtacttat | |
| | cacaggatatattcaaatatgcttttgggtcattgccgcagctcgtcagatacagaaaatgagaa | |
| | aattttactttaggagaataatgagaatggaaatagggtggtttgactgcaattcagtggggga | |
| | gctgaatacaagattctctgatgatattaataaaatcaatgatgccatagctgaccaaatggccc | |
| | ttttcattcagcgcatgacctcgaccatctgtggtttcctgttgggatttttcaggggttggaaact | |
| | gaccttggttattatttctgtcagccctctcattgggattggagcagccaccattggtctgagtgt | |
| | gtccaagtttacggactatgagctgaaggcctatgccaaagcaggggtggtggctgatgaag | |
| | tcatttcatcaatgagaacagtggctgcttttggtggtgagaaaagagaggttgaaaggtatga | |
| | gaaaaatcttgtgttcgcccagcgttggggaattagaaaaggaatagtgatgggattctttactg | |
| | gattcgtgtggtgtctcatctttttgtgttatgcactggccttctggtacggctccacacttgtcctg | |
| | gatgaaggagaatatacaccaggaacccttgtccagattttcctcagtgtcatagtaggagcttt | |
| | aaatcttggcaatgcctctccttgtttggaagcctttgcaactggacgtgcagcagccaccagc | |
| | atttttgagacaatagacaggaaacccatcattgactgcatgtcagaagatggttacaagttgg | |
| | atcgaatcaagggtgaaattgaattccataatgtgaccttccattatccttccagaccagaggtg | |
| | aagattctaaatgacctcaacatggtcattaaaccaggggaaatgacagctctggtaggaccc | |
| | agtggagctggaaaaagtacagcactgcaactcattcagcgattctatgacccctgtgaagga | |
| | atggtgaccgtggatggccatgacattcgctctcttaacattcagtggcttagagatcagattgg | |
| | gatagtggagcaagagccagttctgttctctaccaccattgcagaaaatattcgctatggcaga | |
| | gaagatgcaacaatggaagacatagtccaagctgccaaggaggccaatgcctacaacttcat | |
| | catggacctgccacagcaatttgacacccttgttggagaaggaggaggccagatgagtggtg | |
| | gccagaaacaaagggtagctatcgccagagccctcatccgaaatcccaagattctgcttttgg | |
| | acatggccacctcagctctggacaatgagagtgaagccatggtgcaagaagtgctgagtaag | |
| | attcagcatgggcacacaatcatttcagttgctcatcgcttgtctacggtcagagctgcagatac | |
| | catcattggttttgaacatggcactgcagtggaaagagggacccatgaagaattactggaaag | |
| | gaaaggtgtttacttcactctagtgactttgcaaagccagggaaatcaagctcttaatgaagag | |
| | gacataaaggatgcaactgaagatgacatgcttgcgaggacctttagcagagggagctacca | |
| | ggatagtttaagggcttccatccggcaacgctccaagtctcagctttcttacctggtgcacgaa | |
| | cctccattagctgttgtagatcataagtctacctatgaagaagatagaaaggacaaggacattc | |
| | ctgtgcaggaagaagttgaacctgccccagttaggaggattctgaaattcagtgctccagaat | |
| | ggccctacatgctggtagggtctgtgggtgcagctgtgaacgggacagtcacacccttgtatg | |
| | cctttttattcagccagattcttgggactttttcaattcctgataaagaggaacaaaggtcacagat | |
| | caatggtgtgtgcctactttttgtagcaatgggctgtgtatctcttttcacccaatttctacagggat | |
| | atgcctttgctaaatctggggagctcctaacaaaaaggctacgtaaatttggtttcagggcaatg | |
| | ctggggcaagatattgcctggtttgatgacctcagaaatagccctggagcattgacaacaaga | |
| | cttgctacagatgcttcccaagttcaaggggctgccggctctcagatcgggatgatagtcaatt | |
| | ccttcactaacgtcactgtggccatgatcattgccttctcctttagctggaagctgagcctggtca | |
| | tcttgtgcttcttccccttcttggctttatcaggagccacacagaccaggatgttgacaggatttg | |
| | cctctcgagataagcaggccctggagatggtgggacagattacaaatgaagccctcagtaac | |
| | atccgcactgttgctggaattggaaaggagaggcggttcattgaagcacttgagactgagctg | |
| | gagaagcccttcaagacagccattcagaaagccaatatttacggattctgctttgcctttgccca | |
| | gtgcatcatgtttattgcgaattctgcttcctacagatatggaggttacttaatctccaatgagggg | |
| | ctccatttcagctatgtgttcagggtgatctctgcagttgtactgagtgcaacagctcttggaaga | |
| | gccttctcttacaccccaagttatgcaaaagctaaaatatcagctgcacgcttttttcaactgctg | |
| | gaccgacaacccccaatcagtgtatacaatactgcaggtgaaaaatgggacaacttccaggg | |
| | gaagattgattttgttgattgtaaatttacatatccttctcgacctgactcgcaagttctgaatggtc | |
| | tctcagtgtcgattagtccagggcagacactggcgtttgttgggagcagtggatgtggcaaaa | |
| | gcactagcattcagctgttggaacgtttctatgatcctgatcaagggaaggtgatgatagatggt | |
| | catgacagcaaaaaagtaaatgtccagttcctccgctcaaacattggaattgtttcccaggaac | |
| | cagtgttgtttgcctgtagcataatggacaatatcaagtatggagacaacaccaaagaaattcc | |
| | catggaaagagtcatagcagctgcaaaacaggctcagctgcatgattttgtcatgtcactccca | |
| | gagaaatatgaaactaacgttgggtcccaggggtctcaactctctagaggggagaaacaacg | |
| | cattgctattgctcgggccattgtacgagatcctaaaatcttgctactagatgaagccacttctgc | |
| | cttagacacagaaagtgaaaagacggtgcaggttgctctagacaaagccagagagggtcgg | |
| | acctgcattgtcattgcccatcgcttgtccaccatccagaacgcggatatcattgctgtcatggc | |
| | acagggggtggtgattgaaaaggggacccatgaagaactgatggcccaaaaaggagccta | |
| | ctacaaactagtcaccactggatcccccatcagt | |
| Human ABCB4 | gatcttgaggcggcaaagaacggaacagcctggcgccccacgagcgcggagggcgacttt | 59 |
| | gaactgggcatcagcagcaaacaaaaaaggaaaaaaacgaagacagtgaaaatgattgga | |
| | gtattaacattgtttcgatactccgattggcaggataaattgtttatgtcgctgggtaccatcatgg | |
| | ccatagctcacggatcaggtctccccctcatgatgatagtatttggagagatgactgacaaattt | |
| | gttgatactgcaggaaacttctcctttccagtgaacttttccttgtcgctgctaaatccaggcaaa | |
| | attctggaagaagaaatgactagatatgcatattactactcaggattgggtgctggagttcttgtt | |
| | gctgcctatatacaagtttcattttggactttggcagctggtcgacagatcaggaaaattaggca | |
| | gaagttttttcatgctattctacgacaggaaataggatggtttgacatcaacgacaccactgaac | |
| | tcaatacgcggctaacagatgacatctccaaaatcagtgaaggaattggtgacaaggttggaa | |
| | tgttctttcaagcagtagccacgttttttgcaggattcatagtgggattcatcagaggatggaagc | |
| | tcacccttgtgataatggccatcagccctattctaggactctctgcagccgtttgggcaaagata | |
| | ctctcggcatttagtgacaaagaactagctgcttatgcaaaagcaggcgccgtggcagaaga | |
| | ggctctgggggccatcaggactgtgatagctttcgggggccagaacaaagagctggaaagg | |
| | tatcagaaacatttagaaaatgccaaagagattggaattaaaaaagctatttcagcaaacatttc | |
| | catgggtattgccttcctgttaatatatgcatcatatgcactggccttctggtatggatccactcta | |
| | gtcatatcaaaagaatatactattggaaatgcaatgacagtttttttttcaatcctaattggagcttt | |
| | cagtgttggccaggctgccccatgtattgatgcttttgccaatgcaagaggagcagcatatgtg | |
| | atctttgatattattgataataatcctaaaattgacagtttttcagagagaggacacaaaccagac | |
| | agcatcaaagggaatttggagttcaatgatgttcacttttcttacccttctcgagctaacgtcaag | |
| | atcttgaagggcctcaacctgaaggtgcagagtgggcagacggtggccctggttggaagta | |
| | gtggctgtgggaagagcacaacggtccagctgatacagaggctctatgaccctgatgaggg | |
| | cacaattaacattgatgggcaggatattaggaactttaatgtaaactatctgagggaaatcattg | |
| | gtgtggtgagtcaggagccggtgctgttttccaccacaattgctgaaaatatttgttatggccgt | |
| | ggaaatgtaaccatggatgagataaagaaagctgtcaaagaggccaacgcctatgagtttatc | |
| | atgaaattaccacagaaatttgacaccctggttggagagagaggggcccagctgagtggtgg | |
| | gcagaagcagaggatcgccattgcacgtgccctggttcgcaaccccaagatccttctgctgg | |
| | atgaggccacgtcagcattggacacagaaagtgaagctgaggtacaggcagctctggataa | |
| | ggccagagaaggccggaccaccattgtgatagcacaccgactgtctacggtccgaaatgca | |
| | gatgtcatcgctgggtttgaggatggagtaattgtggagcaaggaagccacagcgaactgat | |
| | gaagaaggaaggggtgtacttcaaacttgtcaacatgcagacatcaggaagccagatccagt | |
| | cagaagaatttgaactaaatgatgaaaaggctgccactagaatggccccaaatggctggaaat | |
| | ctcgcctatttaggcattctactcagaaaaaccttaaaaattcacaaatgtgtcagaagagcctt | |
| | gatgtggaaaccgatggacttgaagcaaatgtgccaccagtgtcctttctgaaggtcctgaaa | |
| | ctgaataaaacagaatggccctactttgtcgtgggaacagtatgtgccattgccaatgggggg | |
| | cttcagccggcattttcagtcatattctcagagatcatagcgatttttggaccaggcgatgatgca | |
| | gtgaagcagcagaagtgcaacatattctctttgattttcttatttctgggaattatttctttttttac | |
| | tttcttccttcagggtttcacgtttgggaaagctggcgagatcctcaccagaagactgcggtcaatg | |
| | gcttttaaagcaatgctaagacaggacatgagctggtttgatgaccataaaaacagtactggtg | |
| | cactttctacaagacttgccacagatgctgcccaagtccaaggagccacaggaaccaggttg | |
| | gctttaattgcacagaatatagctaaccttggaactggtattatcatatcatttatctacggttggc | |
| | agttaaccctattgctattagcagttgttccaattattgctgtgtcaggaattgttgaaatgaaattg | |
| | ttggctggaaatgccaaaagagataaaaaagaactggaagctgctggaaagattgcaacaga | |
| | ggcaatagaaaatattaggacagttgtgtctttgacccaggaaagaaaatttgaatcaatgtatg | |
| | ttgaaaaattgtatggaccttacaggaattctgtgcagaaggcacacatctatggaattactttta | |
| | gtatctcacaagcatttatgtatttttcctatgccggttgttttcgatttggtgcatatctcattgtg | |
| | aatggacatatgcgcttcagagatgttattctggtgttttctgcaattgtatttggtgcagtggctct | |
| | aggacatgccagttcatttgctccagactatgctaaagctaagctgtctgcagcccacttattcatgc | |
| | tgtttgaaagacaacctctgattgacagctacagtgaagaggggctgaagcctgataaatttga | |
| | aggaaatataacatttaatgaagtcgtgttcaactatcccacccgagcaaacgtgccagtgctt | |
| | caggggctgagcctggaggtgaagaaaggccagacactagccctggtgggcagcagtgg | |
| | ctgtgggaagagcacggtggtccagctcctggagcggttctacgaccccttggcggggaca | |
| | gtgcttctcgatggtcaagaagcaaagaaactcaatgtccagtggctcagagctcaactcgga | |
| | atcgtgtctcaggagcctatcctatttgactgcagcattgccgagaatattgcctatggagacaa | |
| | cagccgggttgtatcacaggatgaaattgtgagtgcagccaaagctgccaacatacatcctttc | |
| | atcgagacgttaccccacaaatatgaaacaagagtgggagataaggggactcagctctcagg | |
| | aggtcaaaaacagaggattgctattgcccgagccctcatcagacaacctcaaatcctcctgttg | |
| | gatgaagctacatcagctctggatactgaaagtgaaaaggttgtccaagaagccctggacaa | |
| | agccagagaaggccgcacctgcattgtgattgctcaccgcctgtccaccatccagaatgcag | |
| | acttaatagtggtgtttcagaatgggagagtcaaggagcatggcacgcatcagcagctgctgg | |
| | cacagaaaggcatctatttttcaatggtcagtgtccaggctgggacacagaactta | |
| Human CBS | ccttctgagaccccccaggcagaagtggggcccacaggctgcccccaccgctcagggcca | 60 |
| | cactcggcgaaggggagcctggagaaggggtccccagaggataaggaagccaaggagc | |
| | ccctgtggatccggcccgatgctccgagcaggtgcacctggcagctgggccggcctgcctc | |
| | cgagtccccacatcaccacactgccccggcaaaatctccaaaaatcttgccagatattctgaa | |
| | gaaaatcggggacacccctatggtcagaatcaacaagattgggaagaagttcggcctgaagt | |
| | gtgagctcttggccaagtgtgagttcttcaacgcgggcgggagcgtgaaggaccgcatcagc | |
| | ctgcggatgattgaggatgctgagcgcgacgggacgctgaagcccggggacacgattatcg | |
| | agccgacatccgggaacaccgggatcgggctggccctggctgcggcagtgaggggctatc | |
| | gctgcatcatcgtgatgccagagaagatgagctccgagaaggtggacgtgctgcgggcact | |
| | gggggctgagattgtgaggacgcccaccaatgccaggttcgactccccggagtcacacgtg | |
| | ggggtggcctggcggctgaagaacgaaatccccaattctcacatcctagaccagtaccgcaa | |
| | cgccagcaaccccctggctcactacgacaccaccgctgatgagatcctgcagcagtgtgatg | |
| | ggaagctggacatgctggtggcttcagtgggcacgggcggcaccatcacgggcattgccag | |
| | gaagctgaaggagaagtgtcctggatgcaggatcattggggtggatcccgaagggtccatcc | |
| | tcgcagagccggaggagctgaaccagacggagcagacaacctacgaggtggaagggatc | |
| | ggctacgacttcatccccacggtgctggacaggacggtggtggacaagtggttcaagagca | |
| | acgatgaggaggcgttcacctttgcccgcatgctgatcgcgcaagaggggctgctgtgcggt | |
| | ggcagtgctggcagcacggtggcggtggccgtgaaggccgcgcaggagctgcaggagg | |
| | gccagcgctgcgtggtcattctgcccgactcagtgcggaactacatgaccaagttcctgagcg | |
| | acaggtggatgctgcagaagggctttctgaaggaggaggacctcacggagaagaagccct | |
| | ggtggtggcacctccgtgttcaggagctgggcctgtcagccccgctgaccgtgctcccgacc | |
| | atcacctgtgggcacaccatcgagatcctccgggagaagggcttcgaccaggcgcccgtgg | |
| | tggatgaggcgggggtaatcctgggaatggtgacgcttgggaacatgctctcgtccctgcttg | |
| | ccgggaaggtgcagccgtcagaccaagttggcaaagtcatctacaagcagttcaaacagatc | |
| | cgcctcacggacacgctgggcaggctctcgcacatcctggagatggaccacttcgccctggt | |
| | ggtgcacgagcagatccagtaccacagcaccgggaagtccagtcagcggcagatggtgttc | |
| | ggggtggtcaccgccattgacttgctgaacttcgtggccgcccaggagcgggaccagaag | |
| hMMUT | ctgagagccaaaaaccagctgttcctgctgagcccccactatctgagacaggtcaaagaaag | 61 |
| | ttccgggagtagactgatccagcagagactgctgcaccagcagcagccactgcatcctgagt | |
| | gggccgctctggccaagaaacagctgaagggcaaaaacccagaagacctgatctggcaca | |
| | ctccagaggggatttcaatcaagcccctgtacagcaaaagggacactatggatctgccagag | |
| | gaactgccaggagtgaagcctttcacccgcggaccttacccaactatgtatacctttcgaccct | |
| | ggacaattcggcagtacgccggcttcagtactgtggaggaatcaaacaagttttataaggaca | |
| | acatcaaggctggacagcagggcctgagtgtggcattcgatctggccacacatcgcggctat | |
| | gactcagataatcccagagtcaggggggacgtgggaatggcaggagtcgctatcgacacag | |
| | tggaagatactaagattctgttcgatggaatccctctggagaaaatgtctgtgagtatgacaatg | |
| | aacggcgctgtcattcccgtgctggcaaacttcatcgtcactggcgaggaacagggggtgcc | |
| | taaggaaaaactgaccggcacaattcagaacgacatcctgaaggagttcatggtgcggaata | |
| | cttacatttttccccctgaaccatccatgaaaatcattgccgatatcttcgagtacaccgctaagc | |
| | acatgcccaagttcaactcaattagcatctccgggtatcatatgcaggaagcaggagccgacg | |
| | ctattctggagctggcttacaccctggcagatggcctggaatattctcgaaccggactgcagg | |
| | caggcctgacaatcgacgagttcgctcctagactgagtttcttttggggaattggcatgaactttt | |
| | acatggagatcgccaagatgagggctggccggagactgtgggcacacctgatcgagaagat | |
| | gttccagcctaagaactctaagagtctgctgctgcgggcccattgccagacatccggctggtc | |
| | tctgactgaacaggacccatataacaatattgtcagaaccgcaatcgaggcaatggcagccgt | |
| | gttcggaggaacccagagcctgcacacaaactcctttgatgaggccctggggctgcctaccg | |
| | tgaagtctgctaggattgcacgcaatacacagatcattatccaggaggaatccggaatcccaa | |
| | aggtggccgatccctggggaggctcttacatgatggagtgcctgacaaacgacgtgtatgat | |
| | gctgcactgaagctgattaatgaaatcgaggaaatggggggaatggcaaaggccgtggctg | |
| | agggcattccaaaactgaggatcgaggaatgtgcagctaggcgccaggcacgaattgactc | |
| | aggaagcgaagtgatcgtcggggtgaataagtaccagctggagaaagaagacgcagtcga | |
| | agtgctggccatcgataacacaagcgtgcgcaatcgacagattgagaagctgaagaaaatca | |
| | aaagctcccgcgatcaggcactggccgaacgatgcctggcagccctgactgagtgtgctgc | |
| | aagcggggacggaaacattctggctctggcagtcgatgcctcccgggctagatgcactgtgg | |
| | gggaaatcaccgacgccctgaagaaagtcttcggagagcacaaggccaatgatcggatggt | |
| | gagcggcgcttatagacaggagttcggggaatctaaagagattaccagtgccatcaagagg | |
| | gtgcacaagttcatggagagagaagggcgacggcccaggctgctggtggcaaagatggga | |
| | caggacggacatgatcgcggagcaaaagtcattgccaccgggttcgctgacctgggatttga | |
| | cgtggatatcggccctctgttccagacaccacgagaggtcgcacagcaggcagtcgacgct | |
| | gatgtgcacgcagtcggagtgtccactctggcagctggccataagaccctggtgcctgaact | |
| | gatcaaagagctgaactctctgggcagaccagacatcctggtcatgtgcggcggcgtgatcc | |
| | caccccaggattacgaattcctgtttgaggtcggggtgagcaacgtgttcggaccaggaacc | |
| | aggatccctaaggccgcagtgcaggtcctggatgatattgaaaagtgtctggaaaagaaaca | |
| | gcagtcagtg | |

### Homology Arms

In some embodiments, viral vectors described herein comprise one or more flanking polynucleotide sequences with significant sequence homology to a target integration site (e.g., homology arms). In some embodiments, homology arms flank a polynucleotide sequence encoding a payload (e.g., one homology arm is 5′ to a payload (also referred to herein as a 5′ homology arm) and one homology arm is 3′ to a payload (also referred to herein as a 3′ homology arm)). In some embodiments, homology arms direct site-specific integration of a payload.

In some embodiments, homology arms are of the same length (also referred to herein as balanced homology arms or even homology arms). In some embodiments, viral vectors comprising homology arms of the same length, wherein the homology arms are at least a certain length, provide improved effects (e.g., improved rate of target integration). In some embodiments, homology arms are between 50 nt and 1600 nt in length. In some embodiments, homology arms are between 100 nt and 1000 nt in length. In some embodiments, homology arms are between 200 nt and 1000 nt in length. In some embodiments, homology arms are between 500 nt and 1500 nt in length. In some embodiments, homology arms are between 1000 nt and 2000 nt in length. In some embodiments, homology arms are greater than 2000 nt in length. In some embodiments, each homology arm is at least 50 nt in length. In some embodiments, each homology arm is at least 750 nt in length. In some embodiments, each homology arm is at least 1000 nt in length. In some embodiments, each homology arm is at least 1250 nt in length. In some embodiments, homology arms are less than 1000 nt in length. In some embodiments, homology arms contain at least 70% homology to a target integration site. In some embodiments, homology arms contain at least 80% homology to a target integration site. In some embodiments, homology arms contain at least 90% homology to a target integration site. In some embodiments, homology arms contain at least 95% homology to a target integration site. In some embodiments, homology arms contain at least 99% homology to a target integration site. In some embodiments, homology arms contain 100% homology to a target integration site.

In some embodiments, homology arms are of different lengths (also referred to herein as unbalanced homology arms or uneven homology arms). In some embodiments, viral vectors comprising unbalanced homology arms of different lengths provide improved effects (e.g., increased rate of target site integration) as compared to an appropriate reference sequence. In some embodiments, viral vectors comprising homology arms of different lengths, wherein each homology arm is at least a certain length, provide improved effects (e.g., increased rate of target site integration) as compared to an appropriate reference sequence (e.g., a viral vector comprising homology arms of the same length or a viral vector comprising one or more homology arms less than 1000 nt in length).

In some embodiments, each homology arm is greater than 50 nt in length. In some embodiments, each homology arm is greater than 100 nt in length. In some embodiments, each homology arm is greater than 400 nt in length. In some embodiments, each homology arm is at least 750 nt length. In some embodiments, each homology arm is at least 1000 nt in length. In some embodiments, one homology arm is at least 750 nt in length and another homology arm is at least 1000 nt in length. In some embodiments, one homology arm is at least 750 nt in length and another homology arm is at least 1100 nt in length. In some embodiments, one homology arm is at least 750 nt in length and another homology arm is at least 1200 nt in length. In some embodiments, one homology arm is at least 750 nt in length and another homology arm is at least 1300 nt in length. In some embodiments, one homology arm is at least 750 nt in length and another homology arm is at least 1400 nt in length. In some embodiments, one homology arm is at least 750 nt in length and another homology arm is at least 1500 nt in length. In some embodiments, one homology arm is at least 750 nt in length and another homology arm is at least 1600 nt in length. In some embodiments, one homology arm is at least 750 nt in length and another homology arm is at least 1700 nt in length. In some embodiments, one homology arm is at least 750 nt in length and another homology arm is at least 1800 nt in length. In some embodiments, one homology arm is at least 750 nt in length and another homology arm is at least 1900 nt in length. In some embodiments, one homology arm is at least 750 nt in length and another homology arm is at least 2000 nt in length. In some embodiments, one homology arm is at least 1000 nt in length and another homology arm is at least 1100 nt in length. In some embodiments, one homology arm is at least 1000 nt in length and another homology arm is at least 1200 nt in length. In some embodiments, one homology arm is at least 1000 nt in length and another homology arm is at least 1300 nt in length. In some embodiments, one homology arm is at least 1000 nt in length and another homology arm is at least 1400 nt in length. In some embodiments, one homology arm is at least 1000 nt in length and another homology arm is at least 1500 nt in length. In some embodiments, one homology arm is at least 1000 nt in length and another homology arm is at least 1600 nt in length. In some embodiments, one homology arm is at least 1000 nt in length and another homology arm is at least 1700 nt in length. In some embodiments, one homology arm is at least 1000 nt in length and another homology arm is at least 1800 nt in length. In some embodiments, one homology arm is at least 1000 nt in length and another homology arm is at least 1900 nt in length. In some embodiments, one homology arm is at least 1000 nt in length and another homology arm is at least 2000 nt in length. In some embodiments, one homology arm is at least 1300 nt in length and another homology arm is at least 1400 nt in length. In some embodiments, one homology arm is at least 1600 nt in length and another homology arm is at least 1000 nt in length. In some embodiments, one homology arm is at least 1250 nt in length and another homology arm is at least 1250 nt in length. In some embodiments, one homology arm is at least 400 nt in length and another homology arm is at least 800 nt in length. In some embodiments, one homology arm is at least 600 nt in length and another homology arm is at least 600 nt in length.

In some embodiments, a 5′ homology arm is longer than a 3′ homology arm. In some embodiments, a 3′ homology arm is longer than a 5′ homology arm. For example, in some embodiments, a 5′ homology arm is approximately 1600 nt in length and a 3′ homology arm is approximately 1000 nt in length. In some embodiments, a 5′ homology arm is approximately 1000 nt in length and a 3′ homology arm is approximately 1600 nt in length. In some embodiments, viral vectors comprising homology arms provide improved effects (e.g., increased rate of target site integration) as compared to an appropriate reference sequence (e.g., viral vectors lacking homology arms). In some embodiments, viral vectors comprising homology arms provide rates of target site integration of 0.01% or more (e.g., 0.05% or more, 0.1% or more, 0.2% or more, 0.3% or more, 0.4% or more, 0.5% or more, 0.6% or more, 0.7% or more, 0.8% or more, 0.9% or more, 1% or more, 1.5% or more, 2% or more, 5% or more, 10% or more, 20% or more, 30% or more). In some embodiments, viral vectors comprising homology arms provide increasing rates of target site integration over time. In some embodiments, rates of target site integration increase over time relative to an initial measurement of target site integration. In some embodiments, rates of target site integration over time are at least 1.5× higher than an initial measurement of target site integration (e.g., 1.5×, 2×, 3×, 4×, 5×, 10×, 20×, 30×, 40×, 50×, 60×, 70×, 80×, 90×, 100×, 200×). In some embodiments, rates of target site integration are measured after one or more days. In some embodiments, rates of target site integration are measured after one or more weeks. In some embodiments, rates of target site integration are measured after one or more months. In some embodiments, rates of target site integration are measured after one or more years.

In some embodiments, viral vectors comprising homology arms of different lengths provide improved effects (e.g., increased rate of target site integration) relative to a reference sequence (e.g., viral vectors with homology arms of the same length, viral vectors with at least one homology arm below 500 nt). In some embodiments, viral vectors comprising homology arms of different lengths provide at least 1.1×, at least 1.2×, at least 1.3×, at least 1.4×, at least 1.5×, at least 1.6×, at least 1.7×, at least 1.8×, at least 1.9×, at least 2.0×, at least 2.5×, at least 3.0×, at least 3.5×, or at least 4.0× improved editing activity relative to a reference composition (e.g., viral vectors with homology arms of the same length, viral vectors with at least one homology arm below 500 nt).

In some embodiments, viral vectors comprising homology arms of different lengths provide rates of target site integration of 0.01% or more (e.g., 0.05% or more, 0.1% or more, 0.2% or more, 0.3% or more, 0.4% or more, 0.5% or more, 0.6% or more, 0.7% or more, 0.8% or more, 0.9% or more, 1% or more, 1.5% or more, 2% or more, 5% or more, 10% or more, 20% or more, 30% or more). In some embodiments, viral vectors comprising homology arms of different lengths provide increasing rates of target site integration over time. In some embodiments, rates of target site integration increase over time relative to an initial measurement of target site integration. In some embodiments, rates of target site integration over time are at least 1.5× higher than an initial measurement of target site integration (e.g., 1.5×, 2×, 3×, 4×, 5×, 10×, 20×, 30×, 40×, 50×, 60×, 70×, 80×, 90×, 100×, 200×).

In some embodiments, viral vectors comprising homology arms of different lengths may provide improved gene editing in a species or a model system for a species (e.g., mouse, human, or models thereof). In some embodiments, viral vectors may comprise different combinations of homology arm lengths when optimized for expression in a particular species or a model system for a particular species (e.g., mouse, human, or models thereof). In some embodiments, viral vectors comprising specific combinations of homology arm lengths may provide improved gene editing in one species or a model system of one species (e.g., human, humanized mouse model) as compared to a second species or a model system of a second species (e.g., mouse, pure mouse model). In some embodiments, viral vectors comprising specific combinations of homology arm lengths may be optimized for high levels of gene editing in one species or a model of one species (e.g., human, humanized mouse model) as compared to a second species or a model system of a second species (e.g., mouse, pure mouse model).

In some embodiments, homology arms direct integration of a transgene immediately behind a highly expressed endogenous gene. In some embodiments, homology arms direct integration of a transgene without disrupting endogenous gene expression (non-disruptive integration).

In some embodiments, one or more homology arm sequences may have at least 80%, 85%, 90%, 95%, 99%, or 100% identity to a corresponding wild-type reference nucleotide sequence (e.g., a wild-type genomic sequence). In some embodiments, one or more homology arm sequences may be or comprise a sequence having a least 80%, 85%, 90%, 95%, 99%, or 100% identity to a portion of a corresponding wild-type reference nucleotide sequence (e.g., a wild-type genomic sequence). In some embodiments, one or more homology arm sequences may be or comprise a sequence having at least 80%, 85%, 90%, 95%, 99%, or 100% identity to an exemplary sequence in Table 7 below. In some embodiments, compositions described herein may comprise one or more homology arm sequences selected from Table 7 below.

TABLE 7
Exemplary homology arm sequences

| | | SEQ |
|---|---|---|
| HA Description | Sequence | ID NO |
| Human albumin | GTGATGCTTATGAATATTAATAGGAATATTTGTAAGGCC | 62 |
| 0.4 kb 5′ | TGAAATATTTTGATCATGAAATCAAAACATTAATTTATTT | |
| homology arm | AAACATTTACTTGAAATGTGGTGGTTTGTGATTTAGTTG | |
| | ATTTTATAGGCTAGTGGGAGAATTTACATTCAAATGTCT | |
| | AAATCACTTAAAATTGCCCTTTATGGCCTGACAGTAACT | |
| | TTTTTTTATTCATTTGGGGACAACTATGTCCGTGAGCTTC | |
| | CGTCCAGAGATTATAGTAGTAAATTGTAATTAAAGGATA | |
| | TGATGCACGTGAAATCACTTTGCAATCATCAATAGCTTC | |
| | ATAAATGTTAATTTTGTATCCTAATAGTAATGCTAATATT | |
| | TTCCTAACATCTGTCATGTCTTTGTGTTCAGGGTAAAAAA | |
| | CTTGTTGCTGCAAGTCAAGCTGCCTTAGGCTTA | |
| Human albumin | GCATGTTTGGTTAGGCTAGGGCTTAGGGATTTATATATC | 63 |
| 0.6 kb 5′ | AAAGGAGGCTTTGTACATGTGGGACAGGGATCTTATTTT | |
| homology arm | ACAAACAATTGTCTTACAAAATGAATAAAACAGCACTTT | |
| | GTTTTTATCTCCTGCTCTATTGTGCCATACTGTTAAATGT | |
| | TTATAATGCCTGTTCTGTTTCCAAATTTGTGATGCTTATG | |
| | AATATTAATAGGAATATTTGTAAGGCCTGAAATATTTTG | |
| | ATCATGAAATCAAAACATTAATTTATTTAAACATTTACTT | |
| | GAAATGTGGTGGTTTGTGATTTAGTTGATTTTATAGGCTA | |
| | GTGGGAGAATTTACATTCAAATGTCTAAATCACTTAAAA | |
| | TTGCCCTTTATGGCCTGACAGTAACTTTTTTTTATTCATTT | |
| | GGGGACAACTATGTCCGTGAGCTTCCGTCCAGAGATTAT | |
| | AGTAGTAAATTGTAATTAAAGGATATGATGCACGTGAAA | |
| | TCACTTTGCAATCATCAATAGCTTCATAAATGTTAATTTT | |
| | GTATCCTAATAGTAATGCTAATATTTTCCTAACATCTGTC | |
| | ATGTCTTTGTGTTCAGGGTAAAAAACTTGTTGCTGCAAG | |
| | TCAAGCTGCCTTAGGCTTA | |
| Human albumin | TTCAAACTCAGTGCACTTGTTGAGCTTGTGAAACACAAG | 64 |
| 0.8 kb 5′ | CCCAAGGCAACAAAAGAGCAACTGAAAGCTGTTATGGA | |
| homology arm | TGATTTCGCAGCTTTTGTAGAGAAGTGCTGCAAGGCTGA | |
| | CGATAAGGAGACCTGCTTTGCCGAGGAGGTACTACAGTT | |
| | CTCTTCATTTTAATATGTCCAGTATTCATTTTTGCATGTTT | |
| | GGTTAGGCTAGGGCTTAGGGATTTATATATCAAAGGAGG | |
| | CTTTGTACATGTGGGACAGGGATCTTATTTTACAAACAA | |
| | TTGTCTTACAAAATGAATAAAACAGCACTTTGTTTTTATC | |
| | TCCTGCTCTATTGTGCCATACTGTTAAATGTTTATAATGC | |
| | CTGTTCTGTTTCCAAATTTGTGATGCTTATGAATATTAAT | |
| | AGGAATATTTGTAAGGCCTGAAATATTTTGATCATGAAA | |
| | TCAAAACATTAATTTATTTAAACATTTACTTGAAATGTG | |
| | GTGGTTTGTGATTTAGTTGATTTTATAGGCTAGTGGGAG | |
| | AATTTACATTCAAATGTCTAAATCACTTAAAATTGCCCTT | |
| | TATGGCCTGACAGTAACTTTTTTTTATTCATTTGGGGACA | |
| | ACTATGTCCGTGAGCTTCCGTCCAGAGATTATAGTAGTA | |
| | AATTGTAATTAAAGGATATGATGCACGTGAAATCACTTT | |
| | GCAATCATCAATAGCTTCATAAATGTTAATTTTGTATCCT | |
| | AATAGTAATGCTAATATTTTCCTAACATCTGTCATGTCTT | |
| | TGTGTTCAGGGTAAAAAACTTGTTGCTGCAAGTCAAGCT | |
| | GCCTTAGGCTTA | |
| Human albumin | TAACATCACATTTAAAAGCATCTCAGGTAACTATATTTT | 65 |
| 0.4 kb 3′ | GAATTTTTTAAAAAAGTAACTATAATAGTTATTATTAAA | |
| homology arm | ATAGCAAAGATTGACCATTTCCAAGAGCCATATAGACCA | |
| | GCACCGACCACTATTCTAAACTATTTATGTATGTAAATA | |
| | TTAGCTTTTAAAATTCTCAAAATAGTTGCTGAGTTGGGA | |
| | ACCACTATTATTTCTATTTTGTAGATGAGAAAATGAAGA | |
| | TAAACATCAAAGCATAGATTAAGTAATTTTCCAAAGGGT | |
| | CAAAATTCAAAATTGAAACCAAAGTTTCAGTGTTGCCCA | |
| | TTGTCCTGTTCTGACTTATATGATGCGGTACACAGAGCC | |
| | ATCCAAGTAAGTGATGGCTCAGCAGTGGAATACTCTGGG | |
| | AATTAGGCTGAACCACATGAAAGAGTGCTTTATA | |
| Human albumin | TAACATCACATTTAAAAGCATCTCAGGTAACTATATTTT | 66 |
| 0.6 kb 3′ | GAATTTTTTAAAAAAGTAACTATAATAGTTATTATTAAA | |
| homology arm | ATAGCAAAGATTGACCATTTCCAAGAGCCATATAGACCA | |
| | GCACCGACCACTATTCTAAACTATTTATGTATGTAAATA | |
| | TTAGCTTTTAAAATTCTCAAAATAGTTGCTGAGTTGGGA | |
| | ACCACTATTATTTCTATTTTGTAGATGAGAAAATGAAGA | |
| | TAAACATCAAAGCATAGATTAAGTAATTTTCCAAAGGGT | |
| | CAAAATTCAAAATTGAAACCAAAGTTTCAGTGTTGCCCA | |
| | TTGTCCTGTTCTGACTTATATGATGCGGTACACAGAGCC | |
| | ATCCAAGTAAGTGATGGCTCAGCAGTGGAATACTCTGGG | |
| | AATTAGGCTGAACCACATGAAAGAGTGCTTTATAGGGCA | |
| | AAAACAGTTGAATATCAGTGATTTCACATGGTTCAACCT | |
| | AATAGTTCAACTCATCCTTTCCATTGGAGAATATGATGG | |
| | ATCTACCTTCTGTGAACTTTATAGTGAAGAATCTGCTATT | |
| | ACATTTCCAATTTGTCAACATGCTGAGCTTTAATAGGAC | |
| | TTATCTTCTTATGACAACATTTATTG | |
| Human albumin | TAACATCACATTTAAAAGCATCTCAGGTAACTATATTTT | 67 |
| 0.8 kb 3′ | GAATTTTTTAAAAAAGTAACTATAATAGTTATTATTAAA | |
| homology arm | ATAGCAAAGATTGACCATTTCCAAGAGCCATATAGACCA | |
| | GCACCGACCACTATTCTAAACTATTTATGTATGTAAATA | |
| | TTAGCTTTTAAAATTCTCAAAATAGTTGCTGAGTTGGGA | |
| | ACCACTATTATTTCTATTTTGTAGATGAGAAAATGAAGA | |
| | TAAACATCAAAGCATAGATTAAGTAATTTTCCAAAGGGT | |
| | CAAAATTCAAAATTGAAACCAAAGTTTCAGTGTTGCCCA | |
| | TTGTCCTGTTCTGACTTATATGATGCGGTACACAGAGCC | |
| | ATCCAAGTAAGTGATGGCTCAGCAGTGGAATACTCTGGG | |
| | AATTAGGCTGAACCACATGAAAGAGTGCTTTATAGGGCA | |
| | AAAACAGTTGAATATCAGTGATTTCACATGGTTCAACCT | |
| | AATAGTTCAACTCATCCTTTCCATTGGAGAATATGATGG | |
| | ATCTACCTTCTGTGAACTTTATAGTGAAGAATCTGCTATT | |
| | ACATTTCCAATTTGTCAACATGCTGAGCTTTAATAGGAC | |
| | TTATCTTCTTATGACAACATTTATTGGTGTGTCCCCTTGC | |
| | CTAGCCCAACAGAAGAATTCAGCAGCCGTAAGTCTAGG | |
| | ACAGGCTTAAATTGTTTTCACTGGTGTAAATTGCAGAAA | |
| | GATGATCTAAGTAATTTGGCATTTATTTTAATAGGTTTGA | |
| | AAAACACATGCCATTTTACAAATAAGACTTATATTTGTC | |
| | CTTTTGTTTTTCAGCCTACCATGAG | |
| Human albumin | tttctagatgtaaataattattttaagtttgccctatggtggccccacacatgagacaaaccccca | 68 |
| 1.6 kb 5′ | agatgtgacttttgagaatgagacttggataaaaaacatgtagaaatgcaagccctgaagctca | |
| homology arm | actccctattgctatcacaggggttataattgcataaaatttagctatagaaagttgctgtcatct | |
| | cttgtgggctgtaatcatcgtctaggcttaagagtaatattgcaaaacctgtcatgcccacacaaa | |
| | tctctccctggcattgttgtctttgcagatgtcagtgaaagagaaccagcagctcccatgagtttg | |
| | gatagccttattttctatagcctccccactattagctttgaagggagcaaagtttaagaaccaaat | |
| | ataaagtttctcatctttatagatgagaaaaattttaaataaagtccaagataattaaatttttaagg | |
| | atcatttttagctctttaatagcaataaaactcaatatgacataatatggcacttccaaaatctgaat | |
| | aatatataattgcaatgacatacttcttttcagagatttactgaaaagaaatttgttgacactacata | |
| | acgtgatgagtggtttatactgattgtttcagttggtcttcccaccaactccatgaaagtggatttt | |
| | attatcctcatcatgcagatgagaatattgagacttatagcggtatgcctgagccccaaagtact | |
| | cagagttgcctggctccaagatttataatcttaaatgatgggactaccatccttactctctccatttt | |
| | tctatacgtgagtaatgttttttctgttttttttttttctttttccattcaaactcagtgcacttgtt | |
| | gagctcgtgaaacacaagcccaaggcaacaaaagagcaactgaaagctgttatggatgatttcgcag | |
| | cttttgtagagaagtgctgcaaggctgacgataaggagacctgctttgccgaggaggtactac | |
| | agttctcttcattttaatatgtccagtattcatttttgcatgtttggttaggctagggcttagggatt | |
| | tatatatcaaaggaggctttgtacatgtgggacagggatcttattttacaaacaattgtcttacaaaa | |
| | tgaataaaacagcacttttttttatctcctgctctattgtgccatactgttaaatgtttataatgcct | |
| | gttctgtttccaaatttgtgatgcttatgaatattaataggaatatttgtaaggcctgaaatattttg | |
| | atcatgaaatcaaaacattaatttatttaaacatttacttgaaatgtggtggtttgtgatttagttga | |
| | ttttataggctagtgggagaatttacattcaaatgtctaaatcacttaaaattgccctttatggcctg | |
| | acagtaacttttttttattcatttggggacaactatgtccgtgagcttccgtccagagattatagtag | |
| | taaattgtaattaaaggatatgatgcacgtgaaatcactttgcaatcatcaatagcttcataaatgtt | |
| | aattttgtatcctaatagtaatgctaatattttcctaacatctgtcatgtctttgtgttcagggtaaa | |
| | aaacttgttgctgcaagtcaagctgccttaggctta | |
| Human albumin | Taacatcacatttaaaagcatctcaggtaactatattttgaattttttaaaaaagtaactataatagt | 69 |
| 1kb 3′ | tattattaaaatagcaaagattgaccatttccaagagccatatagaccagcaccgaccactattc | |
| homology arm | taaactatttatgtatgtaaatattagcttttaaaattctcaaaatagttgctgagttgggaaccact | |
| | attatttctattttgtagatgagaaaatgaagataaacatcaaagcatagattaagtaattttccaa | |
| | agggtcaaaattcaaaattgaaaccaaagtttcagtgttgcccattgtcctgttctgacttatatga | |
| | tgcggtacacagagccatccaagtaagtgatggctcagcagtggaatactctgggaattagg | |
| | ctgaaccacatgaaagagtgctttatagggcaaaaacagttgaatatcagtgatttcacatggtt | |
| | caacctaatagttcaactcatcctttccattggagaatatgatggatctaccttctgtgaactttata | |
| | gtgaagaatctgctattacatttccaatttgtcaacatgctgagctttaataggacttatcttcttat | |
| | gacaacatttattggtgtgtccccttgcctagcccaacagaagaattcagcagccgtaagtctag | |
| | gacaggcttaaattgttttcactggtgtaaattgcagaaagatgatctaagtaatttggcatttattt | |
| | taataggtttgaaaaacacatgccattttacaaataagacttatatttgtccttttgtttttcagcct | |
| | accatgagaataagagaaagaaaatgaagatcaaaagcttattcatctgtttttctttttcgttggtg | |
| | taaagccaacaccctgtctaaaaaacataaatttctttaatcattttgcctcttttctctgtgcttca | |
| | attaataaaaaatggaaagaatctaatagagtggtacagcactgttatttttcaaagatgtgttg | |
| Human albumin | Actccatgaaagtggattttattatcctcatcatgcagatgagaatattgagacttatagcggtat | 70 |
| 1kb 5′ | gcctgagccccaaagtactcagagttgcctggctccaagatttataatcttaaatgatgggacta | |
| homology arm | ccatccttactctctccatttttctatacgtgagtaatgttttttctgttttttttttttctttttc | |
| | cattcaaactcagtgcacttgttgagctcgtgaaacacaagcccaaggcaacaaaagagcaactgaa | |
| | agctgttatggatgatttcgcagcttttgtagagaagtgctgcaaggctgacgataaggagacctgct | |
| | ttgccgaggaggtactacagttctcttcattttaatatgtccagtattcatttttgcatgtttggtta | |
| | ggctagggcttagggatttatatatcaaaggaggctttgtacatgtgggacagggatcttattttac | |
| | aaacaattgtcttacaaaatgaataaaacagcactttgtttttatctcctgctctattgtgccatact | |
| | gttaaatgtttataatgcctgttctgtttccaaatttgtgatgcttatgaatattaataggaatattt | |
| | gtaaggcctgaaatattttgatcatgaaatcaaaacattaatttatttaaacatttacttgaaatgtg | |
| | gtggtttgtgatttagttgattttataggctagtgggagaatttacattcaaatgtctaaatcactta | |
| | aaattgccctttatggcctgacagtaacttttttttattcatttggggacaactatgtccgtgagctt | |
| | ccgtccagagattatagtagtaaattgtaattaaaggatatgatgcacgtgaaatcactttgcaatca | |
| | tcaatagcttcataaatgttaattttgtatcctaatagtaatgctaatattttcctaacatctgtcat | |
| | gtctttgtgttcagggtaaaaaacttgttgctgcaagtcaagctgccttaggctta | |

In some embodiments, viral vectors provided herein may comprise a 5′ homology arm and a 3′ homology arm designed to a target an albumin locus. In some embodiments, viral vectors provided herein may comprise a 5′ homology arm sequence that has at least 70%, at least 75%, at least 80%, at least 85%, at least 90%, at least 95%, at least 99%, or 100% sequence identity with SEQ ID NO: 62 and a 3′ homology arm sequence that has at least 70%, at least 75%, at least 80%, at least 85%, at least 90%, at least 95%, at least 99%, or 100% sequence identity with SEQ ID NO: 67. In some embodiments, a viral vector comprises a 5′ homology arm comprising the sequence of SEQ ID NO: 62 and a 3′ homology arm comprising the sequence of SEQ ID NO: 67.

In some embodiments, viral vectors provided herein may comprise a 5′ homology arm sequence that has at least 70%, at least 75%, at least 80%, at least 85%, at least 90%, at least 95%, at least 99%, or 100% sequence identity with SEQ ID NO: 63 and a 3′ homology arm sequence that has at least 70%, at least 75%, at least 80%, at least 85%, at least 90%, at least 95%, at least 99%, or 100% sequence identity with SEQ ID NO: 66. In some embodiments, a viral vector comprises a 5′ homology arm comprising the sequence of SEQ ID NO: 63 and a 3′ homology arm comprising the sequence of SEQ ID NO: 66.

In some embodiments, viral vectors provided herein may comprise a 5′ homology arm sequence that has at least 70%, at least 75%, at least 80%, at least 85%, at least 90%, at least 95%, at least 99%, or 100% sequence identity with SEQ ID NO: 64 and a 3′ homology arm sequence that has at least 70%, at least 75%, at least 80%, at least 85%, at least 90%, at least 95%, at least 99%, or 100% sequence identity with SEQ ID NO: 65. In some embodiments, a viral vector comprises a 5′ homology arm comprising the sequence of SEQ ID NO: 64 and a 3′ homology arm comprising the sequence of SEQ ID NO: 65.

In some embodiments, viral vectors provided herein may comprise a 5′ homology arm sequence that has at least 70%, at least 75%, at least 80%, at least 85%, at least 90%, at least 95%, at least 99%, or 100% sequence identity with SEQ ID NO: 68 and a 3′ homology arm sequence that has at least 70%, at least 75%, at least 80%, at least 85%, at least 90%, at least 95%, at least 99%, or 100% sequence identity with SEQ ID NO: 69. In some embodiments, a viral vector comprises a 5′ homology arm comprising the sequence of SEQ ID NO: 68 and a 3′ homology arm comprising the sequence of SEQ ID NO: 69. In some embodiments, viral vectors provided herein may comprise a 5′ homology arm sequence that has at least 70%, at least 75%, at least 80%, at least 85%, at least 90%, at least 95%, at least 99%, or 100% sequence identity with SEQ ID NO: 70 and a 3′ homology arm sequence that has at least 70%, at least 75%, at least 80%, at least 85%, at least 90%, at least 95%, at least 99%, or 100% sequence identity with SEQ ID NO: 69.

### Measurement of Target Site Integration

As described elsewhere herein, one of the problems with traditional use of nucleases to introduce nucleic acid material into cells is the significant chance of off target integration. Accordingly, it is important to verify correct integration through one or more specifically targeted assays, as described below.

In accordance with various embodiments, the rate of integration may be measured at any of a variety of points in time. In some embodiments, rates of target site integration are measured after one or more days. In some embodiments, rates of target site integration are measured after one or more weeks. In some embodiments, rates of target site integration are measured after one or more months. In some embodiments, rates of target site integration are measured after one or more years. In some embodiments, rates of target site integration are measured through assessment of one or more biomarkers (e.g., biomarkers comprising a 2A peptide). In some embodiments, rates of target site integration are measured through assessment of one or more isolated nucleic acids (e.g., mRNA, gDNA). In some embodiments, rates of target site integration are measured through assessment of gene expression (e.g., through immunohistochemical staining).

TABLE 8
Exemplary methods for assessment of target site integration

| | | Exemplary | |
|---|---|---|---|
| Assay type | Sample analyzed | method | Exemplary protocol |
| Genomic DNA | Liver (frozen) | qPCR | Liver biopsy subjected to genomic DNA |
| integration rate | | | extraction. qPCR method run to detect |
| (gDNA Int %) | | | percentage of allele (e.g., albumin) containing |
| | | | on-target insertion. |
| Fused mRNA | Liver (frozen) | ddPCR | Liver biopsy subjected to RNA extraction. |
| | | | ddPCR method is run to quantify the copy |
| | | | number of fused mRNA (unique chimeric |
| | | | mRNA transcribed from edited allele). This |
| | | | assay measures the transcriptional activity after |
| | | | target insertion. |
| ALB-2A | Plasma | ELISA | Blood is collected and processed for plasma. |
| | | | Proprietary ELISA used to measure 2A-tagged |
| | | | albumin (universal circulating biomarker for |
| | | | targeted integration) This assay measures total |
| | | | protein expression after target insertion. |
| Hepatocyte | Fixed liver | IHC | Fixed liver sectioned and stained against |
| editing % | section | | transgene. Transgene-positive cells counted and |
| | | | used to calculate percentage of hepatocyte |
| | | | editing. For targeted integration into a target |
| | | | integration site in the albumin locus, transgene |
| | | | expression should be hepatocyte-specific. This |
| | | | assay focuses on per-cell target integration and is |
| | | | orthogonal to gDNA Int %, which focuses on per |
| | | | allele target integration. |
| GFP expression | Fixed cells (e.g, | ICC/IHC | Fixed cells counterstained with the nuclear dye. |
| | HepG2) and/or | | GFP+ cells imaged directly or stained using anti- |
| | fixed tissue (e.g., | | HA tag antibody. This assay measures the |
| | liver) section | | percentage of cells that express the GFP |
| | | | transgene and is an indicator of viral vector |
| | | | editing efficiency. |
| UGT1A1 | Fixed cells (e.g, | ICC/IHC | Fixed cells counterstained with the nuclear dye. |
| expression | HepG2) and/or | | Cells stained using anti-UGT1A1 antibody. This |
| | fixed tissue (e.g., | | assay measures the percentage of cells that |
| | liver) section | | express the UGT1A1 and is an indicator of viral |
| | | | vector editing efficiency. |

### Methods of Treatment

Compositions and constructs disclosed herein may be used in any in vitro or in vivo application to cause or enhance expression of a payload (e.g., transgene) from a particular target integration site in a cell while maintaining expression of endogenous genes at and surrounding the target integration site. For example, compositions and constructs disclosed herein may be used to treat a disorder, disease, or medical condition in a subject (e.g., through gene therapy).

In some embodiments, treatment comprises obtaining or maintaining a desired pharmacologic and/or physiologic effect. In some embodiments, a desired pharmacologic and/or physiologic effect may comprise completely or partially preventing a disease (e.g., preventing symptoms of disease). In some embodiments, a desired pharmacologic and/or physiologic effect may comprise completely or partially curing a disease (e.g., curing adverse effects associated with a disease). In some embodiments, a desired pharmacologic and/or physiologic effect may comprise preventing recurrence of a disease. In some embodiments, a desired pharmacologic and/or physiologic effect may comprise slowing progression of a disease. In some embodiments, a desired pharmacologic and/or physiologic effect may comprise relieving symptoms of a disease. In some embodiments, a desired pharmacologic and/or physiologic effect may comprise preventing regression of a disease. In some embodiments, a desired pharmacologic and/or physiologic effect may comprise stabilizing and/or reducing symptoms associated with a disease.

In some embodiments, treatment comprises administering a composition before, during, or after onset of a disease (e.g., before, during, or after appearance of symptoms associated with a disease). In some embodiments, treatment comprises combination therapy (e.g., with one or more therapies, including different types of therapies).

### Diseases of Interest

In some embodiments, compositions and constructs disclosed herein may be used to treat any disease of interest that includes a genetic deficiency or abnormality as a component of the disease.

By way of specific example, in some embodiments, compositions and constructs such as those disclosed herein may be used to treat branched-chain organic acidurias (e.g., Maple Syrup Urine Disease (MSUD), methylmalonic acidemia (MMA), propionic acidemia (PA), isovaleric acidemia (IVA), argininosuccinic aciduria). In some embodiments, treatment comprises introduction of a polynucleotide sequence encoding one or more transgenes of interest (e.g., BCKDH complex (E1a, E1b, and E2 subunits), methylmalonyl-CoA mutase, propionyl-CoA carboxylase (alpha and beta subunits), isovaleryl CoA dehydrogenase, argininosuccinate lyase (ASL), and/or variants thereof). In some embodiments, treatment comprises reduction of aberrant proteins (e.g., non-functional proteins) associated with branched chain organic acidurias. In some embodiments, treatment comprises reduction of signs and/or symptoms associated with branched chain organic acidurias (e.g., hypotonia, developmental delay, seizures, optic atrophy, acute encephalopathy, hyperventilation, respiratory distress, temperature instability, recurrent vomiting, ketoacidosis, pancreatitis, constipation, neutropenia, pancytopenia, secondary hemophagocytosis, cardiac arrhythmia, cardiomyopathy, chronic renal failure, dermatitis, hearing loss).

In some embodiments, compositions and constructs disclosed herein may be used to treat fatty acid oxidation disorders (e.g., trifunctional protein deficiency, Long-chain L-3 hydroxyacyl-CoA dehydrogenase (LCAD) deficiency, Medium-chain acyl-CoA dehydrogenase (MCHAD) deficiency, Very long-chain acyl-CoA dehydrogenase (VLCHAD) deficiency). In some embodiments, treatment comprises introduction of a polynucleotide sequence encoding one or more transgenes of interest (e.g., HADHA, HADHB, LCHAD, ACADM, ACADVL, and/or variants thereof). In some embodiments, treatment comprises reduction of aberrant proteins (e.g., non-functional proteins) associated with fatty acid oxidation disorders. In some embodiments, treatment comprises reduction of signs and/or symptoms associated with fatty acid oxidation disorders (e.g., enlarged liver, delayed mental and physical development, cardiac muscle weakness, cardiac arrhythmia, nerve damage, abnormal liver function, rhabdomyolysis, myoglobinuria, hypoglycemia, metabolic acidosis, respiratory distress, hepatomegaly, hypotonia, cardiomyopathy).

In some embodiments, compositions and constructs disclosed herein may be used to treat glycogen storage diseases (e.g., glycogen storage disease type 1 (GSD1), glycogen storage disease type 2 (Pompe disease, GSD2). In some embodiments, treatment comprises introduction of a polynucleotide sequence encoding one or more transgenes of interest (e.g., G6PC (GSD1a), G6PT1 (GSD1b), SLC17A3, SLC37A4 (GSD1c), acid alpha-glucosidase, and/or variants thereof). In some embodiments, treatment comprises reduction of aberrant proteins (e.g., non-functional proteins) associated with glycogen storage diseases. In some embodiments, treatment comprises reduction of signs and/or symptoms associated with glycogen storage diseases (e.g., enlarged liver, hypoglycemia, muscle weakness, muscle cramps, fatigue, delayed development, obesity, bleeding disorders, abnormal liver function, abnormal kidney function, abnormal respiratory function, abnormal cardiac function, mouth sores, gout, cirrhosis, fibrosis, liver tumors).

In some embodiments, compositions and constructs disclosed herein may be used to treat carnitine cycle disorders. In some embodiments, treatment comprises introduction of a polynucleotide sequence encoding one or more transgenes of interest (e.g., OCTN2, CPT1, CACT, CPT2, and/or variants thereof). In some embodiments, treatment comprises reduction of aberrant proteins (e.g., non-functional proteins) associated with carnitine cycle disorders. In some embodiments, treatment comprises reduction of signs and/or symptoms associated with carnitine cycle disorders (e.g., hypoketotic hypoglycemia, cardiomyopathy, muscle weakness, fatigue, delayed motor development, edema).

In some embodiments, compositions and constructs disclosed herein may be used to treat urea cycle disorders. In some embodiments, treatment comprises introduction of a polynucleotide sequence encoding one or more transgenes of interest (e.g., CPS1, ARG1, ASL, OTC, and/or variants thereof). In some embodiments, treatment comprises reduction of aberrant proteins (e.g., non-functional proteins) associated with urea cycle disorders. In some embodiments, treatment comprises reduction of signs and/or symptoms associated with urea cycle disorders (e.g., vomiting, nausea, behavior abnormalities, fatigue, coma, psychosis, lethargy, cyclical vomiting, myopia, hyperammonemia, elevated ornithine levels).

In some embodiments, compositions and constructs disclosed herein may be used to treat homocystinuria (HCU). In some embodiments, treatment comprises introduction of a polynucleotide sequence encoding one or more transgenes of interest (e.g., cystathionine beta synthase (CBS), and/or variants thereof). In some embodiments, treatment comprises reduction of aberrant proteins (e.g., non-functional proteins) associated with HCU. In some embodiments, treatment comprises reduction of signs and/or symptoms associated with HCU (e.g., ectopia lentis, myopia, iridodenesis, cataracts, optic atrophy, glaucoma, retinal detachment, retinal damage, delayed developmental milestones, intellectual disability, depression, anxiety, obsessive-compulsive disorder, dolichostenomelia, genu valgum, pes cavus, scoliosis, pectus carinatum, pectus excavatum, osteoporosis, increased clot development, thromboembolism, pulmonary embolism, fragile skin, hypopigmentation, malar flushing, inguinal hernia, pancreatitis, kyphosis, spontaneous pneumothorax).

In some embodiments, compositions and constructs disclosed herein may be used to treat Crigler-Najjar syndrome. In some embodiments, treatment comprises introduction of a polynucleotide sequence encoding one or more transgenes of interest (e.g., UGT1A1, and/or variants thereof). In some embodiments, treatment comprises reduction of aberrant proteins (e.g., non-functional proteins) associated with Crigler-Najjar syndrome. In some embodiments, treatment comprises reduction of signs and/or symptoms associated with Crigler-Najjar syndrome (e.g., jaundice, kernicterus, lethargy, vomiting, fever, abnormal reflexes, muscle spasms, opisthotonus, spasticity, hypotonia, athetosis, elevated bilirubin levels, diarrhea, slurred speech, confusion, difficulty swallowing, seizures).

In some embodiments, compositions and constructs disclosed herein may be used to treat hereditary tyrosinemia. In some embodiments, treatment comprises introduction of a polynucleotide sequence encoding one or more transgenes of interest (e.g., FAH, and/or variants thereof). In some embodiments, treatment comprises reduction of aberrant proteins (e.g., non-functional proteins) associated with hereditary tyrosinemia. In some embodiments, treatment comprises reduction of signs and/or symptoms associated with hereditary tyrosinemia (e.g., hepatomegaly, jaundice, liver disease, cirrhosis, hepatocarcinoma, fever, diarrhea, melena, vomiting, splenomegaly, edema, coagulopathy, abnormal kidney function, rickets, weakness, hypertonia, ileus, tachycardia, hypertension, neurological crises, respiratory failure, cardiomyopathy).

In some embodiments, compositions and constructs disclosed herein may be used to treat epidermolysis bullosa. In some embodiments, treatment comprises introduction of a polynucleotide sequence encoding one or more transgenes of interest (e.g., COL7A1, COL17A1, MMP1, KRT5, LAMA3, LAMB3, LAMC2, ITGB4, and/or variants thereof). In some embodiments, treatment comprises reduction of aberrant proteins (e.g., non-functional proteins) associated with epidermolysis bullosa. In some embodiments, treatment comprises reduction of signs and/or symptoms associated with epidermolysis bullosa (e.g., fragile skin, abnormal nail growth, blisters, thickened skin, scarring alopecia, atrophic scarring, milia, dental problems, dysphagia, skin itching and pain).

In some embodiments, compositions and constructs disclosed herein may be used to treat alpha-1 antitrypsin deficiency (A1ATD). In some embodiments, treatment comprises introduction of a polynucleotide sequence encoding one or more transgenes of interest (e.g., alpha-1 antitrypsin (A1AT), and/or variants thereof). In some embodiments, treatment comprises reduction of aberrant proteins (e.g., non-functional proteins) associated with alpha-1 antitrypsin deficiency. In some embodiments, treatment comprises reduction of signs and/or symptoms associated with A1ATD (e.g., emphysema, chronic cough, phlegm production, wheezing, chronic respiratory infections, jaundice, enlarged liver, bleeding, abnormal fluid accumulation, elevated liver enzymes, liver dysfunction, portal hypertension, fatigue, edema, chronic active hepatitis, cirrhosis, hepatocarcinoma, panniculitis).

In some embodiments, compositions and constructs disclosed herein may be used to treat Wilson's disease. In some embodiments, treatment comprises introduction of a polynucleotide sequence encoding one or more transgenes of interest (e.g., ATP7B, and/or variants thereof). In some embodiments, treatment comprises reduction of aberrant proteins (e.g., non-functional proteins) associated with Wilson's disease. In some embodiments, treatment comprises reduction of signs and/or symptoms associated with Wilson's disease (e.g., fatigue, lack of appetite, abdominal pain, jaundice, Kayser-Fleischer rings, edema, speech problems, problems swallowing, loss of physical coordination, uncontrolled movements, muscle stiffness, liver disease, anemia, depression, schizophrenia, ammenorrhea, infertility, kidney stones, renal tubular damage, arthritis, osteoporosis, osteophytes).

In some embodiments, compositions and constructs disclosed herein may be used to treat hematologic diseases (e.g., hemophilia A, hemophilia B). In some embodiments, treatment comprises introduction of a polynucleotide sequence encoding one or more transgenes of interest (e.g., Factor IX (FIX), Factor VIII (FVIII), and/or variants thereof). In some embodiments, treatment comprises reduction of aberrant proteins (e.g., non-functional proteins) associated with hematologic diseases. In some embodiments, treatment comprises reduction of signs and/or symptoms associated with hematologic diseases (e.g., excessive bleeding, abnormal bruising, joint pain and swelling, bloody urine, bloody stool, abnormal nosebleeds, headache, lethargy, vomiting, double vision, weakness, convulsions, seizures).

In some embodiments, compositions and constructs disclosed herein may be used to treat hereditary angioedema. In some embodiments, treatment comprises introduction of a polynucleotide sequence encoding one or more transgenes of interest (e.g., C1 esterase inhibitor (C1-inh)). In some embodiments, treatment comprises reduction of aberrant proteins (e.g., non-functional proteins) associated with hereditary angioedema. In some embodiments, treatment comprises reduction of signs and/or symptoms associated with hereditary angioedema (e.g., edema, pruritus, urticaria, nausea, vomiting, acute abdominal pain, dysphagia, dysphonia, stridor).

In some embodiments, compositions and constructs disclosed herein may be used to treat Parkinson's disease. In some embodiments, treatment comprises introduction of a polynucleotide sequence encoding one or more transgenes of interest (e.g., dopamine decarboxylase (DDC)). In some embodiments, treatment comprises reduction of aberrant proteins (e.g., non-functional proteins) associated with Parkinson's disease. In some embodiments, treatment comprises reduction of signs and/or symptoms associated with Parkinson's disease (e.g., tremors, bradykinesia, muscle stiffness, impaired posture and balance, loss of automatic movements, speech changes, writing changes).

In some embodiments, compositions and constructs disclosed herein may be used to treat muscular diseases. In some embodiments, treatment comprises introduction of a polynucleotide sequence encoding one or more transgenes of interest (e.g., muscular dystrophies, Duchenne's muscular dystrophy (DMD), limb girdle muscular dystrophy). X-linked myotubular myopathy). In some embodiments, treatment comprises reduction of aberrant proteins (e.g., non-functional proteins) associated with muscular diseases. In some embodiments, treatment comprises reduction of signs and/or symptoms associated with muscular diseases (e.g., difficult movement, enlarged calf muscles, muscle pain and stiffness, delayed development, learning disabilities, unusual gait, scoliosis, breathing problems, difficulty swallowing, arrhythmia, cardiomyopathy, abnormal joint function, hypotonia, respiratory distress, absence of reflexes).

In some embodiments, compositions and constructs disclosed herein may be used to treat mucopolysaccharidosis (MPS) (e.g., MPS IH, MPS IH/S, MPS IS, MPS II, MPS IIIA, MPS IIIB, MPS IIIC, MPS IIID, MPS IVA, MPS IVB, MPS V, MPS VI, MPS VII, MPS IX). In some embodiments, treatment comprises introduction of a polynucleotide sequence encoding one or more transgenes of interest (e.g., IDUA, IDS, SGSH, NAGLU, HGSNAT, GNS, GALNS, GLB1, ARSB, GUSB, HYAL1). In some embodiments, treatment comprises reduction of aberrant proteins (e.g., non-functional proteins) associated with mucopolysaccharidosis. In some embodiments, treatment comprises reduction of signs and/or symptoms associated with MPS (e.g., heart abnormalities, breathing irregularities, enlarged liver, enlarged spleen, neurological abnormalities, developmental delays, recurring infections, persistent nasal discharge, noisy breathing, clouding of the cornea, enlarged tongue, spine deformities, joint stiffness, carpal tunnel, aortic regurgitation, progressive hearing loss, seizures, unsteady gait, accumulation of heparan sulfate, enzyme deficiencies, abnormal skeleton and musculature, heart disease, cysts, soft-tissue masses).

In some embodiments, compositions and constructs disclosed herein may be used to treat lysosomal acid lipase deficiency. In some embodiments, treatment comprises introduction of a polynucleotide sequence encoding one or more transgenes of interest (e.g., LIPA and/or variants thereof). In some embodiments, treatment comprises reduction of aberrant proteins (e.g., non-functional proteins) associated with lysosomal acid lipase deficiency. In some embodiments, treatment comprises reductions of signs and/or symptoms associated with lysosomal acid lipase deficiency (e.g., vomiting, diarrhea, swelling of the abdomen, and failure to gain weight, weight loss, jaundice, fever, calcification, anemia, liver dysfunction or failure, cachexia, malabsorption, bile duct problems, cardiac disease, stroke).

In some embodiments, compositions and constructs disclosed herein may be used to treat disorders associated with bile acid metabolism, transport, and/or cholestasis. In some embodiments, treatment comprises introduction of a polynucleotide sequence encoding one or more transgene of interest (e.g., PFIC1, PFIC2, PFIC3, ABCB4, and/or variant thereof). In some embodiments, treatment comprises reduction of aberrant proteins (e.g., non-functional proteins) associated with bile acid metabolism, transport, and/or cholestasis. In some embodiments, treatment comprises reductions of signs and/or symptoms associated with bile acid metabolism, transport, and/or cholestasis (e.g., itching, jaundice, failure to thrive, portal hypertension, hepatosplenomegaly, diarrhea, pancreatitis, hepatocellular carcinoma).

In some embodiments, compositions and constructs disclosed herein may be used to treat phenylketonuria. In some embodiments, treatment comprises introduction of a polynucleotide sequence encoding one or more transgene of interest (e.g., phenylalanine hydroxylase (PAH) and/or variant thereof). In some embodiments, treatment comprises reduction of aberrant proteins (e.g., non-functional proteins) associated with phenylketonuria. In some embodiments, treatment comprises reductions of signs and/or symptoms associated with phenylketonuria (e.g., musty odor in the breath, skin and/or urine, seizures, skin rashes, microcephaly, hyperactivity, intellectual disability, asthma, eczema, anemia, weight gain, renal insufficiency, osteoporosis, gastritis, esophagus, and kidney deficiencies, kidney stones, hypertension, psychiatric problems, dizziness).

In some embodiments, compositions and constructs disclosed herein may be used to treat primary hyperoxaluria. In some embodiments, treatment comprises introduction of a polynucleotide sequence encoding one or more transgene of interest (e.g., AGT, AGXT, GRHPR, HOGA1, and/or variant thereof). In some embodiments, treatment comprises reduction of aberrant proteins (e.g., non-functional proteins) associated with primary hyperoxaluria. In some embodiments, treatment comprises reductions of signs and/or symptoms associated with phenylketonuria (e.g., flank pain, oxalosis, kidney stones and/or stones elsewhere in the urinary tract such as the bladder or urethra, nephrocalcinosis, hematuria, dysuria, the urge to urinate often, renal colic, blockage of the urinary tract, repeated urinary tract infections, kidney damage, kidney failure, failure to thrive).

In some embodiments, compositions and constructs disclosed herein may be used to treat porphyrias. In some embodiments, treatment comprises introduction of a polynucleotide sequence encoding one or more transgene of interest (e.g., ALAD, HMBS, UROS, UROD, CPOX, PPOCX, FECH, ALAS2, and/or variant thereof). In some embodiments, treatment comprises reduction of aberrant proteins (e.g., non-functional proteins) associated with porphyrias. In some embodiments, treatment comprises reductions of signs and/or symptoms associated with porphyrias (e.g., abdominal pain, pain in the arms and leg, generalized weakness, vomiting, confusion, constipation, tachycardia, fluctuating blood pressure, urinary retention, psychosis, hallucinations, seizures, abrasions, blisters, erosions of the skin, skin lesions, nausea, increased blood pressure, confusion).

In some embodiments, compositions and constructs disclosed herein may be used to treat disorders associated with production of antibodies (e.g., autoimmune disorders). In some embodiments, treatment comprises introduction of a polynucleotide sequence encoding one or more transgenes of interest (e.g., POLB, HLA-DRB1, IL7R, CYP27B1, TNFRSF1A, HLA-B, HLA-DPB1, HLA-DRB1, IRF5, PTPN22, RBPJ, RUNX1, STAT4, and/or variants thereof). In some embodiments, treatment comprises reduction of aberrant proteins (e.g., non-functional proteins) associated with production of antibodies. In some embodiments, treatment comprises reduction of signs and/or symptoms associated with production of antibodies (e.g., swollen joints, joint stiffness, fatigue, fever, appetite loss, vision problems, tremor, unsteady gait, dizziness, skin rash, lesions, hyperalgesia).

In some embodiments, compositions and constructs disclosed herein may be used to treat disorders associated with production of secreted proteins. In some embodiments, treatment comprises introduction of a polynucleotide sequence encoding one or more transgenes of interest. In some embodiments, treatment comprises reduction of aberrant proteins (e.g., non-functional proteins) associated with production of secreted proteins. In some embodiments, treatment comprises reduction of signs and/or symptoms associated with production of secreted proteins.

### Targeted Integration

In some embodiments, compositions and constructs provided herein direct integration of a payload (e.g., a transgene and/or functional nucleic acid) at a target integration site (e.g., an endogenous gene). In some embodiments, compositions and constructs provided herein direct integration of a payload (e.g., a transgene and/or functional nucleic acid) at a target integration site (e.g., an endogenous gene) that is found only in a specific tissue. In some embodiments, compositions and constructs provided herein direct integration of a payload (e.g., a transgene and/or functional nucleic acid) at a target integration site (e.g., an endogenous gene) that is found in cells that are present in more than one tissue in a subject (e.g., 2, 3, 4, 5, or all tissues). In some embodiments, compositions and constructs provided herein direct integration of a payload (e.g., a transgene and/or functional nucleic acid) at a target integration site (e.g., an endogenous gene) that may allow for inducible expression of a payload. In some embodiments, inducible expression may be controlled via artificial means (e.g., administration of a drug or other exogenous signal) or via naturally occurring means (e.g., IgH expression from B cells) In some embodiments, compositions and constructs provided herein direct integration of a payload at a target integration site in a specific cell type (e.g., tissue-specific loci). In some embodiments, integration of a payload occurs in a specific tissue (e.g., liver, central nervous system (CNS), muscle, kidney, vascular. lung). In some embodiments, integration of a payload occurs in multiple tissues (e.g., liver, central nervous system (CNS), muscle, kidney, vascular, lung).

In some embodiments, compositions and constructs provided herein direct integration of a payload at a target integration site that is considered a safe-harbor site (e.g., albumin, Apolipoprotein A2 (ApoA2), Haptoglobin, IgH (e.g., B cells), Beta-2 microglobulin, β-Actin (beta-actin), GAPDH). In some embodiments, a target integration site may be selected from any genomic site appropriate for use with methods and compositions provided herein. In some embodiments, a target integration site encodes a polypeptide. In some embodiments, a target integration site encodes a polypeptide that is highly expressed in a subject (e.g., a subject not suffering from a disease, disorder, or condition, or a subject suffering from a disease, disorder, or condition). In some embodiments, integration of a payload occurs at a 5′ or 3′ end of one or more endogenous genes (e.g., genes encoding polypeptides). In some embodiments, integration of a payload occurs between a 5′ or 3′ end of one or more endogenous genes (e.g., genes encoding polypeptides).

In some embodiments, compositions and constructs provided herein direct integration of a payload at a target integration site with minimal or no off-target integration (e.g., integration at a non-target locus). In some embodiments, compositions and constructs provided herein direct integration of a payload at a target integration site with reduced off-target integration compared to a reference composition or construct (e.g., relative to a composition or construct without flanking homology sequences).

In some embodiments, integration of a transgene at a target integration site allows expression of a payload without disrupting endogenous gene expression. In some embodiments, integration of a transgene at a target integration site allows expression of a payload from an endogenous promoter. In some embodiments, integration of a transgene at a target integration site disrupts endogenous gene expression. In some embodiments, integration of a transgene at a target integration site disrupts endogenous gene expression without adversely affecting a target cell and/or subject (e.g., by targeting a safe-harbor site). In some embodiments, integration of a transgene at a target integration site does not require use of a nuclease (e.g., Cas nucleases, TALENs, ZFNs). In some embodiments, integration of a transgene at a target integration site is assisted by use of a nuclease (e.g., Cas nucleases, TALENS, ZFNs).

In some embodiments, integration of a transgene at a target integration site confers a selective advantage (e.g., increased survival rate in a plurality of cells relative to other cells in a tissue). In some embodiments, a selective advantage may produce an increased percentage of cells in one or more tissues expressing a transgene.

### Cut Site

In some embodiments, integration of a transgene at a target integration site is enhanced through use of a nuclease (e.g., Cas nucleases, TALENs, ZFNs) targeting a specific cut site for a single- or double-stranded DNA break. In some embodiments, integration of a transgene at a target integration site is enhanced through use of a nuclease targeting a cut site in a DNA sequence that is distal to the target integration site. In some embodiments, integration of a transgene at a target integration site is assisted by use of a nuclease targeting a cut site in a DNA sequence that does not overlap with the target integration site. In some embodiments, integration of a transgene at a target integration site is enhanced through use of a nuclease targeting a cut site in a DNA sequence that is at least about 100 bp (e.g., about 100 bp, 150 bp, 200 bp, 250 bp, 300 bp, 350 bp, 400 bp, 450 bp, 500 bp, 550 bp, 600 bp, 650 bp, 700 bp, 750 bp, 800 bp, 850 bp, 900 bp, 950 bp, 1000 bp, 1050 bp, 1100 bp, 1150 bp, 1200 bp, 1250 bp, 1300 bp, 1350 bp, 1400 bp, 1450 bp, 1500 bp, 1550 bp, 1600 bp, 1650 bp, 1700 bp, 1800 bp, 1850 bp, 1900 bp, 1950 bp, 2000 bp) away from a target integration site. In some embodiments, integration of a transgene at a target integration site is enhanced through use of a nuclease targeting a cut site in a DNA sequence that is within about 100 bp (e.g., about 1 bp, 3 bp, 5 bp, 10 bp, 20 bp, 30 bp, 40 bp, 50 bp, 60 bp, 70 bp, 80 bp, 90 bp, 100 bp) of a target integration site.

In some embodiments, a cut site is within a non-coding sequence of DNA (e.g., intron, untranslated region, enhancer, promoter, silencer, or insulator). In some embodiments, a cut site is within a non-coding sequence of a human gene. In some embodiments, a cut site is within an intron of a human gene (e.g., collagen, actin, albumin, Beta-2 microglobulin, IgH, GAPDH, G6PC). In some embodiments, a cut site is within intron 13 or intron 14 of a human albumin gene. In some embodiments, a cut site is within intron 5 or a site after exon 6 of a human actin gene. In some embodiments, a cut site is within intron 2 or intron 3 of a human Beta-2 microglobulin gene. In some embodiments, a cut site is within IGHJ6 or between IGHJ6 and IGHM of a human IgH gene.

In some embodiments, a cut site within one or more non-coding sequences of a human gene (e.g., intron 13 and 14 of an albumin gene) may provide improved transgene integration when combined with GENERIDE™ components and one or more appropriate nucleases. In some embodiments, selection of a cut site may be based upon predicted off-target effects (e.g., reduced off-target effects) through use of an external database or software prediction/calculation tool known in the art (e.g., Benchling, CHOPCHOP, IDT, CRISPOR, E-CRISP, TureDesign, CRISPick). In some embodiments, a cut site may reduce unwanted off-target effects (e.g., cytotoxicity, carcinogenicity, immunogenicity, and the like). In some embodiments, a cut site may be within a safe-harbor locus to reduce possible effects of disruptive integration.

### Compositions

In some embodiments, compositions can be produced using methods and constructs provided herein (e.g., viral vectors). In some embodiments, compositions include liquid, solid, and gaseous compositions. In some embodiments, compositions comprise additional ingredients (e.g., diluents, stabilizer, excipients, adjuvants). In some embodiments, additional ingredients can comprise buffers (e.g., phosphate, citrate, organic acid buffers), antioxidants (e.g., ascorbic acid), low molecular weight polypeptides (e.g., less than 10 residues), various proteins (e.g., serum albumin, gelatin, immunoglobulins), hydrophilic polymers (e.g., polyvinylpyrrolidone), amino acids (e.g., glycine, glutamine, asparagine, arginine, lysine), carbohydrates (e.g., monosaccharides, disaccharides, glucose, mannose, dextrins), chelating agents (e.g., EDTA), sugar alcohols (e.g., mannitol, sorbitol), salt-forming counterions (e.g., sodium, potassium), and/or nonionic surfactants (e.g., Tween™, Pluronics™, polyethylene glycol (PEG)), among other things. In some embodiments, an aqueous carrier is an aqueous pH buffered solution.

In some embodiments, compositions provided herein may be provided in a range of dosages. In some embodiments, compositions provided herein may be provided in a single dose. In some embodiments, compositions provided herein may be provided in multiple dosages. In some embodiments, compositions are provided over a period of time. In some embodiments, compositions are provided at specific intervals (e.g., varying intervals, set intervals). In some embodiments, dosages may vary depending upon dosage form and route of administration. In some embodiments, compositions provided herein may be provided in dosages between 1e11 and 1e14 vg/kg. In some embodiments, compositions provided herein may be provided in dosages between 1e12 and 1e13 vg/kg. In some embodiments, compositions provided herein may be provided in dosages between 1e12 and 1e14 vg/kg. In some embodiments, compositions provided herein may be provided in dosages between 1e14 and 1e15 vg/kg. In some embodiments, compositions provided herein may be provided in dosages of no more than 1e14 vg/kg. In some embodiments, compositions provided herein may be provided in dosages of no more than 1e15 vg/kg.

In some embodiments, one of ordinary skill in the art will be able to devise an appropriate dosage level and dosing regimen using the pharmaceutical compositions described herein for treatment of various conditions in various patients. For example, in some embodiments, a selected dosage depends upon a desired therapeutic effect, on a route of administration, and on a duration of treatment desired. In some embodiments, dosage levels of about 0.001 mg to about 6 mg of nucleic acid per kg of body weight are administered during each dosage to a subject (e.g., animal, human). In some embodiments, dosage levels of nucleic acids within disclosed lipid nanoparticles are about 0.1 mg/kg to about 1.0 mg/kg. In some embodiments, dosage levels of nucleic acids within disclosed lipid nanoparticles are about 0.1 mg/kg to about 3.0 mg/kg. In some embodiments, dosage levels of disclosed lipid nanoparticles are about 0.2 mg to about 100 mg of the total of one or more components (e.g., ionizable lipids, sterols, conjugate-linker lipids, phospholipids)/kg of body weight are administered to a subject (e.g., animal, human). In some embodiments, dosage levels of disclosed lipid nanoparticles are about 0.5 mg/kg to about 6 mg/kg of the total of one or more components (e.g., ionizable lipids, sterols, conjugate-linker lipids, phospholipids)/kg of body weight.

In some embodiments, compositions provided herein may be administered to a subject at a particular timepoint (e.g., age of a subject). In some embodiments, compositions provided herein may be administered to a newborn subject. In some embodiments, compositions provided herein may be administered to a neonatal subject. In some embodiments, a neonatal mouse subject is between 0 and 7 days of age. In some embodiments, a neonatal human subject is between 0 days and 1 month of age. In some embodiments compositions provided herein may be administered to a subject between 7 days of age and 30 days of age. In some embodiments, compositions provided herein may be administered to a subject between 3 months of age and 1 year of age. In some embodiments, compositions provided herein may be administered to a subject between 1 year of age and 5 years of age. In some embodiments, compositions provided herein may be administered to a subject between 4 years of age and 7 years of age. In some embodiments, compositions provided herein may be administered to a subject at 5 years of age or older.

In some embodiments, compositions provided herein may be administered to a subject at a particular timepoint based upon growth stage (e.g., percentage of estimated/average adult size or weight) of a particular tissue or organ. In some embodiments, compositions provided herein may be administered to a subject wherein a tissue or organ (e.g., liver, muscle, CNS, lung, and the like) is at least 10%, at least 20%, at least 30%, at least 40%, at least 50%, at least 60%, at least 70%, at least 80%, at least 90%, or at least 99% of estimated/average adult size or weight. In some embodiments, compositions provided herein may be administered to a subject wherein a tissue or organ is approximately 20% (+/−5%) of estimated/average adult size or weight. In some embodiments, compositions provided herein may be administered to a subject wherein a tissue or organ is approximately 50% (+/−5%) of estimated/average adult size or weight. In some embodiments, compositions provided herein may be administered to a subject wherein a tissue or organ is approximately 60% (+/−5%) of estimated/average adult size or weight. In some embodiments, estimated/average adult size or weight of a particular tissue or organ may be determined as described in the art (See, Noda et al. Pediatric radiology, 1997; Johnson et al. Liver transplantation, 2005; and Szpinda et al. Biomed research international, 2015, each of which is incorporated herein by reference in its entirety).

In some embodiments, a composition as described herein may comprise (i) a nuclease or polynucleotide sequence encoding a nuclease capable of inducing a double-stranded break and/or a single-stranded break at a cut site distal from a target integration site; and (ii) a polynucleotide cassette comprising an expression cassette comprising a first nucleic acid sequence that has at least 70%, at least 75%, at least 80%, at least 85%, at least 90%, at least 95%, at least 99%, or 100% sequence identity with a sequence in Table 6 (e.g., SEQ ID NO: 49), a second nucleic acid sequence is positioned 5′ or 3′ sequence to the first nucleic acid sequence that has at least 70%, at least 75%, at least 80%, at least 85%, at least 90%, at least 95%, at least 99%, or 100% sequence identity with a sequence in Table 5 (e.g., SEQ ID NO: 46, SEQ ID NO: 47, SEQ ID NO: 48) a third nucleic acid sequence positioned 5′ to the expression cassette and comprising a sequence that that has at least 70%, at least 75%, at least 80%, at least 85%, at least 90%, at least 95%, at least 99%, or 100% sequence identity with a sequence in Table 7 (e.g., SEQ ID NO: 62, SEQ ID NO: 63, SEQ ID NO: 64, SEQ ID NO: 68, or SEQ ID NO: 70) and a fourth nucleic acid sequence positioned 3′ to expression cassette and comprising a sequence that that has at least 70%, at least 75%, at least 80%, at least 85%, at least 90%, at least 95%, at least 99%, or 100% sequence identity with a sequence in Table 7 (e.g., SEQ ID NO: 65, SEQ ID NO: 66, SEQ ID NO: 67, or SEQ ID NO: 69). In some embodiments, (i) and (ii) may be administered as separate compositions.

In some embodiments, a composition as described herein may comprise (i) a nuclease or polynucleotide sequence encoding a nuclease capable of inducing a double-stranded break and/or a single-stranded break at a cut site distal from a target integration site; and (ii) a polynucleotide cassette comprising an expression cassette comprising a first nucleic acid sequence that has at least 70%, at least 75%, at least 80%, at least 85%, at least 90%, at least 95%, at least 99%, or 100% sequence identity with a sequence in Table 6 (e.g., SEQ ID NO: 50), a second nucleic acid sequence is positioned 5′ or 3′ sequence to the first nucleic acid sequence that has at least 70%, at least 75%, at least 80%, at least 85%, at least 90%, at least 95%, at least 99%, or 100% sequence identity with a sequence in Table 5 (e.g., SEQ ID NO: 46, SEQ ID NO: 47, SEQ ID NO: 48) a third nucleic acid sequence positioned 5′ to the expression cassette and comprising a sequence that that has at least 70%, at least 75%, at least 80%, at least 85%, at least 90%, at least 95%, at least 99%, or 100% sequence identity with a sequence in Table 7 (e.g., SEQ ID NO: 62, SEQ ID NO: 63, SEQ ID NO: 64, SEQ ID NO: 68, or SEQ ID NO: 70) and a fourth nucleic acid sequence positioned 3′ to expression cassette and comprising a sequence that that has at least 70%, at least 75%, at least 80%, at least 85%, at least 90%, at least 95%, at least 99%, or 100% sequence identity with a sequence in Table 7 (e.g., SEQ ID NO: 65, SEQ ID NO: 66, SEQ ID NO: 67, or SEQ ID NO: 69). In some embodiments, (i) and (ii) may be administered as separate compositions.

In some embodiments, a composition as described herein may comprise (i) a nuclease or polynucleotide sequence encoding a nuclease capable of inducing a double-stranded break and/or a single-stranded break at a cut site distal from a target integration site; and (ii) a polynucleotide cassette comprising an expression cassette comprising a first nucleic acid sequence that has at least 70%, at least 75%, at least 80%, at least 85%, at least 90%, at least 95%, at least 99%, or 100% sequence identity with a sequence in Table 6 (e.g., SEQ ID NO: 51), a second nucleic acid sequence is positioned 5′ or 3′ sequence to the first nucleic acid sequence that has at least 70%, at least 75%, at least 80%, at least 85%, at least 90%, at least 95%, at least 99%, or 100% sequence identity with a sequence in Table 5 (e.g., SEQ ID NO: 46, SEQ ID NO: 47, SEQ ID NO: 48) a third nucleic acid sequence positioned 5′ to the expression cassette and comprising a sequence that that has at least 70%, at least 75%, at least 80%, at least 85%, at least 90%, at least 95%, at least 99%, or 100% sequence identity with a sequence in Table 7 (e.g., SEQ ID NO: 62, SEQ ID NO: 63, SEQ ID NO: 64, SEQ ID NO: 68, or SEQ ID NO: 70) and a fourth nucleic acid sequence positioned 3′ to expression cassette and comprising a sequence that that has at least 70%, at least 75%, at least 80%, at least 85%, at least 90%, at least 95%, at least 99%, or 100% sequence identity with a sequence in Table 7 (e.g., SEQ ID NO: 65, SEQ ID NO: 66, SEQ ID NO: 67, or SEQ ID NO: 69). In some embodiments, (i) and (ii) may be administered as separate compositions.

In some embodiments, a composition as described herein may comprise (i) a nuclease or polynucleotide sequence encoding a nuclease capable of inducing a double-stranded break and/or a single-stranded break at a cut site distal from a target integration site; and (ii) a polynucleotide cassette comprising an expression cassette comprising a first nucleic acid sequence that has at least 70%, at least 75%, at least 80%, at least 85%, at least 90%, at least 95%, at least 99%, or 100% sequence identity with a sequence in Table 6 (e.g., SEQ ID NO: 52), a second nucleic acid sequence is positioned 5′ or 3′ sequence to the first nucleic acid sequence that has at least 70%, at least 75%, at least 80%, at least 85%, at least 90%, at least 95%, at least 99%, or 100% sequence identity with a sequence in Table 5 (e.g., SEQ ID NO: 46, SEQ ID NO: 47, SEQ ID NO: 48) a third nucleic acid sequence positioned 5′ to the expression cassette and comprising a sequence that that has at least 70%, at least 75%, at least 80%, at least 85%, at least 90%, at least 95%, at least 99%, or 100% sequence identity with a sequence in Table 7 (e.g., SEQ ID NO: 62, SEQ ID NO: 63, SEQ ID NO: 64, SEQ ID NO: 68, or SEQ ID NO: 70) and a fourth nucleic acid sequence positioned 3′ to expression cassette and comprising a sequence that that has at least 70%, at least 75%, at least 80%, at least 85%, at least 90%, at least 95%, at least 99%, or 100% sequence identity with a sequence in Table 7 (e.g., SEQ ID NO: 65, SEQ ID NO: 66, SEQ ID NO: 67, or SEQ ID NO: 69). In some embodiments, (i) and (ii) may be administered as separate compositions.

In some embodiments, a composition as described herein may comprise (i) a nuclease or polynucleotide sequence encoding a nuclease capable of inducing a double-stranded break and/or a single-stranded break at a cut site distal from a target integration site; and (ii) a polynucleotide cassette comprising an expression cassette comprising a first nucleic acid sequence that has at least 70%, at least 75%, at least 80%, at least 85%, at least 90%, at least 95%, at least 99%, or 100% sequence identity with a sequence in Table 6 (e.g., SEQ ID NO: 53), a second nucleic acid sequence is positioned 5′ or 3′ sequence to the first nucleic acid sequence that has at least 70%, at least 75%, at least 80%, at least 85%, at least 90%, at least 95%, at least 99%, or 100% sequence identity with a sequence in Table 5 (e.g., SEQ ID NO: 46, SEQ ID NO: 47, SEQ ID NO: 48) a third nucleic acid sequence positioned 5′ to the expression cassette and comprising a sequence that that has at least 70%, at least 75%, at least 80%, at least 85%, at least 90%, at least 95%, at least 99%, or 100% sequence identity with a sequence in Table 7 (e.g., SEQ ID NO: 62, SEQ ID NO: 63, SEQ ID NO: 64, SEQ ID NO: 68, or SEQ ID NO: 70) and a fourth nucleic acid sequence positioned 3′ to expression cassette and comprising a sequence that that has at least 70%, at least 75%, at least 80%, at least 85%, at least 90%, at least 95%, at least 99%, or 100% sequence identity with a sequence in Table 7 (e.g., SEQ ID NO: 65, SEQ ID NO: 66, SEQ ID NO: 67, or SEQ ID NO: 69). In some embodiments, (i) and (ii) may be administered as separate compositions.

In some embodiments, a composition as described herein may comprise (i) a nuclease or polynucleotide sequence encoding a nuclease capable of inducing a double-stranded break and/or a single-stranded break at a cut site distal from a target integration site; and (ii) a polynucleotide cassette comprising an expression cassette comprising a first nucleic acid sequence that has at least 70%, at least 75%, at least 80%, at least 85%, at least 90%, at least 95%, at least 99%, or 100% sequence identity with a sequence in Table 6 (e.g., SEQ ID NO: 54), a second nucleic acid sequence is positioned 5′ or 3′ sequence to the first nucleic acid sequence that has at least 70%, at least 75%, at least 80%, at least 85%, at least 90%, at least 95%, at least 99%, or 100% sequence identity with a sequence in Table 5 (e.g., SEQ ID NO: 46, SEQ ID NO: 47, SEQ ID NO: 48) a third nucleic acid sequence positioned 5′ to the expression cassette and comprising a sequence that that has at least 70%, at least 75%, at least 80%, at least 85%, at least 90%, at least 95%, at least 99%, or 100% sequence identity with a sequence in Table 7 (e.g., SEQ ID NO: 62, SEQ ID NO: 63, SEQ ID NO: 64, SEQ ID NO: 68, or SEQ ID NO: 70) and a fourth nucleic acid sequence positioned 3′ to expression cassette and comprising a sequence that that has at least 70%, at least 75%, at least 80%, at least 85%, at least 90%, at least 95%, at least 99%, or 100% sequence identity with a sequence in Table 7 (e.g., SEQ ID NO: 65, SEQ ID NO: 66, SEQ ID NO: 67, or SEQ ID NO: 69). In some embodiments, (i) and (ii) may be administered as separate compositions.

In some embodiments, a composition as described herein may comprise (i) a nuclease or polynucleotide sequence encoding a nuclease capable of inducing a double-stranded break and/or a single-stranded break at a cut site distal from a target integration site; and (ii) a polynucleotide cassette comprising an expression cassette comprising a first nucleic acid sequence that has at least 70%, at least 75%, at least 80%, at least 85%, at least 90%, at least 95%, at least 99%, or 100% sequence identity with a sequence in Table 6 (e.g., SEQ ID NO: 55), a second nucleic acid sequence is positioned 5′ or 3′ sequence to the first nucleic acid sequence that has at least 70%, at least 75%, at least 80%, at least 85%, at least 90%, at least 95%, at least 99%, or 100% sequence identity with a sequence in Table 5 (e.g., SEQ ID NO: 46, SEQ ID NO: 47, SEQ ID NO: 48) a third nucleic acid sequence positioned 5′ to the expression cassette and comprising a sequence that that has at least 70%, at least 75%, at least 80%, at least 85%, at least 90%, at least 95%, at least 99%, or 100% sequence identity with a sequence in Table 7 (e.g., SEQ ID NO: 62, SEQ ID NO: 63, SEQ ID NO: 64, SEQ ID NO: 68, or SEQ ID NO: 70) and a fourth nucleic acid sequence positioned 3′ to expression cassette and comprising a sequence that that has at least 70%, at least 75%, at least 80%, at least 85%, at least 90%, at least 95%, at least 99%, or 100% sequence identity with a sequence in Table 7 (e.g., SEQ ID NO: 65, SEQ ID NO: 66, SEQ ID NO: 67, or SEQ ID NO: 69). In some embodiments, (i) and (ii) may be administered as separate compositions.

In some embodiments, a composition as described herein may comprise (i) a nuclease or polynucleotide sequence encoding a nuclease capable of inducing a double-stranded break and/or a single-stranded break at a cut site distal from a target integration site; and (ii) a polynucleotide cassette comprising an expression cassette comprising a first nucleic acid sequence that has at least 70%, at least 75%, at least 80%, at least 85%, at least 90%, at least 95%, at least 99%, or 100% sequence identity with a sequence in Table 6 (e.g., SEQ ID NO: 56), a second nucleic acid sequence is positioned 5′ or 3′ sequence to the first nucleic acid sequence that has at least 70%, at least 75%, at least 80%, at least 85%, at least 90%, at least 95%, at least 99%, or 100% sequence identity with a sequence in Table 5 (e.g., SEQ ID NO: 46, SEQ ID NO: 47, SEQ ID NO: 48) a third nucleic acid sequence positioned 5′ to the expression cassette and comprising a sequence that that has at least 70%, at least 75%, at least 80%, at least 85%, at least 90%, at least 95%, at least 99%, or 100% sequence identity with a sequence in Table 7 (e.g., SEQ ID NO: 62, SEQ ID NO: 63, SEQ ID NO: 64, SEQ ID NO: 68, or SEQ ID NO: 70) and a fourth nucleic acid sequence positioned 3′ to expression cassette and comprising a sequence that that has at least 70%, at least 75%, at least 80%, at least 85%, at least 90%, at least 95%, at least 99%, or 100% sequence identity with a sequence in Table 7 (e.g., SEQ ID NO: 65, SEQ ID NO: 66, SEQ ID NO: 67, or SEQ ID NO: 69). In some embodiments, (i) and (ii) may be administered as separate compositions.

In some embodiments, a composition as described herein may comprise (i) a nuclease or polynucleotide sequence encoding a nuclease capable of inducing a double-stranded break and/or a single-stranded break at a cut site distal from a target integration site; and (ii) a polynucleotide cassette comprising an expression cassette comprising a first nucleic acid sequence that has at least 70%, at least 75%, at least 80%, at least 85%, at least 90%, at least 95%, at least 99%, or 100% sequence identity with a sequence in Table 6 (e.g., SEQ ID NO: 57), a second nucleic acid sequence is positioned 5′ or 3′ sequence to the first nucleic acid sequence that has at least 70%, at least 75%, at least 80%, at least 85%, at least 90%, at least 95%, at least 99%, or 100% sequence identity with a sequence in Table 5 (e.g., SEQ ID NO: 46, SEQ ID NO: 47, SEQ ID NO: 48) a third nucleic acid sequence positioned 5′ to the expression cassette and comprising a sequence that that has at least 70%, at least 75%, at least 80%, at least 85%, at least 90%, at least 95%, at least 99%, or 100% sequence identity with a sequence in Table 7 (e.g., SEQ ID NO: 62, SEQ ID NO: 63, SEQ ID NO: 64, SEQ ID NO: 68, or SEQ ID NO: 70) and a fourth nucleic acid sequence positioned 3′ to expression cassette and comprising a sequence that that has at least 70%, at least 75%, at least 80%, at least 85%, at least 90%, at least 95%, at least 99%, or 100% sequence identity with a sequence in Table 7 (e.g., SEQ ID NO: 65, SEQ ID NO: 66, SEQ ID NO: 67, or SEQ ID NO: 69). In some embodiments, (i) and (ii) may be administered as separate compositions.

In some embodiments, a composition as described herein may comprise (i) a nuclease or polynucleotide sequence encoding a nuclease capable of inducing a double-stranded break and/or a single-stranded break at a cut site distal from a target integration site; and (ii) a polynucleotide cassette comprising an expression cassette comprising a first nucleic acid sequence that has at least 70%, at least 75%, at least 80%, at least 85%, at least 90%, at least 95%, at least 99%, or 100% sequence identity with a sequence in Table 6 (e.g., SEQ ID NO: 58), a second nucleic acid sequence is positioned 5′ or 3′ sequence to the first nucleic acid sequence that has at least 70%, at least 75%, at least 80%, at least 85%, at least 90%, at least 95%, at least 99%, or 100% sequence identity with a sequence in Table 5 (e.g., SEQ ID NO: 46, SEQ ID NO: 47, SEQ ID NO: 48) a third nucleic acid sequence positioned 5′ to the expression cassette and comprising a sequence that that has at least 70%, at least 75%, at least 80%, at least 85%, at least 90%, at least 95%, at least 99%, or 100% sequence identity with a sequence in Table 7 (e.g., SEQ ID NO: 62, SEQ ID NO: 63, SEQ ID NO: 64, SEQ ID NO: 68, or SEQ ID NO: 70) and a fourth nucleic acid sequence positioned 3′ to expression cassette and comprising a sequence that that has at least 70%, at least 75%, at least 80%, at least 85%, at least 90%, at least 95%, at least 99%, or 100% sequence identity with a sequence in Table 7 (e.g., SEQ ID NO: 65, SEQ ID NO: 66, SEQ ID NO: 67, or SEQ ID NO: 69). In some embodiments, (i) and (ii) may be administered as separate compositions.

In some embodiments, a composition as described herein may comprise (i) a nuclease or polynucleotide sequence encoding a nuclease capable of inducing a double-stranded break and/or a single-stranded break at a cut site distal from a target integration site; and (ii) a polynucleotide cassette comprising an expression cassette comprising a first nucleic acid sequence that has at least 70%, at least 75%, at least 80%, at least 85%, at least 90%, at least 95%, at least 99%, or 100% sequence identity with a sequence in Table 6 (e.g., SEQ ID NO: 59), a second nucleic acid sequence is positioned 5′ or 3′ sequence to the first nucleic acid sequence that has at least 70%, at least 75%, at least 80%, at least 85%, at least 90%, at least 95%, at least 99%, or 100% sequence identity with a sequence in Table 5 (e.g., SEQ ID NO: 46, SEQ ID NO: 47, SEQ ID NO: 48) a third nucleic acid sequence positioned 5′ to the expression cassette and comprising a sequence that that has at least 70%, at least 75%, at least 80%, at least 85%, at least 90%, at least 95%, at least 99%, or 100% sequence identity with a sequence in Table 7 (e.g., SEQ ID NO: 62, SEQ ID NO: 63, SEQ ID NO: 64, SEQ ID NO: 68, or SEQ ID NO: 70) and a fourth nucleic acid sequence positioned 3′ to expression cassette and comprising a sequence that that has at least 70%, at least 75%, at least 80%, at least 85%, at least 90%, at least 95%, at least 99%, or 100% sequence identity with a sequence in Table 7 (e.g., SEQ ID NO: 65, SEQ ID NO: 66, SEQ ID NO: 67, or SEQ ID NO: 69). In some embodiments, (i) and (ii) may be administered as separate compositions.

In some embodiments, a composition as described herein may comprise (i) a nuclease or polynucleotide sequence encoding a nuclease capable of inducing a double-stranded break and/or a single-stranded break at a cut site distal from a target integration site; and (ii) a polynucleotide cassette comprising an expression cassette comprising a first nucleic acid sequence that has at least 70%, at least 75%, at least 80%, at least 85%, at least 90%, at least 95%, at least 99%, or 100% sequence identity with a sequence in Table 6 (e.g., SEQ ID NO: 60), a second nucleic acid sequence is positioned 5′ or 3′ sequence to the first nucleic acid sequence that has at least 70%, at least 75%, at least 80%, at least 85%, at least 90%, at least 95%, at least 99%, or 100% sequence identity with a sequence in Table 5 (e.g., SEQ ID NO: 46, SEQ ID NO: 47, SEQ ID NO: 48) a third nucleic acid sequence positioned 5′ to the expression cassette and comprising a sequence that that has at least 70%, at least 75%, at least 80%, at least 85%, at least 90%, at least 95%, at least 99%, or 100% sequence identity with a sequence in Table 7 (e.g., SEQ ID NO: 62, SEQ ID NO: 63, SEQ ID NO: 64, SEQ ID NO: 68, or SEQ ID NO: 70) and a fourth nucleic acid sequence positioned 3′ to expression cassette and comprising a sequence that that has at least 70%, at least 75%, at least 80%, at least 85%, at least 90%, at least 95%, at least 99%, or 100% sequence identity with a sequence in Table 7 (e.g., SEQ ID NO: 65, SEQ ID NO: 66, SEQ ID NO: 67, or SEQ ID NO: 69). In some embodiments, (i) and (ii) may be administered as separate compositions.

### Routes of Administration

In some embodiments, compositions provided herein may be administered to a subject via anyone (or more) of a variety of routes known in the art (e.g., parenteral, subcutaneous, intravenous, intracranial, intraspinal, intraocular, intramuscular, intravaginal, intraperitoneal, epicutaneous, intradermal, rectal, pulmonary, intraosseous, oral, buccal, intraportal, intra-arterial, intratracheal, or nasal). In some embodiments, compositions provided herein may be introduced into cells, which are then introduced into a subject (e.g., liver, muscle, central nervous system (CNS), lung, hematologic cells). In some embodiments, compositions provided herein may be introduced via delivery methods known in the art (e.g., injection, catheter).

In some embodiments, genome editing with the GENERIDE™ platform differs from conventional gene therapy because it uses HR to deliver a corrective gene to one specific location in the genome. In some embodiments, GENERIDE™ inserts a corrective gene in a precise manner, leading to site-specific integration in the genome.

In some embodiments, provided compositions comprise one or more homology arms, a transgene, and a nucleic acid that promotes the production of two independent gene products. In some embodiments, compositions and methods of the present disclosure comprise a first nucleic acid sequence encoding a transgene. In some embodiments, compositions and methods of the present disclosure comprise a second nucleic acid that promotes the production of two independent gene products (e.g., a 2A peptide). In some embodiments, the present disclosure provides an expression cassette comprising a first nucleic acid sequence and a second nucleic acid sequence as described herein.

In some embodiments, a second nucleic acid comprises a nucleic acid sequence encoding a 2A peptide; a nucleic acid sequence encoding an internal ribosome entry site (IRES); a nucleic acid sequence encoding an N-terminal intein splicing region and C-terminal intein splicing region; and/or a nucleic acid sequence encoding a splice donor and a splice acceptor. In some embodiments, compositions and methods of the present disclosure comprise a polynucleotide cassette comprising an expression cassette comprising said first nucleic acid and said second nucleic acid. In some embodiments, compositions and methods of the present disclosure comprise a third nucleic acid sequence comprising a sequence that is substantially homologous to a genomic sequence. In some embodiments, compositions and methods of the present disclosure comprise a fourth nucleic acid sequence comprising a sequence that is substantially homologous to a genomic sequence. In some embodiments, said third nucleic acid sequence is positioned 5′ to the expression cassette and comprises a sequence that is substantially homologous to a genomic sequence 5′ of a target integration site in a genome of a cell. In some embodiments, said fourth nucleic acid sequence is positioned 3′ to the expression cassette and comprises a sequence that is substantially homologous to a genomic sequence 3′ of a target integration site in the genome of the cell.

In some embodiments, one or more compositions described herein are administered in combination. In some embodiments, a first composition may be administered simultaneously with a second composition. In some embodiments, a first composition and second composition may be administered sequentially (e.g., within minutes, hours, days, weeks, or months of one another). In some embodiments, one or more compositions may be administered via the same route (e.g., parenteral, subcutaneous, intravenous, intracranial, intraspinal, intraocular, intramuscular, intravaginal, intraperitoneal, epicutaneous, intradermal, rectal, pulmonary, intraosseous, oral, buccal, intraportal, intra-arterial, intratracheal, or nasal). In some embodiments, one or more compositions may be administered via different routes (e.g., parenteral, subcutaneous, intravenous, intracranial, intraspinal, intraocular, intramuscular, intravaginal, intraperitoneal, epicutaneous, intradermal, rectal, pulmonary, intraosseous, oral, buccal, intraportal, intra-arterial, intratracheal, or nasal).

In some embodiments, one or more compositions administered in combination may comprise: (i) a first composition comprising one payload (e.g., one or more nucleases mRNA and/or protein (e.g., Cas proteins (e.g., Staphylococcus aureus Cas9 (saCas9), Streptococcus pyogenes (spCas9), AZ Nuclease, HF1-Cas9, HF2-Cas9, or HiFi-Cas9), endonucleases, meganucleases, TALENs, ZFNs)); and (ii) a second composition comprising a second, distinct payload (e.g., a polynucleotide sequence comprising a transgene-encoding region). In some embodiments, the first and second compositions are delivered with the same delivery system (e.g., viral vector, lipid nanoparticle, and the like). In some embodiments, the first and second compositions are delivered with different delivery systems (e.g., viral vector, lipid nanoparticle, and the like). In some embodiments, the first composition is administered prior to the second composition (e.g., by a difference of minutes, hours, days, weeks, or months). In some embodiments, the second composition is administered prior to the first composition (e.g., by a difference of minutes, hours, days, weeks, or months). In some embodiments, the first and second compositions are administered simultaneously. In some embodiments, the first and second compositions are combined prior to administration.

In some embodiments, the first and/or second compositions are administered at a particular dose (e.g., a fixed dose or a weight-based dose) only once. In some embodiments, the first and/or second compositions are administered at a particular dose (e.g., a fixed dose or a weight-based dose) more than once. In some embodiments, where more than one dose is administered (e.g., a fixed dose or a weight-based dose) the first and/or second compositions may be administered simultaneously, substantially simultaneously, or consecutively. In some embodiments, multiple doses (e.g., a fixed dose or a weight-based dose) are administered within a specified period of time (e.g., within minutes, hours, days, weeks, or months).

In some embodiments, the first and/or second compositions are administered in response to a biomarker (e.g., a circulating biomarker as described in WO2020214582A1). For example, the first and/or second compositions are administered at a particular dose (e.g., a fixed dose or a weight-based dose) and within a specified period of time (e.g., within minutes, hours, days, weeks, or months) levels of a biomarker (e.g., as described in WO2020214582A1) are monitored. If levels of a biomarker (e.g., as described in WO2020214582A1) are low (e.g., as compared to an appropriate reference (e.g., levels of a biomarker prior to administration)), then the first and/or second compositions are administered at a particular dose (e.g., a fixed dose or a weight-based dose). If levels of a biomarker (e.g., as described in WO2020214582A1) are high (e.g., as compared to an appropriate reference (e.g., levels of a biomarker after an initial administration)), then subsequent dosing (e.g., a fixed dose or a weight-based dose) of the first and/or second compositions may be reevaluated (e.g., treatment suspension, reduced fixed dose or weight-based dose).

### Methods of Producing Viral Vectors

### Production of Viral Vectors

In some embodiments, production of viral vectors (e.g., AAV viral vectors) may include both upstream steps to generate viral vectors (e.g., cell-based culturing) and downstream steps to process viral vectors (e.g., purification, formulation, and the like). In some embodiments, upstream steps may comprise one or more of cell expansion, cell culture, cell transfection, cell lysis, viral vector production, and/or viral vector harvest.

In some embodiments, downstream steps may comprise one or more of separation, filtration, concentration, clarification, purification, chromatography (e.g., affinity, ion exchange, hydrophobic, mixed-mode), centrifugation (e.g., ultracentrifugation), and/or formulation.

In some embodiments, constructs and methods described herein are designed to increase viral vector yields (e.g., AAV vector yields), reduce levels of replication-competent viral vectors (e.g., replication competent AAV (rcAAV)), improve viral vectors packaging efficiency (e.g., AAV vector capsid packaging), and/or any combinations thereof, relative to a reference construct or method, for example those in Xiao et al. 1998 and Grieger et al. 2015, each of which is incorporated herein by reference in its entirety.

### Cell Lines and Transfection Reagents

In some embodiments, production of viral vectors comprises use of cells (e.g., cell culture). In some embodiments, production of viral vectors comprises use of cell culture of one or more cell lines (e.g., mammalian cell lines). In some embodiments, production of viral vectors comprises use of HEK293 cell lines or variants thereof (e.g., HEK293T, HEK293F cell lines). In some embodiments, cells are capable of being grown in suspension. In some embodiments, cells are comprised of adherent cells. In some embodiments, cells are capable of being grown in media that does not comprise animal components (e.g., animal serum). In some embodiments, cells are capable of being grown in serum-free media (e.g., F17 media, Expi293 media). In some embodiments, production of viral vectors comprises transfection of cells with expression constructs (e.g., plasmids). In some embodiments, cells are selected for high expression of viral vectors (e.g., AAV vectors). In some embodiments, cells are selected for high packaging efficiency of viral vectors (e.g., capsid packaging of AAV vectors). In some embodiments, cells are selected for improved transfection efficiency (e.g., with chemical transfection reagents, including cationic molecules). In some embodiments, cells are engineered for high expression of viral vectors (e.g., AAV vectors). In some embodiments, cells are engineered for high packaging efficiency of viral vectors (e.g., capsid packaging of AAV vectors). In some embodiments, cells are engineered for improved transfection efficiency (e.g., with chemical transfection reagents, including cationic molecules). In some embodiments, cells may be engineered or selected for two or more of the above attributes. In some embodiments, cells are contacted with one or more expression constructs (e.g., plasmids). In some embodiments, cells are contacted with one or more transfection reagents (e.g., chemical transfection reagents, including lipids, polymers, and cationic molecules) and one or more expression constructs. In some embodiments, cells are contacted with one or more cationic molecules (e.g., cationic lipid, PEI reagent) and one or more expression constructs. In some embodiments, cells are contacted with a PEIMAX reagent and one or more expression constructs. In some embodiments, cells are contacted with a Fecto Vir-AAV reagent and one or more expression constructs. In some embodiments, cells are contacted with one or more transfection reagents and one or more expression constructs at particular ratios. In some embodiments, ratios of transfection reagents to expression constructs improves production of viral vectors (e.g., improved vector yield, improved packaging efficiency, and/or improved transfection efficiency).

### Expression Constructs

In some embodiments, expression constructs are or comprise one or more polynucleotide sequences (e.g., plasmids). In some embodiments, expression constructs comprise particular polynucleotide sequence elements (e.g., payloads, promoters, viral genes, and the like). In some embodiments, expression constructs comprise polynucleotide sequences encoding viral genes (e.g., a rep or cap gene or gene variant, one or more helper virus genes or gene variants). In some embodiments, expression constructs of a particular type comprise specific combinations of polynucleotide sequence elements. In some embodiments, expression constructs of a particular type do not comprise specific combinations of polynucleotide sequence elements. In some embodiments, a particular expression construct does not comprise polynucleotide sequence elements encoding both rep and cap genes and/or gene variants.

In some embodiments, expression constructs comprise polynucleotide sequences encoding wild-type viral genes (e.g., wild-type rep genes, cap genes, viral helper genes, or combinations thereof). In some embodiments, expression constructs comprise polynucleotide sequences encoding viral helper genes or gene variants (e.g., herpesvirus genes or gene variants, adenovirus genes or gene variants). In some embodiments, expression constructs comprise polynucleotide sequences encoding one or more gene copies that express one or more wild-type Rep proteins (e.g., 1 copy, 2 copies, 3 copies, 4 copies, 5 copies, and the like). In some embodiments, expression constructs comprise polynucleotide sequences encoding a single gene copy that expresses one or more wild-type Rep proteins (e.g., Rep68, Rep40, Rep52, Rep78, or combinations thereof). In some embodiments, expression constructs comprise polynucleotide sequences encoding one or more wild-type Rep proteins (e.g., Rep68, Rep40, Rep52, Rep78, or combinations thereof). In some embodiments, expression constructs comprise polynucleotide sequences encoding at least four wild-type Rep proteins (e.g., Rep68, Rep40, Rep52, Rep78). In some embodiments, expression constructs comprise polynucleotide sequences encoding each of Rep68, Rep40, Rep52, and Rep78. In some embodiments, expression constructs comprise polynucleotide sequences encoding one or more wild-type adenoviral helper proteins (e.g., E2 and E4).

In some embodiments, expression constructs comprise wild-type polynucleotide sequences encoding wild-type viral genes (e.g., rep genes, cap genes, helper genes). In some embodiments, expression constructs comprise modified polynucleotide sequences (e.g., codon-optimized) encoding wild-type viral genes (e.g., rep genes, cap genes, helper genes). In some embodiments, expression constructs comprise modified polynucleotide sequences encoding modified viral genes (e.g., rep genes, cap genes, helper genes). In some embodiments, modified viral genes are designed and/or engineered for certain improvements (e.g., improved transduction, tissue specificity, reduced size, reduced immune response, improved packaging, reduced rcAAV levels, and the like).

In accordance with various embodiments, expression constructs disclosed herein may offer increased flexibility and modularity as compared to previous technologies. In some embodiments, expression constructs disclosed herein may allow swapping of various polynucleotide sequences (e.g., different rep genes, cap genes, payloads, helper genes, promoters, and the like) while providing certain improvements (e.g., increased viral vector yield, increased packaging, reduced rcAAV levels, and the like). In some embodiments, expression constructs disclosed herein are compatible with various upstream production processes (e.g., different cell culture conditions, different transfection reagents, and the like) while providing certain improvements (e.g., increased viral vector yield, increased packaging, reduced rcAAV levels, and the like).

In some embodiments, expression constructs of different types comprise different combinations of polynucleotide sequences. In some embodiments, an expression construct of one type comprises one or more polynucleotide sequence elements (e.g., payloads, promoters, viral genes, and the like) that is not present in an expression construct of a different type. In some embodiments, an expression construct of one type comprises polynucleotide sequence elements encoding a viral gene (e.g., a rep or cap gene or gene variant) and polynucleotide sequence elements encoding a payload (e.g., a transgene and/or functional nucleic acid). In some embodiments, an expression construct of one type comprises polynucleotide sequence elements encoding one or more viral genes (e.g., a rep or cap gene or gene variant and/or one or more helper virus genes). In some embodiments, an expression construct of one type comprises polynucleotide sequence elements encoding one or more viral genes, wherein the viral genes are from one or more virus types (e.g., genes or gene variants from AAV and adenovirus). In some embodiments, viral genes from adenovirus are genes and/or gene variants. In some embodiments, viral genes from adenovirus are one or more of E2A (e.g., E2A DNA Binding Protein (DBP), E4 (e.g., E4 Open Reading Frame (ORF) 2, ORF3, ORF4, ORF6/7), VA, and/or variants thereof.

In some embodiments, expression constructs are used for production of viral vectors (e.g., through cell culture). In some embodiments, expression constructs are contacted with cells in combination with one or more transfection reagents (e.g., chemical transfection reagents). In some embodiments, expression constructs are contacted with cells at particular ratios in combination with one or more transfection reagents. In some embodiments, expression constructs of different types are contacted with cells at particular ratios (e.g., weight ratios) in combination with one or more transfection reagents. In some embodiments, expression constructs of different types are contacted with cells at about a 10:1, 9:1, 8:1, 7:1, 6:1, 5:1, 4:1, 3:1, 2:1, 1.5:1, 1:1, 1:1.5, 1:2, 1:3, 1:4, 1:5, 1:6, 1:7, 1:8, 1:9, or 1:10 ratio (e.g., weight ratio). In some embodiments, a first expression construct comprising one or more viral helper genes and a second expression construct comprising one or more payloads are contacted with cells at about a 10:1, 9:1, 8:1, 7:1, 6:1, 5:1, 4:1, 3:1, 2:1, 1.5:1, 1:1, 1:1.5, 1:2, 1:3, 1:4, 1:5, 1:6, 1:7, 1:8, 1:9, or 1:10 ratio (e.g., weight ratio) of the first expression construct to the second expression construct. In some embodiments, a first expression construct comprising one or more payloads and a second expression construct comprising one or more viral helper genes are contacted with cells at about a 10:1, 9:1, 8:1, 7:1, 6:1, 5:1, 4:1, 3:1, 2:1, 1.5:1, 1:1, 1:1.5, 1:2, 1:3, 1:4, 1:5, 1:6, 1:7, 1:8, 1:9, or 1:10 ratio (e.g., weight ratio) of the first expression construct to the second expression construct. In some embodiments, particular ratios of expression constructs improve production of AAV (e.g., increased viral vector yields, increased packaging efficiency, and/or increased transfection efficiency. In some embodiments, cells are contacted with two or more expression constructs (e.g., sequentially, or substantially simultaneously). In some embodiments, three or more expression constructs are contacted with cells. In some embodiments, expression constructs comprise one or more promoters (e.g., one or more exogenous promoters). In some embodiments, promoters are or comprise CMV, RSV, CAG, EF1alpha, PGK, A1AT, C5-12, MCK, desmin, p5, p40, or combinations thereof. In some embodiments, expression constructs comprise one or more promoters upstream of a particular polynucleotide sequence element (e.g., a rep or cap gene or gene variant). In some embodiments, expression constructs comprise one or more promoters downstream of a particular polynucleotide sequence element (e.g., a rep or cap gene or gene variant).

In some embodiments, expression constructs comprise one or more polynucleotide sequences encoding elements (e.g., selection markers, origins of replication) necessary for cell culture (e.g., bacterial cell culture, mammalian cell culture). In some embodiments, expression constructs comprise one or more polynucleotide sequences encoding antibiotic resistance genes (e.g., kanamycin resistance genes, ampicillin resistance genes). In some embodiments, expression constructs comprise one or more polynucleotide sequences encoding a bacterial original of replication (e.g., colE1 origin of replication).

In some embodiments, expression constructs comprise one or more transcription termination sequences (e.g., a polyA sequence). In some embodiments, expression constructs comprise one or more of BGH polyA, FIX polyA, SV40 polyA, synthetic polyA, or combinations thereof. In some embodiments, expression constructs comprise one or more transcription termination sequences downstream of a particular sequence element (e.g., a rep or cap gene or gene variant). In some embodiments, expression constructs comprise one or more transcription termination sequences upstream of a particular sequence element (e.g., a rep or cap gene or gene variant).

In some embodiments, expression constructs comprise one or more intron sequences. In some embodiments, expression constructs comprise one or more of introns of different origins (e.g., known genes), including but not limited to FIX intron, Albumin intron, or combinations thereof. In some embodiments, expression constructs comprise one or more introns of different lengths (e.g., 133 bp to 4 kb). In some embodiments, expression constructs comprise one or more intron sequences upstream of a particular sequence element (e.g., a rep or cap gene or gene variant). In some embodiments, expression constructs comprise one or more intron sequences within a particular sequence element (e.g., a rep or cap gene or gene variant). In some embodiments, expression constructs comprise one or more intron sequences downstream of particular sequence element (e.g., a rep or cap gene or gene variant). In some embodiments, expression constructs comprise one or more intron sequences after a promoter (e.g., a p5 promoter). In some embodiments, expression constructs comprise one or more intron sequences before a rep gene or gene variant. In some embodiments, expression constructs comprise one or more intron sequences between a promoter and a rep gene or gene variant.

### EXEMPLIFICATION

### Example 1: Induced Double Stranded Break by Cas9 May Enhance GENERIDE™ Efficiency

This example demonstrates that, among other things, administration of a GENERIDE™ construct to a target integration site in a coding region of an endogenous human locus (e.g., human albumin (ALB)) in combination with one or more Cas9 enzymes targeting a cut site within a non-coding sequence of an endogenous human locus (e.g., human ALB intron) may provide improved transgene integration efficiency.

Viral vectors comprising a viral capsid (e.g., LK03), P2A sequence, a transgene gene (e.g., UGT1A1), a balanced (1 kb/1 kb) flanking 5′ and 3′ homology arm lengths were constructed. Homology arms were designed to be complementary to a human genomic albumin target integration site. HepG2 cells, a human liver hepatocellular carcinoma cell line, were transduced with viral vectors at dose of MOI=1E⁵. Following transduction, cells were transfected with spCas9 and gRNA (designed using IDT gRNA algorithm) using Lipofectamine™ CRISPRMAX (ThermoFisher Scientific, Inc) according to the manufacturer's instructions. gRNA were designed to target ALB gene at either intron 13 or 14 (Table 3). 48 hours after transfection, total RNA was isolated from cells by using the RNeasy kit (Qiagen) following the vendor's instructions and assessed for levels of fused mRNA (e.g., see Table 8).

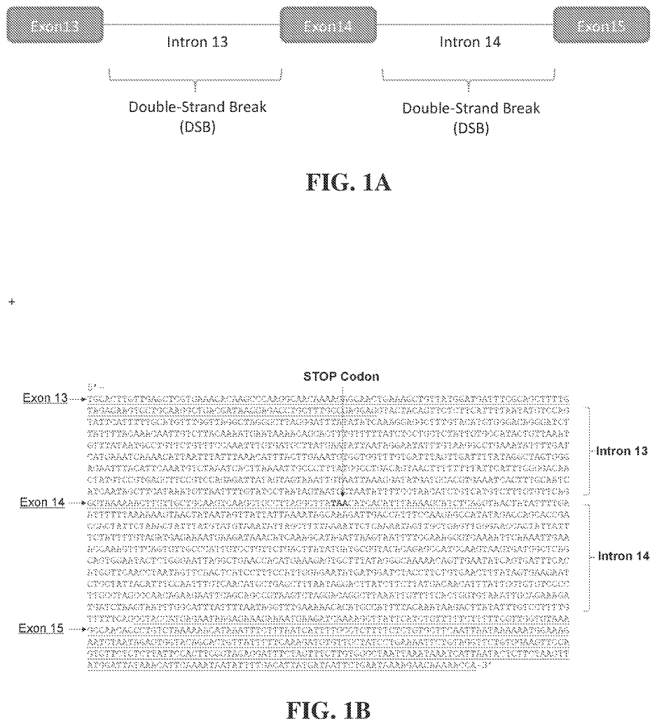

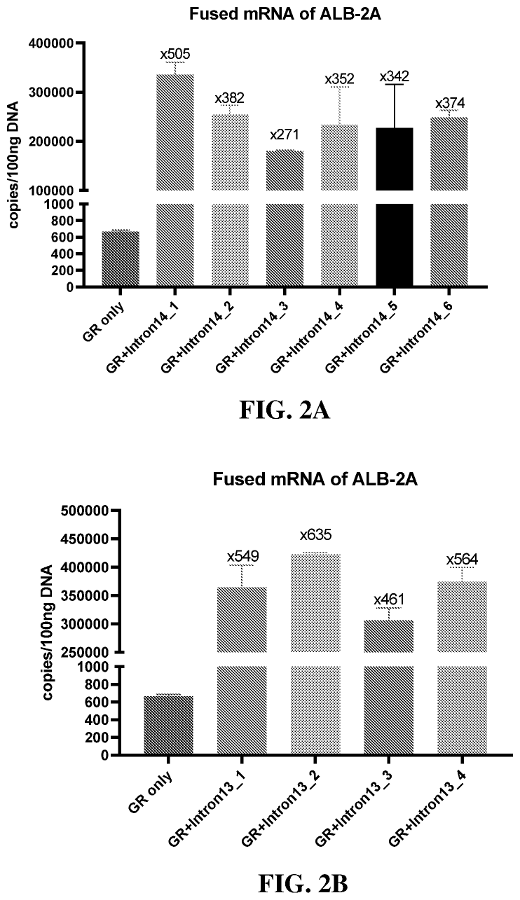

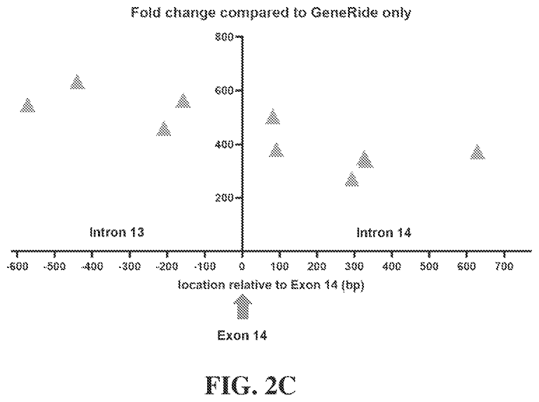

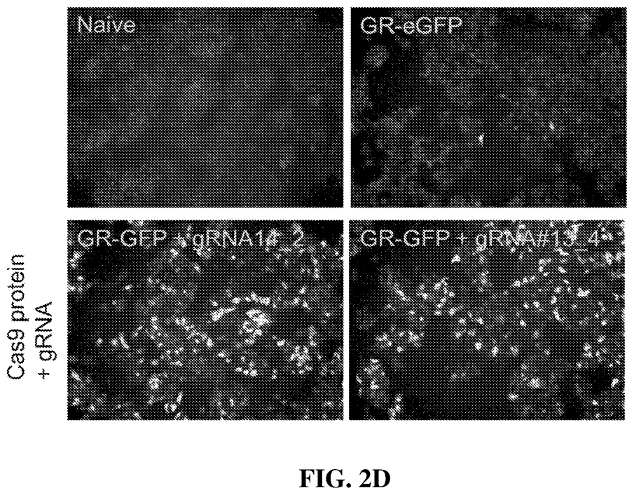

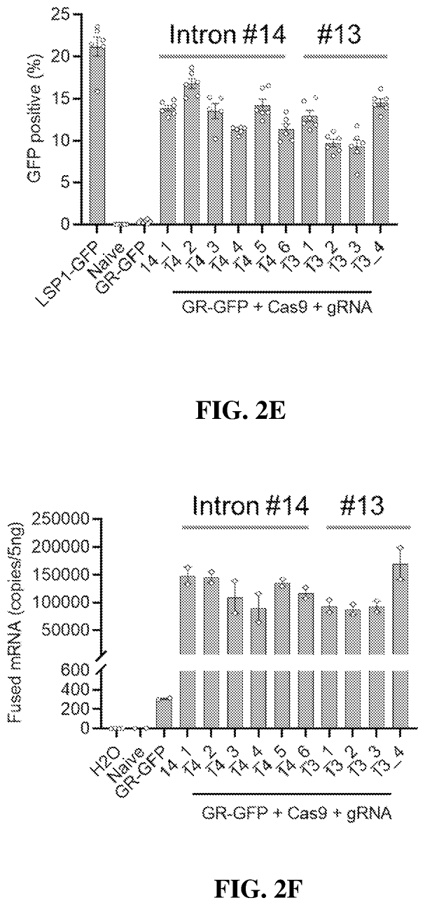

Among other things, this example demonstrates that viral vectors, as described herein, comprising a transgene (e.g., UGT1A1) in combination with spCas9 may provide improved editing activity. In some embodiments, spCas9 may induce a double strand break (DSB) at a cut site within intron 13 and/or 14 of ALB gene (FIGS. 1A and 1B). In some embodiments, as demonstrated in FIG. 2A, induced DSB in intron 13 of human ALB gene may enhance transgene integration efficiency by at least 400-fold as compared to an appropriate reference (e.g., administration of vector without induced DSB). In some embodiments, as demonstrated in FIG. 2B, induced DSB in intron 14 of human ALB gene may enhance transgene integration efficiency by at least 200-fold as compared to an appropriate reference (e.g., administration of vector without induced DSB). In some embodiments, administration of a GENERIDE™ construct in combination with a DSB in any non-coding sequence within an endogenous locus may provide improved transgene integration efficiency relative to a GENERIDE™ construct alone. In some embodiments, administration of a GENERIDE™ construct in combination with a DSB in a particular region of a non-coding sequence within an endogenous locus may provide improved or comparable transgene integration efficiency as compared to a DSB in an alternate region of a non-coding sequence (FIG. 2C). In some embodiments, a DSB is located in a non-coding sequence that is distal from the integration site of the transgene.

Viral vectors comprising a viral capsid (e.g., LK03), P2A sequence, a transgene gene (e.g., GFP), a balanced (1 kb/1 kb) flanking 5′ and 3′ homology arm lengths were constructed. Homology arms were designed to be complementary to a human genomic albumin target integration site. HepG2 cells, a human liver hepatocellular carcinoma cell line, were transduced with viral vectors at dose of MOI=1E⁵. Following transduction, cells were transfected with spCas9 and gRNA (designed using IDT gRNA algorithm) using Lipofectamine™ CRISPRMAX (ThermoFisher Scientific, Inc) according to the manufacturer's instructions. gRNA were designed to target ALB gene at either intron 13 or 14 (Table 3). 48 hours after transfection, total RNA was isolated from cells by using the RNeasy kit (Qiagen) following the vendor's instructions and assessed for levels of fused mRNA. In addition, immunohistochemistry and/or immunocytochemistry was performed (e.g., see Table 8) Images were evaluated using an imaging processing software (e.g., ImageJ).

Among other things, this example demonstrates that viral vectors, as described herein, comprising a transgene (e.g., GFP) in combination with spCas9 may provide improved editing activity and protein expression. As demonstrated in FIGS. 2D and 2E induced DSB in intron 13 and/or 14 may enhance protein expression (e.g., % GFP positive cells) by at least 5-fold. Further, as shown in FIG. 2F, increased levels of fused mRNA may be associated with increased protein expression.

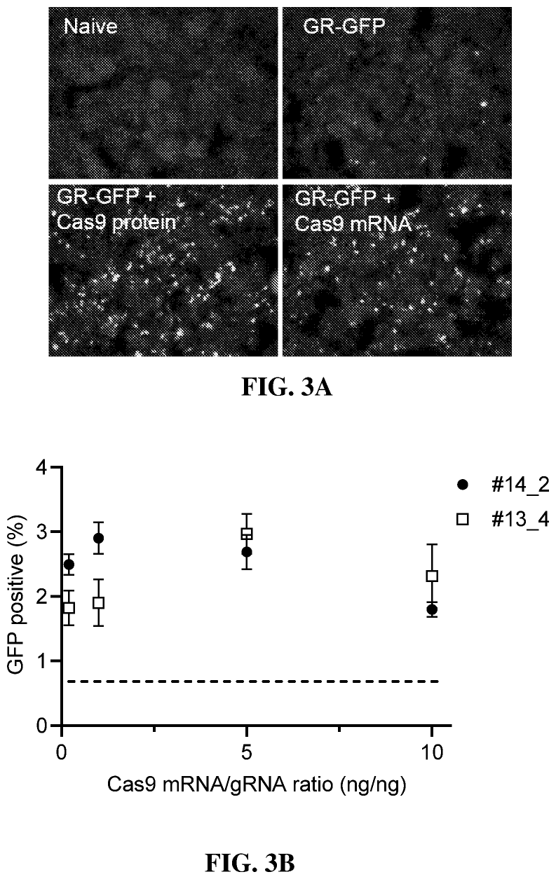

Viral vectors comprising a viral capsid (e.g., LK03), P2A sequence, a transgene gene (e.g., GFP), a balanced (1 kb/1 kb) flanking 5′ and 3′ homology arm lengths were constructed. Homology arms were designed to be complementary to a human genomic albumin target integration site. HepG2 cells, a human liver hepatocellular carcinoma cell line, were transduced with viral vectors at dose of MOI=1E⁵. Following transduction, cells were transfected with spCas9 mRNA and gRNA (designed using IDT gRNA algorithm) were mixed using Lipofectamine™ CRISPRMAX (ThermoFisher Scientific, Inc) according to the manufacturer's instructions. gRNA were designed to target ALB gene at either intron 13 or 14 (Table 8). 72 hours after transfection, cells were fixed and imaged. GFP-positive cells were quantified using softwares (e.g., Image J or CellProfiler) (FIG. 3A).

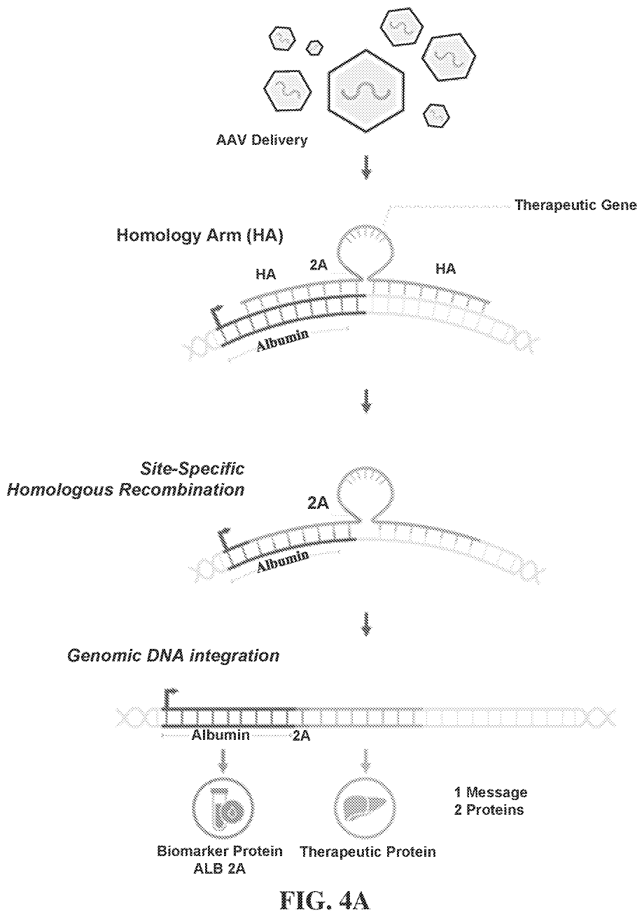

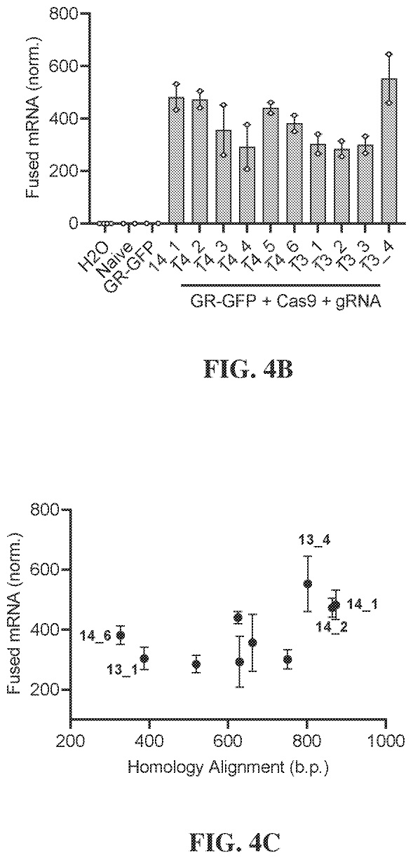

Among other things, this example demonstrates that spCas9 mRNA may also enhance editing activity of viral vectors, as described herein (FIG. 3B). As demonstrated in FIG. 4, a ratio of about 5 of spCas9 mRNA to guide RNA may provide improved editing activity of viral vectors. In some embodiments, the ratio of spCas9 mRNA to guide RNA may be optimized to improve transgene integration efficiency.

Viral vectors comprising a viral capsid (e.g., LK03), P2A sequence, a transgene gene (e.g., GFP, HA tag-conjugated GFP, or UGT1A1), a balanced (1 kb/1 kb) flanking 5′ and 3′ homology arm lengths were constructed. Homology arms were designed to be complementary to a human genomic albumin target integration site. HepG2 cells, a human liver hepatocellular carcinoma cell line, were transduced with viral vectors at dose of MOI=1E⁵. Following transduction, cells were transfected with Cas9 (e.g., spCas9-HF1 and/or spCas9-HF2) and gRNA (designed using IDT gRNA algorithm) using Lipofectamine™ CRISPRMAX (ThermoFisher Scientific, Inc) according to the manufacturer's instructions. gRNA were designed to target ALB gene at either intron 13 or 14 (Table 3). 48 hours after transfection, immunohistochemistry and/or immunocytochemistry was performed using an antibody. Images were evaluated using an imaging processing software (e.g., ImageJ).

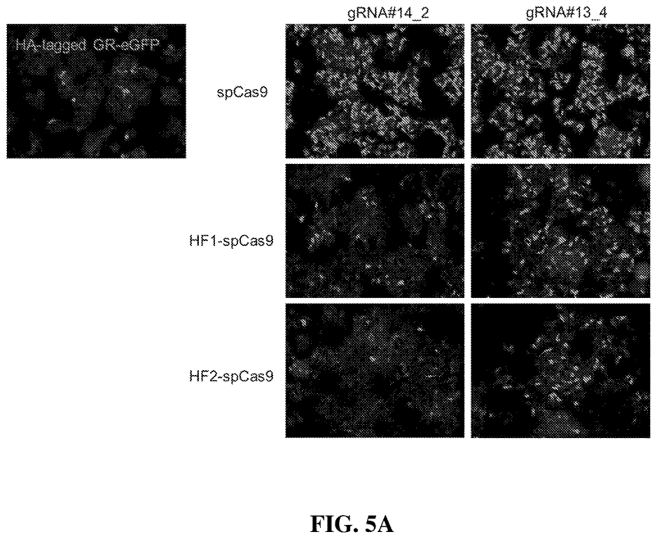

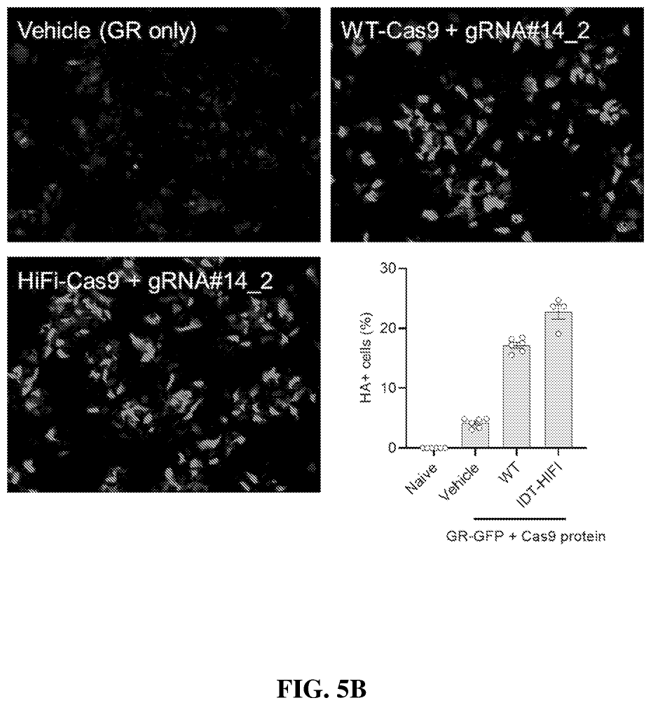

Among other things, this example demonstrates that viral vectors, as described herein, in combination with different spCas9 (e.g., spCas9-HF1 and/or spCas9-HF2) utilizing gRNA designed to target intron 13 and/or 14 of albumin may improve GENERIDE™ editing activity. As demonstrated in FIG. 5, spCas9-HF1 and/or spCas9-HF2 induced DSB in intron 14 may enhance protein expression (e.g., % GFP positive cells) as compared to induced DSB in intron 13.

Viral vectors comprising a viral capsid (e.g., LK03), P2A sequence, a HA conjugated transgene gene (e.g., GFP, HA-conjugated GFP, and/or UGT1A1), a balanced (1 kb/1 kb) flanking 5′ and 3′ homology arm lengths were constructed. Homology arms were designed to be complementary to a human genomic albumin target integration site. HepG2 cells, a human liver hepatocellular carcinoma cell line, were transduced with viral vectors at dose of MOI=1E⁵. Following transduction, cells were transfected with saCas9 and gRNA (designed using IDT gRNA algorithm) using Lipofectamine™ CRISPRMAX (ThermoFisher Scientific, Inc) according to the manufacturer's instructions. gRNA were designed to target ALB gene at either intron 13 or 14 (Table 3). Immunohistochemistry and/or immunocytochemistry was performed (e.g., see Table 8) Images were evaluated using an imaging processing software (e.g., ImageJ).

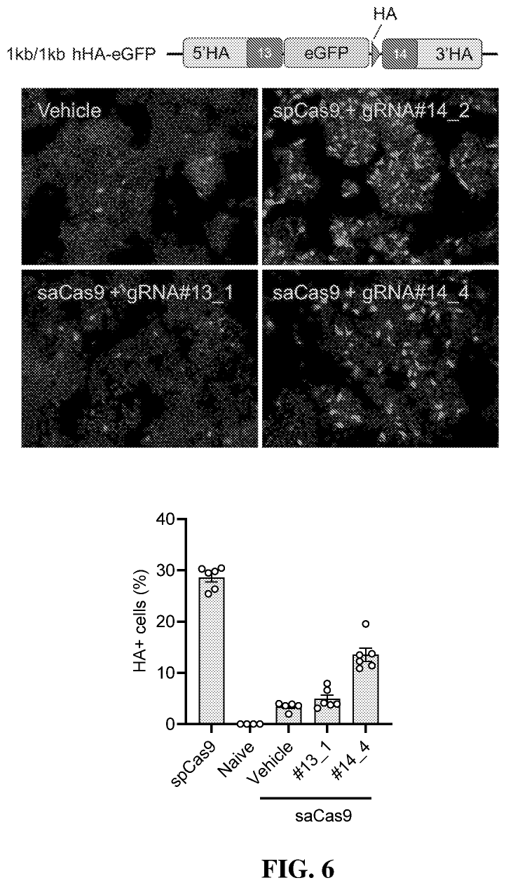

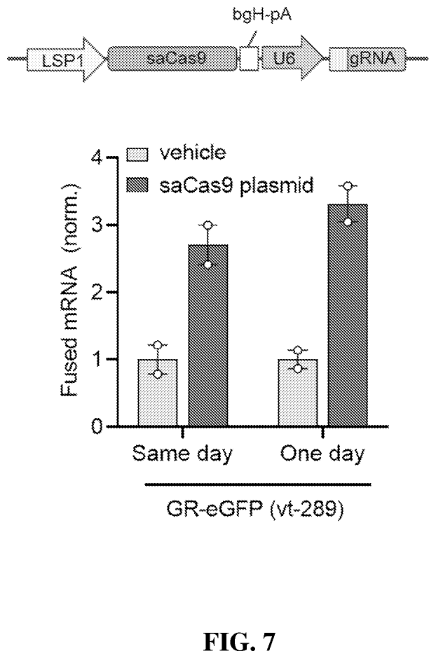

Among other things, this example demonstrates that viral vectors, as described herein, in combination with saCas9 utilizing gRNA designed to target intron 13 and/or 14 of albumin may improve GENERIDE™ editing activity. As demonstrated in FIG. 6, saCas9 induced DSB may enhance protein expression (e.g., % HA+ positive cells).

### Example 2: Induced DNA Break by Cas Nucleases May Enhance GENERIDE™ Efficiency

This example demonstrates that, among other things, administration of a GENERIDE™ construct to a target integration site in a coding region of an endogenous human locus (human albumin) in combination with one or more Cas enzyme (e.g., Cas9, Cas13, Cas12a, Cas9 nickase, and the like) targeting a cut site within a non-coding sequence of an endogenous human locus (e.g., human ALB intron) may provide improved transgene integration efficiency.

A first composition comprising viral vectors comprising a viral capsid, P2A sequence, a transgene gene, and balanced or unbalanced flanking 5′ and 3′ homology arm lengths is constructed. Homology arms are designed to be complementary to a target integration site (e.g., human genomic albumin).

A second composition comprising a Cas enzyme (e.g., Cas9, Cas13, Cas12a, Cas9 nickase, and the like) and a gRNA designed to target a non-coding sequence of an endogenous human locus (e.g., human ALB intron) is constructed. Several methods for delivering such a composition are known in the art (e.g., transfection with plasmids or mRNA encoding a Cas enzymes (e.g., Cas9, Cas13, Cas12a, Cas9 nickase, and the like) and a gRNA, transduction with viral vectors encoding a Cas enzymes (e.g., Cas9, Cas13, Cas12a, Cas9 nickase, and the like) and a gRNA, and/or intracellular delivery of a Cas enzyme (e.g., Cas9, Cas13, Cas12a, Cas9 nickase, and the like)-gRNA ribonucleoprotein (RNP))

Following construction of each composition, several experimental conditions are tested. In one experiment, cells (e.g., HepG2) are concurrently transduced and/or transfected with a first composition and second composition. In another experiment, cells (e.g., HepG2) are sequentially transduced with a first composition and then transduced and/or transfected with a second composition. In another experiment, cells (e.g., HepG2) are sequentially transduced and/or transfected with a second composition and then transduced with a first composition. In another experiment, cells (e.g., HepG2) are concurrently transduced and/or transfected with a first composition and second composition, and then repeatedly (e.g., at least more than once) transduced and/or transfected with a second composition. In another experiment, cells are sequentially transduced with a first composition and then repeatedly (e.g., at least more than once) transduced and/or transfected with a second composition. After transfection and/or transduction, total RNA is isolated from cells by using the RNeasy kit (Qiagen) following the vendor's instructions and assessed for levels of fused mRNA.

Among other things, this example demonstrates that viral vectors, as described herein, in combination with Cas enzymes (e.g., Cas9, Cas13, Cas12a, Cas9 nickase, and the like) may provide improved editing activity. In some embodiments, Cas enzymes (e.g., Cas9, Cas13, Cas12a, Cas9 nickase, and the like) can induce a DNA break (e.g., double strand break (DSB) and/or single strand break (SSB)) within a non-coding sequence of a human ALB gene. In some embodiments, induced DNA break (e.g., DSB and/or SSB) within a non-coding sequence of a human ALB gene may enhance transgene integration efficiency as compared to an appropriate reference (e.g., administration of vector without induced DSB and/or SSB). In some embodiments, administration of a GENERIDE™ construct in combination with an induced DNA break (e.g., DSB and/or SSB) in any non-coding sequence within an endogenous locus may provide improved transgene integration efficiency relative to a GENERIDE™ construct alone. In some embodiments, administration of a GENERIDE™ construct in combination with an induced DNA break (e.g., DSB and/or SSB) in a particular region of a non-coding sequence within an endogenous locus may provide improved or comparable transgene integration efficiency as compared to an induced DNA break (e.g., DSB and/or SSB) in an alternate region of a non-coding sequence. In some embodiments, an induced DNA break (e.g., DSB and/or SSB) is located in a non-coding sequence that is distal from the integration site of the transgene.

### Example 3: Induced DNA Break by ZFNs May Enhance GENERIDE™ Efficiency

This example demonstrates that, among other things, administration of a GENERIDE™ construct to a target integration site in a coding region of an endogenous human locus (e.g., human albumin (ALB)) in combination with one or more Zinc Finger Nuclease (ZFN) (e.g., pairs of ZFNs and/or ZFN Nickase (ZFNickase) targeting a cut site within a non-coding sequence of an endogenous human locus (e.g., human albumin intron) may provide improved transgene integration efficiency.

A first composition comprising viral vectors comprising a viral capsid, P2A sequence, a transgene gene, and balanced or unbalanced flanking 5′ and 3′ homology arm lengths is constructed. Homology arms are designed to be complementary to a target integration site (e.g., human genomic albumin).

A second composition comprising one or more ZFN (e.g., pairs of ZFNs and/or ZFNickase) designed to target a non-coding sequence of an endogenous human locus (e.g., human ALB intron) is constructed. A ZFN/ZFNickase may be prepared such that it can bind to a specific region of a gene (e.g., intron 13 and/or 14 of a human albumin gene) and comprise a nuclease domain (e.g., FokI) capable of nicking (e.g., ZFNickase) or cleaving (e.g., pairs of ZFNs) the target sequence. Several methods for delivering such a composition are known in the art (e.g., transfection with plasmids or mRNA encoding one or more ZFN (e.g., pairs of ZFNs and/or ZFNickase), transduction with viral vectors encoding one or more ZFN (e.g., pairs of ZFNs and/or ZFNickase) and/or electroporation).

Following construction of each composition, several experimental conditions are tested. In one experiment, cells (e.g., HepG2) are concurrently transduced and/or transfected with a first composition and second composition. In a following experiment, cells (e.g., HepG2) are sequentially transduced with a first composition and then transduced and/or transfected with a second composition. In a following experiment, cells (e.g., HepG2) are sequentially transduced and/or transfected with a second composition and then transduced with a first composition. In a following experiment, cells (e.g., HepG2) are concurrently transduced and/or transfected with a first composition and second composition, and then repeatedly (e.g., at least more than once) transduced and/or transfected with a second composition. In the following experiment, cells are sequentially transduced with a first composition and then repeatedly (e.g., at least more than once) transduced and/or transfected with a second composition. After transfection and/or transduction, total RNA is isolated from cells by using the RNeasy kit (Qiagen) following the vendor's instructions and assessed for levels of fused mRNA.

Among other things, this example demonstrates that viral vectors, as described herein, in combination with one or more ZFN (e.g., pairs of ZFNs and/or ZFNickase) may provide improved editing activity. In some embodiments, one or more ZFN (e.g., pairs of ZFNs and/or ZFN ZFNickase) can induce a DNA break (e.g., DSB and/or SSB) within a non-coding sequence of a human ALB gene. In some embodiments, induced DNA break (e.g., DSB and/or SSB) within a non-coding sequence of a human ALB gene may enhance transgene integration efficiency as compared to an appropriate reference (e.g., administration of vector without induced DSB and/or SSB). In some embodiments, administration of a GENERIDE™ construct in combination with an induced DNA break (e.g., DSB and/or SSB) in any non-coding sequence within an endogenous locus may provide improved transgene integration efficiency relative to a GENERIDE™ construct alone. In some embodiments, administration of a GENERIDE™ construct in combination with an induced DNA break (e.g., DSB and/or SSB) in a particular region of a non-coding sequence within an endogenous locus may provide improved or comparable transgene integration efficiency as compared to an induced DNA break (e.g., DSB and/or SSB) in an alternate region of a non-coding sequence. In some embodiments, an induced DNA break (e.g., DSB and/or SSB) is located in a non-coding sequence that is distal from the integration site of the transgene.

### Example 4: Induced DNA Break by Meganucleases May Enhance GENERIDE™ Efficiency

This example demonstrates that, among other things, administration of a GENERIDE™ construct to a target integration site in a coding region of an endogenous human locus (e.g., human albumin (ALB)) in combination with one or more meganuclease targeting a cut site within a non-coding sequence of an endogenous human locus (e.g., human albumin intron) may provide improved transgene integration efficiency.

A first composition comprising viral vectors comprising a viral capsid, P2A sequence, a transgene gene, and balanced or unbalanced flanking 5′ and 3′ homology arm lengths is constructed. Homology arms are designed to be complementary to a target integration site (e.g., human genomic albumin).

A second composition comprising one or more meganuclease designed to target a non-coding sequence of an endogenous human locus (e.g., human ALB intron) is constructed. Several methods for delivering such a composition are known in the art (e.g., transfection with plasmids or mRNA encoding one or more meganuclease and/or transduction with viral vectors encoding one or more meganuclease.

Following construction of each composition, several experimental conditions are tested. In one experiment, cells (e.g., HepG2) are concurrently transduced and/or transfected with a first composition and second composition. In a following experiment, cells (e.g., HepG2) are sequentially transduced with a first composition and then transduced and/or transfected with a second composition. In a following experiment, cells (e.g., HepG2) are sequentially transduced and/or transfected with a second composition and then transduced with a first composition. In a following experiment, cells (e.g., HepG2) are concurrently transduced and/or transfected with a first composition and second composition, and then repeatedly (e.g., at least more than once) transduced and/or transfected with a second composition. In the following experiment, cells are sequentially transduced with a first composition and then repeatedly (e.g., at least more than once) transduced and/or transfected with a second composition. After transfection and/or transduction, total RNA is isolated from cells by using the RNeasy kit (Qiagen) following the vendor's instructions and assessed for levels of fused mRNA.

Among other things, this example demonstrates that viral vectors, as described herein, in combination with one or more meganuclease may provide improved editing activity. In some embodiments, one or more meganuclease can induce a DNA break (e.g., DSB and/or SSB) within a non-coding sequence of a human ALB gene. In some embodiments, induced DNA break (e.g., DSB and/or SSB) within a non-coding sequence of a human ALB gene may enhance transgene integration efficiency as compared to an appropriate reference (e.g., administration of vector without induced DSB and/or SSB). In some embodiments, administration of a GENERIDE™ construct in combination with an induced DNA break (e.g., DSB and/or SSB) in any non-coding sequence within an endogenous locus may provide improved transgene integration efficiency relative to a GENERIDE™ construct alone. In some embodiments, administration of a GENERIDE™ construct in combination with an induced DNA break (e.g., DSB and/or SSB) in a particular region of a non-coding sequence within an endogenous locus may provide improved or comparable transgene integration efficiency as compared to an induced DNA break (e.g., DSB and/or SSB). In some embodiments, an induced DNA break (e.g., DSB and/or SSB) is located in a non-coding sequence that is distal from the integration site of the transgene.

### Example 5: Induced DNA Break by an Exemplary Nuclease May Enhance GENERIDE™ Efficiency

This example demonstrates that, among other things, administration of a GENERIDE™ construct to a target integration site in a coding region of an endogenous human locus (e.g., human albumin (ALB)) in combination with one or more exemplary nucleases, as described herein, targeting a cut site within a non-coding sequence of an endogenous human locus (e.g., human albumin intron) may provide improved transgene integration efficiency.

A first composition comprising viral vectors comprising a viral capsid, P2A sequence, a transgene gene, and balanced or unbalanced flanking 5′ and 3′ homology arm lengths is constructed. Homology arms are designed to be complementary to a target integration site (e.g., human genomic albumin).

A second composition comprising one or more exemplary nucleases, as described herein, designed to target a non-coding sequence of an endogenous human locus (e.g., human ALB intron) is constructed. Several methods for delivering such a composition are known in the art (e.g., transfection with plasmids or mRNA encoding one or more exemplary nucleases, as described herein, transduction with viral vectors encoding one or more exemplary nucleases, as described herein, and/or intracellular delivery of one or more exemplary nucleases RNP, as described herein).

Following construction of each composition, several experimental conditions are tested. In one experiment, cells (e.g., HepG2) are concurrently transduced and/or transfected with a first composition and second composition. In a following experiment, cells (e.g., HepG2) are sequentially transduced with a first composition and then transduced and/or transfected with a second composition. In a following experiment, cells (e.g., HepG2) are sequentially transduced and/or transfected with a second composition and then transduced with a first composition. In a following experiment, cells (e.g., HepG2) are concurrently transduced and/or transfected with a first composition and second composition, and then repeatedly (e.g., at least more than once) transduced and/or transfected with a second composition. In the following experiment, cells are sequentially transduced with a first composition and then repeatedly (e.g., at least more than once) transduced and/or transfected with a second composition. After transfection and/or transduction, total RNA is isolated from cells by using the RNeasy kit (Qiagen) following the vendor's instructions and assessed for levels of fused mRNA.

Among other things, this example demonstrates that viral vectors, as described herein, in combination with or more exemplary nucleases, as described herein, may provide improved editing activity. In some embodiments, one or more exemplary nucleases, as described herein, can induce a DNA break (e.g., DSB and/or SSB) within a non-coding sequence of a human ALB gene. In some embodiments, induced DNA break (e.g., DSB and/or SSB) within a non-coding sequence of a human ALB gene may enhance transgene integration efficiency as compared to an appropriate reference (e.g., administration of vector without induced DSB and/or SSB). In some embodiments, administration of a GENERIDE™ construct in combination with an induced DNA break (e.g., DSB and/or SSB) in any non-coding sequence within an endogenous locus may provide improved transgene integration efficiency relative to a GENERIDE™ construct alone. In some embodiments, administration of a GENERIDE™ construct in combination with an induced DNA break (e.g., DSB and/or SSB) in a particular region of a non-coding sequence within an endogenous locus may provide improved or comparable transgene integration efficiency as compared to an induced DNA break (e.g., DSB and/or SSB) in an alternate region of a non-coding sequence. In some embodiments, an induced DNA break (e.g., DSB and/or SSB) is located in a non-coding sequence that is distal from the integration site of the transgene.

### Example 6: Induced DNA Break May Enhance GENERIDE™ Efficiency In Vivo or Ex Vivo

This example demonstrates that, among other things, administration of a GENERIDE™ construct to a target integration site in a coding region of an endogenous locus (e.g., human albumin) in combination with one or more endonucleases, as described herein, targeting a cut site within a non-coding sequence of an endogenous locus (e.g., human albumin intron) may provide improved transgene integration efficiency in a subject (e.g., a subject suffering from a disease).

In some embodiments, this example includes use of one or more GENERIDE™ constructs, e.g., viral vectors comprising a viral capsid, as described herein, a 2A sequence (e.g., P2A), a transgene, as described herein, and flanking human homology arms (e.g., balanced or unbalanced).

In some embodiments, GENERIDE™ constructs are administered sequentially or concurrently with one or more endonucleases, as described herein. In some embodiments, GENERIDE™ constructs are co-administered with one or more endonucleases to a subject in vivo (e.g., a subject suffering from a disease). In some embodiments, GENERIDE™ constructs are co-administered with one or more endonucleases to a cell (e.g., liver cell) ex vivo, optionally followed by administration of the cell to a subject (e.g., a subject suffering from a disease) after transgene integration. In some embodiments, GENERIDE™ constructs are administered prior to administration with one or more endonuclease. In some embodiments, GENERIDE™ constructs are administered following administration with one or more endonuclease. In some embodiments, GENERIDE constructs are co-administered with one or more endonucleases to a subject in vivo (e.g., a subject suffering from a disease), optionally followed by administration with one or more endonucleases.

Among other things, this example provides insight that induced DNA break (e.g., DSB and/or SSB) may enhance GENERIDE efficacy in-vivo. In some embodiment, levels of fused mRNA may increase in a subject, and/or a cell, and/or a tissue as compared to an appropriate reference (e.g., administration of vector without induced DNA break (e.g., DSB and/or SSB). In some embodiments, levels of ALB-2A may increase in a subject, and/or a cell, and/or a tissue as compared to an appropriate reference (e.g., administration of vector without induced DNA break (e.g., DSB and/or SSB). In some embodiments, a transgene expression may increase in a subject, and/or a cell, and/or a tissue as compared to an appropriate reference (e.g., administration of vector without induced DNA break (e.g., DSB and/or SSB).

Among other things, this example provides insight that GENERIDE™ constructs administered sequentially or concurrently with one or more endonucleases may provide an enhanced selective advantage for cells that have successfully integrated a transgene of interest.

### Example 7: Induced DNA Break with GENERIDE™ May Improve Wilson's Disease Phenotype

The present example demonstrates that, among other things, administration of a GENERIDE™ construct, comprising a therapeutic transgene encoding ATP7B (e.g., a truncated ATP7B), to a target integration site in a coding region of an endogenous locus (e.g., human albumin) in combination with one or more endonucleases, as described herein, targeting a cut site within a non-coding sequence of an endogenous locus (e.g., human albumin intron) may be used to treat or prevent Wilson's disease (e.g., through reduction of phenotypic effects and/or symptoms in-vivo) in a subject (e.g., a subject suffering from a Wilson's disease).

GENERIDE™ constructs, e.g., viral vectors comprising a viral capsid, as described herein, a 2A sequence (e.g., P2A), a human ATP7B (e.g., a truncated ATP7B) transgene, and flanking human homology arms (e.g., balanced or unbalanced) are constructed. GENERIDE™ constructs are administered (e.g., intravenously) at a dose (e.g., a fixed dose or a weight-based dose) to a subject (e.g., a subject suffering from a Wilson's disease). In some embodiments, GENERIDE™ constructs are administered sequentially or concurrently with one or more endonucleases, as described herein. In some embodiments, GENERIDE™ constructs are co-administered with one or more endonucleases to a subject in vivo (e.g., a subject suffering from a Wilson's disease). In some embodiments, GENERIDE™ constructs are co-administered with one or more endonucleases to a cell (e.g., liver cell) ex vivo, optionally followed by administration of the cell to a subject (e.g., a subject suffering from a Wilson's disease) after transgene integration. In some embodiments, GENERIDE™ constructs are co-administered with one or more endonucleases to a subject in vivo (e.g., a subject suffering from Wilson's disease), optionally followed by administration with one or more endonuclease.

Among other things, this example provides insight that induced DNA break (e.g., DSB and/or SSB) may enhance GENERIDE™ efficacy in-vivo. In some embodiments, levels of fused mRNA may increase in a subject, and/or a cell, and/or a tissue as compared to an appropriate reference (e.g., administration of vector without induced DNA break (e.g., DSB and/or SSB). In some embodiments, levels of ALBA-2A may increase in a subject, and/or a cell, and/or a tissue as compared to an appropriate reference (e.g., administration of vector without induced DNA break (e.g., DSB and/or SSB). In some embodiments, a transgene expression may increase in a subject, and/or a cell, and/or a tissue as compared to an appropriate reference (e.g., administration of vector without induced DNA break (e.g., DSB and/or SSB).

Among other things, the present example demonstrates that treatment of a subject (e.g., a subject suffering from Wilson's disease) with viral vectors of the present disclosure may increase a rate and/or a level of integration (e.g., measure through percentage of positive cells) relative to an appropriate reference (e.g., untreated or vehicle). In some embodiments, treatment of a subject (e.g., a subject suffering from Wilson's disease) with viral vectors of the present disclosure may allow or restore normal growth (e.g., measured through percentage body weight changes over time) relative to an appropriate reference (e.g., untreated or vehicle). In some embodiments, treatment of a subject (e.g., a subject suffering from a Wilson's disease) with viral vectors of the present disclosure may provide reduced levels of a biomarker (e.g., copper, hAAT, alanine aminotransferase (ALT)) associated with Wilson's disease. In some embodiments, treatment of a subject (e.g., a subject suffering from a Wilson's disease) with viral vectors of the present disclosure may provide one or more of improved tissue (e.g., liver) function (e.g., measured through assessment of markers of tissue (e.g., liver) function), normal growth (e.g., measured through percentage body weight changes over time) relative to a reference (e.g., untreated or vehicle), reduced levels of a biomarker (e.g., liver or urinary copper, hAAT, alanine aminotransferase (ALT)) associated with Wilson's disease.

### Example 8: Induced DNA Break with GENERIDE™ May Improve Hereditary Tyrosinemia Phenotype

The present example demonstrates that, among other things, administration of a GENERIDE™ construct, comprising a therapeutic transgene encoding FAH and/or variant thereof, to a target integration site in a coding region of an endogenous locus (e.g., human albumin) in combination with one or more endonucleases, as described herein, targeting a cut site within a non-coding sequence of an endogenous locus (e.g., human albumin intron) may be used to treat or prevent hereditary tyrosinemia (e.g., through reduction of phenotypic effects and/or symptoms in-vivo) in a subject (e.g., a subject suffering from a hereditary tyrosinemia).

GENERIDE™ constructs, e.g., viral vectors comprising a viral capsid, as described herein, a 2A sequence (e.g., P2A), a human FAH transgene and/or variant thereof, and flanking human homology arms (e.g., balanced or unbalanced) are constructed. GENERIDE constructs are administered (e.g., intravenously) at a dose (e.g., a fixed dose or a weight-based dose) to a subject (e.g., a subject suffering from a hereditary tyrosinemia). In some embodiments, GENERIDE™ constructs are administered sequentially or concurrently with one or more endonucleases, as described herein. In some embodiments, GENERIDE™ constructs are co-administered with one or more endonucleases to a subject in vivo (e.g., a subject suffering from a hereditary tyrosinemia). In some embodiments, GENERIDE™ constructs are co-administered with one or more endonucleases to a cell (e.g., liver cell) ex vivo, optionally followed by administration of the cell to a subject (e.g., a subject suffering from a hereditary tyrosinemia) after transgene integration. In some embodiments, GENERIDE™ constructs are co-administered with one or more endonucleases to a subject in vivo (e.g., a subject suffering from a hereditary tyrosinemia), optionally followed by administration with one or more endonuclease.

Among other things, this example provides insight that induced DNA break (e.g., DSB and/or SSB) may enhance GENERIDE™ efficacy in-vivo. In some embodiments, levels of fused mRNA may increase in a subject, and/or a cell, and/or a tissue as compared to an appropriate reference (e.g., administration of vector without induced DNA break (e.g., DSB and/or SSB). In some embodiments, levels of ALBA-2A may increase in a subject, and/or a cell, and/or a tissue as compared to an appropriate reference (e.g., administration of vector without induced DNA break (e.g., DSB and/or SSB). In some embodiments, a transgene expression may increase in a subject, and/or a cell, and/or a tissue as compared to an appropriate reference (e.g., administration of vector without induced DNA break (e.g., DSB and/or SSB).

Among other things, this example demonstrates that treatment, treatment of a subject (e.g., a subject suffering from hereditary tyrosinemia) with viral vectors of the present disclosure may increase a rate and/or a level of integration (e.g., measure through percentage of positive cells) relative to an appropriate reference (e.g., untreated or vehicle). In some embodiments, treatment of a subject (e.g., a subject suffering from hereditary tyrosinemia) with viral vectors of the present disclosure may allow or restore normal growth (e.g., measured through percentage body weight changes over time) relative to an appropriate reference (e.g., untreated or vehicle). In some embodiments, treatment of a subject (e.g., a subject suffering from a hereditary tyrosinemia) with viral vectors of the present disclosure may provide reduced levels of a biomarker (e.g., ALT, bilirubin, succinylacetone (SUAC), alfa-fetoprotein (AFP)) associated with hereditary tyrosinemia. In some embodiments, treatment of a subject (e.g., a subject suffering from a hereditary tyrosinemia) with viral vectors of the present disclosure may provide one or more of improved tissue (e.g., liver) function (e.g., measured through assessment of markers of tissue (e.g., liver) function), normal growth (e.g., measured through percentage body weight changes over time) relative to a reference (e.g., untreated or vehicle), reduced levels of a biomarker (e.g., ALT, bilirubin, succinylacetone (SUAC), alfa-fetoprotein (AFP)) associated with hereditary tyrosinemia.

### Example 9: Induced DNA Break with GENERIDE™ May Improve Crigler-Najjar Syndrome

The present example demonstrates that, among other things, administration of a GENERIDE™ construct, comprising a UGT1A1 gene and/or variant thereof, to a target integration site in a coding region of an endogenous locus (e.g., human albumin) in combination with one or more endonucleases, as described herein, targeting a cut site within a non-coding sequence of an endogenous locus (e.g., human albumin intron) may be used to treat or prevent Crigler-Najjar syndrome (e.g., through reduction of phenotypic effects and/or symptoms in-vivo) in a subject (e.g., a subject suffering from Crigler-Najjar syndrome).

GENERIDE™ constructs, e.g., viral vectors comprising a viral capsid, as described herein, a 2A sequence (e.g., P2A), a UGT1A1 gene and/or variant thereof, and flanking human homology arms (e.g., balanced or unbalanced) are constructed. GENERIDE™ constructs are administered (e.g., intravenously) at a dose (e.g., a fixed dose or a weight-based dose) to a subject (e.g., a subject suffering from Crigler-Najjar syndrome). In some embodiments, GENERIDE™ constructs are administered sequentially or concurrently with one or more endonucleases, as described herein. In some embodiments, GENERIDE™ constructs are co-administered with one or more endonucleases to a subject in vivo (e.g., a subject suffering from Crigler-Najjar syndrome). In some embodiments, GENERIDE™ constructs are co-administered with one or more endonucleases to a cell (e.g., liver cell) ex vivo, optionally followed by administration of the cell to a subject (e.g., a subject suffering from Crigler-Najjar syndrome) after transgene integration. In some embodiments, GENERIDE™ constructs are co-administered with one or more endonucleases to a subject in vivo (e.g., a subject suffering from Crigler-Najjar syndrome), optionally followed by administration with one or more endonucleases.

Among other things, this example provides insight that induced DNA break (e.g., DSB and/or SSB) may enhance GENERIDE™ efficacy in-vivo. In some embodiments, levels of fused mRNA may increase in a subject, and/or a cell, and/or a tissue as compared to an appropriate reference (e.g., administration of vector without induced DNA break (e.g., DSB and/or SSB). In some embodiments, levels of ALBA-2A may increase in a subject, and/or a cell, and/or a tissue as compared to an appropriate reference (e.g., administration of vector without induced DNA break (e.g., DSB and/or SSB). In some embodiments, a transgene expression may increase in a subject, and/or a cell, and/or a tissue as compared to an appropriate reference (e.g., administration of vector without induced DNA break (e.g., DSB and/or SSB).

Among other things, this example demonstrates that treatment, treatment of a subject (e.g., a subject suffering from Crigler-Najjar syndrome) with viral vectors of the present disclosure may increase a rate and/or a level of integration (e.g., measure through percentage of positive cells) relative to an appropriate reference (e.g., untreated or vehicle). In some embodiments, treatment of a subject (e.g., a subject suffering from Crigler-Najjar syndrome) with viral vectors of the present disclosure may allow or restore normal growth (e.g., measured through percentage body weight changes over time) relative to an appropriate reference (e.g., untreated or vehicle). In some embodiments, treatment of a subject (e.g., a subject suffering from Crigler-Najjar syndrome) with viral vectors of the present disclosure may provide reduced levels of a biomarker associated with Crigler-Najjar syndrome. In some embodiments, treatment of a subject (e.g., a subject suffering from Crigler-Najjar syndrome) with viral vectors of the present disclosure may provide one or more of improved tissue (e.g., liver) function (e.g., measured through assessment of markers of tissue (e.g., liver) function), normal growth (e.g., measured through percentage body weight changes over time) relative to a reference (e.g., untreated or vehicle), reduced levels of a biomarker associated with Crigler-Najjar syndrome.

### Example 10: Induced DNA Break with GENERIDE™ May Improve Alpha-1 Antitrypsin Deficiency

The present example demonstrates that, among other things, administration of a GENERIDE™ construct, comprising a SERPINA1 transgene and/or variants thereof, to a target integration site in a coding region of an endogenous locus (e.g., human albumin) in combination with one or more endonucleases, as described herein, targeting a cut site within a non-coding sequence of an endogenous locus (e.g., human albumin (ALB) intron) may be used to treat or prevent Alpha-1 Antitrypsin Deficiency (e.g., through reduction of phenotypic effects and/or symptoms in-vivo) in a subject (e.g., a subject suffering from Alpha-1 Antitrypsin Deficiency).

GENERIDE™ constructs, e.g., viral vectors comprising a viral capsid, as described herein, a 2A sequence (e.g., P2A), a SERPINA1 transgene and/or variants thereof, and flanking human homology arms (e.g., balanced or unbalanced) are constructed.

GENERIDE™ constructs are administered (e.g., intravenously) at a dose (e.g., a fixed dose or a weight-based dose) to a subject (e.g., a subject suffering from Alpha-1 Antitrypsin Deficiency). In some embodiments, GENERIDE™ constructs are administered sequentially or concurrently with one or more endonucleases, as described herein. In some embodiments, GENERIDE™ constructs are co-administered with one or more endonucleases to a subject in vivo (e.g., a subject suffering from Alpha-1 Antitrypsin Deficiency). In some embodiments, GENERIDE™ constructs are co-administered with one or more endonucleases to a cell (e.g., liver and/or lung cell) ex vivo, optionally followed by administration of the cell to a subject (e.g., a subject suffering from Alpha-1 Antitrypsin Deficiency) after transgene integration. In some embodiments, GENERIDE™ constructs are co-administered with one or more endonucleases to a subject in vivo (e.g., a subject suffering from Alpha-1 Antitrypsin Deficiency), optionally followed by administration with one or more endonuclease.

Among other things, this example provides insight that induced DNA break (e.g., DSB and/or SSB) may enhance GENERIDE™ efficacy in-vivo. In some embodiments, levels of fused mRNA may increase in a subject, and/or a cell, and/or a tissue as compared to an appropriate reference (e.g., administration of vector without induced DNA break (e.g., DSB and/or SSB). In some embodiments, levels of ALBA-2A may increase in a subject, and/or a cell, and/or a tissue as compared to an appropriate reference (e.g., administration of vector without induced DNA break (e.g., DSB and/or SSB). In some embodiments, a transgene expression may increase in a subject, and/or a cell, and/or a tissue as compared to an appropriate reference (e.g., administration of vector without induced DNA break (e.g., DSB and/or SSB).

Among other things, this example demonstrates that treatment, treatment of a subject (e.g., a subject suffering from Alpha-1 Antitrypsin Deficiency) with viral vectors of the present disclosure may increase a rate and/or a level of integration (e.g., measure through percentage of positive cells) relative to an appropriate reference (e.g., untreated or vehicle). In some embodiments, treatment of a subject (e.g., a subject suffering from Alpha-1 Antitrypsin Deficiency) with viral vectors of the present disclosure may allow or restore lung function relative to an appropriate reference (e.g., untreated or vehicle). In some embodiments, treatment of a subject (e.g., a subject suffering from Alpha-1 Antitrypsin Deficiency) with viral vectors of the present disclosure may provide reduced levels of a biomarker associated with Alpha-1 Antitrypsin Deficiency. In some embodiments, treatment of a subject (e.g., a subject suffering from Alpha-1 Antitrypsin Deficiency) with viral vectors of the present disclosure may provide one or more of improved tissue (e.g., lung) function (e.g., measured through assessment of markers of tissue (e.g., lung) function), normal respiratory function or reduced worsening of respiratory function, reduced levels of a biomarker associated with Alpha-1 Antitrypsin Deficiency.

### Example 11: Induced Double Stranded Break with GENERIDE™ May Improve Tissue Function

The present example demonstrates that, among other things, administration of a GENERIDE™ construct, comprising a therapeutic transgene and/or variant thereof, with a target integration site in a coding region of an endogenous locus (e.g., human albumin) in combination with one or more endonucleases, as described herein, targeting a cut site within a non-coding sequence of an endogenous locus (e.g., human albumin intron) may be used to treat or prevent disease (e.g., through reduction of phenotypic effects and/or symptoms in-vivo) in a subject (e.g., a subject suffering from a disease).

GENERIDE™ constructs, e.g., viral vectors comprising a viral capsid, as described herein, a 2A sequence (e.g., P2A), a therapeutic transgene and/or variant thereof, and flanking human homology arms (e.g., balanced or unbalanced) are constructed. GENERIDE constructs are administered (e.g., intravenously) at a dose (e.g., a fixed dose or a weight-based dose) to a subject (e.g., a subject suffering from a disease). In some embodiments, GENERIDE™ constructs are administered sequentially or concurrently with one or more endonucleases, as described herein. In some embodiments, GENERIDE™ constructs are co-administered with one or more endonucleases to a subject in vivo (e.g., a subject suffering from a disease). In some embodiments, GENERIDE™ constructs are co-administered with one or more endonucleases to a cell (e.g., liver cell) ex vivo, optionally followed by administration of the cell to a subject (e.g., a subject suffering from a disease) after transgene integration. In some embodiments, GENERIDE™ constructs are co-administered with one or more endonucleases to a subject in vivo (e.g., a subject suffering from a disease), optionally followed by administration with one or more endonucleases.

Among other things, this example provides insight that induced DNA break (e.g., DSB and/or SSB) may enhance GENERIDE™ efficacy in-vivo. In some embodiment, levels of fused mRNA may increase in a subject, and/or a cell, and/or a tissue as compared to an appropriate reference (e.g., administration of vector without induced DNA break (e.g., DSB and/or SSB). In some embodiments, levels of ALBA-2A may increase in a subject, and/or a cell, and/or a tissue as compared to an appropriate reference (e.g., administration of vector without induced DNA break (e.g., DSB and/or SSB). In some embodiments, a transgene expression may increase in a subject, and/or a cell, and/or a tissue as compared to an appropriate reference (e.g., administration of vector without induced DNA break (e.g., DSB and/or SSB).

Among other things, this example demonstrates that treatment, treatment of a subject (e.g., a subject suffering from disease) with viral vectors of the present disclosure may increase a rate and/or a level of integration (e.g., measure through percentage of positive cells) relative to an appropriate reference (e.g., untreated or vehicle). In some embodiments, treatment of a subject (e.g., a subject suffering from disease) with viral vectors of the present disclosure may allow or restore normal growth (e.g., measured through percentage body weight changes over time) relative to an appropriate reference (e.g., untreated or vehicle). In some embodiments, treatment of a subject (e.g., a subject suffering from a disease) with viral vectors of the present disclosure may provide reduced levels of a biomarker associated with disease. In some embodiments, treatment of a subject (e.g., a subject suffering from a disease) with viral vectors of the present disclosure may provide one or more of improved tissue (e.g., liver) function (e.g., measured through assessment of markers of tissue (e.g., liver) function), normal growth (e.g., measured through percentage body weight changes over time) relative to a reference (e.g., untreated or vehicle), reduced levels of a biomarker associated with disease.

### Example 12: Longer Homology Alignments May Predict Higher Integration Efficiency

The present example demonstrates that, among other things, administration of a GENERIDE™ construct with longer homology alignments in combination with one or more Cas9 enzymes targeting a cut site within a non-coding sequence of an endogenous human locus (e.g., human albumin (ALB) intron) may provide improved transgene integration efficiency.

Viral vectors comprising a viral capsid (e.g., LK03), P2A sequence, a transgene gene (e.g., GFP), varying flanking 5′ and 3′ homology arm lengths were constructed. Homology arms were designed to be complementary to a human genomic albumin target integration site. HepG2 cells, a human liver hepatocellular carcinoma cell line, were transduced with viral vectors at dose of MOI=1E⁵. Following transduction, cells were transfected with spCas9 and gRNA (designed using IDT gRNA algorithm) using Lipofectamine™ CRISPRMAX (ThermoFisher Scientific, Inc) according to the manufacturer's instructions. gRNA were designed to target ALB gene at either intron 13 or 14 (Table 8). 48 hours after transfection, total RNA was isolated from cells by using the RNeasy kit (Qiagen) following the vendor's instructions and assessed for levels of fused mRNA. In addition, immunohistochemistry and/or immunocytochemistry was performed using anti-GFP antibody. Images were evaluated using an imaging processing software (e.g., ImageJ).

Among other things, this example demonstrates that longer homology alignments may provide higher integration efficiency. In some embodiments, as demonstrated in FIG. 4B, homology alignment of at least 800 bp may enhance transgene integration efficiency (measured as levels of fused mRNA) as compared to an appropriate reference. In some embodiments, as demonstrated in FIG. 4C, homology alignment of at least 800 bp may enhance transgene integration efficiency (measured as levels of GFP positive cells). In some embodiment, homology alignments may be optimized to improve transgene integration efficiency.

### Example 13: Induced Double Stranded Break by Cas9 with Appropriate gRNA Further Demonstrate Enhancement of GENERIDE™ Efficiency

This example further demonstrates that combination of a GENERIDE™ construct to a target integration site in a coding region of an endogenous human locus (e.g., human albumin (ALB)) with one or more Cas9 enzymes targeting a cut site within a non-coding sequence of an endogenous human locus (e.g., human ALB intron) may provide improved transgene integration efficiency.

Viral vectors comprising a viral capsid (e.g., LK03), P2A sequence, a transgene gene (e.g., GFP), a balanced (1 kb/1 kb) flanking 5′ and 3′ homology arm lengths were constructed. Homology arms were designed to be complementary to a human genomic albumin target integration site. HepG2 cells, a human liver hepatocellular carcinoma cell line, were transduced with viral vectors at dose of MOI=1E⁵. Following transduction, cells were transfected with Cas9 and gRNA (designed using IDT gRNA algorithm) using Lipofectamine™ CRISPRMAX (ThermoFisher Scientific, Inc) according to the manufacturer's instructions. gRNA were designed to target ALB gene at either intron 13 or 14 (Table 9). 48 hours after transfection, total RNA was isolated from cells by using the RNeasy kit (Qiagen) following the vendor's instructions and assessed for levels of fused mRNA (e.g., see Table 8). Table 9 shows gRNA spacer sequences that are identical to the cognate sequence in ALB.

TABLE 9
Exemplary gRNA sequence for spCas9.

| | | | SEQ ID |
|---|---|---|---|
| Target locus | Strand | Sequence | NO: |
| Human albumin Intron 13_11 | − | ACAGTATGGCACAATAGAGC | 71 |
| Human albumin Intron 13_12 | − | AGTTACTGTCAGGCCATAAA | 72 |
| Human albumin Intron 13_13 | − | AAGTTACTGTCAGGCCATAA | 73 |
| Human albumin Intron 13_14 | + | AGGGATTTATATATCAAAGG | 74 |
| Human albumin Intron 13_15 | + | AAAGGAGGCTTTGTACATGT | 75 |
| Human albumin Intron 13_16 | − | TTACTACTATAATCTCTGGA | 76 |
| Human albumin Intron 13_17 | + | GGCTTTGTACATGTGGGACA | 77 |
| Human albumin Intron 14_11 | − | AAAGGATGAGTTGAACTATT | 78 |
| Human albumin Intron 14_12 | − | ATAAGTCAGAACAGGACAAT | 79 |
| Human albumin Intron 14_13 | + | ACATGAAAGAGTGCTTTATA | 80 |
| Human albumin Intron 14_14 | − | AAATAGTTTAGAATAGTGGT | 81 |
| Human albumin Intron 14_15 | + | GCTCAGCAGTGGAATACTCT | 82 |
| Human albumin Intron 14_16 | + | CTGTTCTGACTTATATGATG | 83 |
| Human albumin Intron 14_17 | + | AGTAAGTGATGGCTCAGCAG | 84 |
| Human albumin Intron 14_18 | − | TATAAGTCAGAACAGGACAA | 85 |
| Human albumin Intron 14_19 | − | AATGGGCAACACTGAAACTT | 86 |

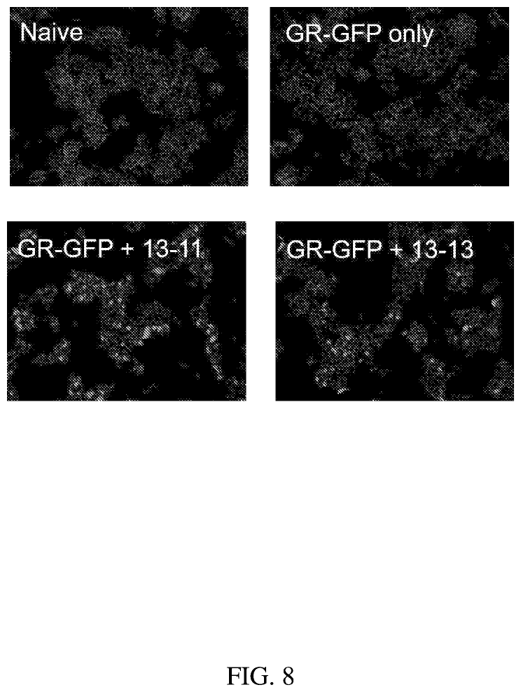

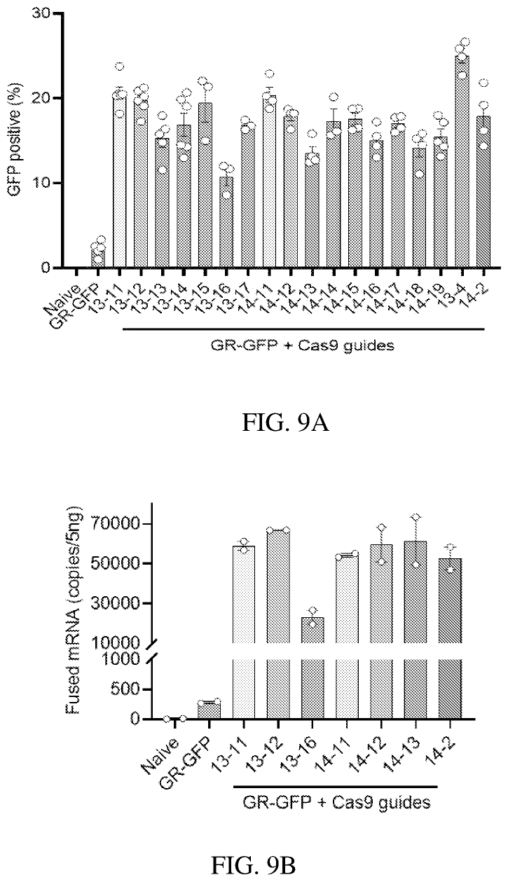

Viral vectors, as described herein, comprising a transgene (e.g., GFP) in combination with Cas9 provide improved editing activity. As demonstrated in FIG. 9A induced DSB in intron 13 and/or 14 enhanced protein expression (e.g., increased % GFP positive cells) by at least 5-fold relative to a GENERIDE™ construct alone. Furthermore, as demonstrated in FIG. 9B, induced DSB in intron 13 and/or 14 enhanced protein expression relative a GENERIDE™ construct alone. Increased levels of fused mRNA may be associated with increased protein expression. There was at least a 600-fold increase in fused mRNA for tested Cas9 gRNAs in combination with GENERIDE™ relative to a GENERIDE™ construct alone.

Thus, this example demonstrates that a GENERIDE™ construct in combination with a DSB in a particular region of a non-coding sequence within an endogenous locus may provide improved transgene integration efficiency.

### Example 14: Administration of Cas9 May Enhance GENERIDE™ Efficiency

The present example demonstrates that administration of a GENERIDE™ construct to a target integration site in a coding region of an endogenous mouse locus (e.g., mouse albumin (ALB)) in combination with one or more Cas9 enzymes targeting a cut site within a non-coding sequence of an endogenous mouse locus (e.g., mouse ALB intron) may provide improved transgene integration efficiency in-vivo.

### Animal Study

Animals are purchased from Jackson Laboratories. General procedures for animal care and housing were approved by the Institutional Animal Care and Use Committee at LogicBio Therapeutics. Four-week-old male FvB animals were treated rAAV.DJ-GR-eGFP at the dosage of 3e13 vg/kg via retro-orbital sinus under anesthesia, followed by the dosing of LNP-formulated spCas9 mRNA and gRNA. SpCas9 14_4 LNP was treated either 1, 3, or 7 days post AAV dosing. During the study, animals were sampled periodically by submental bleed and plasma was collected and stored at −80° C. until further analysis. At sacrifices, blood was collected for plasma via cardiac puncture. For liver dissection, one lobe of liver was fixed by 10% formalin, and the remaining was snap-frozen and stored at −80° C. Next day, formalin-fixed liver was transferred to 70% ethanol for paraffin embedding.

### Plasma Albumin-2A Fusion Protein Quantification

Mouse Albumin-2A in plasma was measured by chemiluminescence ELISA, using a proprietary rabbit polyclonal anti-2A antibody for capture and an HRP-labeled polyclonal goat anti-mouse Albumin antibody (Abcam ab 19195) for detection. Recombinant mouse Albumin-2A expressed in mammalian cells and affinity-purified was used to build the standard curve in 1% control mouse plasma to account for matrix effects. Milk at 1% (Cell Signaling 9999S) in PBS was used for blocking and BSA at 1% for sample dilution in PBST.

### Immunohistochemistry

Immunohistochemistry was performed on a robotic platform (Ventana™ DISCOVERY ULTRA Staining Module, Ventana Co., Tucson, AZ). Tissue sections (4 μm) were deparaffinized and underwent heat-induced antigen retrieval for 64 min. Endogenous peroxidases were blocked with peroxidase inhibitor (CM1) for 8 min before incubating the section with anti-GFP antibody (Novus Biologicals, Centennial, CO) at 1:800 dilution for 60 min at room temperature. Antigen-antibody complex was then detected using DISC. OmniMap anti-rabbit multimer RUO detection system and DISCOVERY ChromoMap DAB Kit Ventana Co., Tucson, AZ). All the slides were counterstained with hematoxylin (Fisher Sci, Waltham, MA) subsequently, dehydrated, cleared and mounted for image scanning using digital slide scanner (Hamamatsu, Bridgewater, NJ). Scanned images were evaluated in a blinded fashion using ImageJ software to quantify the area of positive staining.

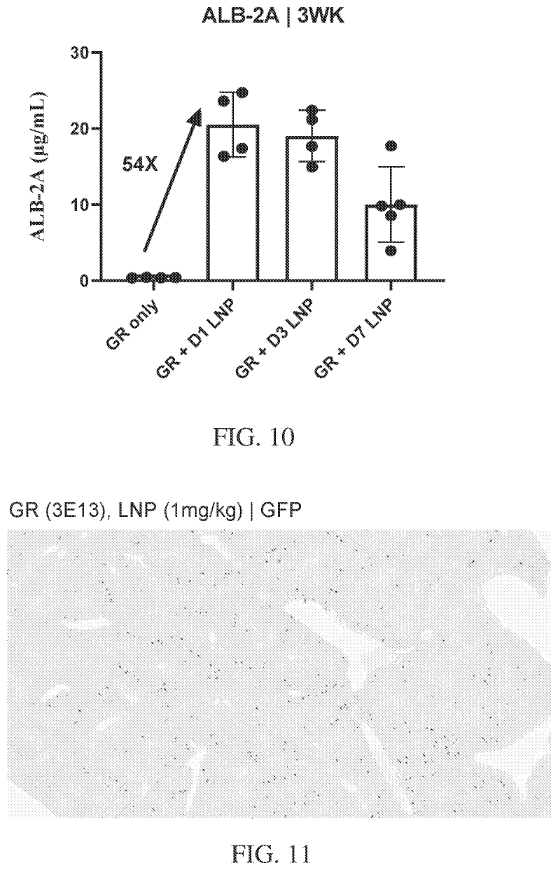

As demonstrated in FIG. 10, administration of LNP-formulated spCas9 (mRNA and gRNA) either 1, 3, or 7 days post GENERIDE™ dosing improved transgene integration efficiency in-vivo. Levels of Albumin-2A (ALB-2A) increased at least 54-fold in mice administered LNP-formulated spCas9 1 day post GENERIDE™ dosing relative to administration of a GENERIDE™ construct alone. This effect was similar in mice administered LNP-formulated spCas9 3 days post GENERIDE™ dosing. Administration of LNP-formulated spCas9 7 days post GENERIDE™ dosing increased levels of ALB-2A at least 20-fold relative to administration of a GENERIDE™ construct alone. FIG. 11 shows images depicting that in vivo administration of Cas9 and appropriate guide RNAs may increase integration efficiency.

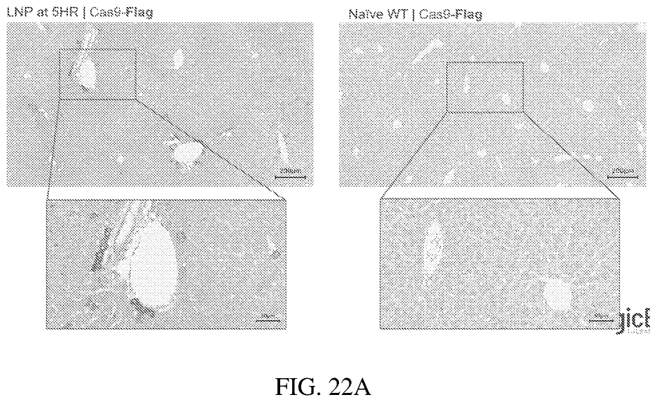

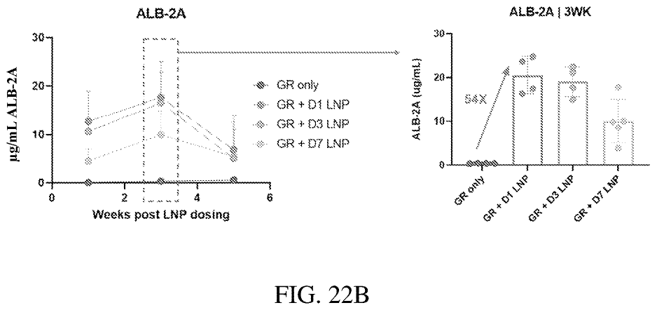

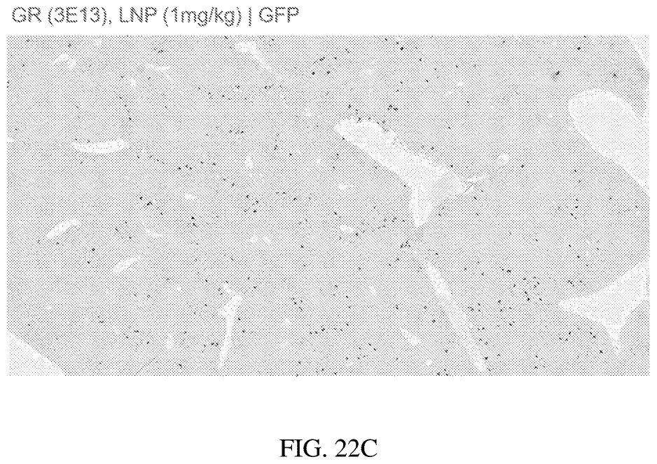

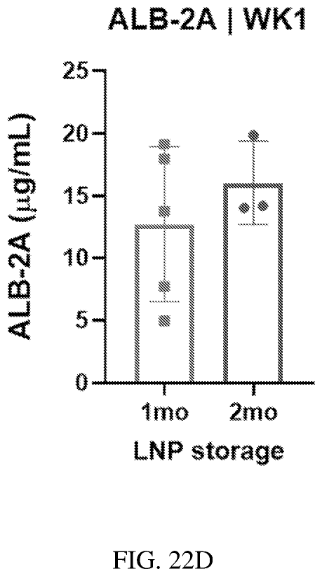

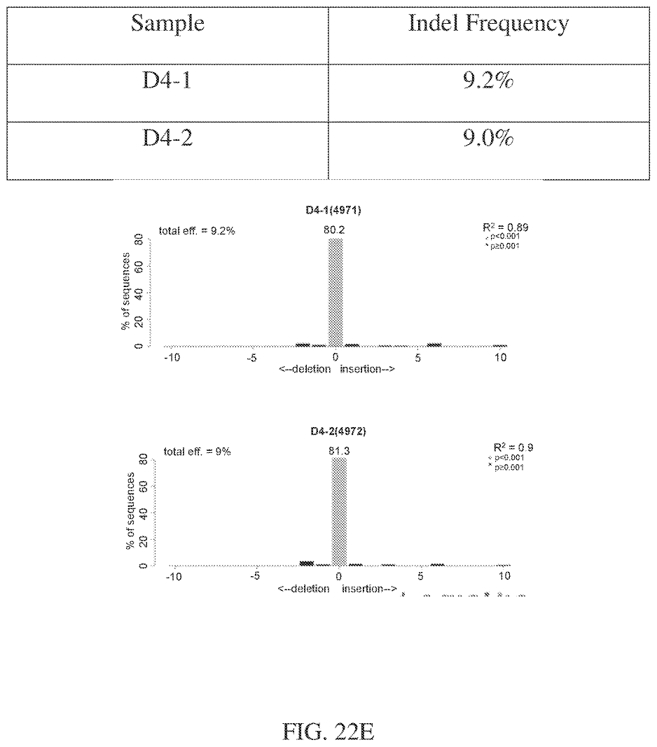

FIGS. 22A-22E shows results of experiments performed to optimize the dosing strategy for GENERIDE™ and Gen Voy-ILM LNP in liver cells, confirm delivery of Cas9 by Gen Voy-ILM LNP, and determine the timing of components for peak integration. Different groups of mice were administered different combinations of either GR (3e13 vg/kg) alone, GR (3e13 vg/kg) in combination with Cas9 (1 mg/kg) after 1, 3, or 7 days, or only LNP containing Cas9+gRNA (1 mg/kg). The GENERIDE™ tool vector used was DJ-GFP (LB-Vt-0298-001). INDELs were assayed by Sanger sequencing followed by TIDE analysis. FIG. 22A shows IHC (IHC targeting Cas9-Flag) images of liver cells confirming Cas9 expression after delivery of LNP containing Cas9, with a naïve WT control. FIG. 22B shows a series of graphs measuring the levels of ALB-2A (μg/ml) for up to 6 weeks post LNP dosing (left panel) and measuring the levels of ALB-2A (μg/ml) at 3 weeks post LNP dosing (right panel) for GR dosed with LNP at different time points (D1, D3, and D7), with a GR only control. An assay was conducted to measure levels of the GENERIDE™ PD biomarker (ALB-2A). FIG. 22C shows an IHC image of hepatocytes confirming improved integration in GR2.0 (Cas9) after delivery of GR (3e13)+LNP (1 mg/kg) containing Cas9. For this experiment, FvB/NJ mice were dosed with 3e13 vk of GR-GFP (1.0/1.0) followed by Day 1 GenVoy-ILM LNP-Cas9 at 1 mg/kg. FIG. 22D shows a bar graph measuring the levels of ALB-2A (μg/ml) 1 week after administration to confirm potency of stored LNP when LNP has been stored for 1 month or 2 months. FIG. 22E shows a series of graphs quantifying the indel frequency via TIDE of D4-1 (top panel) and D4-2 (bottom panel).

Thus, this example demonstrates that a GENERIDE™ construct in combination with a DSB in a particular region of a non-coding sequence within an endogenous locus may provide improved transgene integration efficiency in-vivo. Administration of LNP-formulated spCas9 1 or 3 days post GENERIDE™ dosing may confer greater enhancement of transgene integration efficiency than administration of LNP-formulated spCas9 7 days post GENERIDE™ dosing.

This Example also demonstrates that Cas9 was expressed in most liver cells using Gen Voy-ILM LNP and 1-day post GR dosing is an optimized time point to perform LNP dosing as LNP dosing at D1 and D3 showed comparable ALB-2A levels and higher ALB-2A levels than D7 dosing. This example also shows that ALB-2A significantly improved (~50×) with application of LNP-Cas9 and at 5 weeks, there is a lowering of ALB-2A, potentially due to an immune response to the GOI, however, the GR only group does not demonstrate the same drop of expression. The latter may be due to the lower overall integration levels. This example confirmed improved integration in GR2.0 (Cas9) and that LNP potency remained the same when LNP is stored for 2 months. The current GR2.0 (Cas9) platform (dosing GR-GFP (1.0/1.0) at 3E13 vg/kg) resulted in 1.9% edited rates of hepatocytes. Historically, the same dosage of GR-MMUT (1.0/1.0) edited 0.05% of hepatocytes, suggesting a 40× increase with the current platform. Finally, this example illustrated a 9.2% indel frequency in the D4-1 sample and a 9.0% indel frequency in the D4-2 sample.

### Example 15: Imbalance of Homology Arm Length (0.4-0.8 kb) Did not Significantly Impact Cas9-Mediated Enhancement of GENERIDE™ Integration

The experiment described in this Example demonstrates that an imbalance of homology arm length (0.4-0.8 kb) does not significantly impact Cas9-mediated enhancement of GENERIDE™ integration.

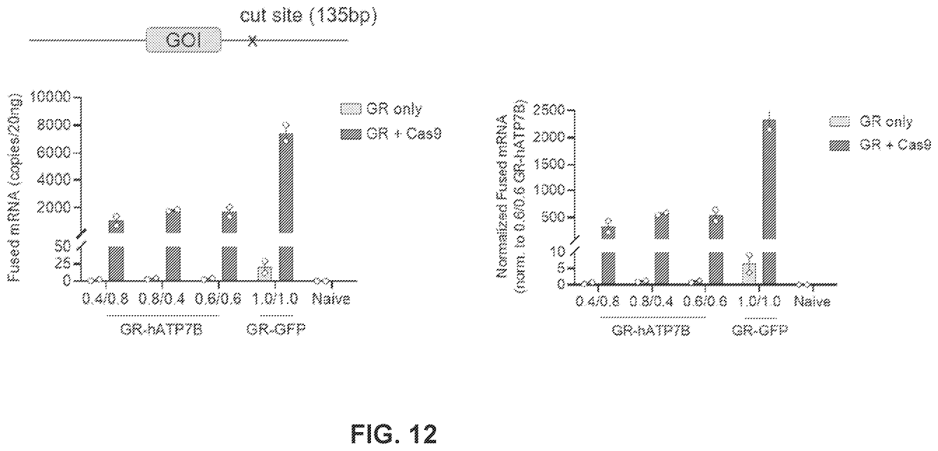

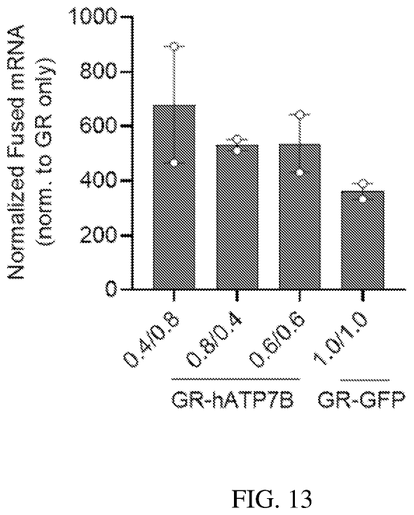

FIG. 12 shows results of an experiment in which two GENERIDE™ constructs (GR-hATP7B or GR-GFP) were administered alone or in combination with Cas9, with a naïve control. The GR-hATP7b construct included varying sizes of homology arms (0.4/0.8 kB; 0.8/0.4 kB, or 0.6/0.6 kB), while the GR-GFP had 1.0/1.0 kB homology arms. GOI, gene of interest. HEPG2 cells were seeded at 100 k/well in 24-well format. The next day, medium was replaced with DMEM containing 2.5% fetal bovine serum (FBS) with or without the constructs (GR-hATP7B or GR-GFP) at the MOI 1E5. Cas9 mRNA (1 μg/well) and sgRNA (250 ng/well) were transfected using the Lipofectamine™ MessengerMAX™ according to the vendor protocol. Cells were harvested 3 days after transfection, and mRNA was extracted for the analysis. FIG. 12 shows a series of graphs measuring levels of fused mRNA (copies/20 mg) (left panel) and normalized fused mRNA (norm. to 0.6/0.6 GR-hATP7B) (right panel). GR-GFP had a higher baseline, which, without wishing to be bound by any particular theory, may be due to longer homology arms or a smaller GOI. FIG. 13 shows results of an experiment in which the fused mRNA fold increase between GOIs was measured as normalized fused mRNA (norm. to GR only) for the indicated constructs. The level of fused mRNA is a measure of integration efficiency.

Thus, this example demonstrates that an imbalance of homology arm length does not have a significant impact on Cas9-mediated enhancement when a GENERIDE™ construct is administered in combination with Cas9.

### Example 16: Integration Efficiency as Measured by the Level of Fused mRNA is Higher when GENERIDE™ is Administered with spCas9 mRNA

The experiment described in this Example demonstrates that the efficiency of integrating the hAAT gene is higher when GENERIDE™ is administered with Streptococcus pyogenes (spCas9) mRNA.

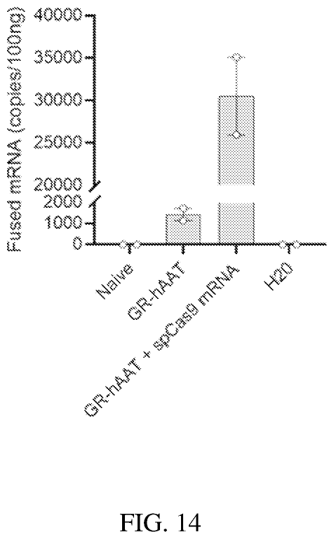

FIG. 14 shows results of an experiment in which a GENERIDE™ construct (GR-hAAT) was administered alone or in combination with spCas9 mRNA, with a naïve and H20 control. HEPG2 cells were seeded at 100 k/well in 24-well format. The next day, medium was replaced with DMEM containing 2.5% fetal bovine serum (FBS) with or without the construct (GR-hAAT) at the MOI 1E5. Cas9 mRNA (1 μg/well) and sgRNA (250 ng/well) were transfected using the Lipofectamine™ MessengerMAX™ according to the vendor protocol. Cells were harvested 3 days after transfection, and mRNA was extracted for the analysis. FIG. 14 shows a graph measuring levels of fused mRNA (copies/100 ng). spCas9 mRNA increased integration efficiency of GR-hAAT.

Thus, this example demonstrates that integration efficiency as measured by the fused mRNA levels are higher when GENERIDE™ is administered with spCas9 mRNA.

### Example 17: Integration of Both Circular and Linear Plasmids can be Enhanced with the Addition of Cas9

The experiment described in this Example demonstrates that the integration of both circular and linear plasmids can be enhanced with the addition of Cas9.

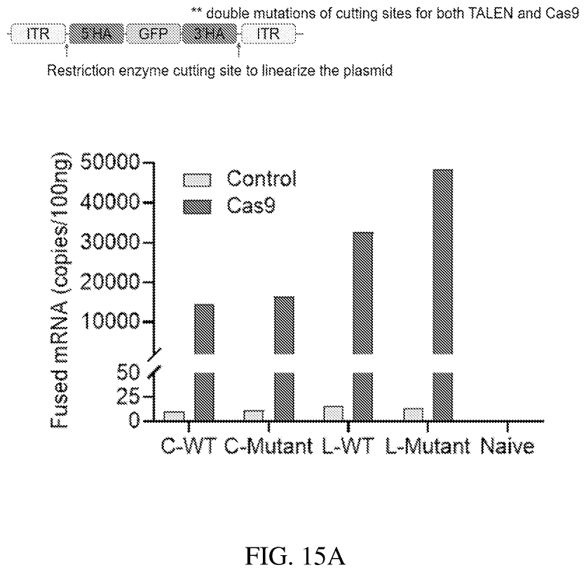

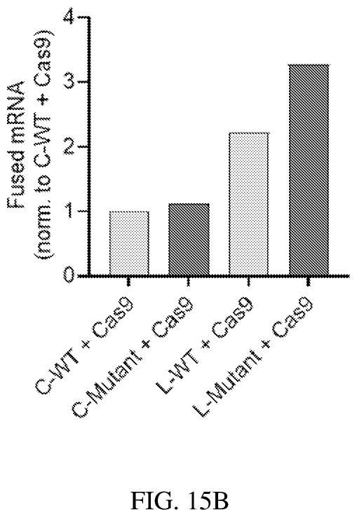

FIGS. 15A and 15B show results of an experiment in which integration efficiency of wild type (WT) versus mutant plasmids in circular or linear format with or without Cas9 was measured. The plasmids had restriction enzyme cutting sites to linearize the plasmid, and mutations on cutting site may further enhance the integration. FIG. 15A shows a graph measuring the levels of fused mRNA (copies/100 ng) in the presence or absence of Cas9 for WT and mutant plasmids in both circular and linear formats, with a naïve control (left panel). FIG. 15B shows a graph measuring the levels of fused mRNA in the presence of Cas9 (norm. to C-WT+Cas9) for WT and mutant plasmids in both circular and linear formats (right panel).

Thus, this example demonstrates that Cas9 enhances the integration of both circular and linear plasmids, with linear plasmids showing better efficiency.

### Example 18: Measurement of Editing Efficiency, Homology-Directed Repair (HDR), and Nonhomologous End Joining (NHEJ) after Transfection with AZ Nuclease and SpCas9 mRNA in HEPG2 Cells

The experiment described in this Example measured the editing efficiency, HDR, and NHEJ after transfection with AZ Nuclease and SpCas9 mRNA in human HEPG2 cells.

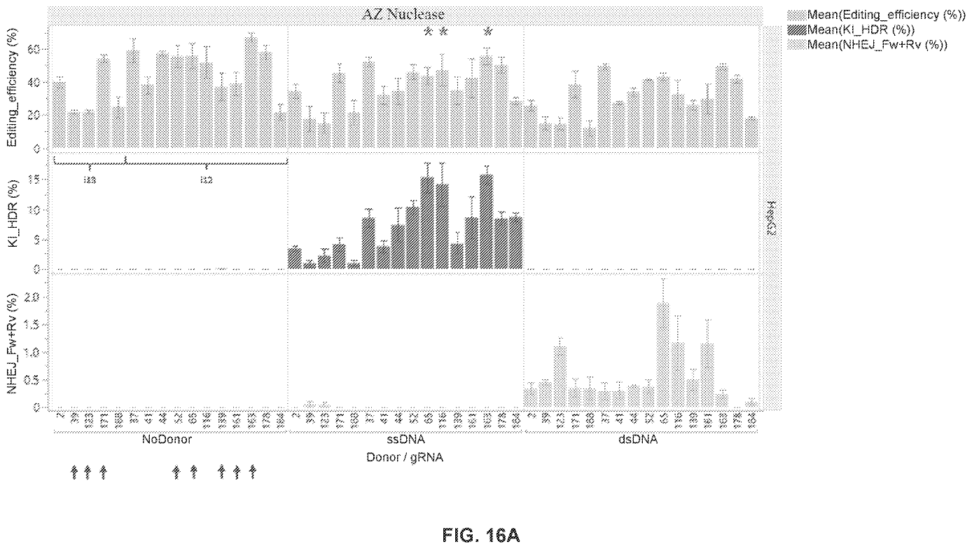

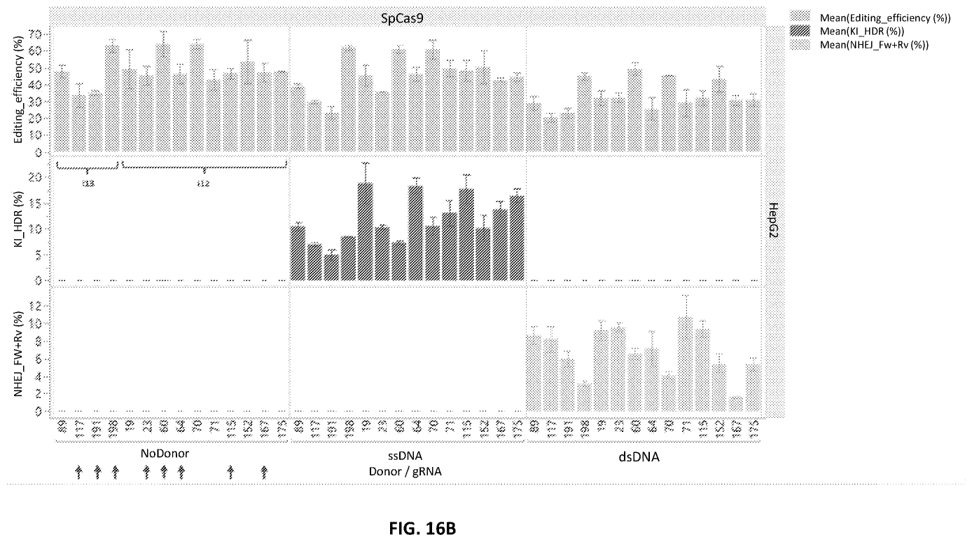

FIGS. 16A and 16B show results of an experiment in which Editing_efficiency, KI_HDR, and NHEJ_Fw+Rv were measured after transfection with AZ Nuclease (FIG. 16A) or SpCas9 (FIG. 16B) mRNA for different ratios of donor to gRNA respectively. HepG2 cells were treated with SpCas9 or AZ Nuclease along with the designated sgRNA. For HDR data, a short ssDNA donor was used with homology arms flanking the cut site. For NHEJ data, a short dsODN donor was used. In all cases, NGS was used to define the rate of INDELs, ssDNA integration, and dsODN integration relative to total reads. Arrows indicate overlapping gRNAs for SpCas9 and AZ Nuclease; asterisks indicate gRNAs with the highest KI-HDR for AZ Nuclease. FIG. 16A shows a series of graphs in which Editing_efficiency (%) (top panel), KI_HDR (%) (middle panel), and NHEJ_Fw+Rv (%) (bottom panel) were measured after transfection with AZ Nuclease mRNA for different ratios of donor to gRNA in HEPG2 cells. Mean KI_HDR (%) was higher for ssDNA compared to dsDNA and vice-versa for NHEJ_Fw+Rv (%). FIG. 16B shows a series of graphs in which Editing_efficiency (%) (top panel), KI_HDR (%) (middle panel), and NHEJ_Fw+Rv (%) (bottom panel) were measured after transfection with SpCas9 mRNA for different ratios of donor to gRNA in HEPG2 cells. Mean KI_HDR (%) was higher for ssDNA compared to dsDNA and vice-versa for NHEJ_Fw+Rv (%). Thus, this example demonstrates that both AZ Nuclease and SpCas9 mRNA can result in GENERIDE™ integration in human HEPG2 cells.

### Example 19: Comparison of gRNAs in Human HEPG2 Cells

The experiment described in this Example compared human gRNAs for spCas9 and AZ Nuclease Cas9 in HEPG2 cells and compares the efficiency of AZ Nuclease Cas9 when cutting within the homology arm (HA) versus outside the HA in human HEPG2 cells.

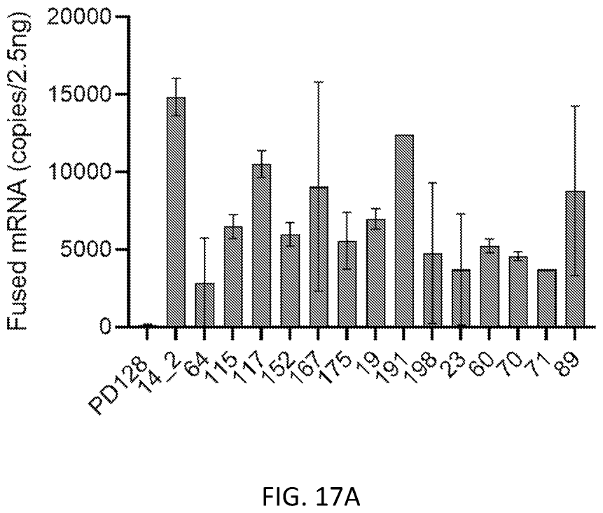

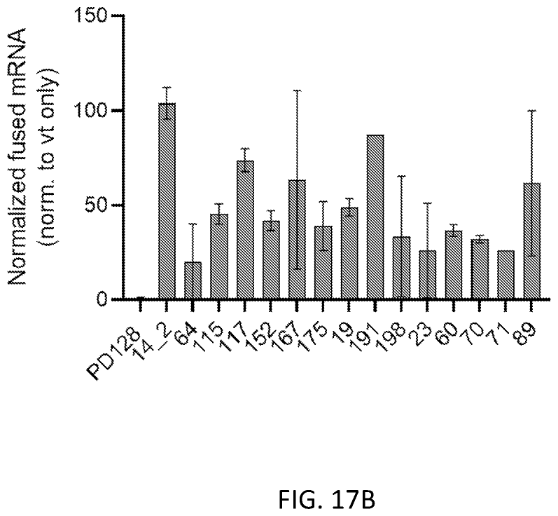

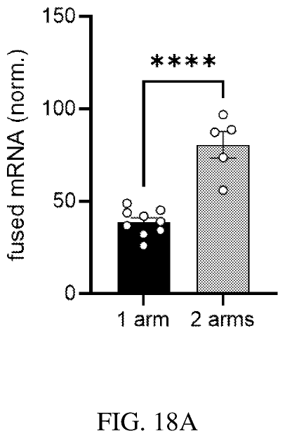

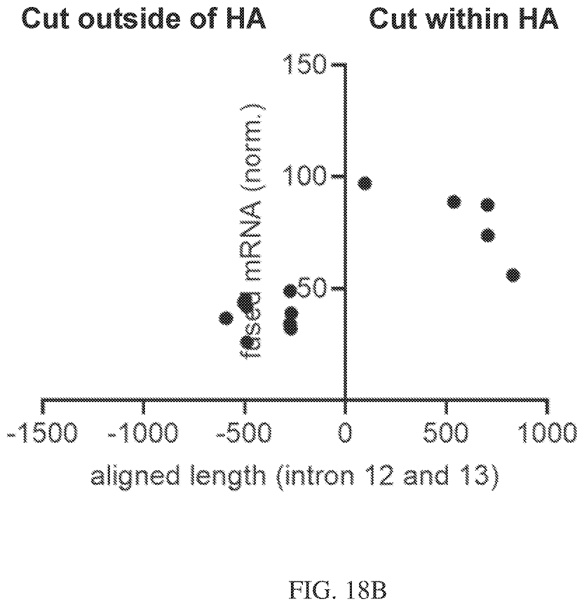

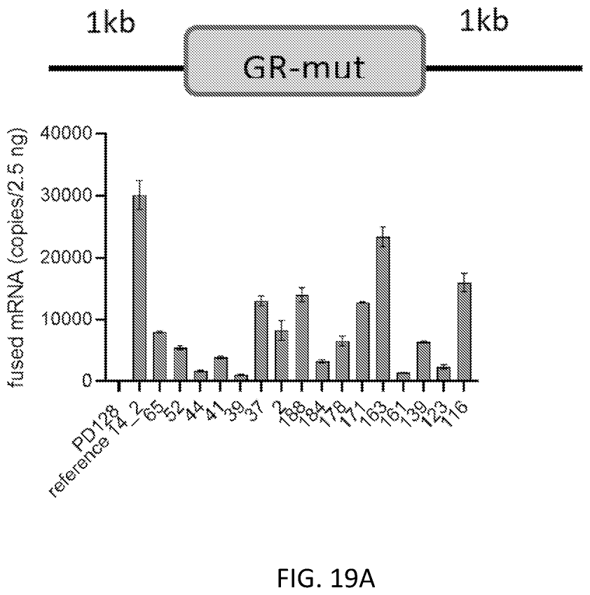

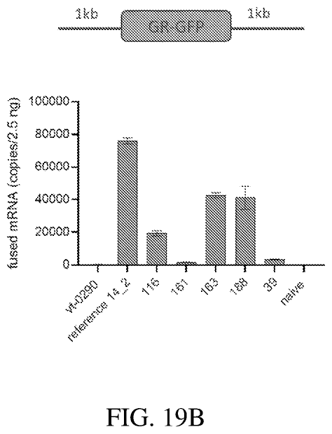

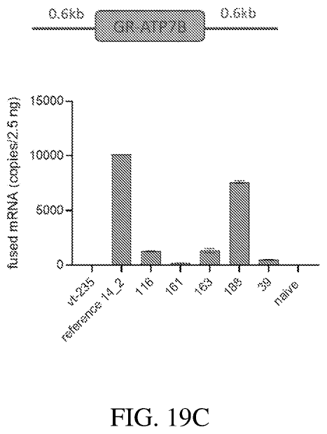

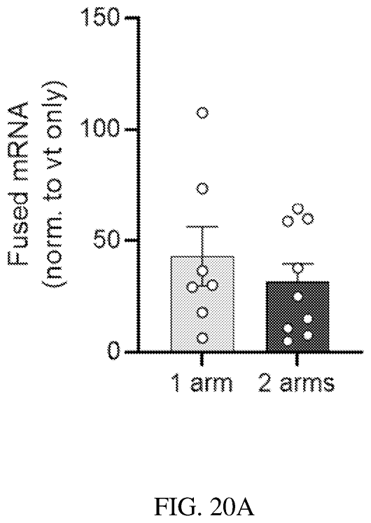

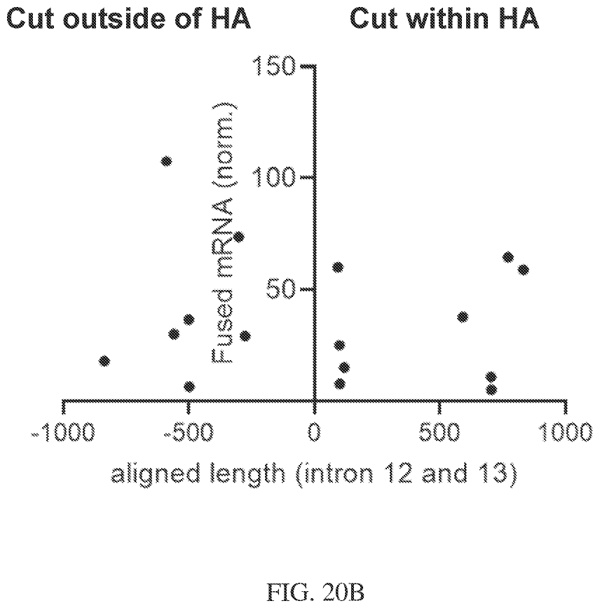

FIGS. 17A and 17B show results of an experiment in which transgene integration efficiency of spCas9 was measured for different human gRNAs in HEPG2 cells. HEPG2 cells were seeded at 100 k/well in 24-well format. The next day, medium was replaced with DMEM containing 2.5% fetal bovine serum (FBS) with or without the constructs (GR-hATP7B or GR-GFP) at the MOI 1E5. Cas9 mRNA (1 μg/well) and sgRNA (250 ng/well) were transfected using the Lipofectamine™ MessengerMAX™ according to the vendor protocol. Cells were harvested 3 days after transfection, and mRNA was extracted for the analysis. FIG. 17A shows a graph measuring the levels of fused mRNA (copies/2.5 ng) in the presence of spCas9 in HEPG2 cells, with a PD128 control (left panel). FIG. 17B shows a graph measuring the levels of normalized fused mRNA (norm. to vt only) in the presence of spCas9 in HEPG2 cells, with a PD128 control (right panel). FIGS. 18A and 18B show results of an experiment evaluating the effect of the cutting location (i.e., cutting within the GR homology arm (HA), in which both the 5′ HA and 3′HA can undergo HDR, versus cutting outside of the GR homology arm, in which only one end can undergo HDR) for spCas9 in human cells. FIG. 18A shows a graph comparing the levels of fused mRNA (norm.) when the cutting is performed outside the HA (1 arm) versus when the cutting is performed within the HA (2 arms); cutting within the HA resulted in significantly higher integration efficiency for spCas9 (left panel). FIG. 18B shows a graph measuring the levels of fused mRNA (norm.) as a function of aligned length (intron 12 and 13) when the cutting is performed outside the HA and within the HA (right panel). FIGS. 19A-19C show results of an experiment in which transgene integration efficiency of GENERIDE™ with AZ Nuclease Cas9 was measured for different human gRNAs in HEPG2 cells. Note that ‘reference 14_2’ is an SpCas9 sgRNA, used with SpCas9 as a positive control. FIG. 19A shows a graph measuring the levels of fused mRNA (copies/2.5 ng) in the presence of GR-mut and AZ Nuclease Cas9 in HEPG2 cells, with a GR only (PD128) control (left panel). FIG. 19B shows a graph measuring the levels of fused mRNA (copies/2.5 ng) in the presence of GR-GFP and AZ Nuclease Cas9 in HEPG2 cells, with a naive control and a GR only (vt-0290) control (middle panel). FIG. 19C shows a graph measuring the levels of fused mRNA (copies/2.5 ng) in the presence of GR-ATP7B and AZ Nuclease Cas9 in HEPG2 cells, with a naive control and GR only (vt-235) control (right panel). FIGS. 20A and 20B show results of an experiment measuring the efficiency of GENERIDE™ integration with AZ Nuclease Cas9 when cutting within the HA versus outside the HA in HEPG2 cells. FIG. 20A shows a graph comparing the levels of fused mRNA (norm. to vt only) when the cutting is performed outside the HA (1 arm) versus when the cutting is performed within the HA (2 arms) (left panel). FIG. 20B shows a graph measuring the levels of fused mRNA (norm.) as a function of aligned length (intron 12 and 13) when the cutting is performed outside the HA and within the HA (right panel).

Thus, this example demonstrates that the gRNA 14-2 for spCas9 resulted in higher integration efficiency in this experiment than other gRNAs, while sgRNA 163 was most-active among AZ Nuclease sgRNA, followed by 37, 188, 171, and 116. We also find that sgRNA 188 works better and more reliably than other AZ Nuclease gRNAs for three different vectors, presumably due to its closer proximity to the integration site resulting in the DSB site being within homology arm bounds for all three vectors. Further, this example illustrates that cutting within the homology arm results in higher integration efficiency in the case of spCas9 but that this does not apply to AZ Nuclease Cas9. Finally, it also shows that the short homology arm ATP7B vector shows less integration compared to the 1 kb homology arm for both spCas9 and AZ Nuclease Cas9.

### Example 20: Measurement of Editing Efficiency, HDR, and NHEJ after Transfection with AZ Nuclease mRNA in HEPG2 Cells

The experiment described in this Example measured the editing efficiency, HDR, and NHEJ after transfection with AZ Nuclease mRNA in HEPG2 cells.

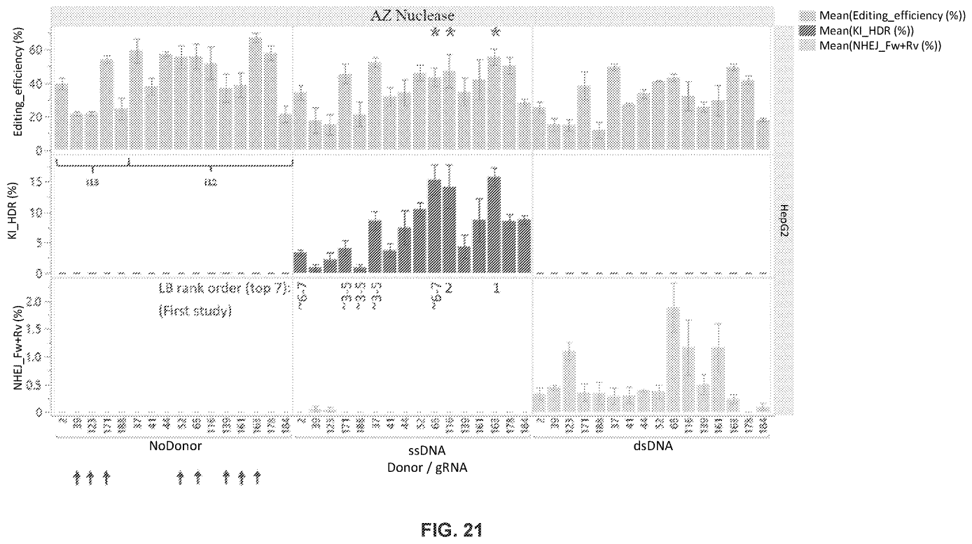

FIG. 21 shows results of an experiment in which Editing_efficiency, KI_HDR, and NHEJ_Fw+Rv were measured after transfection with AZ Nuclease mRNA for the indicated gRNAs and donors (NoDonor, ssDNA, or dsDNA). HepG2 cells were treated with SpCas9 or AZ Nuclease along with the designated sgRNA. For HDR data, a short ssDNA donor was used with homology arms flanking the cut site. For NHEJ data, a short dsODN donor was used. In all cases, NGS was used to define the rate of INDELs, ssDNA integration, and dsODN integration relative to total reads. FIG. 21 shows a series of graphs in which Editing_efficiency (%) (top panel), KI_HDR (%) (middle panel), and NHEJ_Fw+Rv (%) (a measure of integration by NHEJ in either forward or reverse orientations; bottom panel) were measured after transfection with AZ Nuclease mRNA for the indicated gRNAs and donors (NoDonor, ssDNA, or dsDNA) in HEPG2 cells. Mean KI_HDR (%) was higher for ssDNA compared to dsDNA and vice-versa for NHEJ_Fw+Rv (%). Arrows indicate overlapping gRNAs for SpCas9 and AZ Nuclease; asterisks indicate gRNAs with the highest KI-HDR for AZ Nuclease.

Thus, this example demonstrates that AZ Nuclease mRNA leads to gene editing, HDR, and NHEJ in HEPG2 cells. Thus, the data show both which sgRNA promote the highest integration by NHEJ, but also that HDR is generally more efficient than NHEJ, at least in this setting. Additionally, the most active sgRNAs by NHEJ are often but not always among the most active sgRNA by HDR. These trends are more pronounced for AZ Nuclease than SpCas9. Moreover, the exemplary sgRNA sequences in Table 10 were the most active across both data sets—the HDR+NHEJ screening set (NGS data) and the GR-based screening set (fused RNA).

TABLE 10
Exemplary sgRNA sequences

| | | | Target | | |
|---|---|---|---|---|---|
| Species | Nuclease | Name | region | Protospacer | Full sequence as ordered |
| Human | AZ | AZ | Intron 12 | TAGCAATAGGG | mU*mA*mG*rCrArArUrArG |
| | Nuclease | Nuclease_ | | AGTTGAGCTTC | rGrGrArGrUrUrGrArGrCr |
| | | 65/ | | (SEQ ID NO: | UrUrCrGrUrUrUrCrArGrU |
| | | AZ | | 87) | rUrGrArArArGrArArGrUr |
| | | Nuclease_ | | | CrArCrUrCrUrUrArArArG |
| | | 65 | | | rUrGrArGrCrUrGrArArAr |
| | | | | | UrCrArCrUrArArArArArU |
| | | | | | rUrArArGrArUrUrGrArAr |
| | | | | | CrCrCrGrGrCrUrArCrUrG |
| | | | | | rArCrUrCrUrGrUrCrArUr |
| | | | | | CrCrG*mG*mG*mU (SEQ |
| | | | | | ID NO: 93) |
| Human | AZ | AZ | Intron 12 | AAAATAAGGCT | mA*mA*mA*rArUrArArGrG |
| | Nuclease | Nuclease_ | | ATCCAAACTCA | rCrUrArUrCrCrArArArCr |
| | | 116/ | | (SEQ ID NO: | UrCrArGrUrUrUrCrArGrU |
| | | AZ | | 88) | rUrGrArArArGrArArGrUr |
| | | Nuclease_ | | | CrArCrUrCrUrUrArArArG |
| | | 116 | | | rUrGrArGrCrUrGrArArAr |
| | | | | | UrCrArCrUrArArArArArU |
| | | | | | rUrArArGrArUrUrGrArAr |
| | | | | | CrCrCrGrGrCrUrArCrUrG |
| | | | | | rArCrUrCrUrGrUrCrArUr |
| | | | | | CrCrG*mG*mG*mU (SEQ ID |
| | | | | | NO: 94) |
| Human | AZ | AZ | Intron 12 | TCTCATGTGTG | mU*mC*mU*rCrArUrGrUrG |
| | Nuclease | Nuclease_ | | GGGCCACCATA | rUrGrGrGrGrCrCrArCrCr |
| | | 613/ | | (SEQ ID NO: | ArUrArGrUrUrUrCrArGrU |
| | | AZ | | 89) | rUrGrArArArGrArArGrUr |
| | | Nuclease_ | | | CrArCrUrCrUrUrArArArG |
| | | 163 | | | rUrGrArGrCrUrGrArArAr |
| | | | | | UrCrArCrUrArArArArArU |
| | | | | | rUrArArGrArUrUrGrArAr |
| | | | | | CrCrCrGrGrCrUrArCrUrG |
| | | | | | rArCrUrCrUrGrUrCrArUr |
| | | | | | CrCrG*mG*mG*mU (SEQ |
| | | | | | ID NO: 95) |
| Human | AZ | AZ | Intron 13 | AAAAGTTACTG | mA*mA*mA*rArGrUrUrArC |
| | Nuclease | Nuclease_ | | TCAGGCCATAA | rUrGrUrCrArGrGrCrCrAr |
| | | 818/ | | (SEQ ID NO: | UrArArGrUrUrUrCrArGrU |
| | | AZ | | 90) | rUrGrArArArGrArArGrUr |
| | | Nuclease_ | | | CrArCrUrCrUrUrArArArG |
| | | 188 | | | rUrGrArGrCrUrGrArArAr |
| | | | | | UrCrArCrUrArArArArArU |
| | | | | | rUrArArGrArUrUrGrArAr |
| | | | | | CrCrCrGrGrCrUrArCrUrG |
| | | | | | rArCrUrCrUrGrUrCrArUr |
| | | | | | CrCrG*mG*mG*mU (SEQ |
| | | | | | ID NO: 96) |
| Human | AZ | ESOH_Alb_ | Intron 14 | AGTTTAGAATA | mA*mG*mU*rUrUrArGrArA |
| | Nuclease | 14-2 | | GTGGTCGGTGC | rUrArGrUrGrGrUrCrGrGr |
| | | | | (SEQ ID NO: | UrGrCrGrUrUrUrCrArGrU |
| | | | | 91) | rUrGrArArArGrArArGrUr |
| | | | | | CrArCrUrCrUrUrArArArG |
| | | | | | rUrGrArGrCrUrGrArArAr |
| | | | | | UrCrArCrUrArArArArArU |
| | | | | | rUrArArGrArUrUrGrArAr |
| | | | | | CrCrCrGrGrCrUrArCrUrG |
| | | | | | rArCrUrCrUrGrUrCrArUr |
| | | | | | CrCrG*mG*mG*mU (SEQ |
| | | | | | ID NO: 97) |
| Human | SpCas9 | spCas9_14_ | Intron 14 | TTTAGAATAGT | mU*mU*mU*rArGrArArUrA |
| | | 2_HiMod | | GGTCGGTGC | rGrUrGrGrUrCrGrGrUrGr |
| | | | | (SEQ ID NO: | CrGrUrUrUrUrArGrAmGmC |
| | | | | 92) | mUmAmGmAmAmAmUmAmGmCr |
| | | | | | ArArGrUrUrArArArArUrA |
| | | | | | rArGrGrCrUrArGrUrCrCr |
| | | | | | GrUrUrArUrCrAmAmCmUmU |
| | | | | | mGmAmAmAmAmAmGmUmGmGm |
| | | | | | CmAmCmCmGmAmGmUmCmGmG |
| | | | | | mUmGmCmU*mU*mU*mU |
| | | | | | (SEQ ID NO: 98) |

‘m’ is 2′OMe, * indicated as phosphorothioate linkage, and r indicates ribo.

### Example 22: Assessment of RNA from IDT to Ensure Feasibility with AZ LNP

The experiment described in this Example demonstrates a study designed to assess RNA from IDT to ensure feasibility with LNP (obtained from AstraZeneca; “AZ LNP”) where the quality of RNA materials from IDT were assessed to ensure working feasibility with LNP and N:P ratios, formulation preparations, and dosing stagger of LB 1 Day (D1) versus 4 hours (H4) were compared.

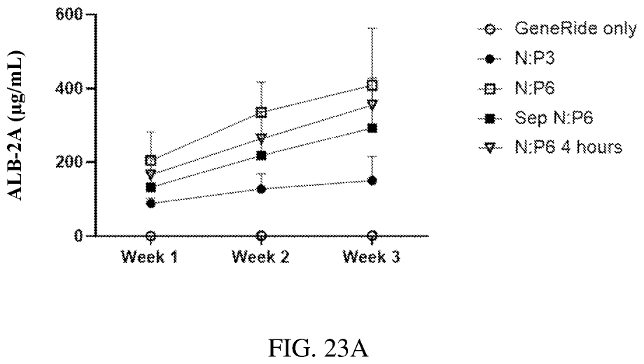

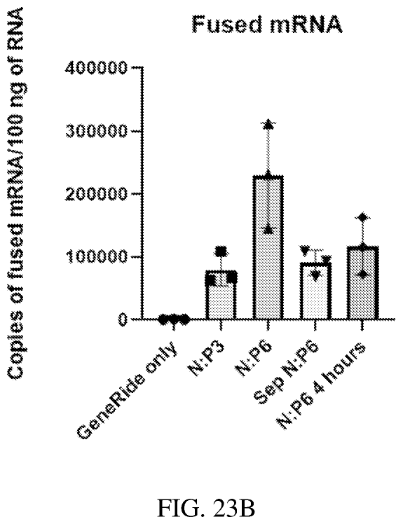

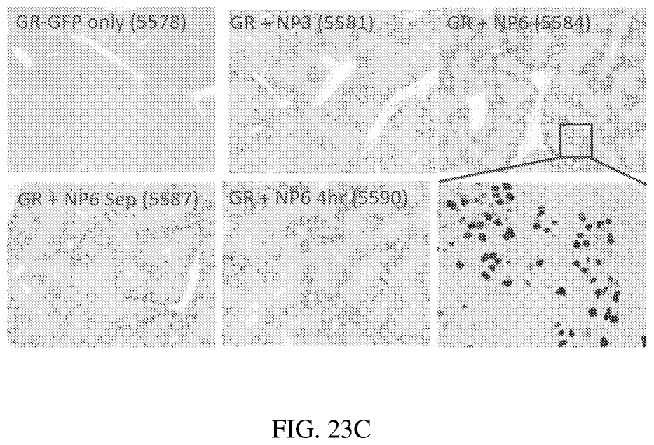

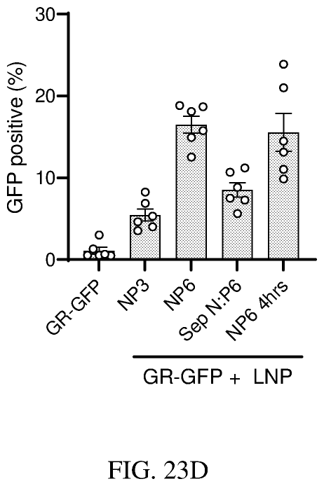

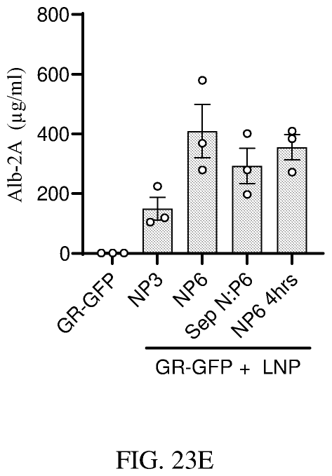

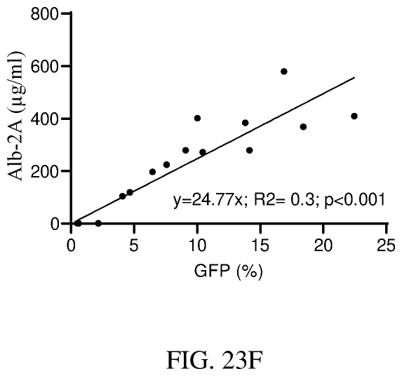

FIGS. 23A-23F show results of experiments performed to assess RNA from IDT to ensure feasibility with AZ LNP. The GR-HDR donor used was DJ-mHA (1.0/1.0) GFP-LB-Vt-0298-001 and the AZ LNP used was from AstraZeneca. 5 different combinations were administered to different groups of WT mice: WT GR only, WT GR+LNP N:P=3 (Co-formulation) D1 Stagger, WT GR+LNP N:P=6 (Co-formulation) D1 Stagger, WT GR+LNP N:P=6 (Sep formulation) D1 Stagger, and WT GR+LNP N:P=6 (Co-formulation) H4 Stagger. FvB mice received DJ-AAV-GFP along with prepared with an N·P ratio of 3 or 6 (as indicated), with the mRNA and sgRNA either formulated together (‘Co-formulation’) or separately (‘September formulation’). The stagger time indicates whether LNP was dose 1 day or 4 hours after AAV dosing. FIG. 23A shows results of an experiment performed to measure the levels of ALB-2A (μg/ml) for up to 3 weeks post LNP dosing for the 5 different combinations listed above, with the GR only as control. FIG. 23B is a bar graph measuring the copies of fused mRNA/100 ng of RNA for the 5 different combinations administered, with the GR only as control. FIG. 23C is a set of IHC images with the IHC being performed to label and measure GFP+ cells after administration of the 5 combinations listed above. FIG. 23D is a bar graph measuring the % of GFP positive cells after administration of the 5 combinations listed above. FIG. 23E is a bar graph measuring the levels of ALB-2A (μg/ml) after administration of the 5 combinations listed above. FIG. 23F shows the levels of ALB-2A (μg/ml) as a function of the % of GFP positive cells after administration of the 5 combinations listed above.

Thus, this example demonstrates that there is improved improved integration, as quantified by expression of ALB-2A and fused mRNA integration when using GR2.0 compared to GR alone and that is true for all GR2.0 groups, with AZ LNP performing better than the historical Genvoy-ILM LNP. The N:P6 resulted in higher expression of ALB-2A than N:P3, however the co-formulated guide and spCas9 RNA combination resulted in higher ALB-2A expression when compared to the separately formulated guide and Cas9 combination. There was comparable ALB-2A expression with both H4 and D1 dose stagger between GR and LNP components, with respect to the administration of components. This example showed that LNP N:P6 co-formulation produced ~16% integration of GFP+ cells in the IHC which correlated with ALB-2A expression. In addition, CD4 staining did not show a difference between groups when compared to GR alone and molecular gDNA integration generally correlated with fused RNA and ALB-2A.

### Example 23: Optimization of the Treatment Timeline: Treatment with GR One Day after mRNA Transfection Further Enhanced Integration

The experiment described in this Example demonstrates that treatment with GR one day after mRNA transfection further enhances GR integration.

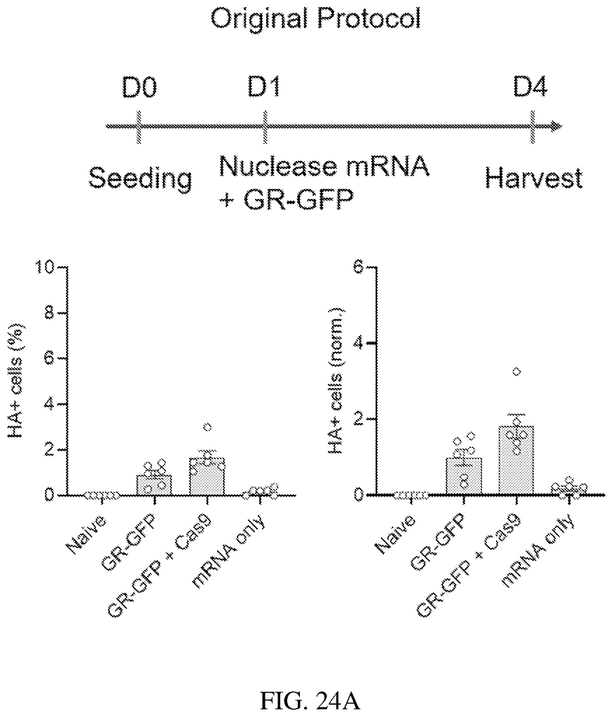

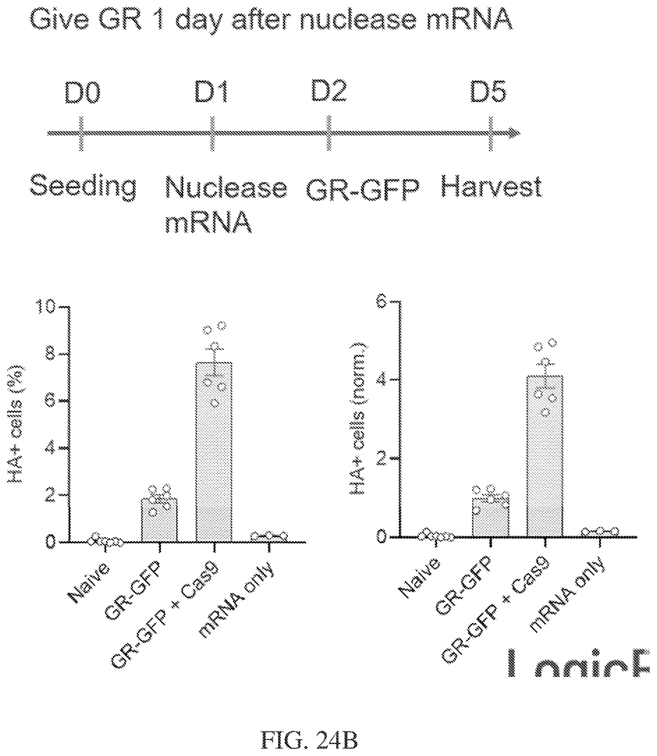

FIGS. 24A and 24B show results of experiments performed to optimize a treatment timeline, in which cells were treated according to 2 different protocols to determine which was better at enhancing integration. FIG. 24A shows results of experiments in which cells were treated with GR on the same day as the mRNA transfection (original protocol). Seeding was performed on D0 followed by nuclease mRNA transfection along with GR-GFP treatment on D1 and finally, harvesting was performed on D4 (top panel). FIG. 24A shows a bar graph measuring the % of HA+ cells for the 4 different combinations administered: naïve (control), GR-GFP, GR-GFP+Cas9, and mRNA only (bottom left panel) and another bar graph measuring the % of HA+ cells (norm.) for the same 4 combinations administered (bottom right panel). FIG. 24B shows results of experiments in which cells were treated with GR on the day after the mRNA transfection. Seeding was performed on D0 followed by nuclease mRNA transfection on D1, followed by GR-GFP treatment on D2 and finally, harvesting was performed on D5 (top panel). FIG. 24B shows a bar graph measuring the % of HA+ cells for the 4 different combinations administered: naïve (control), GR-GFP, GR-GFP+Cas9, and mRNA only (bottom left panel) and another bar graph measuring the % of HA+ cells (norm.) for the same 4 combinations administered (bottom right panel).

Thus, this example demonstrates that treatment with GR one day after mRNA transfection further enhances integration. The data may also suggest that adding dsODN one day after will result in better dsODN annealing efficiency.

### Example 24: Effect of Transfection Reagents on the Transfection Efficiency and Editing Ratio In Vitro

The experiment described in this Example evaluated the effect of different transfection reagents on transfection efficiency and editing ratio in vitro in human cells.

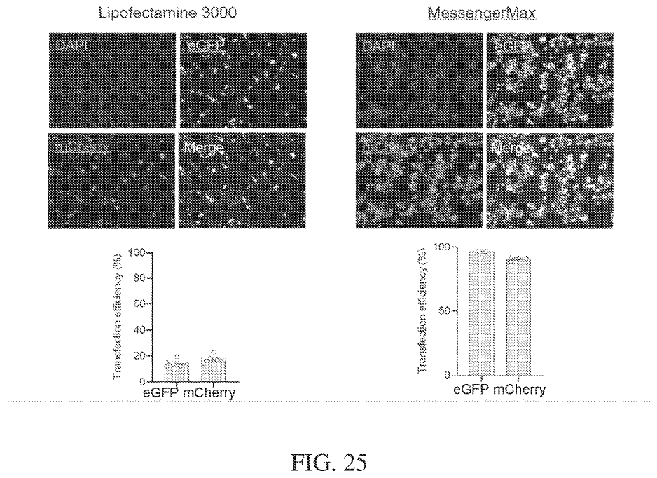

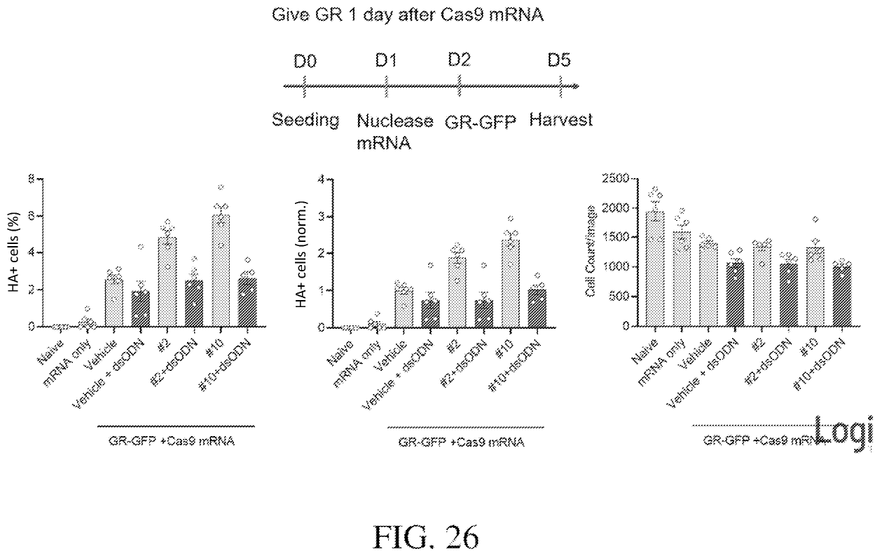

FIG. 25 shows results of experiments performed to compare the transfection efficiency of different transfection reagents via immunofluorescence. FIG. 25 shows 4 immunofluorescence images (top left panel) testing the transfection efficiency of Lipofectamine™ 3000 for transfection of mCherry and eGFP, with a DAPI channel and an eGFP-mCherry merged image showing expression of both and 4 immunofluorescence images (top right panel) testing the transfection efficiency of MessengerMax™ for transfection of mCherry and eGFP, with a DAPI channel and an eGFP-mCherry merged image showing expression of both. FIG. 25 also shows bar graphs measuring the % Transfection efficiency for eGFP and mCherry for both Lipofectamine™ 3000 and MessengerMax™, bottom left and bottom right panels, respectively. FIG. 26 shows a bar graph measuring the % of HA+ cells for the 8 different combinations administered: naïve (control), mRNA only, Vehicle+GR-GFP+Cas9 mRNA, Vehicle+dsODN+GR-GFP+Cas9 mRNA, #2 gRNA+GR-GFP+Cas9 mRNA, #2 gRNA+dsODN+GR-GFP+Cas9 mRNA, #10 gRNA+GR-GFP+Cas9 mRNA, and #10 gRNA+dsODN+GR-GFP+Cas9 mRNA (bottom left panel), a bar graph measuring the % of HA+ cells (norm.) for the same 8 combinations administered (bottom middle panel), and another bar graph measuring the cell count/image for the same 8 combinations administered (bottom right panel).

Thus, this example demonstrates that MessengerMax™ is superior to Lipofectamine 3000 for mRNA transfection, as evident from its much higher transfection efficiency than Lipofectamine 3000, but it did not show much separation from the baseline.

### EQUIVALENTS

Those skilled in the art will recognize or be able to ascertain using no more than routine experimentation, many equivalents to the specific embodiments of the invention described herein. The scope of the present invention is not intended to be limited to the above Description, but rather is as set forth in the following claims:

We claim:

1. A composition or set of compositions comprising:
(i) a nuclease or polynucleotide sequence encoding a nuclease, wherein the nuclease is selected from a clustered regularly interspaced short palindromic repeats (CRISPR)-associated (Cas) enzyme, a transcription activator-like effector (TALE) nuclease (TALEN), a TALE nickase, a zinc finger (ZF) nuclease (ZFN), a ZF nickase, or a meganuclease; and
(ii) a polynucleotide cassette comprising:
an expression cassette comprising a first nucleic acid sequence and a second nucleic acid sequence, wherein the first nucleic acid sequence encodes a transgene; and the second nucleic acid sequence is positioned 5′ or 3′ to the first nucleic acid sequence and promotes the production of two independent gene products upon integration into a target integration site in the genome of a human cell;
a third nucleic acid sequence positioned 5′ to the expression cassette and comprising a sequence that is substantially homologous to a human genomic sequence 5′ of the target integration site in a genome of a human cell; and
a fourth nucleic acid sequence positioned 3′ to expression cassette and comprising a sequence that is substantially homologous to a human genomic sequence 3′ of the target integration site in the genome of the human cell;
wherein:
the polynucleotide cassette does not include a promoter sequence; and
the nuclease is capable of inducing a double-stranded break and/or a single-stranded break at a cut site in the genome of the human cell.

2. The composition or the set of compositions of claim 1, wherein the target integration site is an albumin locus comprising an endogenous albumin promoter and an endogenous albumin gene.

3. A composition or set of compositions comprising:
(i) a nuclease or polynucleotide sequence encoding a nuclease; and
(ii) a polynucleotide cassette comprising:
an expression cassette comprising a first nucleic acid sequence and a second nucleic acid sequence, wherein the first nucleic acid sequence encodes a transgene; and the second nucleic acid sequence is positioned 5′ or 3′ to the first nucleic acid sequence and promotes the production of two independent gene products upon integration into a target integration site in the genome of a human cell, wherein the target integration site is an albumin locus comprising an endogenous albumin promoter and an endogenous albumin gene;
a third nucleic acid sequence positioned 5′ to the expression cassette and comprising a sequence that is substantially homologous to a human genomic sequence 5′ of the target integration site in a genome of a human cell; and
a fourth nucleic acid sequence positioned 3′ to expression cassette and comprising a sequence that is substantially homologous to a human genomic sequence 3′ of the target integration site in the genome of the human cell;
wherein:
the polynucleotide cassette does not include a promoter sequence; and
the nuclease is capable of inducing a double-stranded break and/or a single-stranded break at a cut site in the genome of the human cell.

4. The composition or the set of compositions of any one of claims 1-3, further comprising a recombinant viral vector.

5. The composition or the set of compositions of claim 4, wherein the recombinant viral vector is a recombinant AAV vector.

6. The composition or the set of compositions of claim 5, wherein the recombinant viral vector is or comprises a capsid polypeptide comprising an amino acid sequence having at least 95% sequence identity with the amino acid sequence of sL65, LK03, AAV8, AAV-DJ; AAV-LK03; or AAVNP59.

7. The composition or the set of compositions of claim 6, wherein the recombinant viral vector comprises the capsid polypeptide and the polynucleotide sequence encoding a nuclease and/or the polynucleotide cassette is encapsidated in the recombinant viral vector.

8. The composition or the set of compositions of claim 6, wherein the polynucleotide cassette is encapsidated in the recombinant viral vector.

9. The composition or the set of compositions of any one of claims 1-8, further comprising AAV2 inverted terminal repeat (ITR) sequences.

10. The composition or the set of compositions of claim 9, wherein the AAV2 ITR sequences flank the 5′ and 3′ ends of the polynucleotide sequence encoding the nuclease and/or the polynucleotide cassette.

11. The composition or the set of compositions of any one of claims 1-10, wherein the third and fourth nucleic acid sequence are each between 50 nt and 1600 nt in length.

12. The composition or the set of compositions of any one of claims 1-11, wherein the third and fourth nucleic acid sequence are the same length.

13. The composition or the set of compositions of any one of claims 1-12, wherein the third and fourth nucleic acid sequence are different lengths.

14. The composition or the set of compositions of any one of claims 1-13, wherein upon integration of the polynucleotide cassette into the target integration site in the genome of the cell, the transgene is expressed under control of an endogenous promoter at the target integration site.

15. The composition or the set of compositions of any one of claims 2-14, wherein the target integration site is within a coding sequence of the albumin locus and 5′-adjacent to a stop codon.

16. The composition or the set of compositions of claim 15, wherein the target integration site is 5′-adjacent to a stop codon in exon 14 of the albumin locus.

17. The composition or the set of compositions of any one of claims 1-16, wherein the cut site is within a non-coding sequence of the albumin locus.

18. The composition or the set of compositions of claim 17, wherein the cut site is within an intron, untranslated region, enhancer, promoter, silencer, or insulator of the albumin locus.

19. The composition or the set of compositions of claim 18, wherein the cut site is within intron 12, 13, or 14 of the albumin locus.

20. The composition or the set of compositions of any 3-18, wherein the nuclease is selected from a Cas enzyme, a TALEN, a TALE nickase, a ZFN, a ZF nickase, or a meganuclease.

21. The composition or the set of compositions of any one of claims 1-20, wherein the nuclease is a Cas enzyme or a TALEN.

22. The composition or the set of compositions of any one of claims 1-21, wherein the nuclease is a Cas enzyme.

23. The composition or the set of compositions of any one of claims 1-22, wherein the Cas enzyme is selected from Staphylococcus aureus Cas9 (saCas9), Streptococcus pyogenes (spCas9), AZ Nuclease, HF1-Cas9, HF2-Cas9, or HiFi-Cas9.

24. The composition or the set of compositions of claim 22 or 23, further comprising a guide RNA (gRNA).

25. The composition or the set of compositions of claim 24, wherein the gRNA comprises a nucleic acid sequence of any one of SEQ ID NOs: 27-45, 71-86, or 93-98, or a nucleic acid sequence having at least 80%, 85%, 90%, 95%, 96%, 97%, 98%, or 99% identity to any one of SEQ ID NOs: 27-45, 71-86, or 93-98.

26. The composition or the set of compositions of claim 24 or 25, wherein the nuclease or polynucleotide sequence encoding the nuclease and the gRNA are co-formulated.

27. The composition or the set of compositions of claim 24 or 25, wherein the nuclease or polynucleotide sequence encoding the nuclease and the gRNA are formulated separately.

28. The composition or the set of compositions of any one of claims 1-27, wherein the second nucleic acid sequence is or comprises:
a) a nucleic acid sequence encoding a 2A peptide;
b) a nucleic acid sequence encoding an internal ribosome entry site (IRES);
c) a nucleic acid sequence encoding an N-terminal intein splicing region and C-terminal intein splicing region; or
d) a nucleic acid sequence encoding a splice donor and a splice acceptor.

29. The composition or the set of compositions of any one of claims 1-28, wherein the second nucleic acid sequence is or comprises a nucleic acid sequence encoding a 2A peptide.

30. The composition or the set of compositions of claim 29, wherein the second nucleic acid is or comprises a nucleic acid sequence encoding a 2A peptide selected from the group consisting of P2A, T2A, E2A, and F2A.

31. The composition or the set of compositions of claim 1 or 3, wherein the cut site is between 1 and 2000 bp from the target integration site.

32. The composition or the set of compositions of claim 31, wherein the cut site is up to 100 bp from the target integration site.

33. The composition or the set of compositions of any one of claims 1-32, wherein the transgene is selected from CBS, UGT1A1, MUT, FAH, ATP7B, A1AT, ASL, LIPA, PAH, G6PC, Factor IX, or a variant thereof.

34. The composition or the set of compositions of any one of claims 1-33, which is a set of compositions, wherein the nuclease or polynucleotide sequence encoding the nuclease is formulated in a lipid nanoparticle (LNP) and the polynucleotide cassette is encapsidated in a recombinant AAV vector.

35. A method of integrating a transgene into the genome of a human cell, said method comprising
administering to a subject a composition or set of compositions comprising:
(i) a nuclease or a polynucleotide sequence encoding a nuclease, wherein the nuclease is selected from a Cas enzyme, a TALEN, a TALE nickase, a ZFN, a ZF nickase, or a meganuclease; and
(ii) a polynucleotide cassette comprising:
an expression cassette comprising a first nucleic acid sequence and a second nucleic acid sequence, wherein the first nucleic acid sequence encodes a transgene; and the second nucleic acid sequence is positioned 5′ or 3′ to the first nucleic acid sequence and promotes the production of two independent gene products upon integration into a target integration site in the genome of a human cell;
a third nucleic acid sequence positioned 5′ to the expression cassette and comprising a sequence that is substantially homologous to a human genomic sequence 5′ of the target integration site in a genome of a human cell; and
a fourth nucleic acid sequence positioned 3′ to expression cassette and comprising a sequence that is substantially homologous to a human genomic sequence 3′ of the target integration site in the genome of the human cell;
wherein:
the polynucleotide cassette does not include a promoter sequence; and
the nuclease is capable of inducing a double-stranded break and/or a single-stranded break at a cut site in the genome of the cell;
wherein, after administering the composition or the set of compositions, the transgene is integrated into the genome of the human cell.

36. The method of claim 35, wherein the target integration site is an albumin locus comprising an endogenous albumin promoter and an endogenous albumin gene.

37. A method of integrating a transgene into the genome of a human cell, said method comprising
administering to a subject a composition or set of compositions comprising:
(i) a nuclease or a polynucleotide sequence encoding a nuclease; and
(ii) a polynucleotide cassette comprising:
an expression cassette comprising a first nucleic acid sequence and a second nucleic acid sequence, wherein the first nucleic acid sequence encodes a transgene; and the second nucleic acid sequence is positioned 5′ or 3′ to the first nucleic acid sequence and promotes the production of two independent gene products upon integration into a target integration site in the genome of a human cell, wherein the target integration site is an albumin locus comprising an endogenous albumin promoter and an endogenous albumin gene;
a third nucleic acid sequence positioned 5′ to the expression cassette and comprising a sequence that is substantially homologous to a human genomic sequence 5′ of a target integration site in a genome of a human cell; and
a fourth nucleic acid sequence positioned 3′ to expression cassette and comprising a sequence that is substantially homologous to a human genomic sequence 3′ of a target integration site in the genome of the human cell;
wherein:
the polynucleotide cassette does not include a promoter sequence; and
the nuclease is capable of inducing a double-stranded break and/or a single-stranded break at a cut site in the genome of the cell;
wherein, after administering the composition or the set of compositions, the transgene is integrated into the genome of the human cell.

38. The method of any one of claims 35-37, wherein the composition or the set of compositions further comprises a recombinant viral vector.

39. The method of claim 38, wherein the recombinant viral vector is a recombinant AAV vector.

40. The method of claim 39, wherein the recombinant viral vector is or comprises a capsid polypeptide comprising an amino acid sequence having at least 95% sequence identity with the amino acid sequence of LK03, AAV8, AAV-DJ; AAV-LK03; or AAVNP59.

41. The method of claim 40, wherein the recombinant viral vector comprises the capsid polypeptide and the polynucleotide sequence encoding a nuclease and/or the polynucleotide cassette is encapsidated in the recombinant viral vector.

42. The method of claim 41, wherein the polynucleotide cassette is encapsidated in the recombinant viral vector.

43. The method of any one of claims 35-42, wherein the composition or the set of compositions further comprises AAV2 ITR sequences.

44. The method of claim 43, wherein the AAV2 ITR sequences flank the 5′ and 3′ ends of the polynucleotide sequence encoding the nuclease and/or the polynucleotide cassette.

45. The method of any one of claims 35-44, wherein the third and fourth nucleic acid sequence are between 50 nt and 1600 nt in length.

46. The method of any one of claims 35-45, wherein the third and fourth nucleic acid sequence are the same length.

47. The method of any one of claims 35-45, wherein the third and fourth nucleic acid sequence are different lengths.

48. The method of any one of claims 35-47, wherein upon integration of the polynucleotide cassette into the target integration site in the genome of the cell, the transgene is expressed under control of an endogenous promoter at the target integration site.

49. The method of any one of claims 36-48, wherein the target integration site is within a coding sequence of the albumin locus and 5′-adjacent to a stop codon.

50. The method of claim 49, wherein the target integration site is 5′-adjacent to a stop codon in exon 14 of the albumin locus.

51. The method of any one of claims 36-50, wherein the cut site is within a non-coding sequence of the albumin locus.

52. The method of claim 51, wherein the cut site is within an intron, untranslated region, enhancer, promoter, silencer, or insulator of the albumin locus.

53. The method of claim 52, wherein the cut site is within intron 12, 13, or 14 of the albumin locus.

54. The method of any one of claims 37-53, wherein the nuclease is selected from a Cas enzyme, a TALEN, a TALE nickase, a ZFN, a ZF nickase, or a meganuclease.

55. The method of any one of claims 35-54, wherein the nuclease is a Cas enzyme or a TALEN.

56. The method of any one of claims 35-55, wherein the nuclease is a Cas enzyme.

57. The method of any one of claims 35-56, wherein the Cas enzyme is selected from Staphylococcus aureus Cas9 (saCas9), Streptococcus pyogenes (spCas9), AZ Nuclease, HF1-Cas9, HF2-Cas9, or HiFi-Cas9.

58. The method of claim 56 or 57, further comprising a guide RNA (gRNA).

59. The method of claim 58, wherein the gRNA comprises a nucleic acid sequence of any one of SEQ ID NOs: 27-45, 71-86, or 93-98, or a nucleic acid sequence having at least 80%, 85%, 90%, 95%, 96%, 97%, 98%, or 99% identity to any one of SEQ ID NOs: 27-45, 71-86, or 93-98.

60. The method of claim 58 or 59, wherein the nuclease or polynucleotide sequence encoding the nuclease and the gRNA are co-formulated.

61. The method of claim 58 or 59, wherein the nuclease or polynucleotide sequence encoding the nuclease and the gRNA are formulated separately.

62. The method of any one of claims 35-61, wherein the second nucleic acid sequence is or comprises:
a) a nucleic acid sequence encoding a 2A peptide;
b) a nucleic acid sequence encoding an internal ribosome entry site (IRES);
c) a nucleic acid sequence encoding an N-terminal intein splicing region and C-terminal intein splicing region; or
d) a nucleic acid sequence encoding a splice donor and a splice acceptor.

63. The method of any one of claims 35-62, wherein the second nucleic acid sequence is or comprises a nucleic acid sequence encoding a 2A peptide.

64. The method claim 63, wherein the second nucleic acid sequence is or comprises a nucleic acid sequence encoding a 2A peptide selected from the group consisting of P2A, T2A, E2A, and F2A.

65. The method of claim 35 or 37, wherein the cut site is between 1 and 1000 bp from the target integration site.

66. The method of claim 65, wherein the cut site is up to 100 bp from the target integration site.

67. The method of any one of claims 35-66, wherein the transgene is selected from CBS, UGT1A1, MUT, FAH, ATP7B, A1AT, ASL, LIPA, PAH, G6PC, Factor IX, or a variant thereof.

68. The method of any one of claims 35-67, which is a set of compositions, wherein the nuclease or polynucleotide sequence encoding the nuclease is formulated in a lipid nanoparticle and the polynucleotide cassette is encapisdated in a recombinant AAV vector.

69. The method of any one of claims 35-68, wherein the cell is edited in vivo.

70. The method of any one of claims 35-69, wherein the method of integrating a transgene is conducted ex vivo.

71. The method of claim 69 or 70, wherein the cell is a blood, liver, muscle, or CNS cell.

72. The method of claim 70, wherein the cell is administered in an autologous transplant after transgene integration.

73. The method of claim 70, wherein the cell is administered in an allogeneic transplant after transgene integration.

74. The method of any one of claims 35-73, wherein (i) the nuclease or the polynucleotide sequence encoding the nuclease and (ii) the polynucleotide cassette are administered to the subject on the same day.

75. The method of any one of claims 35-73, wherein (i) the nuclease or the polynucleotide sequence encoding the nuclease and (ii) the polynucleotide cassette are administered to the subject on different days.

76. The method of claim 75, wherein the nuclease or the polynucleotide sequence encoding the nuclease is administered to the subject 1 hour to 3 days after the polynucleotide cassette.

77. The method of claim 76, wherein the nuclease or the polynucleotide sequence encoding the nuclease is administered to the subject 1 hour to 24 hours after the polynucleotide cassette.

78. The method of claim 75, wherein the nuclease or the polynucleotide sequence encoding the nuclease is administered to the subject 1 hour to 3 days before the polynucleotide cassette.

79. The method of claim 76, wherein the nuclease or the polynucleotide sequence encoding the nuclease is administered to the subject 1 hour to 4 hours before the polynucleotide cassette.

80. The method of claim 79, wherein the nuclease or the polynucleotide sequence encoding the nuclease is administered to the subject 4 hours before the polynucleotide cassette.

81. The composition or the set of compositions of any one of claims 1-34, wherein the cut site is distal from the target integration site.

82. The method of any one of claims 35-80, wherein the cut site is distal from the target integration site.


SEQUENCE LISTING (101 sequences)

SEQ ID NO: 1 - DNA, 2211 bp, synthetic construct
  source 1..2211: mol_type = other DNA
atggctgctgacggttatcttccagattggctcgaggacaacctttctgaaggcattcgagagtggtgggcgctgcaacctggagcccctaaacccaaggcaaatcaacaacatcaggacaacgctcggggtcttgtgcttccgggttacaaatacctcggacccggcaacggactcgacaagggggaacccgtcaacgcagcggacgcggcagccctcgagcacgacaaggcctacgaccagcagctcaaggccggtgacaacccctacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagatcagtctcctcaggaaccggactcatcatctggtgttggcaaatcgggcaaacagcctgccagaaaaagactaaatttcggtcagactggcgactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttaaagaggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactagaactgtcaatgatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccccattggcacccgttaccttacccgtcccctgtaa

SEQ ID NO: 2 - protein, 736 aa, synthetic construct
MAADGYLPDWLEDNLSEGIREWWALQPGAPKPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRPL

SEQ ID NO: 3 - DNA, 2217 bp, Adeno-associated virus 8
  source 1..2217: mol_type = genomic DNA
atggctgccgatggttatcttccagattggctcgaggacaacctctctgagggcattcgcgagtggtgggcgctgaaacctggagccccgaagcccaaagccaaccagcaaaagcaggacgacggccggggtctagtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 4 - protein, 738 aa, Adeno-associated virus 8
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 5 - DNA, 2214 bp, synthetic construct
  source 1..2214: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcactctcctgtggagccagactcctcctcgggaaccggaaaggcgggccagcagcctgcaagaaaaagattgaattttggtcagactggagacgcagactcagtcccagaccctcaaccaatcggagaacctcccgcagccccctcaggtgtgggatctcttacaatggctgcaggcggtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaagcaaatctccaacagcacatctggaggatcttcaaatgacaacgcctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacgacaaatacgcagactctgggcttcagccaaggtgggcctaatacaatggccaatcaggcaaagaactggctgccaggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctccgcctcagatcctgatcaagaacacgcctgtacctgcggatcctccgaccaccttcaaccagtcaaagctgaactctttcatcacccagtattctactggccaagtcagcgtggagatcgagtgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 6 - protein, 737 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRLLEPLGLVEEAAKTAPGKKRPVEHSPVEPDSSSGTGKAGQQPARKRLNFGQTGDADSVPDPQPIGEPPAAPSGVGSLTMAAGGGAPMADNNEGADGVGNSSGNWHCDSTWMGDRVITTSTRTWALPTYNNHLYKQISNSTSGGSSNDNAYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTTNTQTLGFSQGGPNTMANQAKNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 7 - DNA, 2208 bp, synthetic construct
  source 1..2208: mol_type = other DNA
atggctgccgatggttatcttccagattggctcgaggacactctctctgaaggcattcgcgagtggtgggcgctgaaacctggagctccacaacccaaggccaaccaacagcatcaggacaacggcaggggtcttgtgcttcctgggtacaagtacctcggacccttcaacggactcgacaagggagagccggtcaacgaggcagacgccgcggccctcgagcacgacaaggcctacgacaagcagctcgagcagggggacaacccgtacctcaagtacaaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccgtcacctcagcgttcccccgactcctccacgggcatcggcaagaaaggccagcagcccgccagaaagagactcaatttcggtcagactggcgactcagagtcagtccccgaccctcaacctctcggagaacctccagcagcgccctctagtgtgggatctggtacagtggctgcaggcggtggcgcaccaatggcagacaataacgaaggtgccgacggagtgggtaatgcctcaggaaattggcattgcgattccacatggctgggcgacagagtcattaccaccagcacccgaacctgggccctgcccacctacaacaaccacctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccccagtacggctacctaacactcaacaacggtagtcaggccgtgggacgctcctccttttactgcctggaatatttcccatcgcagatgctgagaacgggcaataactttgagttcagctacagcttcgaggacgtgcctttccacagcagctacgcacacagccagagcttggaccgactgatgaatcctctcattgaccagtacctgtactacttatccagaactcagtccacaggaggaactcaaggtacccagcaattgttattttctcaagctgggcctgcaaacatgtcggctcaggccaagaactggctgcctggaccttgctaccggcagcagcgagtctccacgacactgtcgcaaaacaacaacagcaactttgcttggactggtgccaccaaatatcacctgaacggcagaaactcgttggttaatcccggcgtcgccatggcaactcacaaggacgacgaggaccgctttttcccatccagcggagtcctgatttttggaaaaactggagcaactaacaaaactacattggaaaatgtgttaatgacaaatgaagaagaaattcgtcctactaatcctgtagccacggaagaatacgggatagtcagcagcaacttacaagcggctaatactgcagcccagacacaagttgtcaacaaccagggagccttacctggcatggtctggcagaaccgggacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttaccttacccgtcccctgtaa

SEQ ID NO: 8 - protein, 735 aa, synthetic construct
MAADGYLPDWLEDTLSEGIREWWALKPGAPQPKANQQHQDNGRGLVLPGYKYLGPFNGLDKGEPVNEADAAALEHDKAYDKQLEQGDNPYLKYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSQSGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFEFSYSFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 9 - DNA, 2208 bp, synthetic construct
  source 1..2208: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggcaagacaggccagcagcccgctaaaaagagactcaattttggtcagactggcgactcagagtcagtcccagaccctcaacctctcggagaaccaccagcagccccctctggtctgggaactaatacgatggctacaggcagtggcgcaccaatggcagacaataacgagggtgccgatggagtgggtaattcctcaggaaattggcattgcgattcccaatggctgggcgacagagtcatcaccaccagcacccgaacctgggccctgcccacctacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttaaagaggtcacgcagaacgatggcacgacgactattgccaataaccttaccagcacggttcaagtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtgggctggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcgacacacaaggcgttcttccaggcatggtatggcaggacagagatgtgtaccttcagggacccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactcaacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 10 - protein, 735 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKTGQQPAKKRLNFGQTGDSESVPDPQPLGEPPAAPSGLGTNTMATGSGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWAGATKYHLNGRDSLVNPGPAMASHKDDEEKFFPQSGVLIFGKQGSEKTNVDIEKVMITDEEEIRTTNPVATEQYGSVSTNLQRGNRQAATADVDTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 11 - DNA, 2208 bp, Adeno-associated virus 2
  source 1..2208: mol_type = genomic DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 12 - protein, 735 aa, Adeno-associated virus 2 [sequence omitted]

SEQ ID NO: 13 - DNA, 2175 bp, Adeno-associated virus 5
  source 1..2175: mol_type = genomic DNA
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaa

SEQ ID NO: 14 - protein, 724 aa, Adeno-associated virus 5
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 15 - DNA, 2211 bp, Adeno-associated virus 6
  source 1..2211: mol_type = genomic DNA
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacgaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaa

SEQ ID NO: 16 - protein, 736 aa, Adeno-associated virus 6
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 17 - DNA, 2211 bp, Adeno-associated virus 9
  source 1..2211: mol_type = genomic DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 18 - protein, 736 aa, Adeno-associated virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 19 - DNA, 2217 bp, synthetic construct
  source 1..2217: mol_type = other DNA
atggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctccaagcgggtgacaatccgtacctgcggtataatcacgccgacgccgagtttcaggagcgtctgcaagaagatacgtcttttgggggcaacctcgggcgcgcagtcttccaggccaaaaagcgggttctcgaacctctgggcctggttgaatcgccggttaagacggctcctggaaagaagagaccggtagagccatcaccccagcgctctccagactcctctacgggcatcggcaagaaaggccagcagcccgcaaaaaagagactcaattttgggcagactggcgactcagagtcagtccccgaccctcaaccaatcggagaaccaccagcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgcacctgggccctgcccacctacaacaaccacctctacaagcaaatctccaacgggacctcgggaggaagcaccaacgacaacacctacttcggctacagcaccccctgggggtattttgacttcaacagattccactgccacttttcaccacgtgactggcagcgactcatcaacaacaactggggattccggcccaagaggctcaacttcaagctcttcaacatccaagtcaaggaggtcacgcagaatgaaggcaccaagaccatcgccaataaccttaccagcacgattcaggtctttacggactcggaataccagctcccgtacgtgctcggctcggcgcaccagggctgcctgcctccgttcccggcggacgtcttcatgattcctcagtacgggtacctgactctgaacaatggcagtcaggctgtgggccggtcgtccttctactgcctggagtactttccttctcaaatgctgagaacgggcaacaactttgaattcagctacaacttcgaggacgtgcccttccacagcagctacgcgcacagccagagcctggaccggctgatgaaccctctcatcgaccagtacttgtactacctgtcccggactcaaagcacgggcggtactgcaggaactcagcagttgctattttctcaggccgggcctaacaacatgtcggctcaggccaagaactggctacccggtccctgctaccggcagcaacgcgtctccacgacactgtcgcagaacaacaacagcaactttgcctggacgggtgccaccaagtatcatctgaatggcagagactctctggtgaatcctggcgttgccatggctacccacaaggacgacgaagagcgattttttccatccagcggagtcttaatgtttgggaaacagggagctggaaaagacaacgtggactatagcagcgtgatgctaaccagcgaggaagaaataaagaccaccaacccagtggccacagaacagtacggcgtggtggccgataacctgcaacagcaaaacgccgctcctattgtaggggccgtcaatagtcaaggagccttacctggcatggtgtggcagaaccgggacgtgtacctgcagggtcccatctgggccaagattcctcatacggacggcaactttcatccctcgccgctgatgggaggctttggactgaagcatccgcctcctcagatcctgattaaaaacacacctgttcccgcggatcctccgaccaccttcaatcaggccaagctggcttctttcatcacgcagtacagtaccggccaggtcagcgtggagatcgagtgggagctgcagaaggagaacagcaaacgctggaacccagagattcagtacacttccaactactacaaatctacaaatgtggactttgctgtcaatactgagggtacttattccgagcctcgccccattggcacccgttacctcacccgtaatctgtaa

SEQ ID NO: 20 - protein, 738 aa, synthetic construct
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 21 - DNA, 145 bp, unidentified
  source 1..145: mol_type = unassigned DNA
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 22 - DNA, 145 bp, unidentified
  source 1..145: mol_type = unassigned DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 23 - DNA, 123 bp, synthetic construct
  source 1..123: mol_type = other DNA
aggaacccctagtgatggagttggccactccctctctgcgcgctcgctcgctcactgaggccgcccgggctttgcccgggcggcctcagtgagcgagcgagcgcgcagagagggagtggccaa

SEQ ID NO: 24 - DNA, 123 bp, synthetic construct
  source 1..123: mol_type = other DNA
aggaacccctagtgatggagttggccactccctctctgcgcgctcgctcgctcactgaggccgggcgaccaaaggtcgcccggcctcagtgagcgagcgagcgcgcagagagggagtggccaa

SEQ ID NO: 25 - DNA, 130 bp, synthetic construct
  source 1..130: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 26 - DNA, 105 bp, synthetic construct
  source 1..105: mol_type = other DNA
ctgcgcgctcgctcgctcactgaggccgcccgggcaaagcccgggcgtcgggcgacctttggtcgcccggcctcagtgagcgagcgagcgcgcagagagggagtg

SEQ ID NO: 27 - DNA, 20 bp, synthetic construct
  source 1..20: mol_type = other DNA
gtggtcggtgctggtctata

SEQ ID NO: 28 - DNA, 20 bp, synthetic construct
  source 1..20: mol_type = other DNA
tttagaatagtggtcggtgc

SEQ ID NO: 29 - DNA, 20 bp, synthetic construct
  source 1..20: mol_type = other DNA
cgcatcatataagtcagaac

SEQ ID NO: 30 - DNA, 20 bp, synthetic construct
  source 1..20: mol_type = other DNA
tgagccatcacttacttgga

SEQ ID NO: 31 - DNA, 20 bp, synthetic construct
  source 1..20: mol_type = other DNA
ctgctgagccatcacttact

SEQ ID NO: 32 - DNA, 20 bp, synthetic construct
  source 1..20: mol_type = other DNA
ttaagcctgtcctagactta

SEQ ID NO: 33 - DNA, 20 bp, synthetic construct
  source 1..20: mol_type = other DNA
ttgcatgtttggttaggcta

SEQ ID NO: 34 - DNA, 20 bp, synthetic construct
  source 1..20: mol_type = other DNA
acagtatggcacaatagagc

SEQ ID NO: 35 - DNA, 20 bp, synthetic construct
  source 1..20: mol_type = other DNA
aagttactgtcaggccataa

SEQ ID NO: 36 - DNA, 20 bp, synthetic construct
  source 1..20: mol_type = other DNA
aatctctggacggaagctca

SEQ ID NO: 37 - DNA, 22 bp, synthetic construct
  source 1..22: mol_type = other DNA
atgtttggttaggctagggctt

SEQ ID NO: 38 - DNA, 22 bp, synthetic construct
  source 1..22: mol_type = other DNA
ggaggctttgtacatgtgggac

SEQ ID NO: 39 - DNA, 22 bp, synthetic construct
  source 1..22: mol_type = other DNA
ttacaaacaattgtcttacaaa

SEQ ID NO: 40 - DNA, 22 bp, synthetic construct
  source 1..22: mol_type = other DNA
cactgaaactttggtttcaatt

SEQ ID NO: 41 - DNA, 22 bp, synthetic construct
  source 1..22: mol_type = other DNA
ccactgctgagccatcacttac

SEQ ID NO: 42 - DNA, 22 bp, synthetic construct
  source 1..22: mol_type = other DNA
caagtaagtgatggctcagcag

SEQ ID NO: 43 - DNA, 21 bp, synthetic construct
  source 1..21: mol_type = other DNA
ggctcagcagtggaatactct

SEQ ID NO: 44 - DNA, 21 bp, synthetic construct
  source 1..21: mol_type = other DNA
ttcaactcatcctttccattg

SEQ ID NO: 45 - DNA, 21 bp, synthetic construct
  source 1..21: mol_type = other DNA
tgacttatatgatgcggtaca

SEQ ID NO: 46 - DNA, 66 bp, Porcine teschovirus
  source 1..66: mol_type = genomic DNA
ggaagcggcgccaccaatttcagcctgctgaaacaggccggcgacgtggaagagaaccctggccct

SEQ ID NO: 47 - protein, 22 aa, Porcine teschovirus
GSGATNFSLLKQAGDVEENPGP

SEQ ID NO: 48 - DNA, 66 bp, Porcine teschovirus
  source 1..66: mol_type = genomic DNA
ggcagcggcgccaccaacttcagcctgctgaaacaggccggcgacgtggaagagaaccctggccct

SEQ ID NO: 49 - DNA, 1254 bp, Homo sapiens
  source 1..1254: mol_type = genomic DNA
tccttcatcccggtggccgaggattccgacttccccatccacaacctgccctacggcgtcttctcgaccagaggcgacccaagaccgaggataggtgtggccattggcgaccagatcctggacctcagcatcatcaagcacctctttactggtcctgtcctctccaaacaccaggatgtcttcaatcagcctacactcaacagcttcatgggcctgggtcaggctgcctggaaggaggcgagagtgttcttgcagaacttgctgtctgtgagccaagccaggctcagagatgacaccgaacttcggaagtgtgcattcatctcccaggcttctgccacgatgcaccttccagccaccataggagactacacagacttctattcctctcggcagcatgctaccaacgtcggaatcatgttcagggacaaggagaatgcgttgatgccaaattggctgcacttaccagtgggctaccatggccgtgcctcctctgtcgtggtgtctggcaccccaatccgaaggcccatgggacagatgaaacctgatgactctaagcctcccgtatatggtgcctgcaagctcttggacatggagctggaaatggctttttttgtaggccctggaaacagattgggagagccgatccccatttccaaggcccatgagcacatttttggaatggtccttatgaacgactggagtgcacgagacattcagaagtgggagtatgtccctctcgggccattccttgggaagagttttgggaccactgtctctccgtgggtggtgcccatggatgctctcatgccctttgctgtgcccaacccgaagcaggaccccaggcccctgccgtatctgtgccatgacgagccctacacatttgacatcaacctctctgttaacctgaaaggagaaggaatgagccaggcggctaccatatgcaagtccaattttaagtacatgtactggacgatgctgcagcagctcactcaccactctgtcaacggctgcaacctgcggccgggggacctcctggcttctgggaccatcagcgggccggagccagaaaacttcggctccatgttggaactgtcgtggaagggaacgaagcccatagacctggggaatggtcagaccaggaagtttctgctggacggggatgaagtcatcataacagggtactgccagggggatggttaccgcatcggctttggccagtgtgctggaaaagtgctgcctgctctcctgccatca

SEQ ID NO: 50 - DNA, 1254 bp, synthetic construct
  source 1..1254: mol_type = other DNA
tccttcattcctgtggccgaggattctgactttcccattcacaacctgccctatggcgtcttttcaaccaggggagatcccaggcccagaatcggggtggccattggagatcagatcctggacctgtcaatcatcaagcacctgtttacaggccctgtgctgagcaagcaccaggatgtgttcaaccagccaactctgaacagcttcatggggctggggcaggctgcctggaaggaggcaagggtgtttctgcagaatctgctgagcgtgtcacaggctaggctgagggatgataccgagctgaggaagtgtgcatttatctcacaggcatcagccacaatgcatctgccagctacaatcggcgactataccgacttttactcaagcaggcagcatgccaccaatgtgggcatcatgttcagggacaaagagaatgccctgatgccaaattggctgcacctgcctgtgggctatcatggcagagccagctcagtggtggtgtcagggacaccaattaggaggcctatgggccagatgaagcctgatgacagcaaaccacctgtctacggcgcctgcaagctcctggatatggaactggagatggctttttttgtgggcccaggcaatagactgggagagcccattccaatctcaaaggcccatgaacatatctttggcatggtcctgatgaatgactggtcagccagagacattcagaagtgggagtacgtgccactgggaccatttctgggaaagagctttggcaccacagtgtcaccatgggtggtgcccatggatgccctgatgccctttgctgtgccaaatccaaaacaagaccccaggcccctcccctatctctgtcatgatgaaccatatacttttgacattaacctgagcgtgaacctgaaaggagaaggcatgagccaagccgccactatctgtaagagcaacttcaaatatatgtactggacaatgctgcagcagctcacccaccatagcgtgaatgggtgtaacctgaggccaggcgacctgctggcatcaggcactatttcagggcctgagcctgaaaattttggatcaatgctggagctgtcatggaagggaactaaacccatcgacctggggaatggccagaccagaaagtttctcctggatggcgatgaggtgatcattacaggctactgtcagggggatggctatagaattggatttggccaatgcgccggcaaagtcctccctgccctgctgcccagc

SEQ ID NO: 51 - DNA, 1254 bp, synthetic construct
  source 1..1254: mol_type = other DNA
tccttcatcccagtggctgaagattctgacttccccattcacaacctcccttatggagtctttagcacaagaggagacccaagacctagaattggcgtggctattggggatcagatcctggacctgagcattatcaaacatctctttacaggcccagtcctcagcaagcaccaagatgtgttcaatcaacccacactgaactcatttatggggctgggccaagctgcctggaaggaggcaagagtgtttctccagaacctcctgtcagtgagccaggccaggctgagagatgacacagagctgaggaagtgtgcctttatctcacaagccagcgccacaatgcacctgccagcaaccatcggggactacacagacttttacagcagcaggcagcacgccactaatgtgggcatcatgtttagagacaaagagaatgccctcatgcctaactggctgcatctgccagtgggctaccatggcagagccagcagcgtggtggtgtctggcacccctatcaggaggcccatgggccagatgaagcctgatgacagcaagccacctgtgtatggggcatgtaagctgctggacatggagctggaaatggctttctttgtggggcctgggaacaggctgggcgaaccaattcccatcagcaaagctcatgaacacatttttgggatggtgctcatgaatgactggtctgccagagacatccagaagtgggagtatgtccccctgggaccattcctgggcaagagctttgggaccactgtgtcaccatgggtggtgcccatggatgccctgatgccatttgctgtgcctaatcccaaacaagatccaaggccactgccctacctctgccacgatgagccctacacatttgatatcaacctctctgtgaatctgaagggagaaggaatgagccaagctgctaccatttgcaaaagcaactttaagtatatgtattggaccatgctgcagcaactcacccaccacagcgtgaatggctgtaacctgaggcctggcgacctgctggcctctggcaccatcagcggccctgaacctgagaactttggcagcatgctggagctgagctggaagggaaccaagcccattgacctgggcaatggacagaccaggaaattcctcctggacggggacgaggtgatcatcacaggctattgccagggggatgggtataggattggctttggccaatgtgccggcaaagtgctgcctgccctgctccctagc

SEQ ID NO: 52 - DNA, 1254 bp, synthetic construct
  source 1..1254: mol_type = other DNA
tcctttattcctgtggccgaagatagcgacttccctatccataacctcccatatggcgtcttctcaaccaggggcgaccccaggcccaggattggggtggcaattggagaccagatcctggacctcagcatcattaagcacctctttacaggacctgtgctgagcaagcaccaggatgtgttcaaccagcctaccctgaatagctttatgggactgggccaggcagcatggaaagaagccagagtgttcctccagaatctgctgagcgtgagccaggccaggctcagggatgacacagaactgaggaaatgtgccttcatctcacaggcatcagccacaatgcacctccctgccacaattggggactacactgacttctacagcagcagacagcatgccactaatgtggggattatgtttagagacaaggaaaatgccctcatgccaaattggctgcacctgcctgtgggctaccacggcagagcctcatcagtggtggtgtcaggcacaccaattaggaggccaatgggacagatgaagcctgacgactcaaagccaccagtctatggcgcctgcaaactgctggacatggagctggagatggctttctttgtggggcctggcaacaggctgggagaaccaatccctatttcaaaggcccacgagcacatttttggcatggtgctcatgaatgactggtctgccagagatatccagaagtgggaatacgtgcccctgggcccttttctgggcaagagctttggcaccacagtgtcaccttgggtggtcccaatggatgccctgatgccctttgccgtgcccaaccccaagcaggatcccagaccactgccctacctgtgccacgatgagccctatacctttgacatcaacctgtcagtcaacctgaagggggagggcatgagccaggccgccactatttgcaagagcaactttaaatatatgtactggactatgctccaacagctcactcaccactcagtgaatggctgcaatctgaggcctggcgacctcctggctagcggcactatctctggccctgaacctgagaactttgggagcatgctggagctgtcatggaaagggacaaagcctattgacctggggaatggccagacaaggaagtttctcctggatggcgatgaggtcatcatcactgggtactgccagggcgatggctacaggattggatttggacagtgtgctggcaaagtgctcccagctctgctgccctca

SEQ ID NO: 53 - DNA, 3102 bp, synthetic construct
  source 1..3102: mol_type = other DNA
cctgagcaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggcccttcttctcagccgcagaagtgcttcttacagatcaaaggcatgacctgtgcatcctgtgtgtctaacatagaaaggaatctgcagaaagaagctggtgttctctccgtgttggttgccttgatggcaggaaaggcagagatcaagtatgacccagaggtcatccagcccctcgagatagctcagttcatccaggacctgggttttgaggcagcagtcatggaggactacgcaggctccgatggcaacattgagctgacaatcacagggatgacctgcgcgtcctgtgtccacaacatagagtccaaactcacgaggacaaatggcatcacttatgcctccgttgcccttgccaccagcaaagcccttgttaagtttgacccggaaattatcggtccacgggatattatcaaaattattgaggaaattggctttcatgcttccctggcccagagaaaccccaacgctcatcacttggaccacaagatggaaataaagcagtggaagaagtctttcctgtgcagcctggtgtttggcatccctgtcatggccttaatgatctatatgctgatacccagcaacgagccccaccagtccatggtcctggaccacaacatcattccaggactgtccattctaaatctcatcttctttatcttgtgtacctttgtccagctcctcggtgggtggtacttctacgttcaggcctacaaatctctgagacacaggtcagccaacatggacgtgctcatcgtcctggccacaagcattgcttatgtttattctctggtcatcctggtggttgctgtggctgagaaggcggagaggagccctgtgacattcttcgacacgccccccatgctctttgtgttcattgccctgggccggtggctggaacacttggcaaagagcaaaacctcagaagccctggctaaactcatgtctctccaagccacagaagccaccgttgtgacccttggtgaggacaatttaatcatcagggaggagcaagtccccatggagctggtgcagcggggcgatatcgtcaaggtggtccctgggggaaagtttccagtggatgggaaagtcctggaaggcaataccatggctgatgagtccctcatcacaggagaagccatgccagtcactaagaaacccggaagcactgtaattgcggggtctataaatgcacatggctctgtgctcattaaagctacccacgtgggcaatgacaccactttggctcagattgtgaaactggtggaagaggctcagatgtcaaaggcacccattcagcagctggctgaccggtttagtggatattttgtcccatttatcatcatcatgtcaactttgacgttggtggtatggattgtaatcggttttatcgattttggtgttgttcagagatactttcctaaccccaacaagcacatctcccagacagaggtgatcatccggtttgctttccagacgtccatcacggtgctgtgcattgcctgcccctgctccctggggctggccacgcccacggctgtcatggtgggcaccggggtggccgcgcagaacggcatcctcatcaagggaggcaagcccctggagatggcgcacaagataaagactgtgatgtttgacaagactggcaccattacccatggcgtccccagggtcatgcgggtgctcctgctgggggatgtggccacactgcccctcaggaaggttctggctgtggtggggactgcggaggccagcagtgaacaccccttgggcgtggcagtcaccaaatactgtaaagaggaacttggaacagagaccttgggatactgcacggacttccaggcagtgccaggctgtggaattgggtgcaaagtcagcaacgtggaaggcatcctggcccacagtgagcgccctttgagtgcaccggccagtcacctgaatgaggctggcagccttcccgcagaaaaagatgcagtcccccagaccttctctgtgctgattggaaaccgtgagtggctgaggcgcaacggtttaaccatttctagcgatgtcagtgacgctatgacagaccacgagatgaaaggacagacagccatcctggtggctattgacggtgtgctctgtgggatgatcgcaatcgcagacgctgtcaagcaggaggctgccctggctgtgcacacgctgcagagcatgggtgtggacgtggttctgatcacgggggacaaccggaagacagccagagctattgccacccaggttggcatcaacaaagtctttgcagaggtgctgccttcgcacaaggtggccaaggtccaggagctccagaataaagggaagaaagtcgccatggtgggggatggggtcaatgactccccggccttggcccaggcagacatgggtgtggccattggcaccggcacggatgtggccatcgaggcagccgacgtcgtccttatcagaaatgatttgctggatgtggtggctagcattcacctttccaagaggactgtccgaaggatacgcatcaacctggtcctggcactgatttataacctggttgggatacccattgcagcaggtgtcttcatgcccatcggcattgtgctgcagccctggatgggctcagcggccatggcagcctcctctgtgtctgtggtgctctcatccctgcagctcaagtgctataagaagcctgacctggagaggtatgaggcacaggcgcatggccacatgaagcccctgacggcatcccaggtcagtgtgcacataggcatggatgacaggtggcgggactcccccagggccacaccatgggaccaggtcagctatgtcagccaggtgtcgctgtcctccctgacgtccgacaagccatctcggcacagcgctgcagcagacgatgatggggacaagtggtctctgctcctgaatggcagggatgaggagcagtacatc

SEQ ID NO: 54 - DNA, 2577 bp, Homo sapiens
  source 1..2577: mol_type = genomic DNA
gggccctggggctggaaattgcgctggaccgtcgccttgctcctcgccgcggcggggactgcagtgggcgacagatgcgaaagaaacgagttccagtgccaagacgggaaatgcatctcctacaagtgggtctgcgatggcagcgctgagtgccaggatggctctgatgagtcccaggagacgtgcttgtctgtcacctgcaaatccggggacttcagctgtgggggccgtgtcaaccgctgcattcctcagttctggaggtgcgatggccaagtggactgcgacaacggctcagacgagcaaggctgtccccccaagacgtgctcccaggacgagtttcgctgccacgatgggaagtgcatctctcggcagttcgtctgtgactcagaccgggactgcttggacggctcagacgaggcctcctgcccggtgctcacctgtggtcccgccagcttccagtgcaacagctccacctgcatcccccagctgtgggcctgcgacaacgaccccgactgcgaagatggctcggatgagtggccgcagcgctgtaggggtctttacgtgttccaaggggacagtagcccctgctcggccttcgagttccactgcctaagtggcgagtgcatccactccagctggcgctgtgatggtggccccgactgcaaggacaaatctgacgaggaaaactgcgctgtggccacctgtcgccctgacgaattccagtgctctgatggaaactgcatccatggcagccggcagtgtgaccgggaatatgactgcaaggacatgagcgatgaagttggctgcgttaatgtgacactctgcgagggacccaacaagttcaagtgtcacagcggcgaatgcatcaccctggacaaagtctgcaacatggctagagactgccgggactggtcagatgaacccatcaaagagtgcgggaccaacgaatgcttggacaacaacggcggctgttcccacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctccatcgcctacctcttcttcaccaaccggcacgaggtcaggaagatgacgctggaccggagcgagtacaccagcctcatccccaacctgaggaacgtggtcgctctggacacggaggtggccagcaatagaatctactggtctgacctgtcccagagaatgatctgcagcacccagcttgacagagcccacggcgtctcttcctatgacaccgtcatcagcagagacatccaggcccccgacgggctggctgtggactggatccacagcaacatctactggaccgactctgtcctgggcactgtctctgttgcggataccaagggcgtgaagaggaaaacgttattcagggagaacggctccaagccaagggccatcgtggtggatcctgttcatggcttcatgtactggactgactggggaactcccgccaagatcaagaaagggggcctgaatggtgtggacatctactcgctggtgactgaaaacattcagtggcccaatggcatcaccctagatctcctcagtggccgcctctactgggttgactccaaacttcactccatctcaagcatcgatgtcaacgggggcaaccggaagaccatcttggaggatgaaaagaggctggcccaccccttctccttggccgtctttgaggacaaagtattttggacagatatcatcaacgaagccattttcagtgccaaccgcctcacaggttccgatgtcaacttgttggctgaaaacctactgtccccagaggatatggttctcttccacaacctcacccagccaagaggagtgaactggtgtgagaggaccaccctgagcaatggcggctgccagtatctgtgcctccctgccccgcagatcaacccccactcgcccaagtttacctgcgcctgcccggacggcatgctgctggccagggacatgaggagctgcctcacagaggctgaggctgcagtggccacccaggagacatccaccgtcaggctaaaggtcagctccacagccgtaaggacacagcacacaaccacccgacctgttcccgacacctcccggctgcctggggccacccctgggctcaccacggtggagatagtgacaatgtctcaccaagctctgggcgacgttgctggcagaggaaatgagaagaagcccagtagcgtgagggctctgtccattgtcctccccatcgtgctcctcgtcttcctttgcctgggggtcttccttctatggaagaactggcggcttaagaacatcaacagcatcaactttgacaaccccgtctatcagaagaccacagaggatgaggtccacatttgccacaaccaggacggctacagctacccctcgagacagatggtcagtctggaggatgacgtggcg

SEQ ID NO: 55 - DNA, 1353 bp, Homo sapiens
  source 1..1353: mol_type = genomic DNA
tccactgcggtcctggaaaacccaggcttgggcaggaaactctctgactttggacaggaaacaagctatattgaagacaactgcaatcaaaatggtgccatatcactgatcttctcactcaaagaagaagttggtgcattggccaaagtattgcgcttatttgaggagaatgatgtaaacctgacccacattgaatctagaccttctcgtttaaagaaagatgagtatgaatttttcacccatttggataaacgtagcctgcctgctctgacaaacatcatcaagatcttgaggcatgacattggtgccactgtccatgagctttcacgagataagaagaaagacacagtgccctggttcccaagaaccattcaagagctggacagatttgccaatcagattctcagctatggagcggaactggatgctgaccaccctggttttaaagatcctgtgtaccgtgcaagacggaagcagtttgctgacattgcctacaactaccgccatgggcagcccatccctcgagtggaatacatggaggaagaaaagaaaacatggggcacagtgttcaagactctgaagtccttgtataaaacccatgcttgctatgagtacaatcacatttttccacttcttgaaaagtactgtggcttccatgaagataacattccccagctggaagacgtttctcagttcctgcagacttgcactggtttccgcctccgacctgtggctggcctgctttcctctcgggatttcttgggtggcctggccttccgagtcttccactgcacacagtacatcagacatggatccaagcccatgtatacccccgaacctgacatctgccatgagctgttgggacatgtgcccttgttttcagatcgcagctttgcccagttttcccaggaaattggccttgcctctctgggtgcacctgatgaatacattgaaaagctcgccacaatttactggtttactgtggagtttgggctctgcaaacaaggagactccataaaggcatatggtgctgggctcctgtcatcctttggtgaattacagtactgcttatcagagaagccaaagcttctccccctggagctggagaagacagccatccaaaattacactgtcacggagttccagcccctctattacgtggcagagagttttaatgatgccaaggagaaagtaaggaactttgctgccacaatacctcggcccttctcagttcgctacgacccatacacccaaaggattgaggtcttggacaatacccagcagcttaagattttggctgattccattaacagtgaaattggaatcctttgcagtgccctccagaaaataaag

SEQ ID NO: 56 - DNA, 1389 bp, Homo sapiens
  source 1..1389: mol_type = genomic DNA
gcctcggagagtgggaagctttggggtggccggtttgtgggtgcagtggaccccatcatggagaagttcaacgcgtccattgcctacgaccggcacctttgggaggtggatgttcaaggcagcaaagcctacagcaggggcctggagaaggcagggctcctcaccaaggccgagatggaccagatactccatggcctagacaaggtggctgaggagtgggcccagggcaccttcaaactgaactccaatgatgaggacatccacacagccaatgagcgccgcctgaaggagctcattggtgcaacggcagggaagctgcacacgggacggagccggaatgaccaggtggtcacagacctcaggctgtggatgcggcagacctgctccacgctctcgggcctcctctgggagctcattaggaccatggtggatcgggcagaggcggaacgtgatgttctcttcccggggtacacccatttgcagagggcccagcccatccgctggagccactggattctgagccacgccgtggcactgacccgagactctgagcggctgctggaggtgcggaagcggatcaatgtcctgcccctggggagtggggccattgcaggcaatcccctgggtgtggaccgagagctgctccgagcagaactcaactttggggccatcactctcaacagcatggatgccactagtgagcgggactttgtggccgagttcctgttctgggcttcgctgtgcatgacccatctcagcaggatggccgaggacctcatcctctactgcaccaaggaattcagcttcgtgcagctctcagatgcctacagcacgggaagcagcctgatgccccagaagaaaaaccccgacagtttggagctgatccggagcaaggctgggcgtgtgtttgggcggtgtgccgggctcctgatgaccctcaagggacttcccagcacctacaacaaagacttacaggaggacaaggaagctgtgtttgaagtgtcagacactatgagtgccgtgctccaggtggccactggcgtcatctctacgctgcagattcaccaagagaacatgggacaggctctcagccccgacatgctggccactgaccttgcctattacctggtccgcaaagggatgccattccgccaggcccacgaggcctccgggaaagctgtgttcatggccgagaccaagggggtcgccctcaaccagctgtcactgcaggagctgcagaccatcagccccctgttctcgggcgacgtgatctgcgtgtgggactacgggcacagtgtggagcagtatggtgccctgggcggcactgcgcgctccagcgtcgactggcagatccgccaggtgcgggcgctactgcaggcacagcaggcc

SEQ ID NO: 57 - DNA, 1068 bp, Homo sapiens
  source 1..1068: mol_type = genomic DNA
gaggaaggaatgaatgttctccatgactttgggatccagtcaacacattacctccaggtgaattaccaagactcccaggactggttcatcttggtgtccgtgatcgcagacctcaggaatgccttctacgtcctcttccccatctggttccatcttcaggaagctgtgggcattaaactcctttgggtagctgtgattggagactggctcaacctcgtctttaagtggattctctttggacagcgtccatactggtgggttttggatactgactactacagcaacacttccgtgcccctgataaagcagttccctgtaacctgtgagactggaccagggagcccctctggccatgccatgggcacagcaggtgtatactacgtgatggtcacatctactctttccatctttcagggaaagataaagccgacctacagatttcggtgcttgaatgtcattttgtggttgggattctgggctgtgcagctgaatgtctgtctgtcacgaatctaccttgctgctcattttcctcatcaagttgttgctggagtcctgtcaggcattgctgttgcagaaactttcagccacatccacagcatctataatgccagcctcaagaaatattttctcattaccttcttcctgttcagcttcgccatcggattttatctgctgctcaagggactgggtgtagacctcctgtggactctggagaaagcccagaggtggtgcgagcagccagaatgggtccacattgacaccacaccctttgccagcctcctcaagaacctgggcacgctctttggcctggggctggctctcaactccagcatgtacagggagagctgcaaggggaaactcagcaagtggctcccattccgcctcagctctattgtagcctccctcgtcctcctgcacgtctttgactccttgaaacccccatcccaagtcgagctggtcttctacgtcttgtccttctgcaagagtgcggtagtgcccctggcatccgtcagtgtcatcccctactgcctcgcccaggtcctgggccagccgcacaagaagtcgttg

SEQ ID NO: 58 - DNA, 3960 bp, Homo sapiens
  source 1..3960: mol_type = genomic DNA
tctgactcagtaattcttcgaagtataaagaaatttggagaggagaatgatggttttgagtcagataaatcatataataatgataagaaatcaaggttacaagatgagaagaaaggtgatggcgttagagttggcttctttcaattgtttcggttttcttcatcaactgacatttggctgatgtttgtgggaagtttgtgtgcatttctccatggaatagcccagccaggcgtgctactcatttttggcacaatgacagatgtttttattgactacgacgttgagttacaagaactccagattccaggaaaagcatgtgtgaataacaccattgtatggactaacagttccctcaaccagaacatgacaaatggaacacgttgtgggttgctgaacatcgagagcgaaatgatcaaatttgccagttactatgctggaattgctgtcgcagtacttatcacaggatatattcaaatatgcttttgggtcattgccgcagctcgtcagatacagaaaatgagaaaattttactttaggagaataatgagaatggaaatagggtggtttgactgcaattcagtgggggagctgaatacaagattctctgatgatattaataaaatcaatgatgccatagctgaccaaatggcccttttcattcagcgcatgacctcgaccatctgtggtttcctgttgggatttttcaggggttggaaactgaccttggttattatttctgtcagccctctcattgggattggagcagccaccattggtctgagtgtgtccaagtttacggactatgagctgaaggcctatgccaaagcaggggtggtggctgatgaagtcatttcatcaatgagaacagtggctgcttttggtggtgagaaaagagaggttgaaaggtatgagaaaaatcttgtgttcgcccagcgttggggaattagaaaaggaatagtgatgggattctttactggattcgtgtggtgtctcatctttttgtgttatgcactggccttctggtacggctccacacttgtcctggatgaaggagaatatacaccaggaacccttgtccagattttcctcagtgtcatagtaggagctttaaatcttggcaatgcctctccttgtttggaagcctttgcaactggacgtgcagcagccaccagcatttttgagacaatagacaggaaacccatcattgactgcatgtcagaagatggttacaagttggatcgaatcaagggtgaaattgaattccataatgtgaccttccattatccttccagaccagaggtgaagattctaaatgacctcaacatggtcattaaaccaggggaaatgacagctctggtaggacccagtggagctggaaaaagtacagcactgcaactcattcagcgattctatgacccctgtgaaggaatggtgaccgtggatggccatgacattcgctctcttaacattcagtggcttagagatcagattgggatagtggagcaagagccagttctgttctctaccaccattgcagaaaatattcgctatggcagagaagatgcaacaatggaagacatagtccaagctgccaaggaggccaatgcctacaacttcatcatggacctgccacagcaatttgacacccttgttggagaaggaggaggccagatgagtggtggccagaaacaaagggtagctatcgccagagccctcatccgaaatcccaagattctgcttttggacatggccacctcagctctggacaatgagagtgaagccatggtgcaagaagtgctgagtaagattcagcatgggcacacaatcatttcagttgctcatcgcttgtctacggtcagagctgcagataccatcattggttttgaacatggcactgcagtggaaagagggacccatgaagaattactggaaaggaaaggtgtttacttcactctagtgactttgcaaagccagggaaatcaagctcttaatgaagaggacataaaggatgcaactgaagatgacatgcttgcgaggacctttagcagagggagctaccaggatagtttaagggcttccatccggcaacgctccaagtctcagctttcttacctggtgcacgaacctccattagctgttgtagatcataagtctacctatgaagaagatagaaaggacaaggacattcctgtgcaggaagaagttgaacctgccccagttaggaggattctgaaattcagtgctccagaatggccctacatgctggtagggtctgtgggtgcagctgtgaacgggacagtcacacccttgtatgcctttttattcagccagattcttgggactttttcaattcctgataaagaggaacaaaggtcacagatcaatggtgtgtgcctactttttgtagcaatgggctgtgtatctcttttcacccaatttctacagggatatgcctttgctaaatctggggagctcctaacaaaaaggctacgtaaatttggtttcagggcaatgctggggcaagatattgcctggtttgatgacctcagaaatagccctggagcattgacaacaagacttgctacagatgcttcccaagttcaaggggctgccggctctcagatcgggatgatagtcaattccttcactaacgtcactgtggccatgatcattgccttctcctttagctggaagctgagcctggtcatcttgtgcttcttccccttcttggctttatcaggagccacacagaccaggatgttgacaggatttgcctctcgagataagcaggccctggagatggtgggacagattacaaatgaagccctcagtaacatccgcactgttgctggaattggaaaggagaggcggttcattgaagcacttgagactgagctggagaagcccttcaagacagccattcagaaagccaatatttacggattctgctttgcctttgcccagtgcatcatgtttattgcgaattctgcttcctacagatatggaggttacttaatctccaatgaggggctccatttcagctatgtgttcagggtgatctctgcagttgtactgagtgcaacagctcttggaagagccttctcttacaccccaagttatgcaaaagctaaaatatcagctgcacgcttttttcaactgctggaccgacaacccccaatcagtgtatacaatactgcaggtgaaaaatgggacaacttccaggggaagattgattttgttgattgtaaatttacatatccttctcgacctgactcgcaagttctgaatggtctctcagtgtcgattagtccagggcagacactggcgtttgttgggagcagtggatgtggcaaaagcactagcattcagctgttggaacgtttctatgatcctgatcaagggaaggtgatgatagatggtcatgacagcaaaaaagtaaatgtccagttcctccgctcaaacattggaattgtttcccaggaaccagtgttgtttgcctgtagcataatggacaatatcaagtatggagacaacaccaaagaaattcccatggaaagagtcatagcagctgcaaaacaggctcagctgcatgattttgtcatgtcactcccagagaaatatgaaactaacgttgggtcccaggggtctcaactctctagaggggagaaacaacgcattgctattgctcgggccattgtacgagatcctaaaatcttgctactagatgaagccacttctgccttagacacagaaagtgaaaagacggtgcaggttgctctagacaaagccagagagggtcggacctgcattgtcattgcccatcgcttgtccaccatccagaacgcggatatcattgctgtcatggcacagggggtggtgattgaaaaggggacccatgaagaactgatggcccaaaaaggagcctactacaaactagtcaccactggatcccccatcagt

SEQ ID NO: 59 - DNA, 3834 bp, Homo sapiens
  source 1..3834: mol_type = genomic DNA
gatcttgaggcggcaaagaacggaacagcctggcgccccacgagcgcggagggcgactttgaactgggcatcagcagcaaacaaaaaaggaaaaaaacgaagacagtgaaaatgattggagtattaacattgtttcgatactccgattggcaggataaattgtttatgtcgctgggtaccatcatggccatagctcacggatcaggtctccccctcatgatgatagtatttggagagatgactgacaaatttgttgatactgcaggaaacttctcctttccagtgaacttttccttgtcgctgctaaatccaggcaaaattctggaagaagaaatgactagatatgcatattactactcaggattgggtgctggagttcttgttgctgcctatatacaagtttcattttggactttggcagctggtcgacagatcaggaaaattaggcagaagttttttcatgctattctacgacaggaaataggatggtttgacatcaacgacaccactgaactcaatacgcggctaacagatgacatctccaaaatcagtgaaggaattggtgacaaggttggaatgttctttcaagcagtagccacgttttttgcaggattcatagtgggattcatcagaggatggaagctcacccttgtgataatggccatcagccctattctaggactctctgcagccgtttgggcaaagatactctcggcatttagtgacaaagaactagctgcttatgcaaaagcaggcgccgtggcagaagaggctctgggggccatcaggactgtgatagctttcgggggccagaacaaagagctggaaaggtatcagaaacatttagaaaatgccaaagagattggaattaaaaaagctatttcagcaaacatttccatgggtattgccttcctgttaatatatgcatcatatgcactggccttctggtatggatccactctagtcatatcaaaagaatatactattggaaatgcaatgacagtttttttttcaatcctaattggagctttcagtgttggccaggctgccccatgtattgatgcttttgccaatgcaagaggagcagcatatgtgatctttgatattattgataataatcctaaaattgacagtttttcagagagaggacacaaaccagacagcatcaaagggaatttggagttcaatgatgttcacttttcttacccttctcgagctaacgtcaagatcttgaagggcctcaacctgaaggtgcagagtgggcagacggtggccctggttggaagtagtggctgtgggaagagcacaacggtccagctgatacagaggctctatgaccctgatgagggcacaattaacattgatgggcaggatattaggaactttaatgtaaactatctgagggaaatcattggtgtggtgagtcaggagccggtgctgttttccaccacaattgctgaaaatatttgttatggccgtggaaatgtaaccatggatgagataaagaaagctgtcaaagaggccaacgcctatgagtttatcatgaaattaccacagaaatttgacaccctggttggagagagaggggcccagctgagtggtgggcagaagcagaggatcgccattgcacgtgccctggttcgcaaccccaagatccttctgctggatgaggccacgtcagcattggacacagaaagtgaagctgaggtacaggcagctctggataaggccagagaaggccggaccaccattgtgatagcacaccgactgtctacggtccgaaatgcagatgtcatcgctgggtttgaggatggagtaattgtggagcaaggaagccacagcgaactgatgaagaaggaaggggtgtacttcaaacttgtcaacatgcagacatcaggaagccagatccagtcagaagaatttgaactaaatgatgaaaaggctgccactagaatggccccaaatggctggaaatctcgcctatttaggcattctactcagaaaaaccttaaaaattcacaaatgtgtcagaagagccttgatgtggaaaccgatggacttgaagcaaatgtgccaccagtgtcctttctgaaggtcctgaaactgaataaaacagaatggccctactttgtcgtgggaacagtatgtgccattgccaatggggggcttcagccggcattttcagtcatattctcagagatcatagcgatttttggaccaggcgatgatgcagtgaagcagcagaagtgcaacatattctctttgattttcttatttctgggaattatttctttttttactttcttccttcagggtttcacgtttgggaaagctggcgagatcctcaccagaagactgcggtcaatggcttttaaagcaatgctaagacaggacatgagctggtttgatgaccataaaaacagtactggtgcactttctacaagacttgccacagatgctgcccaagtccaaggagccacaggaaccaggttggctttaattgcacagaatatagctaaccttggaactggtattatcatatcatttatctacggttggcagttaaccctattgctattagcagttgttccaattattgctgtgtcaggaattgttgaaatgaaattgttggctggaaatgccaaaagagataaaaaagaactggaagctgctggaaagattgcaacagaggcaatagaaaatattaggacagttgtgtctttgacccaggaaagaaaatttgaatcaatgtatgttgaaaaattgtatggaccttacaggaattctgtgcagaaggcacacatctatggaattacttttagtatctcacaagcatttatgtatttttcctatgccggttgttttcgatttggtgcatatctcattgtgaatggacatatgcgcttcagagatgttattctggtgttttctgcaattgtatttggtgcagtggctctaggacatgccagttcatttgctccagactatgctaaagctaagctgtctgcagcccacttattcatgctgtttgaaagacaacctctgattgacagctacagtgaagaggggctgaagcctgataaatttgaaggaaatataacatttaatgaagtcgtgttcaactatcccacccgagcaaacgtgccagtgcttcaggggctgagcctggaggtgaagaaaggccagacactagccctggtgggcagcagtggctgtgggaagagcacggtggtccagctcctggagcggttctacgaccccttggcggggacagtgcttctcgatggtcaagaagcaaagaaactcaatgtccagtggctcagagctcaactcggaatcgtgtctcaggagcctatcctatttgactgcagcattgccgagaatattgcctatggagacaacagccgggttgtatcacaggatgaaattgtgagtgcagccaaagctgccaacatacatcctttcatcgagacgttaccccacaaatatgaaacaagagtgggagataaggggactcagctctcaggaggtcaaaaacagaggattgctattgcccgagccctcatcagacaacctcaaatcctcctgttggatgaagctacatcagctctggatactgaaagtgaaaaggttgtccaagaagccctggacaaagccagagaaggccgcacctgcattgtgattgctcaccgcctgtccaccatccagaatgcagacttaatagtggtgtttcagaatgggagagtcaaggagcatggcacgcatcagcagctgctggcacagaaaggcatctatttttcaatggtcagtgtccaggctgggacacagaactta

SEQ ID NO: 60 - DNA, 1650 bp, Homo sapiens
  source 1..1650: mol_type = genomic DNA
ccttctgagaccccccaggcagaagtggggcccacaggctgcccccaccgctcagggccacactcggcgaaggggagcctggagaaggggtccccagaggataaggaagccaaggagcccctgtggatccggcccgatgctccgagcaggtgcacctggcagctgggccggcctgcctccgagtccccacatcaccacactgccccggcaaaatctccaaaaatcttgccagatattctgaagaaaatcggggacacccctatggtcagaatcaacaagattgggaagaagttcggcctgaagtgtgagctcttggccaagtgtgagttcttcaacgcgggcgggagcgtgaaggaccgcatcagcctgcggatgattgaggatgctgagcgcgacgggacgctgaagcccggggacacgattatcgagccgacatccgggaacaccgggatcgggctggccctggctgcggcagtgaggggctatcgctgcatcatcgtgatgccagagaagatgagctccgagaaggtggacgtgctgcgggcactgggggctgagattgtgaggacgcccaccaatgccaggttcgactccccggagtcacacgtgggggtggcctggcggctgaagaacgaaatccccaattctcacatcctagaccagtaccgcaacgccagcaaccccctggctcactacgacaccaccgctgatgagatcctgcagcagtgtgatgggaagctggacatgctggtggcttcagtgggcacgggcggcaccatcacgggcattgccaggaagctgaaggagaagtgtcctggatgcaggatcattggggtggatcccgaagggtccatcctcgcagagccggaggagctgaaccagacggagcagacaacctacgaggtggaagggatcggctacgacttcatccccacggtgctggacaggacggtggtggacaagtggttcaagagcaacgatgaggaggcgttcacctttgcccgcatgctgatcgcgcaagaggggctgctgtgcggtggcagtgctggcagcacggtggcggtggccgtgaaggccgcgcaggagctgcaggagggccagcgctgcgtggtcattctgcccgactcagtgcggaactacatgaccaagttcctgagcgacaggtggatgctgcagaagggctttctgaaggaggaggacctcacggagaagaagccctggtggtggcacctccgtgttcaggagctgggcctgtcagccccgctgaccgtgctcccgaccatcacctgtgggcacaccatcgagatcctccgggagaagggcttcgaccaggcgcccgtggtggatgaggcgggggtaatcctgggaatggtgacgcttgggaacatgctctcgtccctgcttgccgggaaggtgcagccgtcagaccaagttggcaaagtcatctacaagcagttcaaacagatccgcctcacggacacgctgggcaggctctcgcacatcctggagatggaccacttcgccctggtggtgcacgagcagatccagtaccacagcaccgggaagtccagtcagcggcagatggtgttcggggtggtcaccgccattgacttgctgaacttcgtggccgcccaggagcgggaccagaag

SEQ ID NO: 61 - DNA, 2247 bp, Homo sapiens
  source 1..2247: mol_type = genomic DNA
ctgagagccaaaaaccagctgttcctgctgagcccccactatctgagacaggtcaaagaaagttccgggagtagactgatccagcagagactgctgcaccagcagcagccactgcatcctgagtgggccgctctggccaagaaacagctgaagggcaaaaacccagaagacctgatctggcacactccagaggggatttcaatcaagcccctgtacagcaaaagggacactatggatctgccagaggaactgccaggagtgaagcctttcacccgcggaccttacccaactatgtatacctttcgaccctggacaattcggcagtacgccggcttcagtactgtggaggaatcaaacaagttttataaggacaacatcaaggctggacagcagggcctgagtgtggcattcgatctggccacacatcgcggctatgactcagataatcccagagtcaggggggacgtgggaatggcaggagtcgctatcgacacagtggaagatactaagattctgttcgatggaatccctctggagaaaatgtctgtgagtatgacaatgaacggcgctgtcattcccgtgctggcaaacttcatcgtcactggcgaggaacagggggtgcctaaggaaaaactgaccggcacaattcagaacgacatcctgaaggagttcatggtgcggaatacttacatttttccccctgaaccatccatgaaaatcattgccgatatcttcgagtacaccgctaagcacatgcccaagttcaactcaattagcatctccgggtatcatatgcaggaagcaggagccgacgctattctggagctggcttacaccctggcagatggcctggaatattctcgaaccggactgcaggcaggcctgacaatcgacgagttcgctcctagactgagtttcttttggggaattggcatgaacttttacatggagatcgccaagatgagggctggccggagactgtgggcacacctgatcgagaagatgttccagcctaagaactctaagagtctgctgctgcgggcccattgccagacatccggctggtctctgactgaacaggacccatataacaatattgtcagaaccgcaatcgaggcaatggcagccgtgttcggaggaacccagagcctgcacacaaactcctttgatgaggccctggggctgcctaccgtgaagtctgctaggattgcacgcaatacacagatcattatccaggaggaatccggaatcccaaaggtggccgatccctggggaggctcttacatgatggagtgcctgacaaacgacgtgtatgatgctgcactgaagctgattaatgaaatcgaggaaatggggggaatggcaaaggccgtggctgagggcattccaaaactgaggatcgaggaatgtgcagctaggcgccaggcacgaattgactcaggaagcgaagtgatcgtcggggtgaataagtaccagctggagaaagaagacgcagtcgaagtgctggccatcgataacacaagcgtgcgcaatcgacagattgagaagctgaagaaaatcaaaagctcccgcgatcaggcactggccgaacgatgcctggcagccctgactgagtgtgctgcaagcggggacggaaacattctggctctggcagtcgatgcctcccgggctagatgcactgtgggggaaatcaccgacgccctgaagaaagtcttcggagagcacaaggccaatgatcggatggtgagcggcgcttatagacaggagttcggggaatctaaagagattaccagtgccatcaagagggtgcacaagttcatggagagagaagggcgacggcccaggctgctggtggcaaagatgggacaggacggacatgatcgcggagcaaaagtcattgccaccgggttcgctgacctgggatttgacgtggatatcggccctctgttccagacaccacgagaggtcgcacagcaggcagtcgacgctgatgtgcacgcagtcggagtgtccactctggcagctggccataagaccctggtgcctgaactgatcaaagagctgaactctctgggcagaccagacatcctggtcatgtgcggcggcgtgatcccaccccaggattacgaattcctgtttgaggtcggggtgagcaacgtgttcggaccaggaaccaggatccctaaggccgcagtgcaggtcctggatgatattgaaaagtgtctggaaaagaaacagcagtcagtg

SEQ ID NO: 62 - DNA, 427 bp, Homo sapiens
  source 1..427: mol_type = genomic DNA
gtgatgcttatgaatattaataggaatatttgtaaggcctgaaatattttgatcatgaaatcaaaacattaatttatttaaacatttacttgaaatgtggtggtttgtgatttagttgattttataggctagtgggagaatttacattcaaatgtctaaatcacttaaaattgccctttatggcctgacagtaacttttttttattcatttggggacaactatgtccgtgagcttccgtccagagattatagtagtaaattgtaattaaaggatatgatgcacgtgaaatcactttgcaatcatcaatagcttcataaatgttaattttgtatcctaatagtaatgctaatattttcctaacatctgtcatgtctttgtgttcagggtaaaaaacttgttgctgcaagtcaagctgccttaggctta

SEQ ID NO: 63 - DNA, 612 bp, Homo sapiens
  source 1..612: mol_type = genomic DNA
gcatgtttggttaggctagggcttagggatttatatatcaaaggaggctttgtacatgtgggacagggatcttattttacaaacaattgtcttacaaaatgaataaaacagcactttgtttttatctcctgctctattgtgccatactgttaaatgtttataatgcctgttctgtttccaaatttgtgatgcttatgaatattaataggaatatttgtaaggcctgaaatattttgatcatgaaatcaaaacattaatttatttaaacatttacttgaaatgtggtggtttgtgatttagttgattttataggctagtgggagaatttacattcaaatgtctaaatcacttaaaattgccctttatggcctgacagtaacttttttttattcatttggggacaactatgtccgtgagcttccgtccagagattatagtagtaaattgtaattaaaggatatgatgcacgtgaaatcactttgcaatcatcaatagcttcataaatgttaattttgtatcctaatagtaatgctaatattttcctaacatctgtcatgtctttgtgttcagggtaaaaaacttgttgctgcaagtcaagctgccttaggctta

SEQ ID NO: 64 - DNA, 800 bp, Homo sapiens
  source 1..800: mol_type = genomic DNA
ttcaaactcagtgcacttgttgagcttgtgaaacacaagcccaaggcaacaaaagagcaactgaaagctgttatggatgatttcgcagcttttgtagagaagtgctgcaaggctgacgataaggagacctgctttgccgaggaggtactacagttctcttcattttaatatgtccagtattcatttttgcatgtttggttaggctagggcttagggatttatatatcaaaggaggctttgtacatgtgggacagggatcttattttacaaacaattgtcttacaaaatgaataaaacagcactttgtttttatctcctgctctattgtgccatactgttaaatgtttataatgcctgttctgtttccaaatttgtgatgcttatgaatattaataggaatatttgtaaggcctgaaatattttgatcatgaaatcaaaacattaatttatttaaacatttacttgaaatgtggtggtttgtgatttagttgattttataggctagtgggagaatttacattcaaatgtctaaatcacttaaaattgccctttatggcctgacagtaacttttttttattcatttggggacaactatgtccgtgagcttccgtccagagattatagtagtaaattgtaattaaaggatatgatgcacgtgaaatcactttgcaatcatcaatagcttcataaatgttaattttgtatcctaatagtaatgctaatattttcctaacatctgtcatgtctttgtgttcagggtaaaaaacttgttgctgcaagtcaagctgccttaggctta

SEQ ID NO: 65 - DNA, 424 bp, Homo sapiens
  source 1..424: mol_type = genomic DNA
taacatcacatttaaaagcatctcaggtaactatattttgaattttttaaaaaagtaactataatagttattattaaaatagcaaagattgaccatttccaagagccatatagaccagcaccgaccactattctaaactatttatgtatgtaaatattagcttttaaaattctcaaaatagttgctgagttgggaaccactattatttctattttgtagatgagaaaatgaagataaacatcaaagcatagattaagtaattttccaaagggtcaaaattcaaaattgaaaccaaagtttcagtgttgcccattgtcctgttctgacttatatgatgcggtacacagagccatccaagtaagtgatggctcagcagtggaatactctgggaattaggctgaaccacatgaaagagtgctttata

SEQ ID NO: 66 - DNA, 612 bp, Homo sapiens
  source 1..612: mol_type = genomic DNA
taacatcacatttaaaagcatctcaggtaactatattttgaattttttaaaaaagtaactataatagttattattaaaatagcaaagattgaccatttccaagagccatatagaccagcaccgaccactattctaaactatttatgtatgtaaatattagcttttaaaattctcaaaatagttgctgagttgggaaccactattatttctattttgtagatgagaaaatgaagataaacatcaaagcatagattaagtaattttccaaagggtcaaaattcaaaattgaaaccaaagtttcagtgttgcccattgtcctgttctgacttatatgatgcggtacacagagccatccaagtaagtgatggctcagcagtggaatactctgggaattaggctgaaccacatgaaagagtgctttatagggcaaaaacagttgaatatcagtgatttcacatggttcaacctaatagttcaactcatcctttccattggagaatatgatggatctaccttctgtgaactttatagtgaagaatctgctattacatttccaatttgtcaacatgctgagctttaataggacttatcttcttatgacaacatttattg

SEQ ID NO: 67 - DNA, 807 bp, Homo sapiens
  source 1..807: mol_type = genomic DNA
taacatcacatttaaaagcatctcaggtaactatattttgaattttttaaaaaagtaactataatagttattattaaaatagcaaagattgaccatttccaagagccatatagaccagcaccgaccactattctaaactatttatgtatgtaaatattagcttttaaaattctcaaaatagttgctgagttgggaaccactattatttctattttgtagatgagaaaatgaagataaacatcaaagcatagattaagtaattttccaaagggtcaaaattcaaaattgaaaccaaagtttcagtgttgcccattgtcctgttctgacttatatgatgcggtacacagagccatccaagtaagtgatggctcagcagtggaatactctgggaattaggctgaaccacatgaaagagtgctttatagggcaaaaacagttgaatatcagtgatttcacatggttcaacctaatagttcaactcatcctttccattggagaatatgatggatctaccttctgtgaactttatagtgaagaatctgctattacatttccaatttgtcaacatgctgagctttaataggacttatcttcttatgacaacatttattggtgtgtccccttgcctagcccaacagaagaattcagcagccgtaagtctaggacaggcttaaattgttttcactggtgtaaattgcagaaagatgatctaagtaatttggcatttattttaataggtttgaaaaacacatgccattttacaaataagacttatatttgtccttttgtttttcagcctaccatgag

SEQ ID NO: 68 - DNA, 1645 bp, Homo sapiens
  source 1..1645: mol_type = genomic DNA
tttctagatgtaaataattattttaagtttgccctatggtggccccacacatgagacaaacccccaagatgtgacttttgagaatgagacttggataaaaaacatgtagaaatgcaagccctgaagctcaactccctattgctatcacaggggttataattgcataaaatttagctatagaaagttgctgtcatctcttgtgggctgtaatcatcgtctaggcttaagagtaatattgcaaaacctgtcatgcccacacaaatctctccctggcattgttgtctttgcagatgtcagtgaaagagaaccagcagctcccatgagtttggatagccttattttctatagcctccccactattagctttgaagggagcaaagtttaagaaccaaatataaagtttctcatctttatagatgagaaaaattttaaataaagtccaagataattaaatttttaaggatcatttttagctctttaatagcaataaaactcaatatgacataatatggcacttccaaaatctgaataatatataattgcaatgacatacttcttttcagagatttactgaaaagaaatttgttgacactacataacgtgatgagtggtttatactgattgtttcagttggtcttcccaccaactccatgaaagtggattttattatcctcatcatgcagatgagaatattgagacttatagcggtatgcctgagccccaaagtactcagagttgcctggctccaagatttataatcttaaatgatgggactaccatccttactctctccatttttctatacgtgagtaatgttttttctgttttttttttttctttttccattcaaactcagtgcacttgttgagctcgtgaaacacaagcccaaggcaacaaaagagcaactgaaagctgttatggatgatttcgcagcttttgtagagaagtgctgcaaggctgacgataaggagacctgctttgccgaggaggtactacagttctcttcattttaatatgtccagtattcatttttgcatgtttggttaggctagggcttagggatttatatatcaaaggaggctttgtacatgtgggacagggatcttattttacaaacaattgtcttacaaaatgaataaaacagcactttgtttttatctcctgctctattgtgccatactgttaaatgtttataatgcctgttctgtttccaaatttgtgatgcttatgaatattaataggaatatttgtaaggcctgaaatattttgatcatgaaatcaaaacattaatttatttaaacatttacttgaaatgtggtggtttgtgatttagttgattttataggctagtgggagaatttacattcaaatgtctaaatcacttaaaattgccctttatggcctgacagtaacttttttttattcatttggggacaactatgtccgtgagcttccgtccagagattatagtagtaaattgtaattaaaggatatgatgcacgtgaaatcactttgcaatcatcaatagcttcataaatgttaattttgtatcctaatagtaatgctaatattttcctaacatctgtcatgtctttgtgttcagggtaaaaaacttgttgctgcaagtcaagctgccttaggctta

SEQ ID NO: 69 - DNA, 1000 bp, Homo sapiens
  source 1..1000: mol_type = genomic DNA
taacatcacatttaaaagcatctcaggtaactatattttgaattttttaaaaaagtaactataatagttattattaaaatagcaaagattgaccatttccaagagccatatagaccagcaccgaccactattctaaactatttatgtatgtaaatattagcttttaaaattctcaaaatagttgctgagttgggaaccactattatttctattttgtagatgagaaaatgaagataaacatcaaagcatagattaagtaattttccaaagggtcaaaattcaaaattgaaaccaaagtttcagtgttgcccattgtcctgttctgacttatatgatgcggtacacagagccatccaagtaagtgatggctcagcagtggaatactctgggaattaggctgaaccacatgaaagagtgctttatagggcaaaaacagttgaatatcagtgatttcacatggttcaacctaatagttcaactcatcctttccattggagaatatgatggatctaccttctgtgaactttatagtgaagaatctgctattacatttccaatttgtcaacatgctgagctttaataggacttatcttcttatgacaacatttattggtgtgtccccttgcctagcccaacagaagaattcagcagccgtaagtctaggacaggcttaaattgttttcactggtgtaaattgcagaaagatgatctaagtaatttggcatttattttaataggtttgaaaaacacatgccattttacaaataagacttatatttgtccttttgtttttcagcctaccatgagaataagagaaagaaaatgaagatcaaaagcttattcatctgtttttctttttcgttggtgtaaagccaacaccctgtctaaaaaacataaatttctttaatcattttgcctcttttctctgtgcttcaattaataaaaaatggaaagaatctaatagagtggtacagcactgttatttttcaaagatgtgttg

SEQ ID NO: 70 - DNA, 1000 bp, Homo sapiens
  source 1..1000: mol_type = genomic DNA
actccatgaaagtggattttattatcctcatcatgcagatgagaatattgagacttatagcggtatgcctgagccccaaagtactcagagttgcctggctccaagatttataatcttaaatgatgggactaccatccttactctctccatttttctatacgtgagtaatgttttttctgttttttttttttctttttccattcaaactcagtgcacttgttgagctcgtgaaacacaagcccaaggcaacaaaagagcaactgaaagctgttatggatgatttcgcagcttttgtagagaagtgctgcaaggctgacgataaggagacctgctttgccgaggaggtactacagttctcttcattttaatatgtccagtattcatttttgcatgtttggttaggctagggcttagggatttatatatcaaaggaggctttgtacatgtgggacagggatcttattttacaaacaattgtcttacaaaatgaataaaacagcactttgtttttatctcctgctctattgtgccatactgttaaatgtttataatgcctgttctgtttccaaatttgtgatgcttatgaatattaataggaatatttgtaaggcctgaaatattttgatcatgaaatcaaaacattaatttatttaaacatttacttgaaatgtggtggtttgtgatttagttgattttataggctagtgggagaatttacattcaaatgtctaaatcacttaaaattgccctttatggcctgacagtaacttttttttattcatttggggacaactatgtccgtgagcttccgtccagagattatagtagtaaattgtaattaaaggatatgatgcacgtgaaatcactttgcaatcatcaatagcttcataaatgttaattttgtatcctaatagtaatgctaatattttcctaacatctgtcatgtctttgtgttcagggtaaaaaacttgttgctgcaagtcaagctgccttaggctta

SEQ ID NO: 71 - DNA, 20 bp, synthetic construct
  source 1..20: mol_type = other DNA
acagtatggcacaatagagc

SEQ ID NO: 72 - DNA, 20 bp, synthetic construct
  source 1..20: mol_type = other DNA
agttactgtcaggccataaa

SEQ ID NO: 73 - DNA, 20 bp, synthetic construct
  source 1..20: mol_type = other DNA
aagttactgtcaggccataa

SEQ ID NO: 74 - DNA, 20 bp, synthetic construct
  source 1..20: mol_type = other DNA
agggatttatatatcaaagg

SEQ ID NO: 75 - DNA, 20 bp, synthetic construct
  source 1..20: mol_type = other DNA
aaaggaggctttgtacatgt

SEQ ID NO: 76 - DNA, 20 bp, synthetic construct
  source 1..20: mol_type = other DNA
ttactactataatctctgga

SEQ ID NO: 77 - DNA, 20 bp, synthetic construct
  source 1..20: mol_type = other DNA
ggctttgtacatgtgggaca

SEQ ID NO: 78 - DNA, 20 bp, synthetic construct
  source 1..20: mol_type = other DNA
aaaggatgagttgaactatt

SEQ ID NO: 79 - DNA, 20 bp, synthetic construct
  source 1..20: mol_type = other DNA
ataagtcagaacaggacaat

SEQ ID NO: 80 - DNA, 20 bp, synthetic construct
  source 1..20: mol_type = other DNA
acatgaaagagtgctttata

SEQ ID NO: 81 - DNA, 20 bp, synthetic construct
  source 1..20: mol_type = other DNA
aaatagtttagaatagtggt

SEQ ID NO: 82 - DNA, 20 bp, synthetic construct
  source 1..20: mol_type = other DNA
gctcagcagtggaatactct

SEQ ID NO: 83 - DNA, 20 bp, synthetic construct
  source 1..20: mol_type = other DNA
ctgttctgacttatatgatg

SEQ ID NO: 84 - DNA, 20 bp, synthetic construct
  source 1..20: mol_type = other DNA
agtaagtgatggctcagcag

SEQ ID NO: 85 - DNA, 20 bp, synthetic construct
  source 1..20: mol_type = other DNA
tataagtcagaacaggacaa

SEQ ID NO: 86 - DNA, 20 bp, synthetic construct
  source 1..20: mol_type = other DNA
aatgggcaacactgaaactt

SEQ ID NO: 87 - DNA, 22 bp, synthetic construct
  source 1..22: mol_type = other DNA
tagcaatagggagttgagcttc

SEQ ID NO: 88 - DNA, 22 bp, synthetic construct
  source 1..22: mol_type = other DNA
aaaataaggctatccaaactca

SEQ ID NO: 89 - DNA, 22 bp, synthetic construct
  source 1..22: mol_type = other DNA
tctcatgtgtggggccaccata

SEQ ID NO: 90 - DNA, 22 bp, synthetic construct
  source 1..22: mol_type = other DNA
aaaagttactgtcaggccataa

SEQ ID NO: 91 - DNA, 22 bp, synthetic construct
  source 1..22: mol_type = other DNA
agtttagaatagtggtcggtgc

SEQ ID NO: 92 - DNA, 20 bp, synthetic construct
  source 1..20: mol_type = other DNA
tttagaatagtggtcggtgc

SEQ ID NO: 93 - RNA, 109 nt, synthetic construct
  source 1..109: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 106: mod_base = OTHER; note = Nucleotide with phosphorothioate linkage
  modified_base 107: mod_base = OTHER; note = 2'-O-methyl modified nucleotide with phosphorothioate linkage
  modified_base 108: mod_base = OTHER; note = 2'-O-methyl modified nucleotide with phosphorothioate linkage
  modified_base 109: mod_base = OTHER; note = 2'-O-methyl modified nucleotide
tagcaatagggagttgagcttcgtttcagttgaaagaagtcactcttaaagtgagctgaaatcactaaaaattaagattgaacccggctactgactctgtcatccgggt

SEQ ID NO: 94 - RNA, 109 nt, synthetic construct
  source 1..109: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 106: mod_base = OTHER; note = Nucleotide with phosphorothioate linkage
  modified_base 107: mod_base = OTHER; note = 2'-O-methyl modified nucleotide with phosphorothioate linkage
  modified_base 108: mod_base = OTHER; note = 2'-O-methyl modified nucleotide with phosphorothioate linkage
  modified_base 109: mod_base = OTHER; note = 2'-O-methyl modified nucleotide
aaaataaggctatccaaactcagtttcagttgaaagaagtcactcttaaagtgagctgaaatcactaaaaattaagattgaacccggctactgactctgtcatccgggt

SEQ ID NO: 95 - RNA, 109 nt, synthetic construct
  source 1..109: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 106: mod_base = OTHER; note = Nucleotide with phosphorothioate linkage
  modified_base 107: mod_base = OTHER; note = 2'-O-methyl modified nucleotide with phosphorothioate linkage
  modified_base 108: mod_base = OTHER; note = 2'-O-methyl modified nucleotide with phosphorothioate linkage
  modified_base 109: mod_base = OTHER; note = 2'-O-methyl modified nucleotide
tctcatgtgtggggccaccatagtttcagttgaaagaagtcactcttaaagtgagctgaaatcactaaaaattaagattgaacccggctactgactctgtcatccgggt

SEQ ID NO: 96 - RNA, 109 nt, synthetic construct
  source 1..109: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 106: mod_base = OTHER; note = Nucleotide with phosphorothioate linkage
  modified_base 107: mod_base = OTHER; note = 2'-O-methyl modified nucleotide with phosphorothioate linkage
  modified_base 108: mod_base = OTHER; note = 2'-O-methyl modified nucleotide with phosphorothioate linkage
  modified_base 109: mod_base = OTHER; note = 2'-O-methyl modified nucleotide
aaaagttactgtcaggccataagtttcagttgaaagaagtcactcttaaagtgagctgaaatcactaaaaattaagattgaacccggctactgactctgtcatccgggt

SEQ ID NO: 97 - RNA, 109 nt, synthetic construct
  source 1..109: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 106: mod_base = OTHER; note = Nucleotide with phosphorothioate linkage
  modified_base 107: mod_base = OTHER; note = 2'-O-methyl modified nucleotide with phosphorothioate linkage
  modified_base 108: mod_base = OTHER; note = 2'-O-methyl modified nucleotide with phosphorothioate linkage
  modified_base 109: mod_base = OTHER; note = 2'-O-methyl modified nucleotide
agtttagaatagtggtcggtgcgtttcagttgaaagaagtcactcttaaagtgagctgaaatcactaaaaattaagattgaacccggctactgactctgtcatccgggt

SEQ ID NO: 98 - RNA, 100 nt, synthetic construct
  source 1..100: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 29: mod_base = OTHER; note = 2'-O-methyl modified nucleotide
  modified_base 30: mod_base = OTHER; note = 2'-O-methyl modified nucleotide
  modified_base 31: mod_base = OTHER; note = 2'-O-methyl modified nucleotide
  modified_base 32: mod_base = OTHER; note = 2'-O-methyl modified nucleotide
  modified_base 33: mod_base = OTHER; note = 2'-O-methyl modified nucleotide
  modified_base 34: mod_base = OTHER; note = 2'-O-methyl modified nucleotide
  modified_base 35: mod_base = OTHER; note = 2'-O-methyl modified nucleotide
  modified_base 36: mod_base = OTHER; note = 2'-O-methyl modified nucleotide
  modified_base 37: mod_base = OTHER; note = 2'-O-methyl modified nucleotide
  modified_base 38: mod_base = OTHER; note = 2'-O-methyl modified nucleotide
  modified_base 39: mod_base = OTHER; note = 2'-O-methyl modified nucleotide
  modified_base 40: mod_base = OTHER; note = 2'-O-methyl modified nucleotide
  modified_base 69: mod_base = OTHER; note = 2'-O-methyl modified nucleotide
  modified_base 70: mod_base = OTHER; note = 2'-O-methyl modified nucleotide
  modified_base 71: mod_base = OTHER; note = 2'-O-methyl modified nucleotide
  modified_base 72: mod_base = OTHER; note = 2'-O-methyl modified nucleotide
  modified_base 73: mod_base = OTHER; note = 2'-O-methyl modified nucleotide
  modified_base 74: mod_base = OTHER; note = 2'-O-methyl modified nucleotide
  modified_base 75: mod_base = OTHER; note = 2'-O-methyl modified nucleotide
  modified_base 76: mod_base = OTHER; note = 2'-O-methyl modified nucleotide
  modified_base 77: mod_base = OTHER; note = 2'-O-methyl modified nucleotide
  modified_base 78: mod_base = OTHER; note = 2'-O-methyl modified nucleotide
  modified_base 79: mod_base = OTHER; note = 2'-O-methyl modified nucleotide
  modified_base 80: mod_base = OTHER; note = 2'-O-methyl modified nucleotide
  modified_base 81: mod_base = OTHER; note = 2'-O-methyl modified nucleotide
  modified_base 82: mod_base = OTHER; note = 2'-O-methyl modified nucleotide
  modified_base 83: mod_base = OTHER; note = 2'-O-methyl modified nucleotide
  modified_base 84: mod_base = OTHER; note = 2'-O-methyl modified nucleotide
  modified_base 85: mod_base = OTHER; note = 2'-O-methyl modified nucleotide
  modified_base 86: mod_base = OTHER; note = 2'-O-methyl modified nucleotide
  modified_base 87: mod_base = OTHER; note = 2'-O-methyl modified nucleotide
  modified_base 88: mod_base = OTHER; note = 2'-O-methyl modified nucleotide
  modified_base 89: mod_base = OTHER; note = 2'-O-methyl modified nucleotide
  modified_base 90: mod_base = OTHER; note = 2'-O-methyl modified nucleotide
  modified_base 91: mod_base = OTHER; note = 2'-O-methyl modified nucleotide
  modified_base 92: mod_base = OTHER; note = 2'-O-methyl modified nucleotide
  modified_base 93: mod_base = OTHER; note = 2'-O-methyl modified nucleotide
  modified_base 94: mod_base = OTHER; note = 2'-O-methyl modified nucleotide
  modified_base 95: mod_base = OTHER; note = 2'-O-methyl modified nucleotide
  modified_base 96: mod_base = OTHER; note = 2'-O-methyl modified nucleotide
  modified_base 97: mod_base = OTHER; note = 2'-O-methyl modified nucleotide with phosphorothioate linkage
  modified_base 98: mod_base = OTHER; note = 2'-O-methyl modified nucleotide with phosphorothioate linkage
  modified_base 99: mod_base = OTHER; note = 2'-O-methyl modified nucleotide with phosphorothioate linkage
  modified_base 100: mod_base = OTHER; note = 2'-O-methyl modified nucleotide
tttagaatagtggtcggtgcgttttagagctagaaatagcaagttaaaataaggctagtccgttatcaacttgaaaaagtggcaccgagtcggtgctttt

SEQ ID NO: 99 - DNA, 1976 bp, Homo sapiens
  source 1..1976: mol_type = genomic DNA
tgcacttgttgagctcgtgaaacacaagcccaaggcaacaaaagagcaactgaaagctgttatggatgatttcgcagcttttgtagagaagtgctgcaaggctgacgataaggagacctgctttgccgaggaggtactacagttctcttcattttaatatgtccagtattcatttttgcatgtttggttaggctagggcttagggatttatatatcaaaggaggctttgtacatgtgggacagggatcttattttacaaacaattgtcttacaaaatgaataaaacagcactttgtttttatctcctgctctattgtgccatactgttaaatgtttataatgcctgttctgtttccaaatttgtgatgcttatgaatattaataggaatatttgtaaggcctgaaatattttgatcatgaaatcaaaacattaatttatttaaacatttacttgaaatgtggtggtttgtgatttagttgattttataggctagtgggagaatttacattcaaatgtctaaatcacttaaaattgccctttatggcctgacagtaacttttttttattcatttggggacaactatgtccgtgagcttccgtccagagattatagtagtaaattgtaattaaaggatatgatgcacgtgaaatcactttgcaatcatcaatagcttcataaatgttaattttgtatcctaatagtaatgctaatattttcctaacatctgtcatgtctttgtgttcagggtaaaaaacttgttgctgcaagtcaagctgccttaggcttataacatcacatttaaaagcatctcaggtaactatattttgaattttttaaaaaagtaactataatagttattattaaaatagcaaagattgaccatttccaagagccatatagaccagcaccgaccactattctaaactatttatgtatgtaaatattagcttttaaaattctcaaaatagttgctgagttgggaaccactattatttctattttgtagatgagaaaatgaagataaacatcaaagcatagattaagtaattttccaaagggtcaaaattcaaaattgaaaccaaagtttcagtgttgcccattgtcctgttctgacttatatgatgcggtacacagagccatccaagtaagtgatggctcagcagtggaatactctgggaattaggctgaaccacatgaaagagtgctttatagggcaaaaacagttgaatatcagtgatttcacatggttcaacctaatagttcaactcatcctttccattggagaatatgatggatctaccttctgtgaactttatagtgaagaatctgctattacatttccaatttgtcaacatgctgagctttaataggacttatcttcttatgacaacatttattggtgtgtccccttgcctagcccaacagaagaattcagcagccgtaagtctaggacaggcttaaattgttttcactggtgtaaattgcagaaagatgatctaagtaatttggcatttattttaataggtttgaaaaacacatgccattttacaaataagacttatatttgtccttttgtttttcagcctaccatgagaataagagaaagaaaatgaagatcaaaagcttattcatctgtttttctttttcgttggtgtaaagccaacaccctgtctaaaaaacataaatttctttaatcattttgcctcttttctctgtgcttcaattaataaaaaatggaaagaatctaatagagtggtacagcactgttatttttcaaagatgtgttgctatcctgaaaattctgtaggttctgtggaagttccagtgttctctcttattccacttcggtagaggatttctagtttcttgtgggctaattaaataaatcattaatactcttctaagttatggattataaacattcaaaataatattttgacattatgataattctgaataaaagaacaaaaacca

SEQ ID NO: 100 - protein, 9 aa, synthetic construct
LAGLIDADG

SEQ ID NO: 101 - protein, 8 aa, synthetic construct
  VARIANT 4: note = Any amino acid
DVEXNPGP